(19) United States
(12) Patent Application Publication
(10) Pub. No.: US 2022/0372014 A1
(43) Pub. Date: Nov. 24, 2022

(54) NEUROPROTECTIVE COMPOUNDS AND METHODS OF USE THEREOF

(71) Applicants: The Johns Hopkins University, Baltimore, MD (US); Rapafusyn Research and Development, Inc., Baltimore, MD (US)

(72) Inventors: Ted M. Dawson, Baltimore, MD (US); Valina L. Dawson, Baltimore, MD (US); Jun Liu, Baltimore, MD (US); Hyejin Park, Baltimore, MD (US); Tae-In Kam, Baltimore, MD (US); Hanjing Peng, Baltimore, MD (US); Venkata Subbarao Ayinampudi, Baltimore, MD (US); Sam Hong, Baltimore, MD (US); Brett Ullman, Baltimore, MD (US); Puneet Kumar, Baltimore, MD (US); Magesh Sadagopan, Baltimore, MD (US)

(21) Appl. No.: 17/765,418

(22) PCT Filed: Sep. 30, 2020
(86) PCT No.: PCT/US2020/053486

§ 371 Date: Mar. 30, 2022

Related U.S. Application Data
(60) Provisional application No. 62/908,954, filed on Oct. 1, 2019.

(51) Int. Cl. C07D 401/06; C07D 401/14; C07D 405/14; C07D 413/14; C07D 409/14; A61P 25/28

(52) U.S. Cl. CPC C07D 401/06; C07D 401/14; C07D 405/14; C07D 413/14; C07D 409/14; A61P 25/28

(57) ABSTRACT

The present disclosure provides neuroprotective compounds along with their use in treating disease such as Parkinson's disease (PD). The compounds can be used as inhibitors for Parthanatos Associated AIF (apoptosis-inducing factor) Nuclease (PAAN), also known as macrophage migration inhibitor factor (MIF).

### CROSS-REFERENCE TO RELATED APPLICATIONS

The application claims benefit of priority under 35 U.S.C. § 119(e) of U.S. Ser. No. 62/908,954, filed Oct. 1, 2019, the entire content of which is incorporated by reference in its entirety.

### GOVERNMENT SUPPORT

The invention was made with government support under NS067525 awarded by the National Institutes of Health. The Government has certain rights in this invention.

### INCORPORATION OF SEQUENCE LISTING

The material in the accompanying sequence listing is hereby incorporated by reference into this application. The accompanying sequence listing text file, name JHU4120_1WO_Sequence_Listing.txt, was created on Sep. 21, 2020, and is 2 kb. The file can be accessed using Microsoft Word on a computer that uses Windows OS.

### BACKGROUND OF THE INVENTION

### Field of the Disclosure

The disclosure relates generally to chemical compounds and more specifically to the use of such compounds in the treatment of neurodegenerative diseases.

### Background Information

Poly(ADP-ribose) (PAR) polymerase-1 (PARP-1) is an important nuclear enzyme that is activated by DNA damage where it facilitates DNA repair. Excessive activation of PARP-1 causes an intrinsic caspase-independent cell death program designated parthanatos, which plays a prominent role following a number of toxic insults in many organ systems, including ischemia-reperfusion injury after stroke and myocardial infarction, inflammatory injury, reactive oxygen species-induced injury, glutamate excitotoxicity and neurodegenerative diseases such as Parkinson disease and Alzheimer disease. Consistent with the idea that PARP-1 is a key cell death mediator, PARP inhibitors or genetic deletion of PARP-1 are profoundly protective against these and other cellular injury paradigms and models of human disease.

Molecular mechanisms underlying parthanatos involve PAR-dependent apoptosis-inducing factor (AIF) release from the mitochondria and translocation to the nucleus resulting in fragmentation of DNA into 20-50 kb fragments. AIF itself has no obvious nuclease activity. Although it has been suggested that CED-3 Protease Suppressor (CPS)-6, an endonuclease G (EndoG) homolog in Caenorhabditis elegans (C. elegans), cooperates with the worm AIF Homolog (WAH-1) to promote DNA degradation (Wang (2002) Science 298:1587-92). EndoG does not seem to play an essential role in PARP-dependent chromatinolysis and cell death after transient focal cerebral ischemia in mammals. The nuclease responsible for the chromatinolysis during parthanatos is not known.

MIF was previously determined to be an AIF interacting protein. Molecular 3-D modeling of prior crystallization studies of MIF demonstrated MIF is structurally similar to PD-D/E(X)K nucleases protein. Proteins containing PD-D/E(X)K domains belong to the nuclease-like superfamily, providing further evidence that MIF is a nuclease. This nuclease superfamily contains nucleases from all kingdoms of life. The majority of these proteins belong to prokaryotic organisms, but this domain is contained with a variety of vertebrate nucleases. The PD-D/E(X)K domains in MIF are highly conserved in vertebrates. The glutamic acid residue (E22) in the first α-helix of MIF is critical for its nuclease activity, which is consistent with prior reports that this glutamic acid in the first α-helix of many Exonuclease-Endonuclease-Phosphatase (EEP) domain superfamily nucleases is highly conserved and it is the active site for nuclease activity (Wang (2016) Science 354:82).

The core PD-D/E(x)K structure consists of 4 β-strands next to two α-helices. Two of the β-strands are parallel to each other whereas the other two are antiparallel. Interestingly, the MIF monomer, which has pseudo 2-fold symmetry does not contain the core PD-D/E(x)K structure since the MIF monomer has 4 β-strands next to the 2 α-helices, and the orientations of the β-strands within an isolated monomer do not fit the requirement of the PD-D/E(x)K topology. However, the structure-activity analyses based on the MIF trimer, which has 3-fold symmetry indicate that the interactions of the β-strands of each monomer with the other monomers results in a MIF PD-D/E(x)K structure that consists of 4 β-strands next to 2 α-strands. Two of the β-strands are parallel (β-4 and 0-5) whereas the other two strands (β-6 and β-7) (from the adjacent monomer) are anti-parallel. This topology exquisitely supports the idea that MIF's nuclease activity requires the trimer as the monomers do not support the required topology and is consistent with MIF existing as a trimer. This topology of the MIF trimer places the α-1 helix, which contains the active residue, glutamate 22, next to the β-strands, but this is not unprecedented.

For example, EcoRV, a well characterized endonuclease has PD-D/E(x)K motifs with orientations of the beta-strands relative to the alpha helices different from the classical PD-D/E(x)K motif and similar to that of MIF. The similarity in the topology of MIF versus EcoRV suggests that MIF is highly similar to the well characterized restriction endonucleases. Indeed conserved acidic residues from the core α-helices (usually) glutamic acid from the first α-helix often contributes to active site formation at least in a subset of PD-D/E(x)K families similar to what was reported for MIF. The PD-D/E(x)K motif based on MIF's trimer structure also has a very similar structure to the nucleases ExoIII, EcoRI and EcoRV. Moreover, MIF has a similar topology to the PvuII endonuclease and MIF's β-7 strand is of similar size to PvuII endonuclease β-strand at the same position in its PD-D/E(x)K motif. Based on the structural analysis, MIF was classified as nuclease (Wang (2016) Science 354:82).

MIF has a variety of pleiotropic actions. It functions as a non-classically secreted cytokine where it may play important roles in cancer biology, immune responses and inflammation. MIF also has important roles in cellular stress and apoptosis. Knockout of MIF has also been shown to be neuroprotective in focal ischemia. Our prior results confirmed that knockout of MIF protects against focal ischemia and showed that MIF contributes to the neuronal damage in focal ischemia via its binding to AIF and its nuclease activity consistent with its function as a PARP-1 dependent AIF-associated nuclease (PAAN) (Wang (2016) Science 354:82). MIF also has thiol-protein oxidoreductase activity and tautomerase activity. Both EMSA and ChTP indicate that MIF binds DNA. Although MIF binds a highly related family of overlapping sequences, the structure-activity experiment indicates that MIF preferentially binds to ssDNA based on its structure and that it relies less on sequence specificity. MIF binds at 5′ unpaired bases of ssDNA with stem loop structure and it has both 3′ exonuclease and endonuclease activities and cleaves unpaired bases at the 3′ end of stem loop ssDNA. The 3-dimensional computational modeling shows that the catalytic E22 is close to the modeled binding domain of ssDNA. As shown here, MIF's nuclease activity is clearly separable from it oxidoreductase and tautomerase activities.

Previous attempts to identify the AIF associated nuclease initially focused on EndoG, a mitochondrial matrix protein. In C. elegans, CPS-6 the homolog of mammalian EndoG is required for WAH-1's (AIF homolog) cell death inducing properties. However, in mammals, EndoG is dispensable in many models of cell death including PARP-1 dependent ischemic cell death. Importantly there was an equivalent amount of DNA fragmentation in EndoG knockout mice compared to wild type controls following middle cerebral artery occlusion. Consistent with these observations, it was confirmed that knockout of endoG failed to block MNNG induced parthanatos and large DNA fragmentation confirming that EndoG is not required for parthanatos. In contrast, knockout of MIF, a MIF nuclease-deficient mutant and a MIF AIF binding deficient mutant prevent cell death and large DNA fragmentation both in vitro and in vivo following activation of PARP-1 (Wang (2016) Science 354:82). Thus, EndoG is not the PAAN in mammals, whereas MIF fits all the criteria for this role.

### SUMMARY

The present disclosure is based on the identification of neuroprotective compounds that prevent or inhibit neurodegeneration. The compounds inhibit parthanatos mediated cell death. Additionally, some compounds also inhibit MIF nuclease activity

Disclosed herein is a compound according to Formula (I) or an optically pure stereoisomer or pharmaceutically acceptable salt thereof.

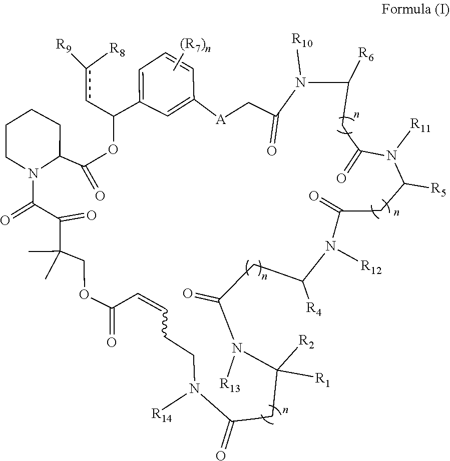

In some embodiments, each R₁ and R₂ is independently selected from the group consisting of H, halogen, hydroxyl, C₁₋₂₀ alkyl, substituted C₁₋₂₀ alkyl, OC₁₋₂₀ alkyl, substituted OC₁₋₂₀ alkyl, N₃, NH₂, NO₂, CF₃, OCF₃, OCHF₂, COC₁₋₂₀alkyl, CO₂C₁₋₂₀alkyl, C₃₋₈cycloalkyl, substituted C₃₋₈cycloalkyl, C₆₋₁₅aryl, substituted C₆₋₁₅aryl, and a cyclic substituent formed with the adjacent nitrogen, the cyclic substituent is selected from the group consisting of

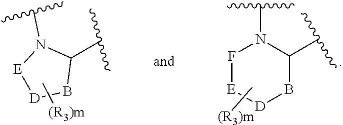

In some embodiments, R₃ is selected from H, halogen, hydroxyl, N₃, NH₂, NO₂, CF₃, C₁₋₁₀alkyl, substituted C₁₋₁₀alkyl, C₁₋₁₀alkoxy, substituted C₁₋₁₀alkoxy, acyl, acylamino, acyloxy, acyl C₁₋₁₀alkyloxy, amino, substituted amino, aminoacyl, aminocarbonyl C₁₋₁₀alkyl, aminocarbonylamino, aminodicarbonylamino, aminocarbonyloxy, aminosulfonyl, C₆₋₁₅aryl, substituted C₆₋₁₅aryl, C₆₋₁₅aryloxy, substituted C₆₋₁₅aryloxy, C₆₋₁₅arylthio, substituted C₆₋₁₅arylthio, carboxyl, carboxyester, (carboxyester)amino, (carboxyester)oxy, cyano, C₃₋₈cycloalkyl, substituted C₃₋₈cycloalkyl, (C₃₋₈cycloalkyl)oxy, substituted (C₃₋₈cycloalkyl)oxy, (C₃₋₈cycloalkyl)thio, substituted (C₃₋₈cycloalkyl)thio, C₁₋₁₀heteroaryl, substituted C₁₋₁₀heteroaryl, C₁₋₁₀heteroaryloxy, substituted C₁₋₁₀heteroaryloxy, C₁₋₁₀heteroarylthio, substituted C₁₋₁₀heteroarylthio, C₂₋₁₀heterocyclyl, C₂₋₁₀substituted heterocyclyl, C₂₋₁₀heterocyclyloxy, substituted C₂₋₁₀heterocyclyloxy, C₂₋₁₀heterocyclylthio, substituted C₂₋₁₀heterocyclylthio, imino, oxo, sulfonyl, sulfonylamino, thiol, C₁₋₁₀alkylthio, substituted C₁₋₁₀alkylthio, and thiocarbonyl.

Or two R3 substituents, together with the atom to which each is bound, may form ring selected from a C₆₋₁₅aryl, substituted C₆₋₁₅aryl, C₃₋₈cycloalkyl, substituted C₃₋₈cycloalkyl, C₁₋₁₀heteroaryl, substituted C₁₋₁₀heteroaryl, C₂₋₁₀substituted heterocyclyl, C₂₋₁₀heterocyclyloxy, and substituted C₂₋₁₀heterocyclyloxy.

In some embodiments, each n and m can be independently selected from 0-5. A can be oxygen or —NH—CO—CH₂—. Each of B, D, E, and F independently can be selected from the group consisting of carbon, nitrogen, oxygen, and sulfur.

In some embodiments, R₄ is selected from the group consisting of H, halogen, hydroxyl, C₁₋₂₀ alkyl, N₃, NH₂, NO₂, CF₃, OCF₃, OCHF₂, COC₁₋₂₀alkyl, CO₂C₁₋₂₀alkyl, and a cyclic substituent formed with the adjacent nitrogen, the cyclic substituent is selected from the group consisting of

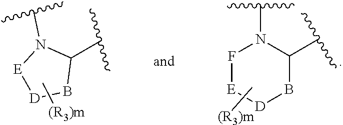

In some embodiments, R₅ is selected from the group consisting of C₆₋₁₅aryl and C₁₋₁₀heteroaryl optionally substituted with H, halogen, hydroxyl, N₃, NH₂, NO₂, CF₃, C₁₋₁₀alkyl, substituted C₁₋₁₀alkyl, C₁₋₁₀alkoxy, substituted C₁₋₁₀alkoxy, acyl, acylamino, acyloxy, acyl C₁₋₁₀alkyloxy, amino, substituted amino, aminoacyl, aminocarbonyl C₁₋₁₀alkyl, aminocarbonylamino, aminodicarbonylamino, aminocarbonyloxy, aminosulfonyl, C₆₋₁₅aryl, substituted C₆₋₁₅aryl, C₆₋₁₅aryloxy, substituted C₆₋₁₅aryloxy, C₆₋₁₅arylthio, substituted C₆₋₁₅arylthio, carboxyl, carboxyester, (carboxyester)amino, (carboxyester)oxy, cyano, C₃₋₈cycloalkyl, substituted C₃₋₈cycloalkyl, (C₃₋₈cycloalkyl)oxy, substituted (C₃₋₈cycloalkyl)oxy, (C₃₋₈cycloalkyl)thio, substituted (C₃₋₈cycloalkyl)thio, halo, hydroxyl, C₁₋₁₀heteroaryl, substituted C₁₋₁₀heteroaryl, C₁₋₁₀heteroaryloxy, substituted C₁₋₁₀heteroaryloxy, C₁₋₁₀heteroarylthio, substituted C₁₋₁₀heteroarylthio, C₂₋₁₀heterocyclyl, C₂₋₁₀substituted heterocyclyl, C₂₋₁₀heterocyclyloxy, substituted C₂₋₁₀heterocyclyloxy, C₂₋₁₀heterocyclylthio, substituted C₂₋₁₀heterocyclylthio, imino, oxo, sulfonyl, sulfonylamino, thiol, C₁₋₁₀alkylthio, substituted C₁₋₁₀alkylthio, and thiocarbonyl.

In some embodiments, R₆ is selected from the group consisting of H, halogen, hydroxyl, C₁₋₂₀ alkyl, C₁₋₁₀alkyloxy, substituted C₁₋₁₀alkyloxy, N₃, NH₂, NO₂, CF₃, OCF₃, OCHF₂, COC₁₋₂₀alkyl, CO₂C₁₋₂₀alkyl, C₃₋₈cycloalkyl, substituted C₃₋₈cycloalkyl, amino, substituted amino, aminoacyl, aminocarbonyl C₁₋₁₀alkyl, aminocarbonylamino, aminodicarbonylamino, aminocarbonyloxy, aminosulfonyl, thiol, C₁₋₁₀alkylthio, substituted C₁₋₁₀alkylthio, and a cyclic substituent formed with the adjacent nitrogen, the cyclic substituent is selected from the group consisting of

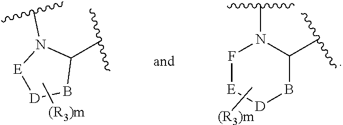

In some embodiments, each R₇ and R₈ is independently selected from the group consisting of H, halogen, hydroxyl, C₁₋₂₀ alkyl, N₃, NH₂, NO₂, CF₃, OCF₃, OCHF₂, COC₁₋₂₀alkyl, and CO₂C₁₋₂₀alkyl. [symbol] can represent a single or a double bond.

In some embodiments, R₉ is selected from the group consisting of C₆₋₁₅aryl and C₁₋₁₀heteroaryl optionally substituted with H, halogen, hydroxyl, N₃, NH₂, NO₂, CF₃, C₁₋₁₀alkyl, substituted C₁₋₁₀alkyl, C₁₋₁₀alkoxy, substituted C₁₋₁₀alkoxy, acyl, acylamino, acyloxy, acyl C₁₋₁₀alkyloxy, amino, substituted amino, aminoacyl, aminocarbonyl C₁₋₁₀alkyl, aminocarbonylamino, aminodicarbonylamino, aminocarbonyloxy, aminosulfonyl, C₆₋₁₅aryl, substituted C₆₋₁₅aryl, C₆₋₁₅aryloxy, substituted C₆₋₁₅aryloxy, C₆₋₁₅arylthio, substituted C₆₋₁₅arylthio, carboxyl, carboxyester, (carboxyester)amino, (carboxyester)oxy, cyano, C₃₋₈cycloalkyl, substituted C₃₋₈cycloalkyl, (C₃₋₈cycloalkyl)oxy, substituted (C₃₋₈cycloalkyl)oxy, (C₃₋₈cycloalkyl)thio, substituted (C₃₋₈cycloalkyl)thio, C₁₋₁₀heteroaryl, substituted C₁₋₁₀heteroaryl, C₁₋₁₀heteroaryloxy, substituted C₁₋₁₀heteroaryloxy, C₁₋₁₀heteroarylthio, substituted C₁₋₁₀heteroarylthio, C₂₋₁₀heterocyclyl, C₂₋₁₀substituted heterocyclyl, C₂₋₁₀heterocyclyloxy, substituted C₂₋₁₀heterocyclyloxy, C₂₋₁₀heterocyclylthio, substituted C₂₋₁₀heterocyclylthio, imino, oxo, sulfonyl, sulfonylamino, thiol, C₁₋₁₀alkylthio, substituted C₁₋₁₀alkylthio, and thiocarbonyl.

In some embodiments, each R₁₀, R₁₁, R₁₂, R₁₃, and R₁₄ is independently selected from the group consisting of H, methyl, ethyl, propyl, and isopropyl.

In some embodiments, each R₁ and R₂ can be independently selected from the group consisting of

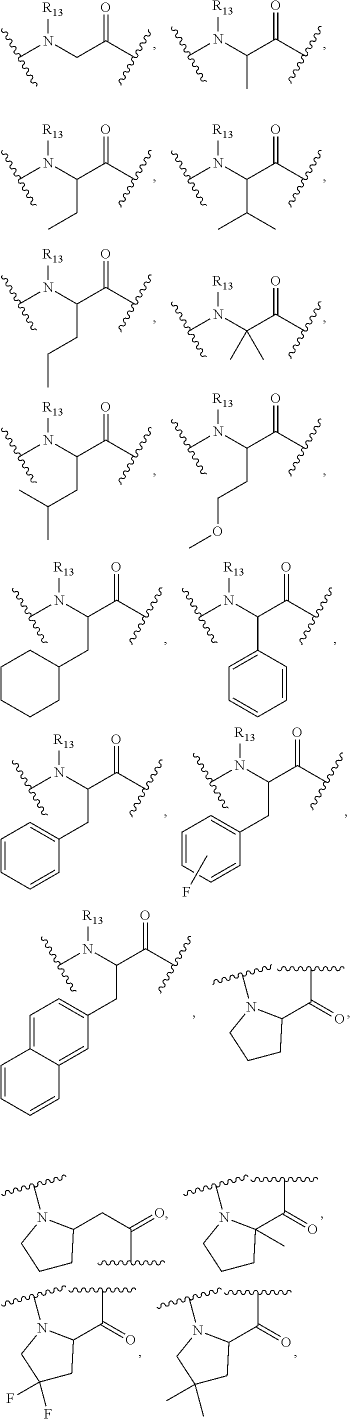

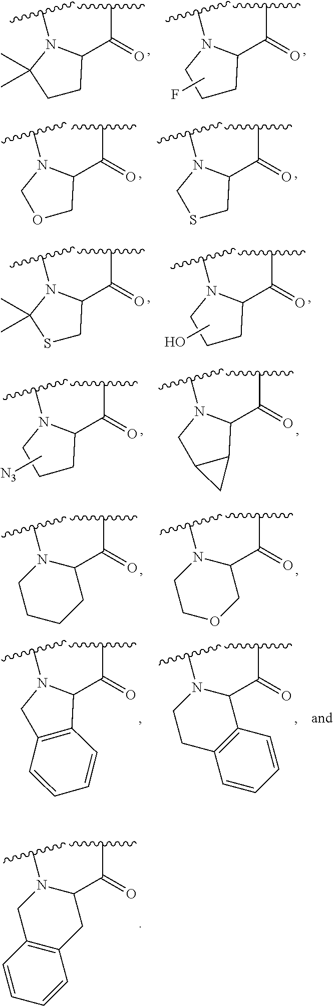

In some embodiments, R₄ can be selected from the group consisting of

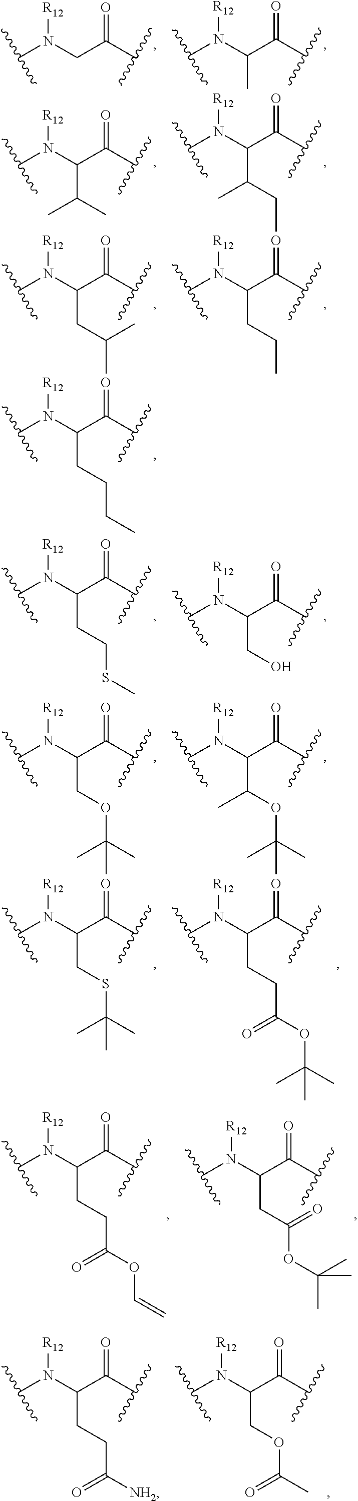

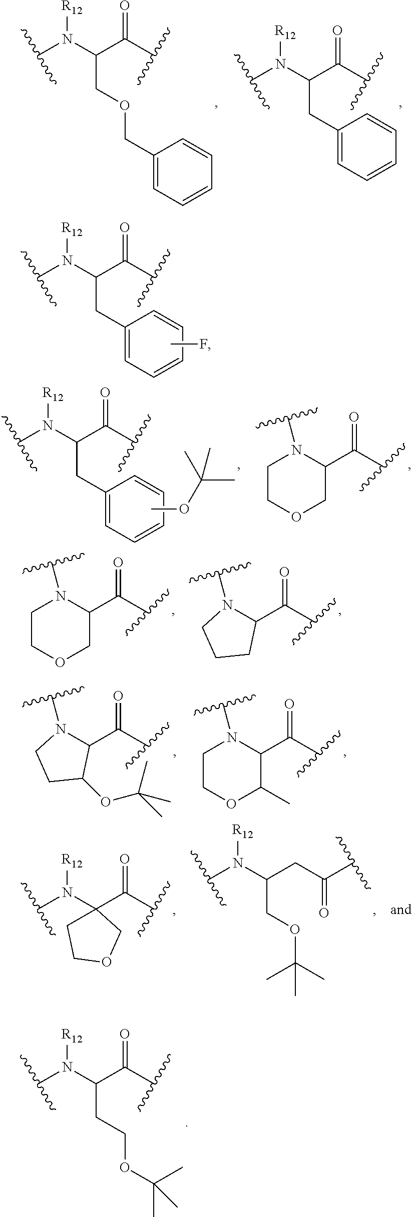

In some embodiments, R₅ can be selected from the group consisting of

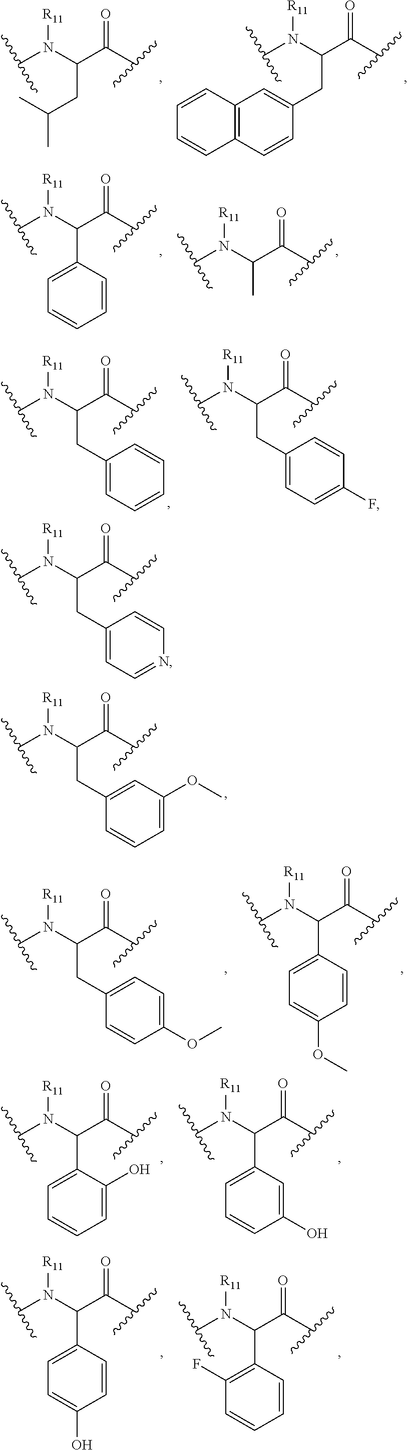

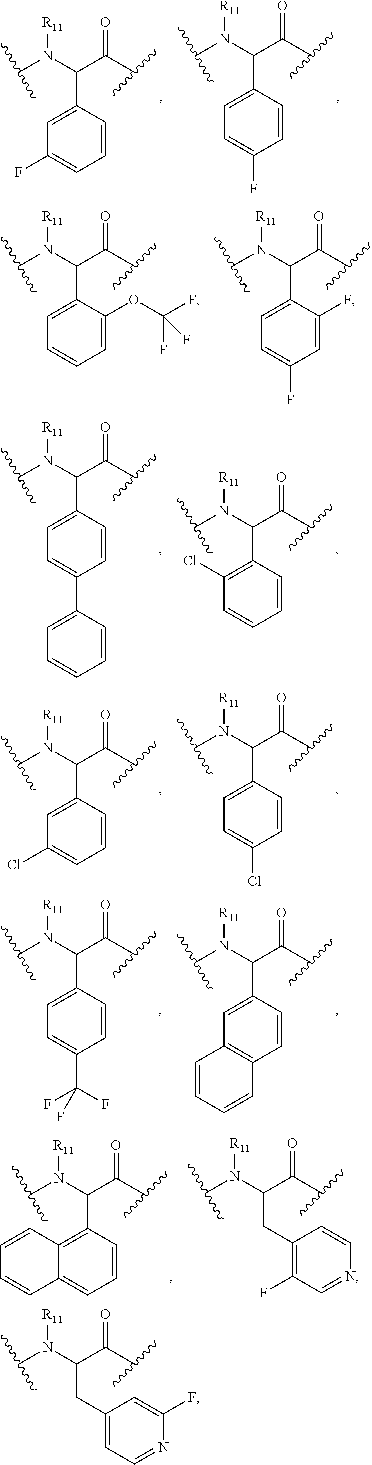

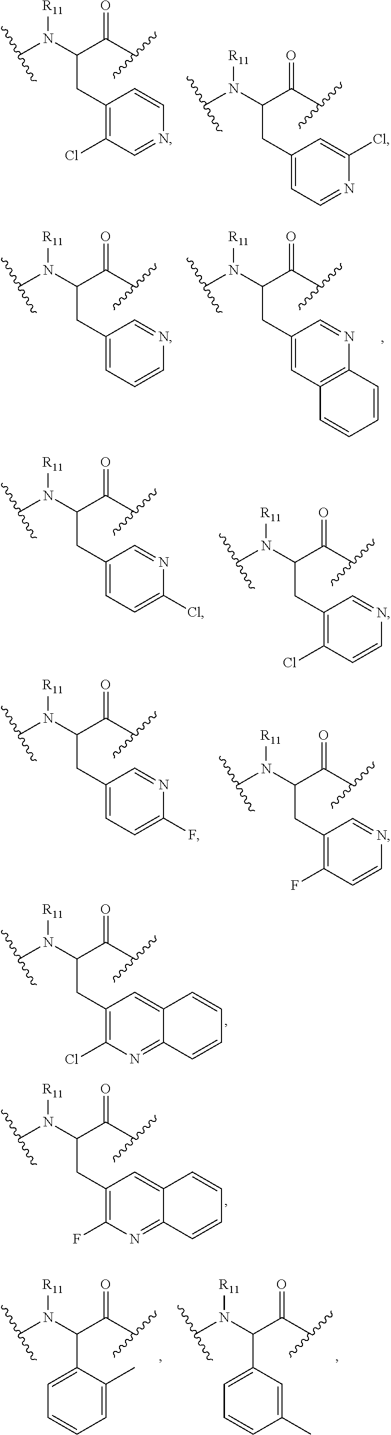

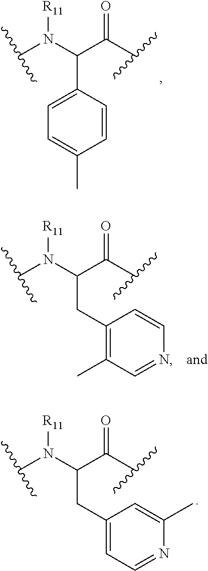

In some embodiments, R₆ can be selected from the group consisting of

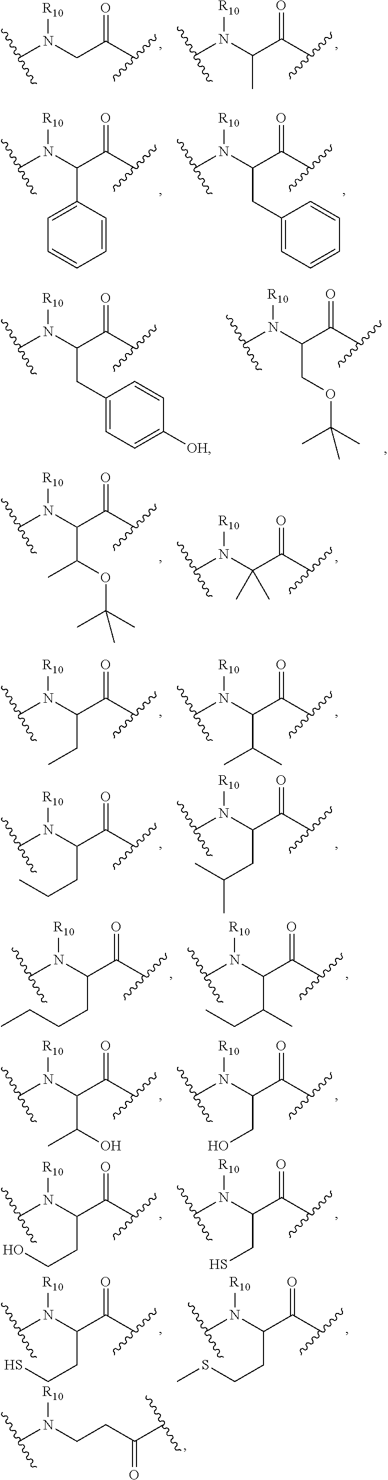

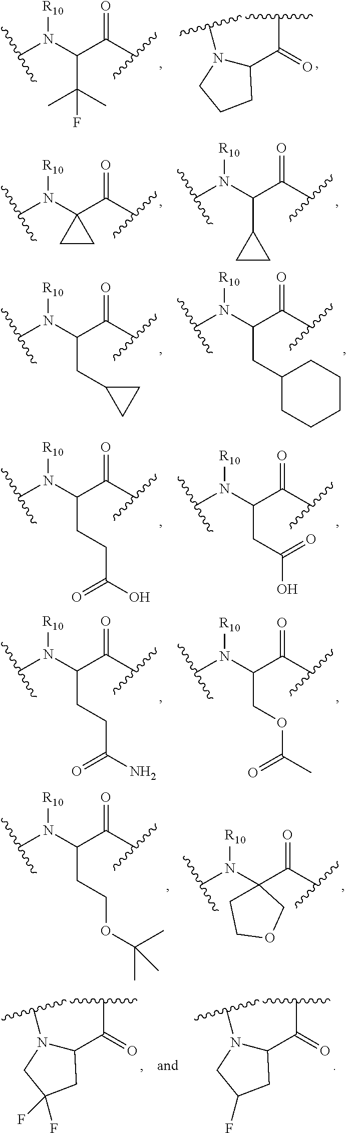

In some embodiments, R₉ can be selected from the group consisting of

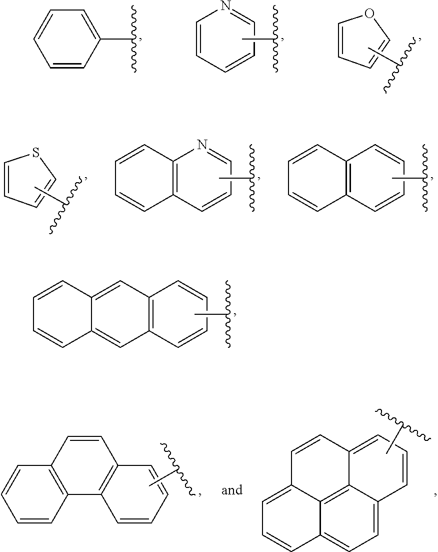

optionally substituted with halogen, hydroxyl, or alkoxyl.

In some embodiments,

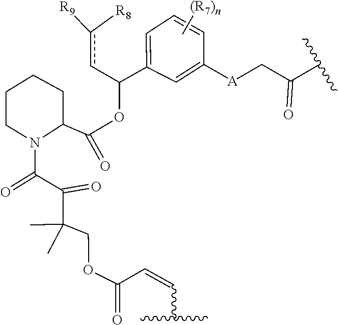

in Formula (I) can be selected from the group consisting of

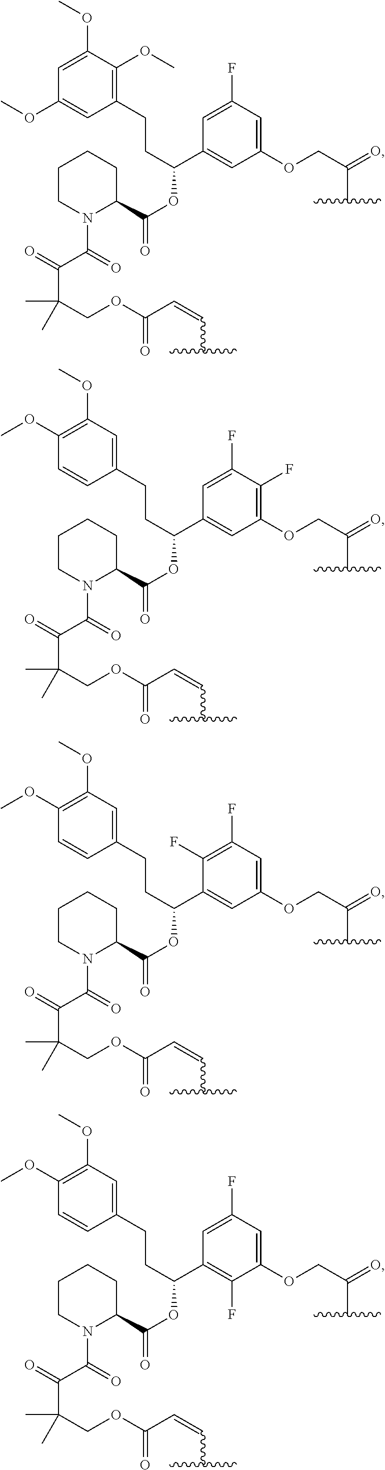

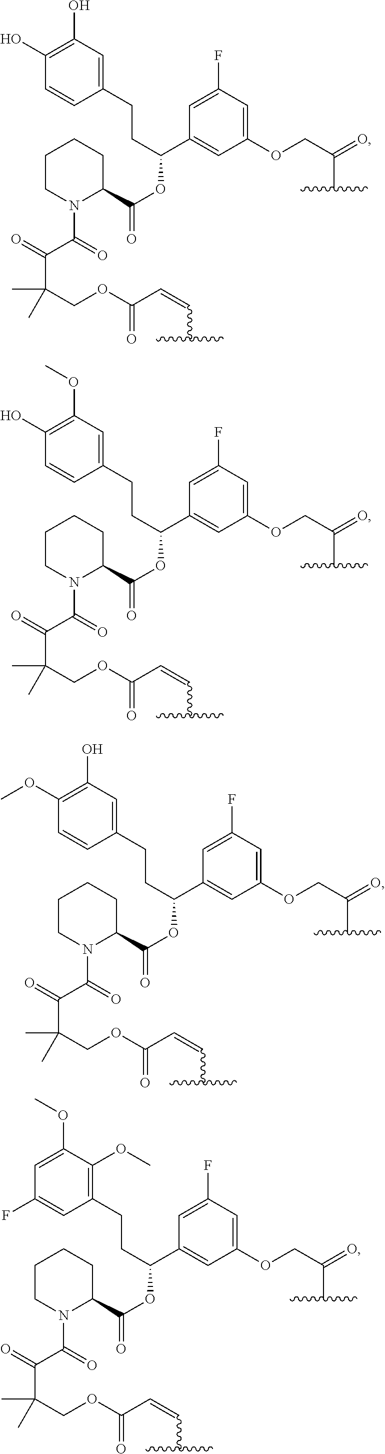

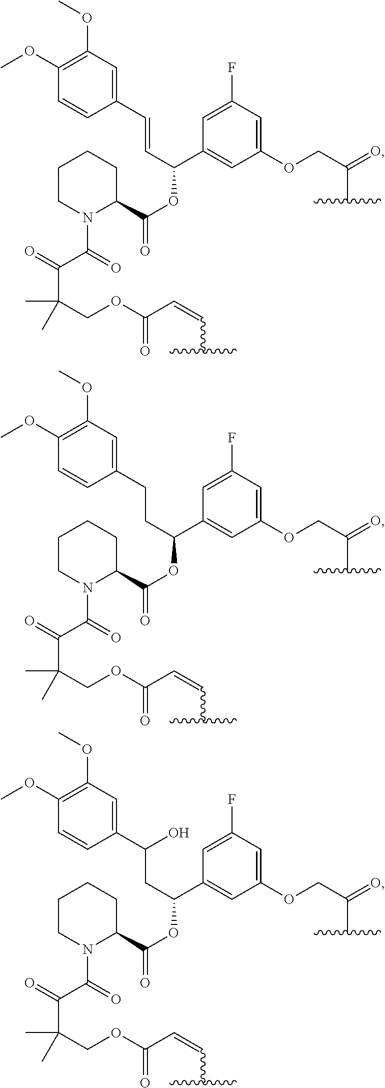

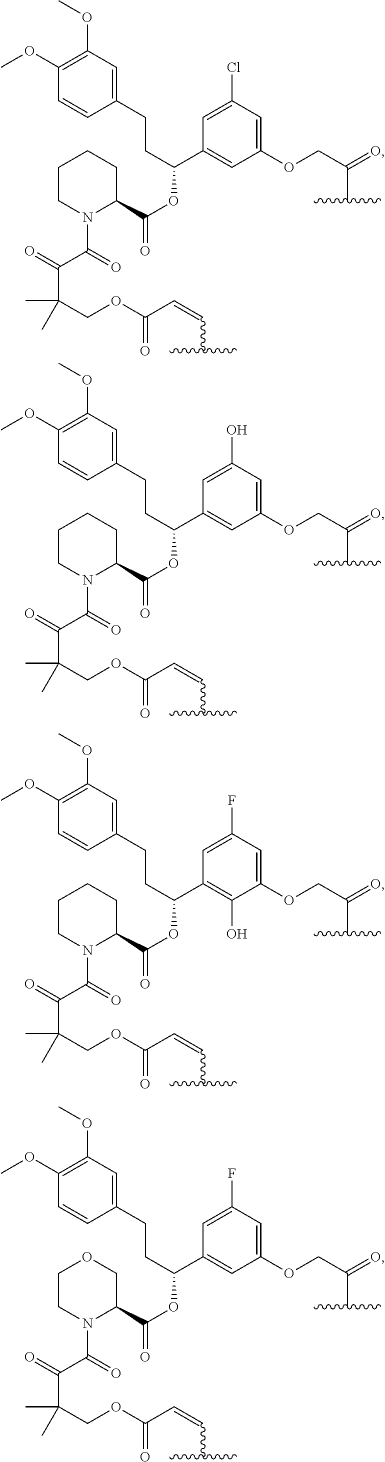

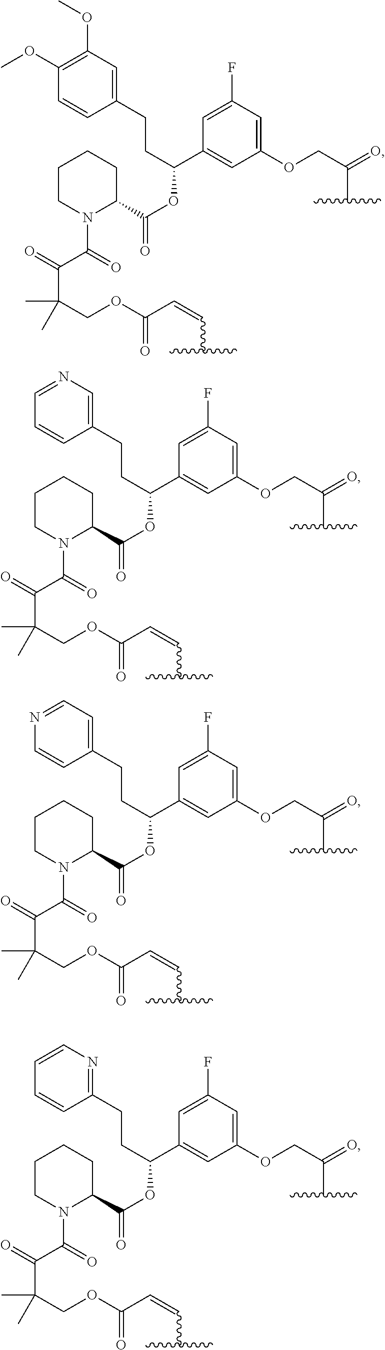

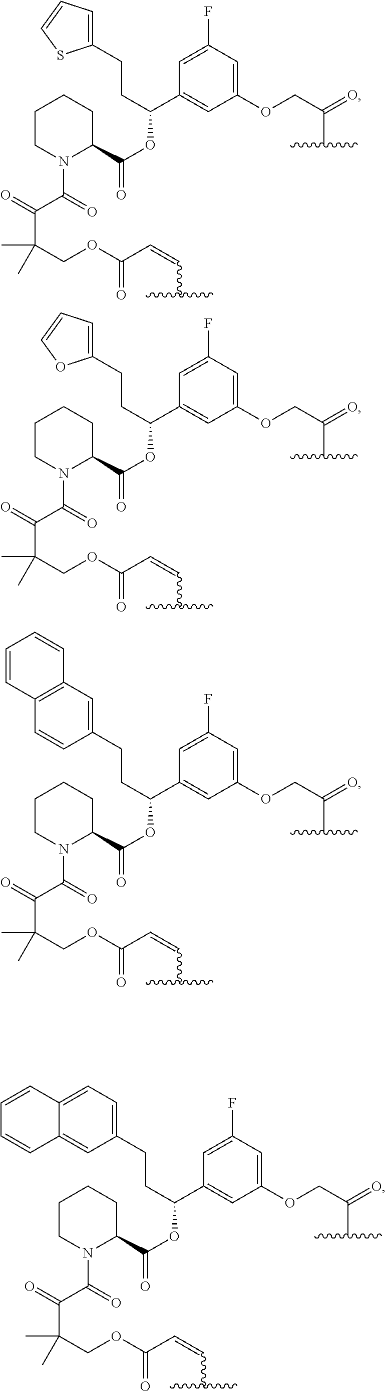

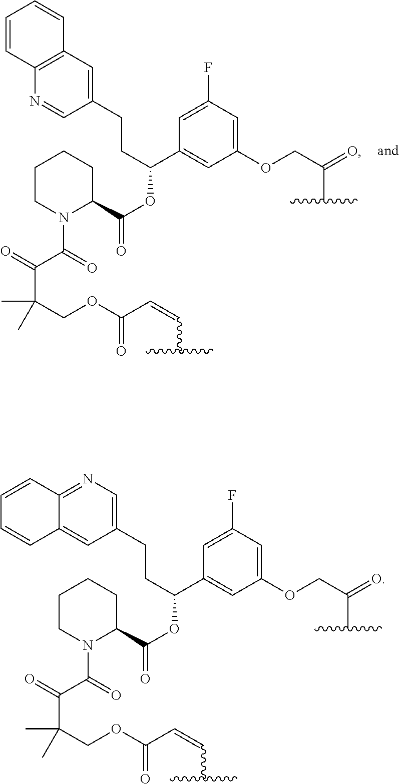

In some embodiments, the compounds disclosed herein can be selected from compounds that inhibit MIF nuclease activity and parthanatos mediated cell death consisting of compounds 121, 153, and 154 with the following structures:

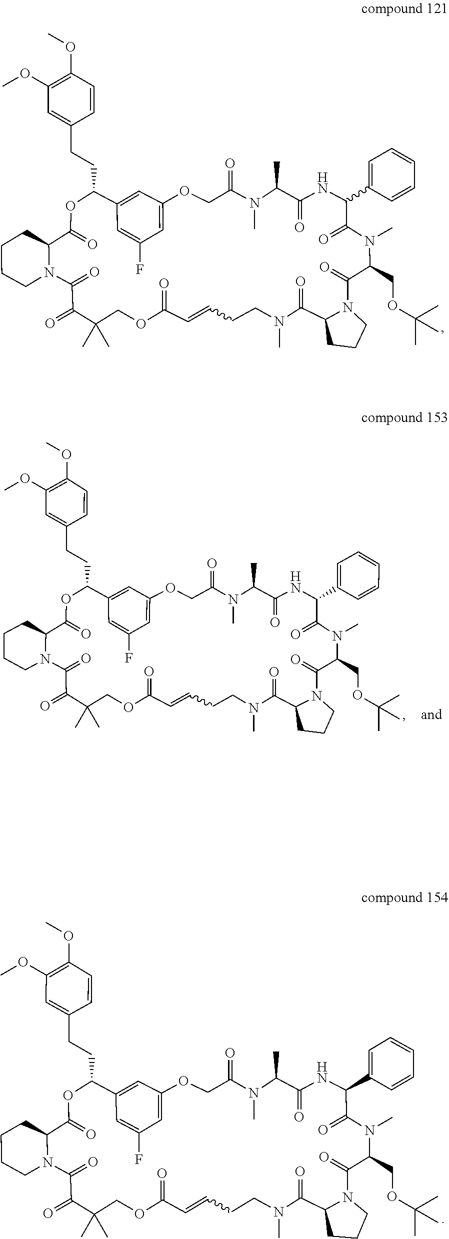

In some embodiments, also disclosed herein is a pharmaceutical composition including the chemical compound with Formula (I). In one embodiment the disclosure provides a compound with the structure of Formula (I) which selectively inhibits parthanatos mediated cell death. In one embodiment the disclosure provides a compound with the structure of Formula (I) which selectively binds MIF to inhibit MIF nuclease activity as well as parthanatos mediated cell death. In one embodiment the disclosure provides a neuroprotective compound with the structure of Formula (I) which inhibits neurodegeneration.

In one embodiment, the disclosure provides a method of inducing a neuroprotective response in a cell. The method includes administering to the subject a therapeutically effective amount of a compound of the disclosure, thereby inducing a neuroprotective response in the cell. In some embodiments, the compound has the structure of Formula (I). In one embodiment, the compound is selected from those compounds set forth in Table 2.

In some embodiment, the disclosure provides a method of treating a neurodegenerative disease in a subject. The method includes administering to the subject a therapeutically effective amount of a compound of the disclosure, thereby treating the disease. In some embodiments, the compound has the structure of Formula (I). In one embodiment, the compound is selected from those compounds set forth in Table 2. In some embodiments, the disease is Alzheimer's disease, Parkinson's disease, Lewy Body Dementia, Multi-Systems Atrophy, Gehrig's disease (Amyotrophic Lateral Sclerosis), Huntington's disease, Multiple Sclerosis, senile dementia, subcortical dementia, arteriosclerotic dementia, AIDS-associated dementia, other dementias, cerebral vasculitis, epilepsy, Tourette's syndrome, Guillain Bane Syndrome, Wilson's disease, Pick's disease, encephalitis, encephalomyelitis, meningitis, prion diseases, cerebellar ataxias, cerebellar degeneration, spinocerebellar degeneration syndromes, Friedrich's ataxia, ataxia telangiectasia, spinal dysmyotrophy, progressive supranuclear palsy, dystonia, muscle spasticity, tremor, retinitis pigmentosa, striatonigral degeneration, mitochondrial encephalomyopathies or neuronal ceroid lipofuscinosis.

Further disclosed herein is a compound according to Formula (VII) or an optically pure stereoisomer, pharmaceutically acceptable salt, or solvate thereof:

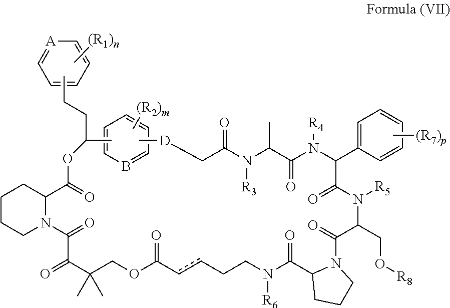

Each A and B can be independently CH or N. Each n, m, and p can be independently an integer selected from 0 to 4. [symbol] can be a single bond or double bond.

D can be selected from the group consisting of —O(CH₂)_{q}—, —S(CH₂)_{q}—, —COO(CH₂)_{q}—, —CONR₃(CH₂)_{q}—, and —NR₃(CH₂)_{q}—. q can be an integer selected from 0 to 4.

Each R₁, R₂, and R₇ can be independently selected from the group consisting of H, F, Br, Cl, CF₃, CN, N₃, NH₂, NO₂, OH, OCH₃, methyl, ethyl, and propyl.

Each R₃, R₄, R₅, and R₆ can be independently selected from the group consisting of H, methyl, ethyl, propyl, and isopropyl.

R₈ can be selected from the group consisting of H, methyl, ethyl, n-propyl, isopropyl, n-butyl, isobutyl, sec-butyl, and tert-butyl.

In some aspects, the carbon connected to

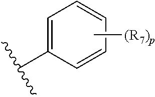

can adopt R or S stereochemistry.

### BRIEF DESCRIPTION OF FIGURES

FIG. 1 is a scheme illustrating the establishment of MIF inhibitor screening using macrocyclic compound library. The schematic representation of macrocyclic screening for MIF inhibitors based on cleavage assay. Single-strand amine-modified oligonucleotides (MIF target DNA) were immobilized on DNA-BIND plates and incubated in MIF protein with or without inhibitors. After MIF cleavage, the fragments were hybridized with biotin-labeled complementary oligonucleotides and detected by monitoring absorbance at 450 nm.

FIG. 2A is an imaging showing representative TH and Nissl staining of SNpc DA neurons of α-syn PFF injected WT and PAAN/MIF KO at 6 months after α-syn PFF or PBS injection.

FIG. 2B is is a graph showing stereological counts.

FIG. 2C is a graph showing dopamine concentrations in the striatum of WT and PAAN/MIF KO at 6 months after α-syn PFF or PBS injection measured by high performance liquid chromatography (HPLC).

FIG. 2D is a graph showing DOPAC concentrations in the striatum of WT and PAAN/MIF KO at 6 months after α-syn PFF or PBS injection measured by high performance liquid chromatography (HPLC).

FIG. 2E is a graph showing pole test result 180 days after intrastriatal α-syn PBS or PFF injection, were performed in WT or PAAN/MIF KO.

FIG. 2F is a graph showing grip test result 180 days after intrastriatal α-syn PBS or PFF injection, were performed in WT or PAAN/MIF KO.

FIG. 2G is an image illustrating representative TH and Nissl staining of SNpc DA neurons of WT, E22Q and P1G knock-in mice at 6 months after intrastriatal α-syn PFF or PBS injection.

FIG. 2H is a graph showing stereological counts of TH cells.

FIG. 2I is a graph showing dopamine concentrations in the striatum of PAAN/MIF WT, E22Q and P1G knock-in mice at 6 months after α-syn PFF or PBS injection measured by high performance liquid chromatography (HPLC).

FIG. 2J is a graph showing pole test result 180 days after intrastriatal PBS or α-syn PFF injection in PAAN/MIF WT, E22Q and P1G knock-in mice.

FIG. 2K is a graph showing grip test result 180 days after intrastriatal PBS or α-syn PFF injection in PAAN/MIF WT, E22Q and P1G knock-in mice.

FIG. 3A is a graph showing nuclear translocation of AIF and PAAN/MIF after α-syn PFF treatment in the presence of the PARP inhibitor, ABT-888, in cortical neurons. Intensity of PAAN/MIF and AIF signal is shown in the graph.

FIG. 3B is a graph showing immunoprecipitation (IP) of PAAN/MIF and AIF in PBS or α-syn PFF-treated cortical neurons. Intensity of AIF-bound PAAN/MIF is shown in the graph.

FIG. 3C is an image showing nuclear translocation of PAAN/MIF (green) and AIF (red) after α-syn PFF treatment in primary cortical neurons.

FIG. 3D is a graph showing pulsed-field gel electrophoresis of α-syn PFF-induced DNA damage in PAAN/MIF WT and KO neurons and KO neurons expressing PAAN/MIF WT, E22Q, E22A or P1G. Intensity of noncleaved genomic DNA is shown in the graph.

FIG. 3E shows representative images of Hoechst and PI staining from primary cortical neurons transduced with AAV containing PAAN/MIF WT, E22Q, E22A or P1G and further incubated with α-syn PFF.

FIG. 3F illustrates quantification of cell death.

FIG. 3G illustrates quantification of cell death from Hoechst and PI staining of primary cortical neurons from PAAN/MIF WT, KO, E22Q and P1G KI mice.

FIG. 3H shows pulsed-field gel electrophoresis of α-syn PFF-induced DNA damage in PAAN/MIF WT, KO, E22Q and P1G KI neurons treated with PBS or α-syn PFF. Intensity of noncleaved genomic DNA is shown in the graph.

FIG. 4A shows scatter plot of percentage inhibition of PAAN/MIF cleavage from 45,000 compounds with 3,000 pools in 38 plates of the macrocyclic library.

FIG. 4B shows representative images of Hoechst and PI staining from human cortical neurons pre-incubated with compound 56, 77 or ABT-888 for 1 h and further incubated with α-syn PFF for 14 days.

FIG. 4C shows pulsed-field gel electrophoresis of α-syn PFF-induced DNA damage in human cortical neurons treated with compound 56 or 77. Intensity of noncleaved genomic DNA is shown in the graph.

FIG. 4D shows SH-SY5Y cells were pre-incubated compound 56 or 121 with concentrations as indicated for 1 h, followed by 50 μM MNNG for 15 min. After 24 h, cell viability was measured by Alamar blue.

FIG. 4E shows binding affinities of PAAN/MIF WT for compound 121 determined by biolayer interferometry (ForteBio Octet) assay.

FIG. 4F shows in vitro PAAN/MIF's nuclease assay using PAAN/MIF or PAAN/MIF mutants with compound 121.

FIG. 4G shows WT or PAAN/MIF KO SH-SY5Y cells expressing Flag-PAAN/MIF WT or mutants were pre-incubated 1 μM of compound 121 for 1 h, followed by 50 μM MNNG for 15 min.

FIG. 4H shows binding of PAAN/MIF WT or mutants for compound 121 determined by biolayer interferometry (ForteBio Octet) assay.

FIG. 5A shows schematic diagram of the experimental design to demonstrate compound 121 protects against α-syn PFF-induced pathology in vivo.

FIG. 5B shows representative TH and Nissl staining of SNpc DA neurons of PBS or α-syn PFF injected WT mice treated with vehicle or compound 121.

FIG. 5C shows stereological counts of TH-positive cells.

FIG. 5D shows stereological counts of Nissl-positive cells.

FIG. 5E shows Dopamine concentrations in the striatum of PBS or α-syn PFF injected WT mice treated with vehicle of compound 121 as assessed by HPLC.

FIG. 5F shows DOPAC concentrations in the striatum of PBS or α-syn PFF injected WT mice treated with vehicle of compound 121 as assessed by HPLC.

FIG. 5G shows pole test result 180 days after intrastriatal α-syn PFF or PBS injection in WT mice treated with vehicle or compound 121.

FIG. 5H shows grip strength test result 180 days after intrastriatal α-syn PFF or PBS injection in WT mice treated with vehicle or compound 121.

FIG. 5I shows DNA fragmentation determined by pulsed-field gel electrophoresis in PBS or α-syn PFF injected WT mice treated with vehicle or compound 121. Intensity of noncleaved genomic DNA is shown in the graph.

FIG. 6A shows representative immunoblots and quantification of TH and DAT levels in the (c) contralateral and (i) ipsilateral striatum of PBS-injected WT or MIF/PAAN KO mice.

FIG. 6B shows representative immunoblots and quantification of TH and DAT levels in the (c) contralateral and (i) ipsilateral striatum of α-syn PFF-injected WT or MIF/PAAN KO mice.

FIG. 6C shows behavioral abnormalities of PBS and α-syn PFF-injected mice at 6 months as measured by clasping test.

FIG. 6D shows behavioral abnormalities of PBS and α-syn PFF-injected mice at 6 months as measured by pole test.

FIG. 6E shows behavioral abnormalities of PBS and α-syn PFF-injected mice at 6 months as measured by grip strength test.

FIG. 7A shows representative immunoblots of TH and 3-actin in the contralateral and ipsilateral striatum of PBS and α-syn PFF-injected MIF/PAAN WT, MIF/PAAN E22Q KI or MIF P1G KI mice at 6 months.

FIG. 7B shows quantification of TH levels in the striatum normalized to β-actin.

FIG. 7C shows stereological counts of TH Nissl* cells.

FIG. 7D shows DOPAC concentrations in the striatum of WT, E22Q and P1G knock-in mice at 6 months after α-syn PFF or PBS injection measured by HPLC.

FIG. 7E shows behavioral abnormalities of PBS and α-syn PFF-injected MIF/PAAN WT, MIF/PAAN E22Q or MIF P1G KI mice at 6 months measured by (e) pole test.

FIG. 7F shows behavioral abnormalities of PBS and α-syn PFF-injected MIF/PAAN WT, MIF/PAAN E22Q or MIF P1G KI mice at 6 months measured by grip strength test.

FIG. 7G shows pulsed-field gel electrophoresis in PBS or α-syn PFF injected MIF/PAAN WT, MIF/PAAN E22Q KI or MIF P1G KI mice at 6 months.

FIG. 7H shows intensity of noncleaved genomic DNA.

FIG. 8A shows representative TH and Nissl staining of SNpc DA neurons of MIF/PAAN WT, MIF/PAAN KO and MIF/PAAN KO mice injected with AAV2-Flag-MIF/PAAN WT, E22Q or P1G at 6 months after α-syn PFF or PBS injection.

FIG. 8B shows stereological counts.

FIG. 8C shows time to turn and bottom pole test result 180 days after intrastiriatal α-syn PBS or PFF injection, performed in WT, MIF/PAAN KO and MIF/PAAN KO mice injected with AAV2-MIF/PAAN WT, E22Q or P1G-Flag.

FIG. 8D shows time to turn pole test result 180 days after intrastiriatal α-syn PBS or PFF injection, performed in WT, MIF/PAAN KO and MIF/PAAN KO mice injected with AAV2-MIF/PAAN WT, E22Q or P1G-Flag.

FIG. 8E shows forelimb grip strength test result 180 days after intrastiriatal α-syn PBS or PFF injection, performed in WT, MIF/PAAN KO and MIF/PAAN KO mice injected with AAV2-MIF/PAAN WT, E22Q or P1G-Flag.

FIG. 8F shows forelimb and hindlimb grip strength test result 180 days after intrastiriatal α-syn PBS or PFF injection, performed in WT, MIF/PAAN KO and MIF/PAAN KO mice injected with AAV2-MIF/PAAN WT, E22Q or P1G-Flag.

FIG. 8G shows representative immunoblots of TH, Flag, MIF/PAAN and j-actin in the contralateral and ipsilateral striatum of PBS and α-syn PFF-injected MIF/PAAN WT, MIF/PAAN KO and MIF/PAAN KO mice injected with AAV2-Flag-MIF/PAAN WT, E22Q or P1G at 6 months.

FIG. 8H shows representative immunostaining images of expression of AAV2-Flag-MIF/PAAN WT, MIF/PAAN-E22Q and MIF P1G in cortex, hippocampus, striatum and substantia nigra 6 month after injection.

FIG. 9A shows nuclear translocation of AIF and MIF/PAAN variants determined by western blot analysis from post nuclear (PN) and nuclear (N) fraction.

FIG. 9B shows co-immunoprecipitation (IP) of Flag-tagged MIF/PAAN variants and AIF in cortical neurons after α-syn PFF treatment.

FIG. 9C shows images of nuclear translocation of AIF and Flag-tagged MIF/PAAN variants after α-syn PFF treatment in MIF/PAAN KO cortical neurons.

FIG. 10A shows schematic diagram for MIF/PAAN substrate, PS30 and RF.

FIG. 10B shows MIF/PAAN cleavage assay without or with MIF/PAAN protein in a concentration dependent manner.

FIG. 10C shows plate—to plate and day to day variability of the parameters (CV, S/B and Z′ factor) of MIF's cleavage screening assay.

FIG. 10D shows the result of secondary screening from the primary screening pools that inhibited MIF/PAAN nuclease activity by at least 60%.

FIG. 10E shows Alamar blue cell viability assay from SH-SY5Y cells pre-incubated with 9 pools selected by secondary screening in a concentration-dependent manner (0.1 μM, 0.2 μM, 0.5 μM and 1 μM) in response to 50 μM MNNG for 15 min.

FIG. 10F shows the result of the individual compounds screening (˜90) of 6 pools candidates from the secondary screening.

FIG. 11A shows Alamar blue cell viability assay from SH-SY5Y cells pre-incubated with 12 MIF/PAAN inhibitors selected by individual screening in a concentration-dependent manner (0.1 μM, 0.2 μM, 0.5 μM and 1 μM) in response to 50 μM MNNG for 15 min.

FIG. 11B shows in vitro MIF's nuclease assay with 12 MIF/PAAN inhibitors using RF substrates.

FIG. 11C shows in vitro MIF's nuclease assay with 12 MIF/PAAN inhibitors using PS30 substrates.

FIG. 12A shows Representative western blot analysis and quantification of the levels of MNNG-induced PAR accumulation in the presence and absence of MIF/PAAN inhibitors (compounds 41, 56, 76, and 77).

FIG. 12B shows in vitro oxidoreductase of MIF/PAAN with compound 41, 56, 76, or 77.

FIG. 12C shows in vitro tautomerase activity of MIF/PAAN with compound 41, 56, 76, or 77.

FIG. 12D shows Alamar blue cell viability assay from SH-SY5Y cells pre-incubated with compound 41, 56, 76, or 77 in response to 0.5 μM staurosporine (STS) incubated with 1 ng/ml of TNF-α with 50 μM z-VAD.

FIG. 12E shows Alamar blue cell viability assay from SH-SY5Y cells pre-incubated with compound 41, 56, 76, or 77 from HT 22 cells incubated with 1 ng/ml of TNF-α with 50 μM z-VAD.

FIG. 12F shows in vitro nuclease assay of EcoRI and EcoRV with pcDNA3 as a substrate and ExoIII with 18 bp of double-strand DNA as a substrate in the absence or presence of MIF/PAAN inhibitors (compound 41, 56, 76, or 77).

FIG. 12G shows quantification of cell death from Hoechst and propidium iodide (PI) staining from primary cortical neurons pre-treated with 1 μM of compound 41, 56, 76, or 77, followed by further incubation with α-syn PFF.

FIG. 13A shows C57BL/6 mice orally administrated with 10 mg/kg of vehicle, compound 56, 77, or 121 for 2h.

FIG. 13B shows quantification of cell death.

FIG. 13C shows in vitro MIF's nuclease assay with compound 56 or 121.

FIG. 13D shows pulsed-field gel electrophoresis of α-syn PFF-induced DNA damage in mouse cortical neurons treated with compound 56 or 121.

FIG. 14A shows Quanqification of noncleaved substrate DNA from in vitro MIF's nuclease assay with indicated dose of rapamycin, FK506 or MIF/PAAN inhibitors (compounds 56, 77 and 121) using the PS 30 substrate.

FIG. 14B shows Alamar blue cell viability assay from SH-SY5Y cells pre-incubated with 0.5 or 1 μM of rapamycin, FK506 or MIF/PAAN inhibitors (compounds 56, 77 and 121) in response to 0 μM MNNG for 15 min.

FIG. 14C shows representative immunoblots (left) and quantification (right) of pS6K, pS6 and p4E-BP1 levels in SH-SY5Y cells incubated with 0.5 or 1 μM of Rapamycin or compound 121 for 3 h.

FIG. 14D shows calcineurin activity assay from SH-SY5Y cells treated with 1 μM of FK506 or compound 121 for 6 h.

FIG. 14E shows in vitro ribosylation assay (IVRA) of PARP-1 with compound 56, 121 or ABT-888.

FIG. 14F shows Alamar blue cell viability assay from SH-SY5Y cells pre-incubated with either compound 56 or 121 in response to 0.5 μM staurosporine (STS) incubated with 1 ng/ml of TNF-α with 50 μM z-VAD.

FIG. 14G shows Alamar blue cell viability assay from SH-SY5Y cells pre-incubated with either compound 56 or 121 in response from HT 22 cells incubated with 1 ng/ml of TNF-α with 50 μM z-VAD.

FIG. 15A shows effect of compound 121 on binding of MIF/PAAN with biotin-labeled small DNA substrate (PS30) in an EMSA assay.

FIG. 15B shows effect of compound 97 on MIF nuclease activity.

FIG. 15C shows effect of compound 97 on MNNG-induced cell death.

FIG. 15D shows binding affinities of MIF/PAAN WT to non-bound compound 97 as determined by biolayer interferometry (ForteBio Octet) assay.

FIG. 15E shows 3D model of MIF/PAAN with compound 121.

FIG. 15F shows effect of compound 121 in MIF/PAAN nuclease assay with different MIF/PAAN mutants using PS30 substrate.

FIG. 15G shows effect of modification of position of N-methylalanine in compound 56 on its activities.

FIG. 16A shows measurement of weight from PBS or α-syn PFF-injected WT mice before or after administration of vehicle and compound 121.

FIG. 16B shows representative immunoblots of TH and f-actin in the contralateral and ipsilateral striatum of PBS or α-syn PFF-injected WT mice delivered vehicle or compound 121 for 5 months.

FIG. 16C shows quantification of TH levels in the striatum normalized to β-actin.

FIG. 16D shows behavioral defects of PBS and α-syn PFF-injected mice administrated vehicle or compound 121 for 5 months measured by pole test.

FIG. 16E shows behavioral defects of PBS and α-syn PFF-injected mice administrated vehicle or compound 121 for 5 months measured by grip strength test.

FIG. 17A shows colorimetric assay for MIF nuclease activity using 10 μM of compound 121, which is a racemic mixture of compound 153 and 154, compound 153, and compound 154.

FIG. 17B shows MNNG-induced cell death assay for compound 121, which is a racemic mixture of compound 153 and 154, compound 153, and compound 154.

FIG. 18A shows activation of PARP-1 in H202-treated primary rat cardiac myocytes.

FIG. 18B shows cardiac function characterized by LY systolic diameter (LVID) using two-dimensional echocardiography at indicated time after surgery.

FIG. 18C shows cardiac function characterized by ejection fraction (EF) using two-dimensional echocardiography at indicated time after surgery.

FIG. 18D shows cardiac function characterized by shortening fraction (SF) using two-dimensional echocardiography at indicated time after surgery.

FIG. 19A shows serum creatinine levels in MIF WT, MIF KO, and MIF KI male or female mice after kidney I/R (45 min of ischemia and 48 h of reperfusion).

FIG. 19B shows blood urea nitrogen (BUN) levels in MIF WF, MIF KO, and MIF KI male or female mice after kidney I/R (45 min of ischemia and 48 h of reperfusion).

### DETAILED DESCRIPTION

In the following detailed description of the embodiments of the instant disclosure, numerous specific details are set forth in order to provide a thorough understanding of the disclosed embodiments. However, it will be obvious to one skilled in the art that the embodiments of this disclosure may be practiced without these specific details. In other instances, well known methods, procedures, components, and circuits have not been described in detail so as not to unnecessarily obscure aspects of the embodiments of the instant disclosure.

Inhibition or genetic deletion of poly(ADP-ribose) polymerase-1 (PARP-1) is profoundly protective against a number of toxic insults in many organ systems. The molecular mechanisms underlying PARP-1-dependent cell death involve mitochondrial apoptosis-inducing factor (AIF) release and translocation to the nucleus resulting in chromatinolysis. How AIF induces chromatinolysis and cell death is not known. The present disclosure is based on the identification of Macrophage Migration Inhibitory Factor (MIF) as a PARP-1 dependent AIF-associated nuclease (PAAN) that possesses Mg²⁺/Ca²⁺-dependent nuclease activity. AIF is required for recruitment of MIF to the nucleus where MIF cleaves genomic DNA into 20-50 kb fragments. Depletion of MIF, disruption of the AIF-MIF interaction or mutation of E22 to Q22 in the catalytic nuclease domain blocks MIF nuclease activity, inhibits chromatinolysis and cell death following glutamate excitotoxicity in neuronal cultures and focal stroke in mice. Inhibition of MIF's nuclease activity is a potential critical therapeutic target for diseases that are due to excessive PARP-1 activation.

A rapafucin library was synthesized as described in WO2017/136708 which is incorporated herein by reference in its entirety. Approximately 45,000 compounds were generated, and ongoing screening of the library as described in WO2018/045250 identified several compounds as being inhibitors of MIF nuclease activity. WO2018/045250 is incorporated herein by reference in its entirety. Structure-activity relationship (SAR) analysis was then conducted on the identified compounds and from this analysis, compounds that exhibit neuroprotective activity were synthesized and have the structure of Formula (I). Illustrative compounds that were synthesized are set forth in Table 2.

As such, disclosed herein is a compound according to Formula (I) or an optically pure stereoisomer or pharmaceutically acceptable salt thereof.

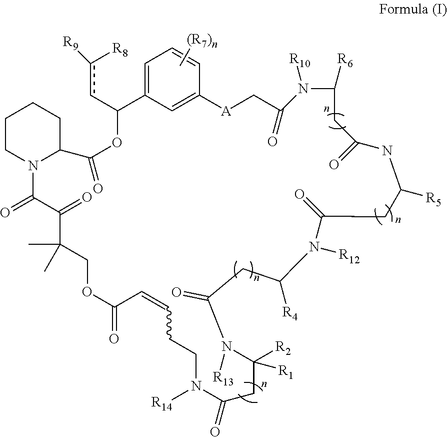

Each R₁ and R₂ is independently selected from the group consisting of H, halogen, hydroxyl, C₁₋₂₀ alkyl, substituted C₁₋₂₀ alkyl, OC₁₋₂₀ alkyl, substituted OC₁₋₂₀ alkyl, N₃, NH₂, NO₂, CF₃, OCF₃, OCHF₂, COC₁₋₂₀alkyl, CO₂C₁₋₂₀alkyl, C₃₋₈cycloalkyl, substituted C₃₋₈cycloalkyl, C₆₋₁₅aryl, substituted C₆₋₁₅aryl, and a cyclic substituent formed with the adjacent nitrogen, the cyclic substituent is selected from the group consisting of

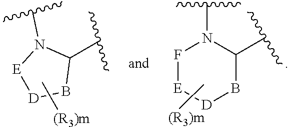

R₃ is selected from H, halogen, hydroxyl, N₃, NH₂, NO₂, CF₃, C₁₋₁₀alkyl, substituted C₁₋₁₀alkyl, C₁₋₁₀alkoxy, substituted C₁₋₁₀alkoxy, acyl, acylamino, acyloxy, acyl C₁₋₁₀alkyloxy, amino, substituted amino, aminoacyl, aminocarbonyl C₁₋₁₀alkyl, aminocarbonylamino, aminodicarbonylamino, aminocarbonyloxy, aminosulfonyl, C₆₋₁₅aryl, substituted C₆₋₁₅aryl, C₆₋₁₅aryloxy, substituted C₆₋₁₅aryloxy, C₆₋₁₅arylthio, substituted C₆₋₁₅arylthio, carboxyl, carboxyester, (carboxyester)amino, (carboxyester)oxy, cyano, C₃₋₈cycloalkyl, substituted C₃₋₈cycloalkyl, (C₃₋₈cycloalkyl)oxy, substituted (C₃₋₈cycloalkyl)oxy, (C₃₋₈cycloalkyl)thio, substituted (C₃₋₈cycloalkyl)thio, C₁₋₁₀heteroaryl, substituted C₁₋₁₀heteroaryl, C₁₋₁₀heteroaryloxy, substituted C₁₋₁₀heteroaryloxy, C₁₋₁₀heteroarylthio, substituted C₁₋₁₀heteroarylthio, C₂₋₁₀heterocyclyl, C₂₋₁₀substituted heterocyclyl, C₂₋₁₀heterocyclyloxy, substituted C₂₋₁₀heterocyclyloxy, C₂₋₁₀heterocyclylthio, substituted C₂₋₁₀heterocyclylthio, imino, oxo, sulfonyl, sulfonylamino, thiol, C₁₋₁₀alkylthio, substituted C₁₋₁₀alkylthio, and thiocarbonyl.

Or two R₃ substituents, together with the atom to which each is bound, may form ring selected from a C₆₋₁₅aryl, substituted C₆₋₁₅aryl, C₃₋₈cycloalkyl, substituted C₃₋₈cycloalkyl, C₁₋₁₀heteroaryl, substituted C₁₋₁₀heteroaryl, C₂₋₁₀substituted heterocyclyl, C₂₋₁₀heterocyclyloxy, and substituted C₂₋₁₀heterocyclyloxy.

Each n and m can be independently selected from 0-5. A can be oxygen or —NH—CO—CH₂—. Each of B, D, E, and F independently can be selected from the group consisting of carbon, nitrogen, oxygen, and sulfur.

R₄ is selected from the group consisting of H, halogen, hydroxyl, C₁₋₂₀ alkyl, N₃, NH₂, NO₂, CF₃, OCF₃, OCHF₂, COC₁₋₂₀alkyl, CO₂C₁₋₂₀alkyl, and a cyclic substituent formed with the adjacent nitrogen, the cyclic substituent is selected from the group consisting of

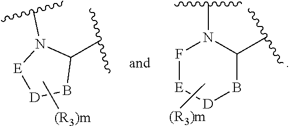

R₅ is selected from the group consisting of C₆₋₁₅aryl and C₁₋₁₀heteroaryl optionally substituted with H, halogen, hydroxyl, N₃, NH₂, NO₂, CF₃, C₁₋₁₀alkyl, substituted C₁₋₁₀alkyl, C₁₋₁₀alkoxy, substituted C₁₋₁₀alkoxy, acyl, acylamino, acyloxy, acyl C₁₋₁₀alkyloxy, amino, substituted amino, aminoacyl, aminocarbonyl C₁₋₁₀alkyl, aminocarbonylamino, aminodicarbonylamino, aminocarbonyloxy, aminosulfonyl, C₆₋₁₅aryl, substituted C₆₋₁₅aryl, C₆₋₁₅aryloxy, substituted C₆₋₁₅aryloxy, C₆₋₁₅arylthio, substituted C₆₋₁₅arylthio, carboxyl, carboxyester, (carboxyester)amino, (carboxyester)oxy, cyano, C₃₋₈cycloalkyl, substituted C₃₋₈cycloalkyl, (C₃₋₈cycloalkyl)oxy, substituted (C₃₋₈cycloalkyl)oxy, (C₃₋₈cycloalkyl)thio, substituted (C₃₋₈cycloalkyl)thio, halo, hydroxyl, C₁₋₁₀heteroaryl, substituted C₁₋₁₀heteroaryl, C₁₋₁₀heteroaryloxy, substituted C₁₋₁₀heteroaryloxy, C₁₋₁₀heteroarylthio, substituted C₁₋₁₀heteroarylthio, C₂₋₁₀heterocyclyl, C₂₋₁₀substituted heterocyclyl, C₂₋₁₀heterocyclyloxy, substituted C₂₋₁₀heterocyclyloxy, C₂₋₁₀heterocyclylthio, substituted C₂₋₁₀heterocyclylthio, imino, oxo, sulfonyl, sulfonylamino, thiol, C₁₋₁₀alkylthio, substituted C₁₋₁₀alkylthio, and thiocarbonyl.

R₆ is selected from the group consisting of H, halogen, hydroxyl, C₁₋₂₀ alkyl, C₁₋₁₀alkyloxy, substituted C₁₋₁₀alkyloxy, N₃, NH₂, NO₂, CF₃, OCF₃, OCHF₂, COC₁₋₂₀alkyl, CO₂C₁₋₂₀alkyl, C₃₋₈cycloalkyl, substituted C₃₋₈cycloalkyl, amino, substituted amino, aminoacyl, aminocarbonyl C₁₋₁₀alkyl, aminocarbonylamino, aminodicarbonylamino, aminocarbonyloxy, aminosulfonyl, thiol, C₁₋₁₀alkylthio, substituted C₁₋₁₀alkylthio, and a cyclic substituent formed with the adjacent nitrogen, the cyclic substituent is selected from the group consisting of

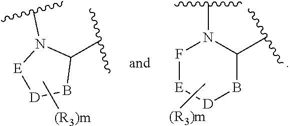

Each R₇ and R₈ is independently selected from the group consisting of H, halogen, hydroxyl, C₁₋₂₀ alkyl, N₃, NH₂, NO₂, CF₃, OCF₃, OCHF₂, COC₁₋₂₀alkyl, and CO₂C₁₋₂₀alkyl. can represent a single or a double bond.

R₉ is selected from the group consisting of C₆₋₁₅aryl and C₁₋₁₀heteroaryl optionally substituted with H, halogen, hydroxyl, N₃, NH₂, NO₂, CF₃, C₁₋₁₀alkyl, substituted C₁₋₁₀alkyl, C₁₋₁₀alkoxy, substituted C₁₋₁₀alkoxy, acyl, acylamino, acyloxy, acyl C₁₋₁₀alkyloxy, amino, substituted amino, aminoacyl, aminocarbonyl C₁₋₁₀alkyl, aminocarbonylamino, aminodicarbonylamino, aminocarbonyloxy, aminosulfonyl, C₆₋₁₅aryl, substituted C₆₋₁₅aryl, C₆₋₁₅aryloxy, substituted C₆₋₁₅aryloxy, C₆₋₁₅arylthio, substituted C₆₋₁₅arylthio, carboxyl, carboxyester, (carboxyester)amino, (carboxyester)oxy, cyano, C₃₋₈cycloalkyl, substituted C₃₋₈cycloalkyl, (C₃₋₈cycloalkyl)oxy, substituted (C₃₋₈cycloalkyl)oxy, (C₃₋₈cycloalkyl)thio, substituted (C₃₋₈cycloalkyl)thio, C₁₋₁₀heteroaryl, substituted C₁₋₁₀heteroaryl, C₁₋₁₀heteroaryloxy, substituted C₁₋₁₀heteroaryloxy, C₁₋₁₀heteroarylthio, substituted C₁₋₁₀heteroarylthio, C₂₋₁₀heterocyclyl, C₂₋₁₀substituted heterocyclyl, C₂₋₁₀heterocyclyloxy, substituted C₂₋₁₀heterocyclyloxy, C₂₋₁₀heterocyclylthio, substituted C₂₋₁₀heterocyclylthio, imino, oxo, sulfonyl, sulfonylamino, thiol, C₁₋₁₀alkylthio, substituted C₁₋₁₀alkylthio, and thiocarbonyl.

Each R₁₀, R₁₁, R₁₂, R₁₃, and R₁₄ is independently selected from the group consisting of H, methyl, ethyl, propyl, and isopropyl.

In some embodiments, each R₁ and R₂ can be independently selected from the group consisting- of

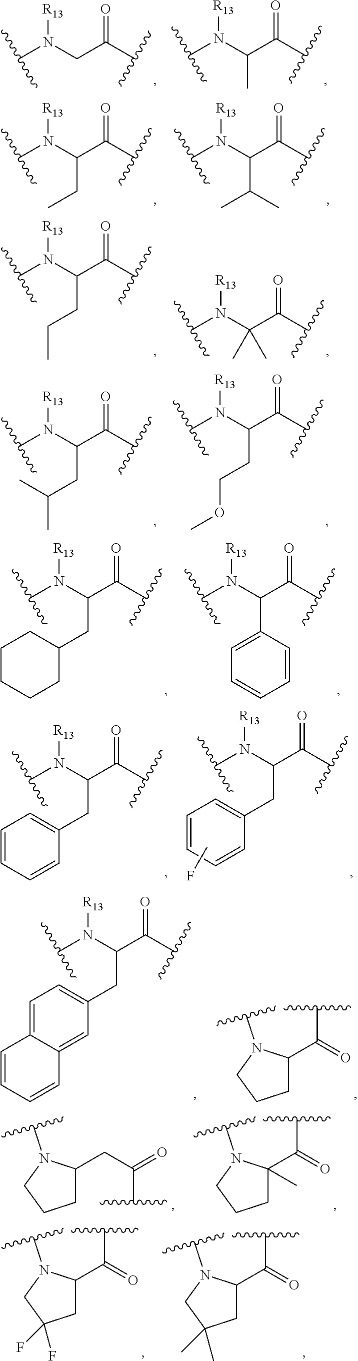

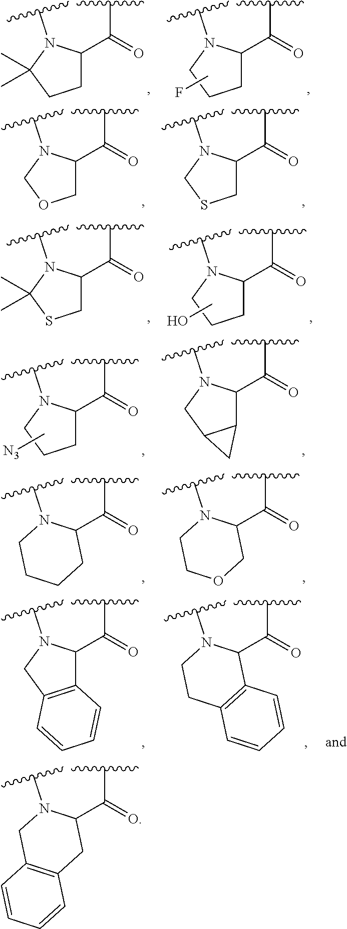

In some embodiments, R₄ can be selected from the group consisting of

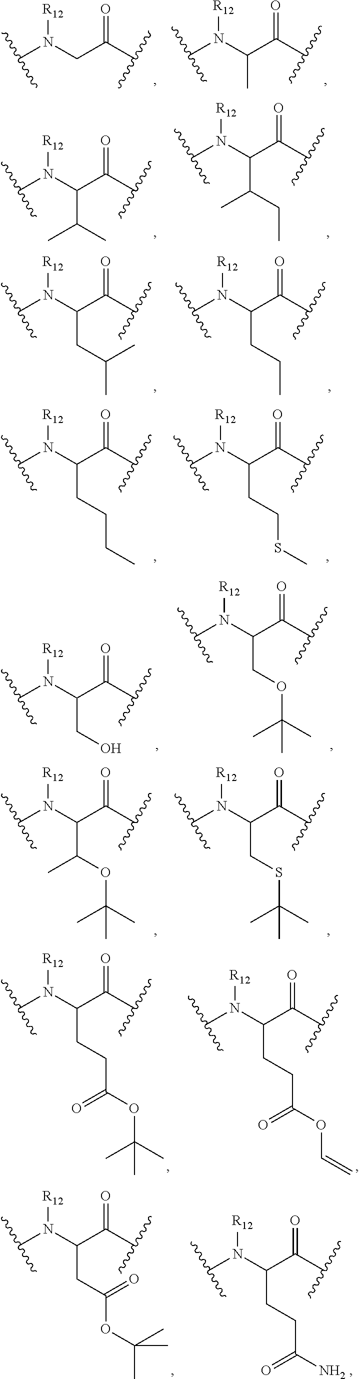

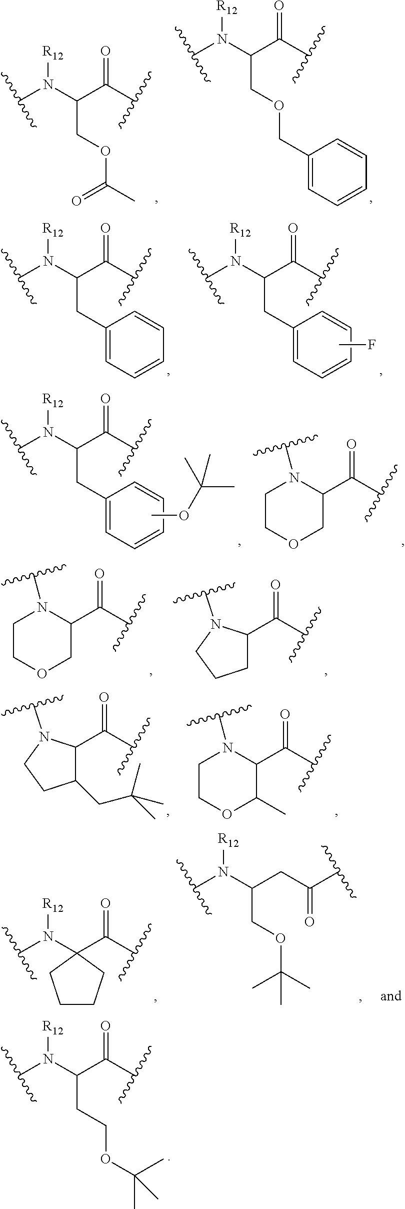

In some embodiments, R₅ can be selected from the group consisting of

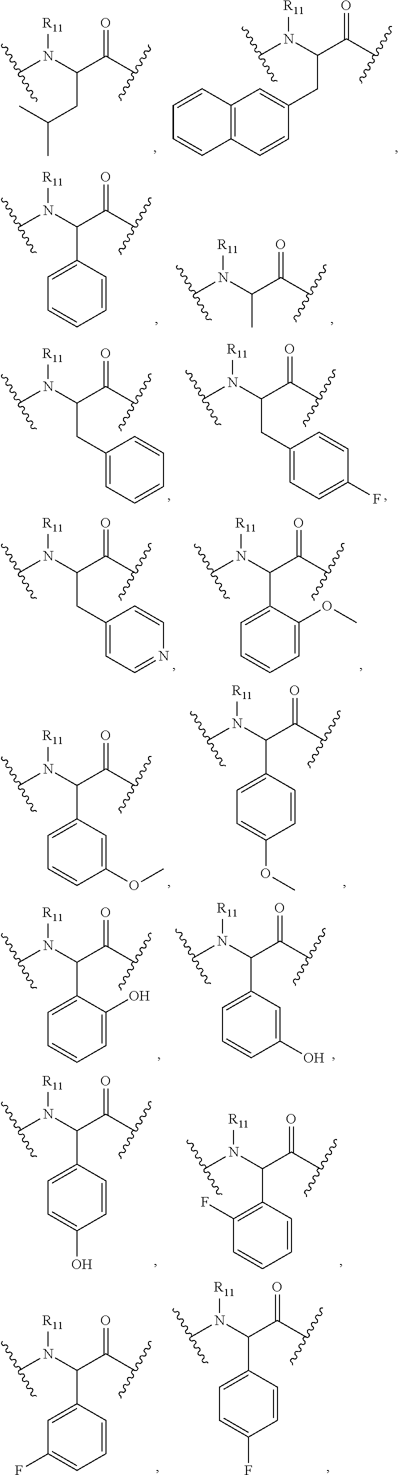

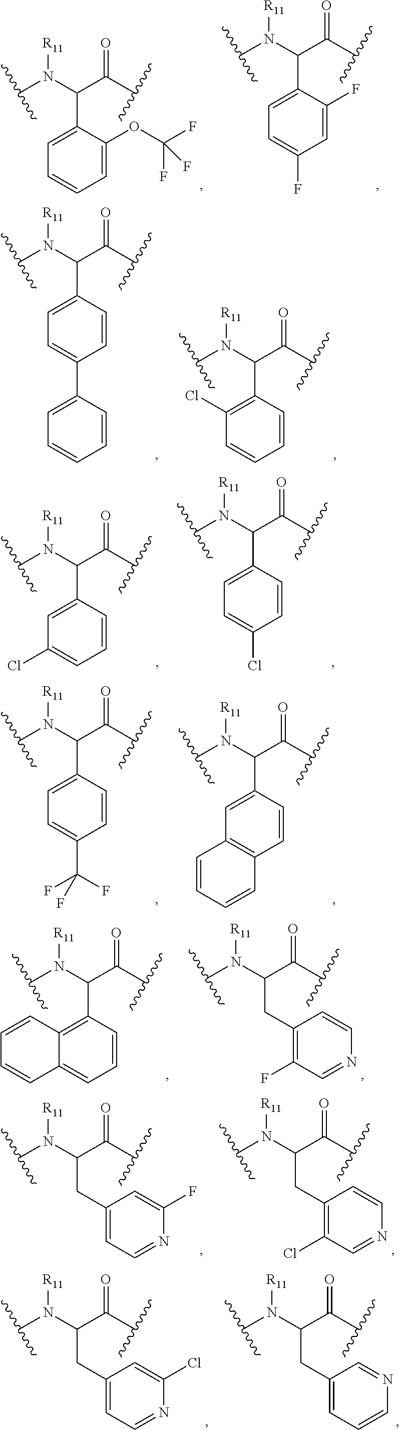

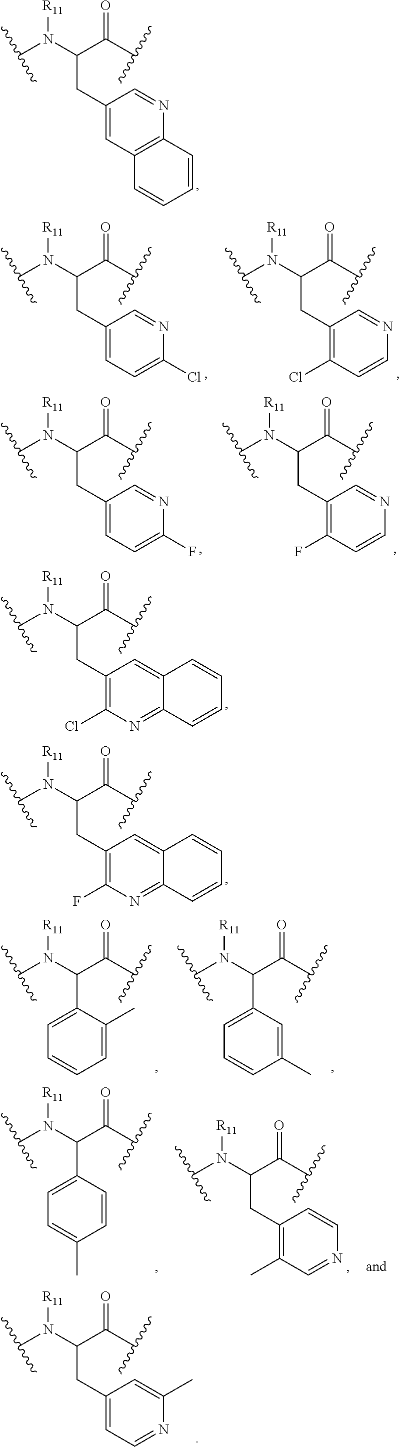

In some embodiments, R₆ can be selected from the group consisting of

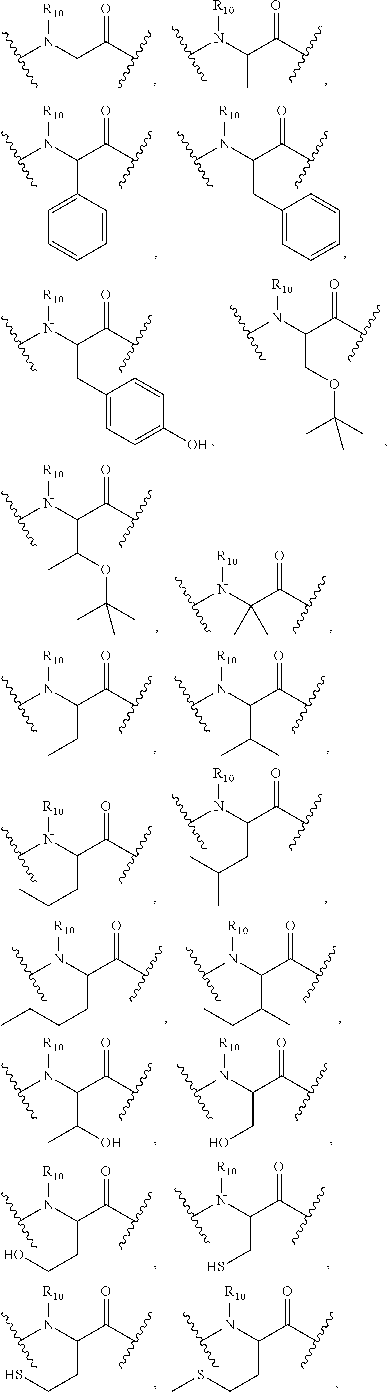

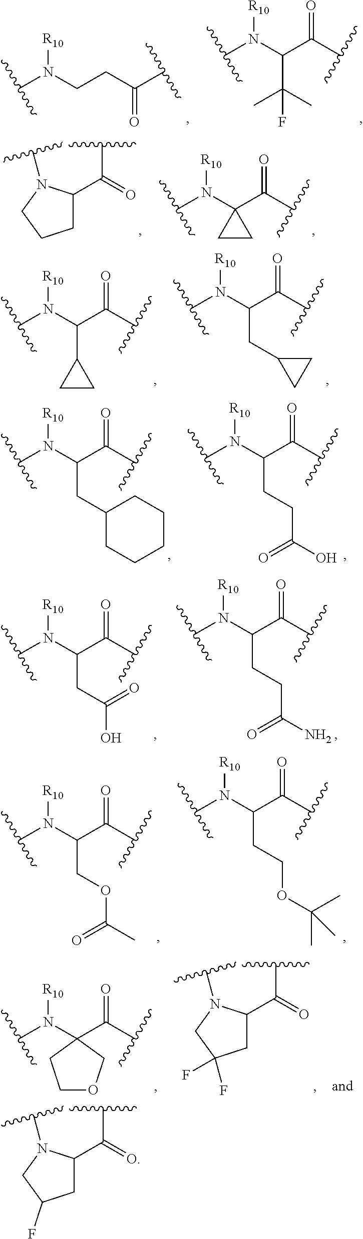

In some embodiments, R₉ can be selected from the group consisting of

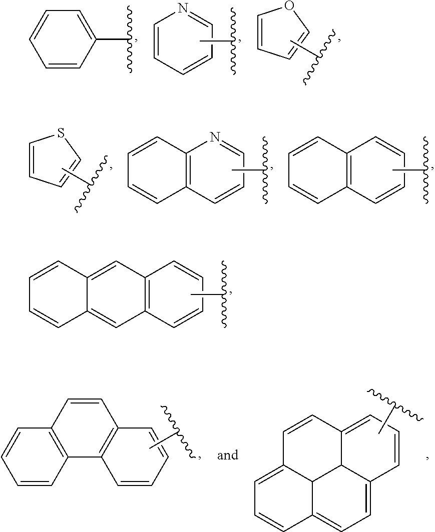

optionally substituted with halogen, hydroxyl, or alkoxyl.

In some embodiments, FKBD is used to represents the structural moiety

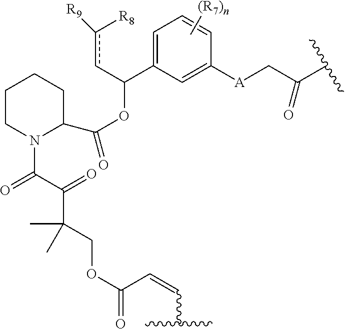

in Formula (I), which can be selected from the group consisting of

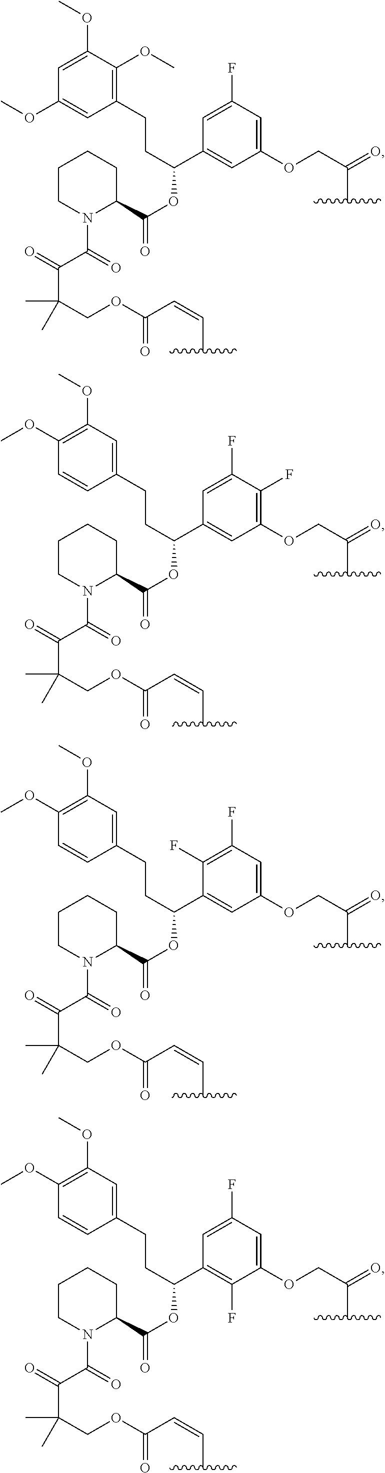

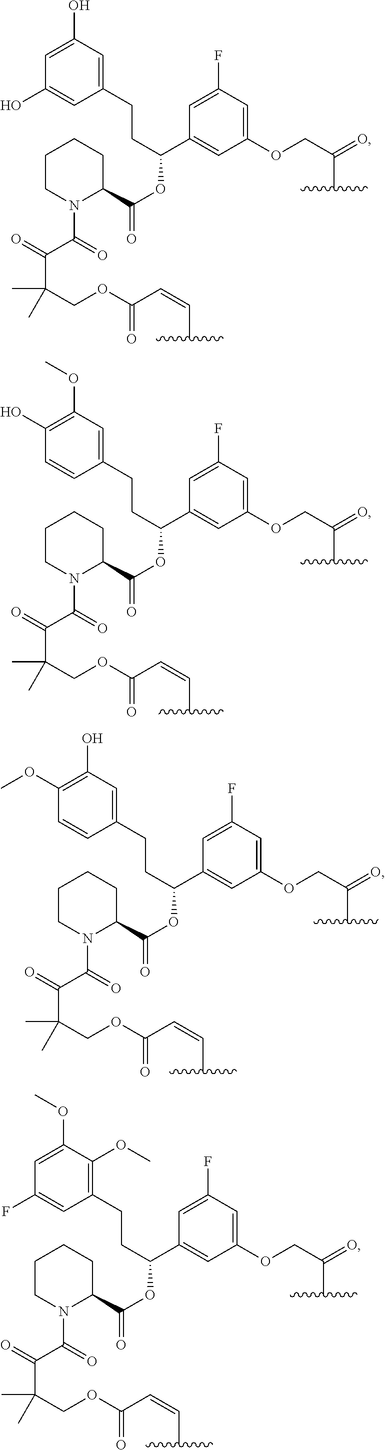

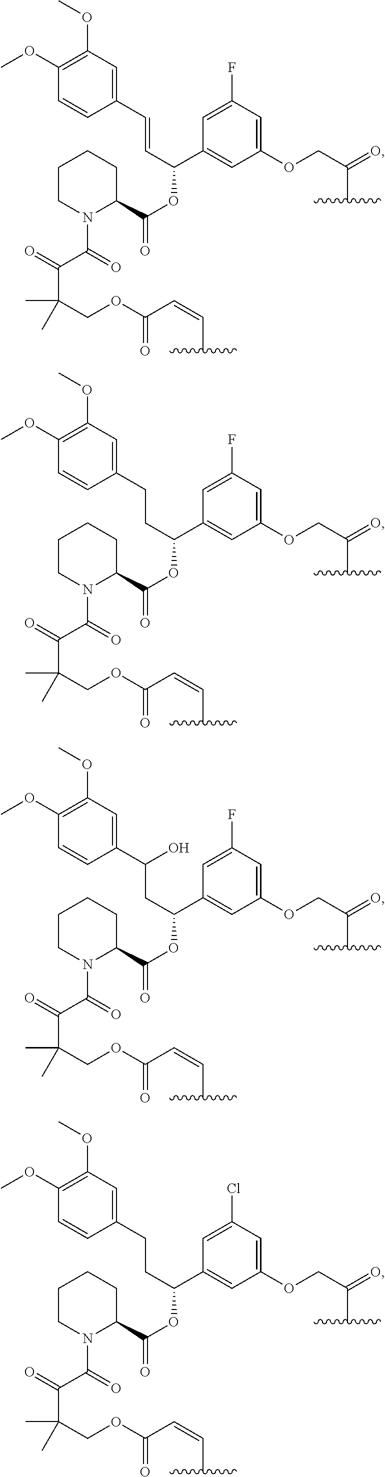

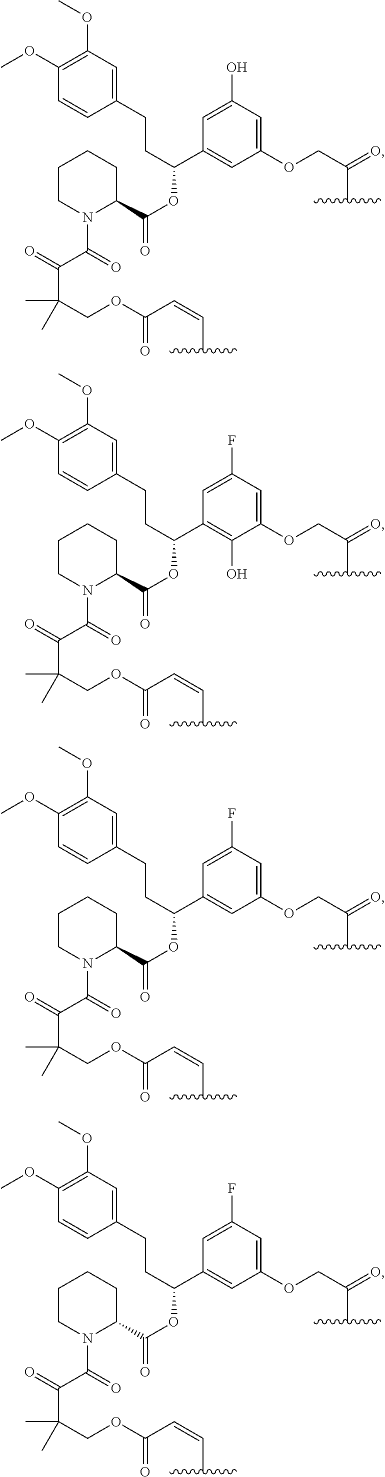 [SMILES: */C=C\C(=O)OCC(C)(C)C(=O)C(=O)N1CCCC[C@H]1C(=O)O[C@H](CCc1ccc(OC)c(OC)c1)c1cc(O)cc(OCC(*)=O)c1 ; */C=C\C(=O)OCC(C)(C)C(=O)C(=O)N1CCCC[C@H]1C(=O)O[C@H](CCc1ccc(OC)c(OC)c1)c1cc(F)cc(OCC(*)=O)c1O ; */C=C\C(=O)OCC(C)(C)C(=O)C(=O)N1CCCC[C@H]1C(=O)O[C@H](CCc1ccc(OC)c(OC)c1)c1cc(F)cc(OCC(*)=O)c1 ; */C=C\C(=O)OCC(C)(C)C(=O)C(=O)N1CCCC[C@@H]1C(=O)O[C@H](CCc1ccc(OC)c(OC)c1)c1cc(F)cc(OCC(*)=O)c1]

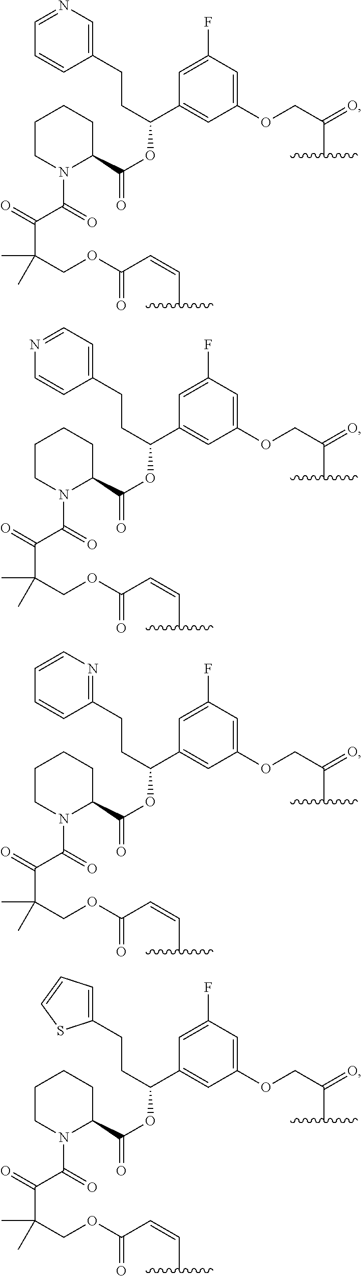

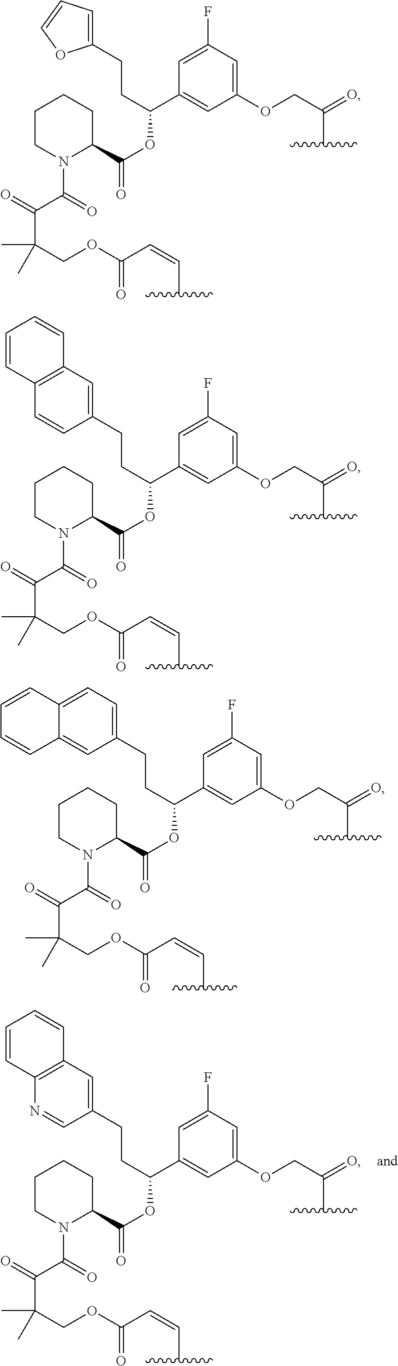

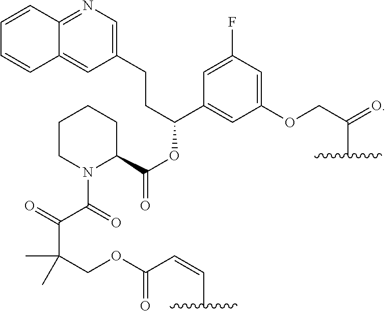 [SMILES: */C=C\C(=O)OCC(C)(C)C(=O)C(=O)N1CCCC[C@H]1C(=O)O[C@H](CCc1cnc2ccccc2c1)c1cc(F)cc(OCC(*)=O)c1]

In some embodiments, the compounds disclosed herein can be selected from the chemical compounds consisting of compounds 121, 3, and 1 with the following structures

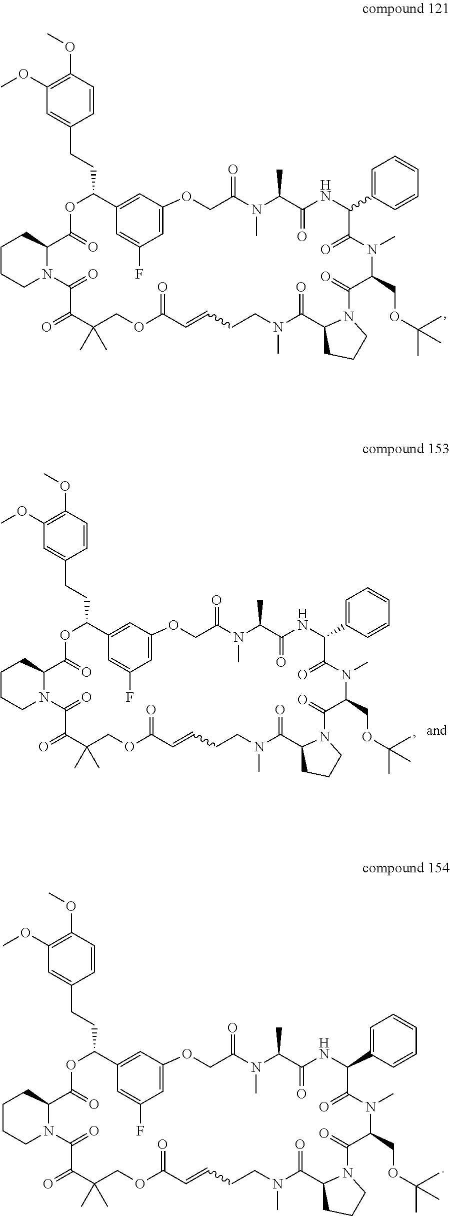

The macrocyclic natural products FK506 and rapamycin are approved immunosuppressive drugs with important biological activities. Both have been shown to inhibit T-cell activation, each with distinct mechanisms. In addition, rapamycin has been shown to have strong anti-proliferative activity. FK506 and rapamycin share an extraordinary mode of action; they act by recruiting an abundant and ubiquitously expressed cellular protein, the prolyl cis-trans isomerase FKBP, and the binary complexes subsequently bind to and allosterically inhibit their target proteins calcineurin and mTOR, respectively. Structurally, FK506 and rapamycin share a similar FKBP-binding domain but differ in their effector domains. In FK506 and rapamycin, nature has taught us that switching the effector domain of FK506 to that in rapamycin, it is possible to change the targets from calcineurin to mTOR. The generation of a rapafucin library of macrocyles that contain FK506 and rapamycin binding domains should have great potential as new leads for developing drugs to be used for treating diseases.

A variety of methods exist for the generation of compound libraries for developing and screening potentially useful compounds in treating diseases. One such method is the development of encoded libraries, and particularly libraries in which each compound includes an amplifiable tag. Such libraries include DNA-encoded libraries in which a DNA tag identifying a library member can be amplified using molecular biology techniques, such as the polymerase chain reaction (PCR). The use of such methods for producing libraries of rapafucin macrocyles that contain FK506-like and rapamycin-like binding domains has yet to be demonstrated. Thus, there remains a need for DNA-encoded rapafucin libraries of macrocyles that contain FK506-like and rapamycin-like binding domains.

In one aspect, provided herein is a tagged macrocyclic compound that comprises: an FK506 binding protein binding domain (FKBD); an effector domain; a first linking region; and a second linking region; wherein the FKBD, the effector domain, the first linking region, and the second linking region together form a macrocycle; and wherein at least one of the FKBD, the effector domain, the first linker, and the second linker can be operatively linked to one or more oligonucleotides (D) which can identify the structure of at least one of the FKBD, the effector domain, the first linker, and the second linker.

In certain embodiments, provided herein is a tagged macrocyclic compound of Formula (II):

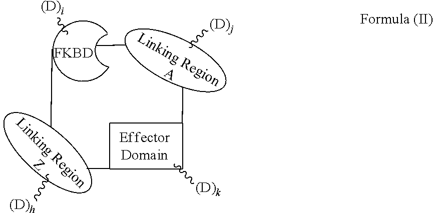

In some embodiments, h, i, j, and k are each independently an integer from 0-20, provided that at least one of h, i, j, and k is not 0; and D is an oligonucleotide that can identify at least one of the FKBD, the Effector Domain, the Linking Region A, or the Linking Region Z, where the solid lines linking the FKBD, the Effector Domain, the Linking Region A, and/or the Linking Region Z indicate an operative linkage and the squiggle lines indicate an operative linkage. In certain embodiments, oligonucleotide (D) can be operatively linked to at least one of the FKBD, the Effector Domain, the Linking Region A, or the Linking Region Z.

In some embodiments, provided herein is a tagged macrocyclic compound of Formula (III) or a stereoisomer, pharmaceutically acceptable salt, or solvate thereof:

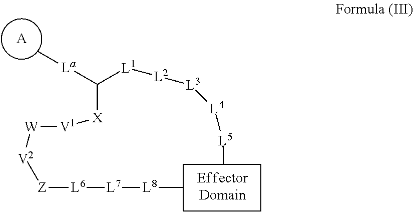

In some embodiments, Ring A is a 5-10 membered aryl, cycloalkyl, heteroaryl or heterocycloalkyl, optionally substituted with 1-17 substituents, each of which is independently selected from the group consisting of hydrogen, hydroxy, halo, alkyl, alkoxy, cyano, haloalkyl, haloalkoxy, alkylthio, oxo, amino, alkylamino, dialkylamino,

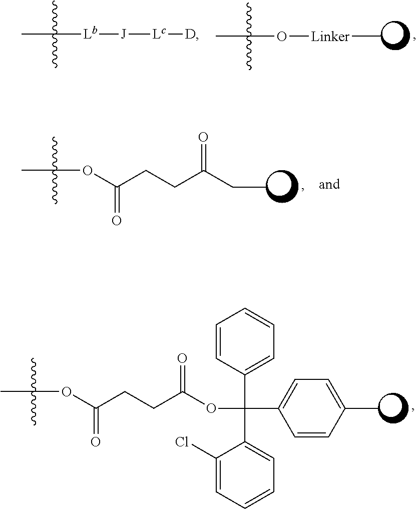

wherein [symbol] is a resin; J is independently at each occurrence selected from the group consisting of —C(O)NR⁶—.

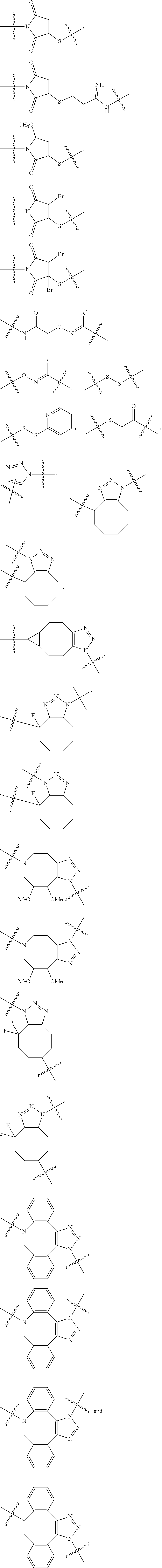

wherein R⁶ is each hydrogen, alkyl, arylalkyl,

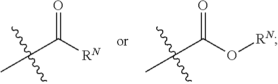

wherein R^{N} is aryl, alkyl, or arylalkyl; R′ is hydrogen, alkyl, arylalkyl, or haloalkyl; D is independently at each occurrence an oligonucleotide; L^{b} and L^{c} are independently at each occurrence selected from the group consisting of bond, —O—, —S—, —OC(O)—, —C(O)O—, —(CH₂)ₙC(O)—, —(CH₂)ₙC(O)C(O)—, —(CH₂)ₙNR⁵C(O)C(O)—, —NR⁵(CH₂)ₙC(O)C(O)—, optionally substituted (CH₂)ₙC₁₋₆ alkylene (CH₂)ₙ—, optionally substituted (CH₂)ₙC(O)C₁₋₆ alkylene (CH₂)ₙ—, optionally substituted (CH₂)ₙNR⁵C₁₋₆ alkylene (CH₂)ₙ—, optionally substituted (CH₂)ₙC(O)NR⁵C₁₋₆ alkylene (CH₂)ₙ—, optionally substituted (CH₂)ₙNR⁵C(O)C₁₋₆ alkylene (CH₂)ₙ—, optionally substituted (CH₂)ₙC(O)OC₁₋₆ alkylene (CH₂)ₙ—, optionally substituted (CH₂)ₙOC(O)C₁₋₆ alkylene (CH₂)ₙ—, optionally substituted (CH₂)ₙOC₁₋₆ alkylene (CH₂)ₙ—, optionally substituted (CH₂)ₙNR⁵C₁₋₆ alkylene (CH₂)ₙ—, optionally substituted (CH₂)ₙ—S—C₁₋₆ alkylene (CH₂)ₙ—, and optionally substituted (CH₂CH₂O)ₙ; wherein each alkylene is optionally substituted with 1 or 2 groups independently selected from the group consisting of of halo, hydroxy, haloalkyl, haloalkoxy, alkyl, alkoxy, amino, carboxyl, cyano, nitro, NHFmoc; wherein each R⁵ is independently hydrogen, alkyl, arylalkyl,

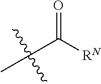

or and

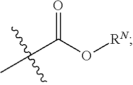

wherein R^{N} is aryl, alkyl, or arylalkyl; X is O, S or NR⁸, wherein R⁸ is hydrogen, hydroxy, OR⁹, NR¹⁰R¹¹, alkyl, arylalkyl,

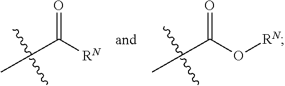

wherein R^{N} is aryl, alkyl, or arylalkyl; wherein R⁹, R¹⁰ and R¹¹ are each independently hydrogen or alkyl; V¹ and V² are each independently

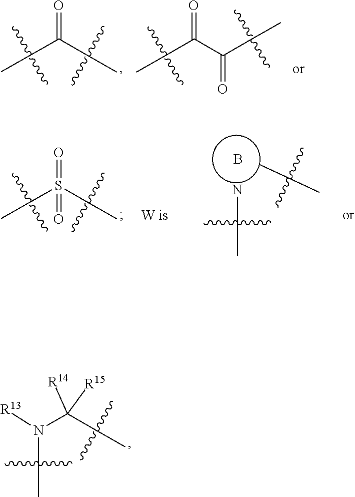

wherein Ring B is a 4-10 membered heterocycloalkyl, optionally substituted with 1-10 substituents, each of which is selected from the group consisting of hydrogen, hydroxy, halo, alkyl, alkoxy, cyano, haloalkyl, haloalkoxy, alkylthio, oxo, amino, alkylamino, dialkylamino, arylalkyl,

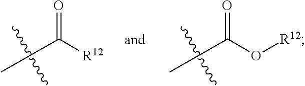

wherein R¹² is aryl, alkyl, or arylalkyl; wherein R¹³ is hydrogen, hydroxy, OR¹⁶, NR¹⁷R¹⁸, alkyl, arylalkyl,

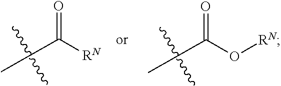

wherein R^{N} is aryl, alkyl, or arylalkyl; R¹⁴ and R¹⁵ is each independently hydrogen, hydroxy, halo, alkyl, alkoxy, haloalkyl, haloalkoxy, aryl, arylalkyl, or heteroaryl; Z is bond,

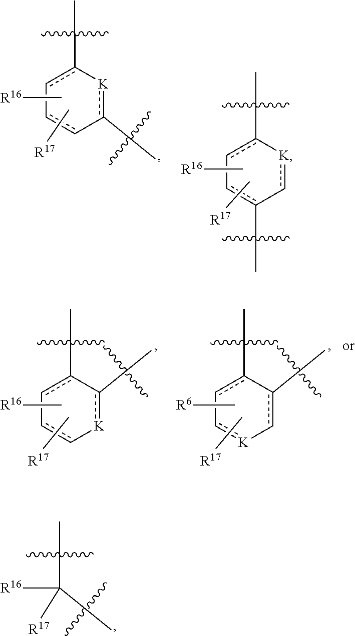

wherein R¹⁶ and R¹⁷ are each independently selected from the group consisting of of hydrogen, hydroxy, halo, alkyl, alkoxy, cycloalkyl, cyano, alkylthio, amino, alkylamino, and dialkylamino; K is O, CHR¹⁸, CR¹⁸, N, or and NR¹⁸, wherein R⁸ is hydrogen or alkyl;

L^{a}, L¹, L², L³, L⁴, L⁵, L⁶, L⁷ and L⁸ are each independently a bond, —O—, —NR¹⁹—, —SO—, —SO₂—, (CH₂)ₙ—,

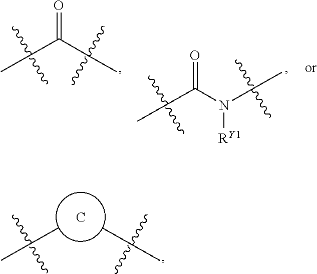

or a linking group selected from Table 1; wherein Ring C is a 5-6 membered heteroaryl, optionally substituted with 1-4 substituents, each of which is independently selected from the group consisting of hydrogen, hydroxyl, halo, alkyl, alkoxy, haloalkyl, haloalkoxy, cyano, alkylthio, amino, alkylamino, dialkylamino and

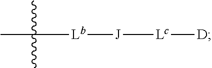

wherein each R¹⁹, R²⁰, and R²¹ is independently is selected from the group consisting of hydrogen, hydroxy, OR²², NR²³R²⁴, alkyl, arylalkyl,

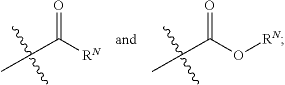

wherein R^{N} is aryl, alkyl, or arylalkyl; wherein R²², R²³, and R²⁴ are each independently hydrogen or alkyl;

n is 0, 1, 2, 3, 4, 5 or 6; wherein the Effector Domain has Formula (IIIa):

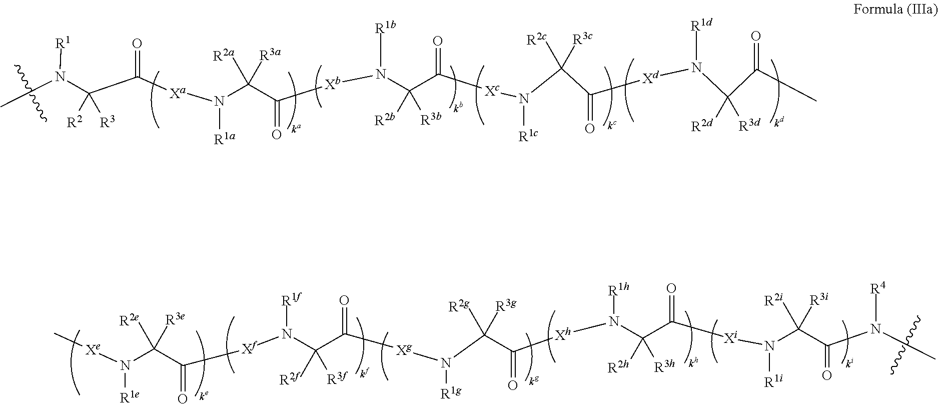

In some embodiments, each k^{a}, k^{b}, k^{c}, k^{d}, k^{e}, k^{f}, k^{g}, k^{h}, and kⁱ is independently 0 or 1; each X^{a}, X^{b}, X^{c}, X^{d}, X^{e}, X^{f}, X^{g}, X^{h}, and Xⁱ is independently a bond, —S—, —S—S—, —S(O)—, —S(O)₂—, substituted or substituted —(C₁-C₃)alkylene-, —(C₂-C₄) alkenylene-, —(C₂-C₄) alkynylene-, or

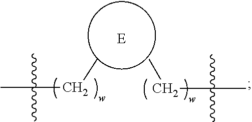

wherein Ring E is phenyl or a 5-6 heteroaryl or heterocycloalkyl; wherein each w is independently 0, 1, or 2; each R¹, R^{1a}, R^{1b}, R^{1c}, R^{1d}, R^{1e}, R^{1f}, R^{1g}, R^{1h}, R¹ⁱ, and R⁴ is independently hydrogen, alkyl, arylalkyl or NR²⁵, wherein R²⁵ is hydrogen, hydroxy, OR²⁶, NR²⁷R²⁸, alkyl, arylalkyl,

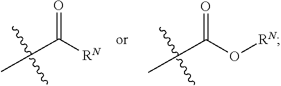

wherein R^{N} is aryl, alkyl, or arylalkyl; wherein R²⁶, R²⁷, and R²⁸ are each independently hydrogen or alkyl; each R², R³, R^{2a}, R^{3a}, R^{2b}, R^{3b}, R^{2c}, R^{3c}, R^{2d}, R^{3d}, R^{2e}, R^{3e}, R^{2f}, R^{3f}, R^{2g}, R^{3g}, R^{2h} R^{3h}, R²ⁱ, and R³ⁱ is independently selected from the group consisting of hydrogen, halo, amino, cyano, nitro, haloalkyl, optionally substituted alkyl, optionally substituted alkoxy, optionally substituted alkenyl, optionally substituted alkynyl, optionally substituted cycloalkyl, optionally substituted heterocycloalkyl, optionally substituted alkylamino, optionally substituted dialkylamino, optionally substituted aryl, optionally substituted heteroaryl, optionally substituted arylalkyl, optionally substituted heteroarylalkyl and

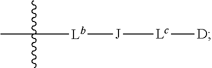

or wherein the Effector Domain has Formula (IIIb):

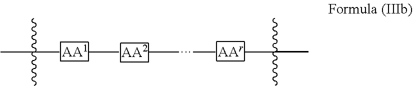

wherein each of AA¹, AA², . . . , and AA^{r} is an natural or unnatural amino acid residue; and r is 3, 4,5,6,7,8,9, or 10;

or wherein the Effector Domain has Formula (Ic):

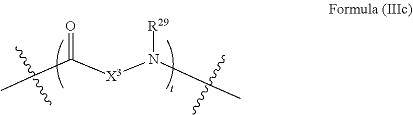

wherein each t is independently an integer selected from 0, 1, 2, 3, 4, 5, 6, 7, 8, 9, and 10; R²⁹ is a hydrogen, hydroxy, OR³⁰, NR³¹R³², alkyl, arylalkyl,

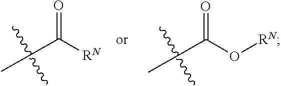

wherein R^{N} is aryl, alkyl, or arylalkyl; wherein R³⁰, R³¹, and R³² are each independently hydrogen or alkyl; X³ is substituted or unsubstituted —(C₁-C₆) alkylene-, —(C₂-C₆) alkenylene-, —(C₂-C₆) alkynylene-, or

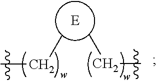

wherein Ring E is phenyl or a 5-6 heteroaryl or heterocycloalkyl; wherein each w is independently 0, 1, or 2;

or wherein the Effector Domain has Formula (IIId):

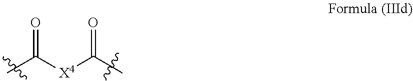

wherein X⁴ is substituted or unsubstituted —(C₁-C₆) alkylene-, —(C₂-C₆) alkenylene-, —(C₂-C₆) alkynylene-, or

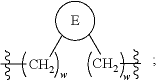

wherein Ring E is phenyl or a 5-6 heteroaryl or heterocycloalkyl; wherein each w is independently 0, 1, or 2;

or wherein the Effector Domain has Formula (IIIe):

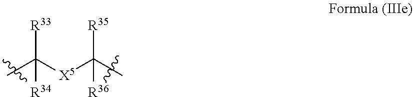

wherein R³³, R³⁴, R³⁵ and R³⁶ are each hydrogen or alkyl; X⁵ is substituted or unsubstituted —(C₁-C₆) alkylene-, —(C₂-C₆) alkenylene-, —(C₂-C₆) alkynylene-, or

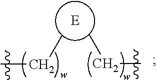

wherein Ring E is phenyl or a 5-6 heteroaryl or heterocycloalkyl; wherein each w is independently 0, 1, or 2;

or wherein the Effector Domain has Formula (IIIf):

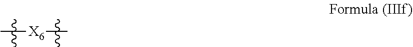

X⁶ is substituted or unsubstituted —(C₁-C₆) alkylene-, —(C₂-C₆) alkenylene-, —(C₂-C₆) alkynylene-, or

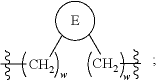

wherein Ring E is phenyl or a 5-6 heteroaryl or heterocycloalkyl; wherein each w is independently 0, 1, or 2; provided that when R is

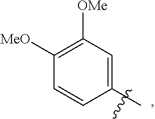

L is ethylene, X is O, W is

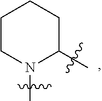

### V is

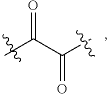

### Z is

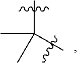

—L⁶—L⁷—L⁸— is

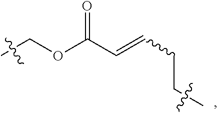

then—L¹—L²—L³—L⁴—L⁵— is not

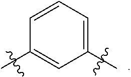

and; wherein Ring A is substituted with at least one

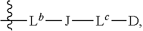

or at least one of R² R³ R^{2a}, R^{3a}, R^{2b}, R^{3b}, R^{2c}, R^{3c}, R^{2d}, R^{3d}, R^{2e}, R^{3e}, R^{2f}, R^{3f}, R^{2g}, R^{3g}, R^{2h}, R^{3h}, R²ⁱ, and R³ⁱ is

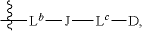

or at least one of L^{a}, L¹, L², L³, L⁴, L⁵, L⁶, L⁷ and L⁸ is Ring C substituted with at least one

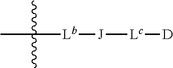

or wherein at least one of the linking groups selected from Table 1 is substituted with at least one

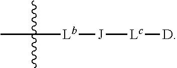

TABLE 1
The linker structures.

| | | | |
|---|---|---|---|
| optionally substituted | optionally substituted | optionally substituted | optionally substituted |
| —(CH₂)ₙC₁₋₆ alkylene | —(CH₂)ₙC₂₋₆ alkenylene | —(CH₂)ₙC₃₋₆ cycloalkylene | —(CH₂)ₙC₃₋₆ cycloalkenylene |
| optionally substituted | optionally substituted | optionally substituted | optionally substituted |
| —(CH₂)ₙOC₁₋₆ alkylene | —(CH₂)ₙOC₂₋₆ | —(CH₂)ₙOC₃₋₆ | —(CH₂)ₙOC₃₋₆ |
| | alkenylene | cycloalkylene | cycloalkenylene |
| optionally substituted | optionally substituted | optionally substituted | optionally substituted |
| —(CH₂)ₙC(O)C₁₋₆ | —(CH₂)ₙC(O)C₂₋₆ | —(CH₂)ₙC(O)C₃₋₆ | —(CH₂)ₙC(O)C₃₋₆ |
| alkylene | alkenylene | cycloalkylene | cycloalkenylene |
| optionally substituted | optionally substituted | optionally substituted | optionally substituted |
| —(CH₂)ₙC(O)OC₁₋₆ | —(CH₂)ₙC(O)OC₂.₆ | —(CH₂)ₙC(O)OC₃.₆ | —(CH₂)ₙC(O)OC₃.₆ |
| alkylene | alkenylene | C₃-6 cycloalkylene | cycloalkenylene |
| optionally substituted | optionally substituted | optionally substituted | optionally substituted |
| —(CH₂)ₙOC(O)C₁₋₆ | —(CH₂)ₙOC(O)C₂₋₆ | —(CH₂)ₙOC(O)C₃₋₆ | —(CH₂)ₙOC(O)C₃₋₆ |
| alkylene | alkenylene | cycloalkylene | cycloalkenylene |
| optionally substituted | optionally substituted | optionally substituted | optionally substituted |
| —(CH₂)ₙNR²⁰C₁₋₆ | —(CH₂)ₙNR²⁰C₂₋₆ | —(CH₂)ₙNR²⁰C₃₋₆ | —(CH₂)ₙNR²⁰C₃₋₆ |
| alkylene | alkenylene | cycloalkylene | cycloalkenylene |
| optionally substituted | optionally substituted | optionally substituted | optionally substituted |
| —(CH₂)ₙNR²⁰C(O)C₁₋₆ | —(CH₂)ₙNR²⁰C(O)C₂₋₆ | —(CH₂)ₙNR²⁰C(O)C₃₋₆ | —(CH₂)ₙNR²⁰C(O)C₃₋₆ |
| alkylene | alkenylene | cycloalkylene | C3-6 cycloalkenylene |
| optionally substituted | optionally substituted | optionally substituted | optionally substituted |
| —(CH₂)ₙC(O)NR²⁰C₁₋₆ | —(CH₂)ₙC(O)NR²⁰C₂₋₆ | —(CH₂)ₙC(O)NR²⁰C₃₋₆ | —(CH₂)ₙC(O)NR²⁰C₃₋₆ |
| alkylene | alkenylene | cycloalkylene | cycloalkenylene |
| optionally substituted | optionally substituted | optionally substituted | optionally substituted |
| —(CH₂)ₙ—S—C₁₋₆ | —(CH₂)ₙ—S—C₂₋₆ | —(CH₂)ₙ—S—C₃₋₆ | —(CH₂)ₙ—S—C₃₋₆ |
| alkylene | alkenylene | cycloalkylene | cycloalkenylene |
| optionally substituted | optionally substituted | optionally substituted | optionally substituted |
| —(CH₂)ₙC(O)(CH₂)ₙ—S— | —(CH₂)ₙC(O)(CH₂)ₙ—S— | —(CH₂)ₙC(O)(CH₂)ₙ—S— | —(CH₂)ₙC(O)(CH₂)ₙ—S— |
| C₁₋₆ alkylene | C₂₋₆ alkenylene | C₃₋₆ cycloalkylene | C₃₋₆ cycloalkenylene |
| optionally substituted | optionally substituted | optionally substituted | optionally substituted |
| —(CH₂)ₙ—SO₂—C₁₋₆ | —(CH₂)ₙ—SO₂—C₂₋₆ | —(CH₂)ₙ—SO₂—C₃₋₆ | —(CH₂)ₙ—SO₂—C₃₋₆ |
| alkylene | alkenylene | cycloalkylene | cycloalkenylene |
| optionally substituted | optionally substituted | optionally substituted | optionally substituted |
| —(CH₂)ₙC(O)(CH₂)ₙ— | —(CH₂)ₙC(O)(CH₂)ₙ— | —(CH₂)ₙC(O)(CH₂)ₙ— | —(CH₂)ₙC(O)(CH₂)ₙ— |
| SO₂—C₁₋₆ | SO₂—C₂₋₆ | SO₂—C₃₋₆ cycloalkylene | SO₂—C₃₋₆ cycloalkenylene |
| alkylene | alkenylene | | |
| optionally substituted | optionally substituted | optionally substituted | optionally substituted |
| —(CH₂)ₙ—SO—C₁₋₆ | —(CH₂)ₙ—SO—C₂₋₆ | —(CH₂)ₙ—SO—C₃₋₆ | —(CH₂)ₙ—SO—C₃₋₆ |
| alkylene | alkenylene | cycloalkylene | cycloalkenylene |
| optionally substituted | optionally substituted | optionally substituted | optionally substituted |
| —(CH₂)ₙC(O)(CH₂)ₙ— | —(CH₂)ₙC(O)(CH₂)ₙ— | —(CH₂)ₙC(O)(CH₂)ₙ—SO— | —(CH₂)ₙC(O)(CH₂)ₙ—SO— |
| SO—C₁₋₆ alkylene | SO—C₂₋₆ alkenylene | C₃₋₆ cycloalkylene | C₃₋₆ cycloalkenylene |
| optionally substituted | optionally substituted | optionally substituted | optionally substituted |
| —(CH₂)ₙ—S—S—C₁₋₆ | —(CH₂)ₙ—S—S—C₂₋₆ | —(CH₂)ₙ—S—S—C₃₋₆ | —(CH₂)ₙ—S—S—C₃₋₆ |
| alkylene | alkenylene | cycloalkylene | cycloalkenylene |
| optionally substituted | optionally substituted | optionally substituted | optionally substituted |
| —(CH₂)ₙC(O)(CH₂)ₙ—S— | —(CH₂)ₙC(O)(CH₂)ₙ—S— | —(CH₂)ₙC(O)(CH₂)ₙ—S— | —(CH₂)ₙC(O)(CH₂)ₙ—S— |
| S—C₁₋₆ | S—C₂₋₆ | S—C₃₋₆ cycloalkylene | S—C₃₋₆ cycloalkenylene |
| alkylene | alkenylene | | |
| optionally substituted | optionally substituted | optionally substituted | optionally substituted |
| —(CH₂)ₙC₁₋₆ alkylene- | —(CH₂)ₙC₂₋₆ alkenylene- | —(CH₂)ₙC₃₋₆ cycloalkylene- | —(CH₂)ₙC₃₋₆ cycloalkenylene- |
| NR²¹— | NR²¹— | NR²¹— | NR²¹— |
| optionally substituted | optionally substituted | optionally substituted | optionally substituted |
| —(CH₂)ₙOC₁₋₆ alkylene- | —(CH₂)ₙOC₂₋₆ | —(CH₂)ₙOC₃₋₆ | —(CH₂)ₙOC₃₋₆ |
| NR²¹— | alkenylene-NR²¹— | cycloalkylene-NR²¹— | cycloalkenylene-NR²¹— |
| optionally substituted | optionally substituted | optionally substituted | optionally substituted |
| —(CH₂)ₙC(O)C₁₋₆ | —(CH₂)ₙC(O)C₂₋₆ | —(CH₂)ₙC(O)C₃₋₆ | —(CH₂)ₙC(O)C₃₋₆ |
| alkylene-NR²¹— | alkenylene-NR²¹— | cycloalky lene-NR²¹— | cycloalkenylene-NR²¹— |
| optionally substituted | optionally substituted | optionally substituted | optionally substituted |
| —(CH₂)ₙC(O)OC₁₋₆ | —(CH₂)ₙC(O)OC₂.₆ | —(CH₂)ₙC(O)OC₃.₆ | —(CH₂)ₙC(O)OC₃.₆ |
| alkylene-NR²¹— | alkenylene-NR²¹— | cycloalkylene-NR²¹— | cycloalkenylene-NR²¹— |
| optionally substituted | optionally substituted | optionally substituted | optionally substituted |
| —(CH₂)ₙOC(O)C₁₋₆ | —(CH₂)ₙOC(O)C₂.₆ | —(CH₂)ₙOC(O)C₃.₆ | —(CH₂)ₙOC(O)C₃.₆ |
| alkylene-NR²¹— | alkenylene-NR²¹— | cycloalkylene-NR²¹— | cycloalkenylene-NR²¹— |
| optionally substituted | optionally substituted | optionally substituted | optionally substituted |
| —(CH₂)ₙNR²⁰C₁₋₆ | —(CH₂)ₙNR²⁰C₂.₆ | —(CH₂)ₙNR²⁰C₃.₆ | —(CH₂)ₙNR²⁰C₃.₆ |
| alkylene-NR²¹— | alkenylene-NR²¹— | cycloalkylene-NR²¹— | cycloalkenylene-NR²¹— |
| optionally substituted | optionally substituted | optionally substituted | optionally substituted |
| —(CH₂)ₙNR²⁰C(O)C₁₋₆ | —(CH₂)ₙNR²⁰C(O)C₂₋₆ | —(CH₂)ₙNR²⁰C(O)C₃₋₆ | —(CH₂)ₙNR²⁰C(O)C₃₋₆ |
| alkylene-NR²¹— | alkenylene-NR²¹— | cycloalkylene-NR²¹— | cycloalkenylene-NR²¹— |
| optionally substituted | optionally substituted | optionally substituted | optionally substituted |
| —(CH₂)ₙC(O)NR²⁰C₁₋₆ | —(CH₂)ₙC(O)NR²⁰C₁₋₆ | —(CH₂)ₙC(O)NR²⁰C₃₋₆ | —(CH₂)ₙC(O)NR²⁰C₃₋₆ |
| alkylene-NR²¹— | alkenylene-NR²¹— | cycloalkylene-NR²¹— | cycloalkenylene-NR²¹— |
| optionally substituted | optionally substituted | optionally substituted | optionally substituted |
| —(CH₂)ₙ—S—C₁₋₆ alkylene- | —(CH₂)ₙ—S—C₂₋₆ | —(CH₂)ₙ—S—C₃₋₆ | —(CH₂)ₙ—S—C₃₋₆ |
| NR²¹— | alkenylene | cycloalkylene-NR²¹— | cycloalkenylene-NR²¹— |
| optionally substituted | optionally substituted | optionally substituted | optionally substituted |
| —(CH₂)ₙC(O)(CH₂)ₙ—S— | —(CH₂)ₙC(O)(CH₂)ₙ—S— | —(CH₂)ₙC(O)(CH₂)ₙ—S— | —(CH₂)ₙC(O)(CH₂)ₙ—S— |
| C₁₋₆ alkylene-NR²¹— | C₂₋₆ alkenylene-NR²¹— | C₃₋₆ cycloalkylene-NR²¹— | C₃₋₆ cycloalkenylene-NR²¹— |
| optionally substituted | optionally substituted | optionally substituted | optionally substituted |
| —(CH₂)ₙ—SO₂—C₁₋₆ | —(CH₂)ₙ—SO₂—C₂₋₆ | —(CH₂)ₙ—SO₂—C₃₋₆ | —(CH₂)ₙ—SO₂—C₃₋₆ |
| alkylene-NR²¹— | alkenylene-NR²¹— | cycloalkylene-NR²¹— | cycloalkenylene-NR²¹— |
| optionally substituted | optionally substituted | optionally substituted | optionally substituted |
| —(CH₂)ₙC(O)(CH₂)ₙ— | —(CH₂)ₙC(O)(CH₂)ₙ— | —(CH₂)ₙC(O)(CH₂)ₙ— | —(CH₂)ₙC(O)(CH₂)ₙ— |
| SO₂—C₁₋₆ | SO₂— | SO₂—C₃₋₆ | SO₂—C₃₋₆ |
| alkylene-NR²¹— | C₂₋₆ alkenylene-NR²¹— | cycloalkylene-NR²¹— | cycloalkenylene-NR²¹— |
| optionally substituted | optionally substituted | optionally substituted | optionally substituted |
| —(CH₂)ₙ—SO—C₁₋₆ | —(CH₂)ₙ—SO—C₁₋₆ | —(CH₂)ₙ—SO—C₃₋₆ | —(CH₂)ₙ—SO—C₃₋₆ |
| alkylene-NR²¹— | alkenylene-NR²¹— | cycloalkylene-NR²¹— | cycloalkenylene-NR²¹— |
| optionally substituted | optionally substituted | optionally substituted | optionally substituted |
| —(CH₂)ₙC(O)(CH₂)ₙ— | —(CH₂)ₙC(O)(CH₂)ₙ— | —(CH₂)ₙC(O)(CH₂)ₙ—SO— | —(CH₂)ₙC(O)(CH₂)ₙ—SO— |
| SO—C₁₋₆ | SO—C₂₋₆ | C₃₋₆ cycloalkylene-NR²¹— | C₃₋₆ cycloalkenylene-NR²¹— |
| alkylene-NR²¹— | alkenylene-NR²¹— | | |
| optionally substituted | optionally substituted | optionally substituted | optionally substituted |
| —(CH₂)ₙ—S—S—C₁₋₆ | —(CH₂)ₙ—S—S—C₂₋₆ | —(CH₂)ₙ—S—S—C₃₋₆ | —(CH₂)ₙ—S—S—C₃₋₆ |
| alkylene-NR²¹— | alkenylene-NR²¹— | C3-6 cycloalkylene-NR²¹— | cycloalkenylene-NR²¹— |
| optionally substituted | optionally substituted | optionally substituted | optionally substituted |
| —(CH₂)ₙC(O)(CH₂)ₙ—S— | —(CH₂)ₙC(O)(CH₂)ₙ—S— | —(CH₂)ₙC(O)(CH₂)ₙ—S— | —(CH₂)ₙC(O)(CH₂)ₙ—S— |
| S—C₁₋₆ | S—C₂₋₆ | S—C₃₋₆ | S—C₃₋₆ |
| alkylene-NR²¹— | alkenylene-NR²¹— | cycloalkylene-NR²¹— | cycloalkenylene-NR²¹— |
| optionally substituted | optionally substituted | optionally substituted | optionally substituted |
| —(CH₂)ₙC₁₋₆ alkylene- | —(CH₂)ₙC₂₋₆ alkenylene- | —(CH₂)ₙC₃₋₆ cycloalkylene- | —(CH₂)ₙC₃₋₆ cycloalkenylene- |
| C(O)— | C(O)— | C(O)— | C(O)— |
| optionally substituted | optionally substituted | optionally substituted | optionally substituted |
| —(CH₂)ₙOC₁₋₆ alkylene- | —(CH₂)ₙOC₂₋₆ | —(CH₂)ₙOC₃₋₆ | —(CH₂)ₙOC₃₋₆ |
| C(O)— | alkenylene-C(O)— | cycloalkylene-C(O)— | cycloalkenylene-C(O)— |
| optionally substituted | optionally substituted | optionally substituted | optionally substituted |
| —(CH₂)ₙC(O)C₁₋₆ | —(CH₂)ₙC(O)C₂₋₆ | —(CH₂)ₙC(O)C₃₋₆ | —(CH₂)ₙC(O)C₃₋₆ |
| alkylene-C(O)— | alkenylene-C(O)— | cycloalkylene-C(O)— | cycloalkenylene-C(O)— |
| optionally substituted | optionally substituted | optionally substituted | optionally substituted |
| —(CH₂)ₙC(O)OC₁₋₆ | —(CH₂)ₙC(O)OC₂₋₆ | —(CH₂)ₙC(O)OC₃₋₆ | —(CH₂)ₙC(O)OC₃₋₆ |
| alkylene-C(O)— | alkenylene-C(O)— | cycloalkylene-C(O)— | cycloalkenylene-C(O)— |
| optionally substituted | optionally substituted | optionally substituted | optionally substituted |
| —(CH₂)ₙOC(O)C₁₋₆ | —(CH₂)ₙOC(O)C₂₋₆ | —(CH₂)ₙOC(O)C₃₋₆ | —(CH₂)ₙOC(O)C₃₋₆ |
| alkylene-C(O)— | alkenylene-C(O)— | cycloalkylene-C(O)— | cycloalkenylene-C(O)— |
| optionally substituted | optionally substituted | optionally substituted | optionally substituted |
| —(CH₂)ₙNR²⁰C₁₋₆ | —(CH₂)ₙNR²⁰C₂.₆ | —(CH₂)ₙNR²⁰C₃.₆ | —(CH₂)ₙNR²⁰C₃.₆ |
| alkylene-C(O)— | alkenylene-C(O)— | cycloalkylene-C(O)— | cycloalkenylene-C(O)— |
| optionally substituted | optionally substituted | optionally substituted | optionally substituted |
| —(CH₂)ₙNR²⁰C(O)C₁₋₆ | —(CH₂)ₙNR²⁰C(O)C₂₋₆ | —(CH₂)ₙNR²⁰C(O)C₃₋₆ | —(CH₂)ₙNR²⁰C(O)C₃₋₆ |
| alkylene-C(O)— | alkenylene-C(O)— | cycloalkylene-C(O)— | cycloalkenylene-C(O)— |
| optionally substituted | optionally substituted | optionally substituted | optionally substituted |
| —(CH₂)ₙNR²⁰C₁₋₆ | —(CH₂)ₙNR²⁰C₂.₆ | —(CH₂)ₙNR²⁰C₃.₆ | —(CH₂)ₙNR²⁰C₃.₆ |
| alkylene-C(O)— | alkenylene-C(O)— | cycloalkylene-C(O)— | cycloalkenylene-C(O)— |
| optionally substituted | optionally substituted | optionally substituted | optionally substituted |
| —(CH₂)ₙC(O)NR²⁰C₁₋₆ | —(CH₂)ₙC(O)NR²⁰C₂₋₆ | —(CH₂)ₙC(O)NR²⁰C₃₋₆ | —(CH₂)ₙC(O)NR²⁰C₋₆ |
| alkylene-C(O)— | alkenylene-C(O)— | cycloalkylene-C(O)— | cycloalkenylene-C(O)— |
| optionally substituted | optionally substituted | optionally substituted | optionally substituted |
| —(CH₂)ₙ—S—C₁₋₆ alkylene- | —(CH₂)ₙ—S—C₂₋₆ | —(CH₂)ₙ—S—C₃₋₆ | —(CH₂)ₙ—S—C₃₋₆ |
| C(O)— | alkenylene-C(O)— | cycloalkylene-C(O)— | cycloalkenylene-C(O)— |
| optionally substituted | optionally substituted | optionally substituted | optionally substituted |
| —(CH₂)ₙC(O)(CH₂)ₙ—S— | —(CH₂)ₙC(O)(CH₂)ₙ—S— | —(CH₂)ₙC(O)(CH₂)ₙ—S— | —(CH₂)ₙC(O)(CH₂)ₙ—S— |
| C₁₋₆ alkylene-C(O) | C₂₋₆ alkenylene-C(O) | C₃₋₆ cycloalkylene-C(O) | C₃₋₆ cycloalkenylene-C(O) |
| optionally substituted | optionally substituted | optionally substituted | optionally substituted |
| —(CH₂)ₙ—SO₂—C₁₋₆ | —(CH₂)ₙ—SO₂—C₂₋₆ | —(CH₂)ₙ—SO₂—C₃₋₆ | —(CH₂)ₙ—SO₂—C₃₋₆ |
| alkylene-C(O)— | alkenylene-C(O)— | cycloalkylene-C(O)— | cycloalkenylene-C(O)— |
| optionally substituted | optionally substituted | optionally substituted | optionally substituted |
| —(CH₂)ₙC(O)(CH₂)ₙ— | —(CH₂)ₙC(O)(CH₂)ₙ— | —(CH₂)ₙC(O)(CH₂)ₙ— | —(CH₂)ₙC(O)(CH₂)ₙ— |
| SO₂—C₁₋₆ | SO₂—C₂₋₆ | SO₂—C₃₋₆ | SO₂—C₃₋₆ |
| alkylene-C(O) | C₂-6 alkenylene-C(O) | cycloalkylene-C(O) | cycloalkenylene-C(O) |
| optionally substituted | optionally substituted | optionally substituted | optionally substituted |
| —(CH₂)ₙ—SO—C₁₋₆ | —(CH₂)ₙ—SO—C₂₋₆ | —(CH₂)ₙ—SO—C₃₋₆ | —(CH₂)ₙ—SO—C₃₋₆ |
| alkylene-C(O)— | alkenylene-C(O)— | cycloalkylene-C(O)— | cycloalkenylene-C(O)— |
| optionally substituted | optionally substituted | optionally substituted | optionally substituted |
| —(CH₂)ₙC(O)(CH₂)ₙ— | —(CH₂)ₙC(O)(CH₂)ₙ— | —(CH₂)ₙC(O)(CH₂)ₙ—SO— | —(CH₂)ₙC(O)(CH₂)ₙ—SO— |
| SO—C₁₋₆ alkylene-C(O) | SO—C₂₋₆ alkenylene-C(O) | C₃₋₆ cycloalkylene-C(O) | C₃₋₆ cycloalkenylene-C(O) |
| optionally substituted | optionally substituted | optionally substituted | optionally substituted |
| —(CH₂)ₙ—S—S—C₁₋₆ | —(CH₂)ₙ—S—S—C₂₋₆ | —(CH₂)ₙ—S—S—C₃₋₆ | —(CH₂)ₙ—S—S—C₃₋₆ |
| alkylene-C(O)— | alkenylene-C(O)— | cycloalkylene-C(O)— | cycloalkenylene-C(O)— |
| optionally substituted | optionally substituted | optionally substituted | optionally substituted |
| —(CH₂)ₙC(O)(CH₂)ₙ—S— | —(CH₂)ₙC(O)(CH₂)ₙ—S— | —(CH₂)ₙC(O)(CH₂)ₙ—S— | —(CH₂)ₙC(O)(CH₂)ₙ—S— |
| S—C₁₋₆ | S—C₂₋₆ | S—C₃₋₆ | S—C₃₋₆ |
| alkylene-C(O) | alkenylene-C(O) | cycloalkylene-C(O) | cycloalkenylene-C(O) |
| optionally substituted | optionally substituted | optionally substituted | optionally substituted |
| —NR²⁰C(O)(CH₂)ₙOC₁₋₆ | —NR²⁰C(O)(CH₂)ₙO— | —NR²⁰C(O)(CH₂)ₙO— | —NR²⁰C(O)(CH₂)ₙO— |
| alkylene-(CO) | C₂₋₆ alkenylene-(CO) | C₃₋₆ cycloalkylene-(CO) | C₃₋₆ cycloalkenylene-(CO) |
| optionally substituted | optionally substituted | optionally substituted | optionally substituted |
| —NR²⁰C(O)(CH₂)ₙ—S— | —NR²⁰C(O)(CH₂)ₙ—S— | —NR²⁰C(O)(CH₂)ₙ—S— | —NR²⁰C(O)(CH₂)ₙ—S— |
| C₁₋₆ alkylene-(CO) | C₂₋₆ alkenylene-(CO) | C₃₋₆ cycloalkylene-(CO) | C₃₋₆ cycloalkenylene-(CO) |
| optionally substituted | optionally substituted | optionally substituted | optionally substituted |
| —NR²⁰C(O)(CH₂)ₙNR²¹ | —NR²⁰C(O)(CH₂)ₙNR²¹ | —NR²⁰C(O)(CH₂)ₙNR²¹ | —NR²⁰C(O)(CH₂)ₙNR²¹ |
| C₁₋₆ alkylene-(CO) | C₂₋₆ alkenylene-(CO) | C₃₋₆ cycloalkylene-(CO) | C₃₋₆ cycloalkenylene-(CO) |
| optionally substituted | optionally substituted | optionally substituted | optionally substituted |
| C(O)NR²⁰(CH₂)ₙOC₁₋₆ | C(O)NR²⁰(CH₂)ₙOC₂₋₆ | C(O)NR²⁰(CH₂)ₙOC₃₋₆ | C(O)NR²⁰(CH₂)ₙOC₃₋₆ |
| alkylene-(CO) | alkenylene-(CO) | cycloalkylene-(CO) | cycloalkenylene-(CO) |
| optionally substituted | optionally substituted | optionally substituted | optionally substituted |
| —C(O)NR²⁰(CH₂)ₙ—S— | —C(O)NR²⁰(CH₂)ₙ—S— | —C(O)NR²⁰(CH₂)ₙ—S— | —C(O)NR²⁰(CH₂)ₙ—S— |
| C₁₋₆ alkylene-(CO) | C₂₋₆ alkenylene-(CO) | C₃₋₆ cycloalkylene-(CO) | C₃₋₆ cycloalkenylene-(CO) |
| optionally substituted | optionally substituted | optionally substituted | optionally substituted |
| —C(O)NR²⁰(CH₂)ₙ— | —C(O)NR²⁰(CH₂)ₙ— | —C(O)NR²⁰(CH₂)ₙ— | —C(O)NR²⁰(CH₂)ₙ— |
| NR²¹C₁₋₆ alkylene-(CO) | NR²¹C₂₋₆ alkenylene- | NR²¹C₃₋₆ cycloalkylene- | NR²¹C₃₋₆ cycloalkenylene- |
| | (CO) | (CO) | (CO) |
| optionally substituted | optionally substituted | optionally substituted | optionally substituted |
| —C(O)(CH₂)ₙC₁₋₆ | —C(O)(CH₂)ₙC₁₋₆ alkenyl- | —C(O)(CH₂)ₙC₃₋₆ | —C(O)(CH₂)ₙC₃₋₆ |
| alkylene-(CH₂)ₙ- | ene-(CH₂)ₙ— | cycloalkylene-(CH₂)ₙ— | cycloalkenylene-(CH₂)ₙ— |
| optionally substituted | optionally substituted | optionally substituted | optionally substituted |
| —C(O)O(CH₂)ₙC₁₋₆ | —C(O)O(CH₂)ₙC₁₋₆ | —C(O)O(CH₂)ₙC₃₋₆ | —C(O)O(CH₂)ₙC₃₋₆ |
| alkylene-(CH₂)ₙ— | alkenylene-(CH₂)ₙ— | cycloalkylene- | cycloalkenylene- |
| | | (CH₂)ₙ— | (CH₂)ₙ— |
| optionally substituted | optionally substituted | optionally substituted | optionally substituted |
| —C(O)(CH₂)ₙC₁₋₆ | —C(O)(CH₂)ₙC₁₋₆ alkenyl- | —C(O)(CH₂)ₙC₃₋₆ | —C(O)(CH₂)ₙC₃₋₆ |
| alkylene-(CH₂)ₙ—O— | ene- | cycloalkylene- | cycloalkenylene- |
| | (CH₂)ₙ—O— | (CH₂)ₙ—O— | (CH₂)ₙ—O— |
| optionally substituted | optionally substituted | optionally substituted | optionally substituted |
| —C(O)O(CH₂)ₙC₁₋₆ | —C(O)O(CH₂)ₙC₁₋₆ | —C(O)O(CH₂)ₙC₃₋₆ | —C(O)O(CH₂)ₙC₃₋₆ |
| alkylene-(CH₂)ₙ—O— | alkenylene- | cycloalkylene- | cycloalkenylene- |
| | (CH₂)ₙ—O— | (CH₂)ₙ—O— | (CH₂)ₙ—O— |
| optionally substituted | optionally substituted | optionally substituted | optionally substituted |
| —C(O)(CH₂)ₙC₁₋₆ | —C(O)(CH₂)ₙC₁₋₆ | —C(O)(CH₂)ₙC₃₋₆ | —C(O)(CH₂)ₙC₃₋₆ |
| alkylene- | alkenylene- | cycloalkylene- | cycloalkenylene- |
| (CH₂)ₙ—C(O)— | (CH₂)ₙ—C(O)— | (CH₂)ₙ—C(O)— | (CH₂)ₙ—C(O)— |
| optionally substituted | optionally substituted | optionally substituted | optionally substituted |
| —C(O)O(CH₂)ₙC₁₋₆ | —C(O)O(CH₂)ₙC₁₋₆ | —C(O)O(CH₂)ₙC₃₋₆ | —C(O)O(CH₂)ₙC₃₋₆ |
| alkylene- | alkenylene- | cycloalkylene- | cycloalkenylene- |
| —(CH₂)ₙ—C(O)— | (CH₂)ₙ—C(O)— | (CH₂)ₙ—C(O)— | (CH₂)ₙ—C(O)— |
| optionally substituted | optionally substituted | optionally substituted | optionally substituted |
| —OC(O)(CH₂)ₙC₁₋₆ | —OC(O)(CH₂)ₙC₁₋₆ | —OC(O)(CH₂)ₙC₃₋₆ | —OC(O)(CH₂)ₙC₃₋₆ |
| alkylene- | alkenylene- | cycloalkylene- | cycloalkenylene- |
| (CH₂)ₙ— | (CH₂)ₙ— | (CH₂)ₙ— | (CH₂)ₙ— |
| optionally substituted | optionally substituted | optionally substituted | optionally substituted |
| —O(CH₂)ₙC₁₋₆ alkylene- | —O(CH₂)ₙC₂₋₆ alkenylene | —O(CH₂)ₙC₃₋₆ cycloalkylene | —O(CH₂)ₙC₃₋₆ cycloalkenylene |
| (CH₂)ₙ— | (CH₂)ₙ— | (CH₂)ₙ— | (CH₂)ₙ— |
| optionally substituted | optionally substituted | optionally substituted | optionally substituted |
| —OC(O)(CH₂)ₙC₁₋₆ | —OC(O)(CH₂)ₙC₁₋₆ | —OC(O)(CH₂)ₙC₃₋₆ | —OC(O)(CH₂)ₙC₃₋₆ |
| alkylene- | alkenylene- | cycloalkylene- | cycloalkenylene- |
| (CH₂)ₙ—O— | (CH₂)ₙ—O— | (CH₂)ₙ—O— | (CH₂)ₙ—O— |
| optionally substituted | optionally substituted | optionally substituted | optionally substituted |
| —O(CH₂)ₙC₁₋₆ alkylene- | —O(CH₂)ₙC₁₋₆ alkenylene | —O(CH₂)ₙC₃₋₆ cycloalkylene | —O(CH₂)ₙC₃₋₆ cycloalkenylene |
| (CH₂)ₙ—O— | (CH₂)ₙ—O— | (CH₂)ₙ—O— | (CH₂)ₙ—O— |
| optionally substituted | optionally substituted | optionally substituted | optionally substituted |
| —OC(O)(CH₂)ₙC₁₋₆ | —OC(O)(CH₂)ₙC₁₋₆ | —OC(O)(CH₂)ₙC₃₋₆ | —OC(O)(CH₂)ₙC₃₋₆ |
| alkylene- | alkenylene- | cycloalkylene- | cycloalkenylene- |
| (CH₂)ₙ—C(O)— | (CH₂)ₙ—C(O)— | (CH₂)ₙ—C(O)— | (CH₂)ₙ—C(O)— |
| optionally substituted | optionally substituted | optionally substituted | optionally substituted |
| —O(CH₂)ₙC₁₋₆ alkylene- | —O(CH₂)ₙC₁₋₆ alkenylene | —O(CH₂)ₙC₃₋₆ cycloalkylene | —O(CH₂)ₙC₃₋₆ cycloalkenylene |
| (CH₂)ₙ—C(O)— | (CH₂)ₙ—C(O)— | (CH₂)ₙ—C(O)— | (CH₂)ₙ—C(O)— |
| optionally substituted | optionally substituted | optionally substituted | optionally substituted |
| —C(O)NR²⁰(CH₂)ₙ—C₁₋₆ | —C(O)NR²⁰(CH₂)ₙ—C₁₋₆ | —C(O)NR²⁰(CH₂)ₙ—C₃₋₆ | —C(O)NR²⁰(CH₂)ₙ—C₃₋₆ |
| alkylene-(CH₂)ₙ— | alkenylene-(CH₂)ₙ— | cycloalkylene-(CH₂)ₙ— | cycloalkenylene-(CH₂)ₙ— |
| optionally substituted | optionally substituted | optionally substituted | optionally substituted |
| NR²⁰C(O)(CH₂)ₙ—C₁₋₆ | —NR²⁰C(O)(CH₂)ₙ—C₁₋₆ | —NR²⁰C(O)(CH₂)ₙ—C₃₋₆ | —NR²⁰C(O)(CH₂)ₙ—C₃₋₆ |
| alkylene-(CH₂)ₙ— | alkenylene-(CH₂)ₙ— | cycloalkylene-(CH₂)ₙ— | cycloalkenylene- |
| | | | (CH₂)ₙ— |
| optionally substituted | optionally substituted | optionally substituted | optionally substituted |
| —C(O)NR²⁰(CH₂)ₙC₁₋₆ | —C(O)NR²⁰(CH₂)ₙC₁₋₆ | —C(O)NR²⁰(CH₂)ₙC₃₋₆ | —C(O)NR²⁰(CH₂)ₙC₃₋₆ |
| alkylene- | alkenylene- | cycloalkylene- | cycloalkenylene- |
| (CH₂)ₙ—O— | (CH₂)ₙ—O— | (CH₂)ₙ—O— | (CH₂)ₙ—O— |
| optionally substituted | optionally substituted | optionally substituted | optionally substituted |
| —NR²⁰C(O)(CH₂)ₙC₁₋₆ | —NR²⁰C(O)(CH₂)ₙC₁₋₆ | —NR²⁰C(O)(CH₂)ₙC₃₋₆ | —NR²⁰C(O)(CH₂)ₙC₁₋₆ |
| alkylene- | alkenylene- | cycloalkylene- | cycloalkenylene- |
| (CH₂)ₙ—O— | (CH₂)ₙ—O— | (CH₂)ₙ—O— | (CH₂)ₙ—O— |
| optionally substituted | optionally substituted | optionally substituted | optionally substituted |
| —C(O)NR²⁰(CH₂)ₙC₁₋₆ | —C(O)NR²⁰(CH₂)ₙC₁₋₆ | —C(O)NR²⁰(CH₂)ₙC₃₋₆ | —C(O)NR²⁰(CH₂)ₙC₃₋₆ |
| alkylene- | alkenylene- | cycloalkylene- | cycloalkenylene- |
| (CH₂)ₙ—C(O)— | (CH₂)ₙ—C(O)— | (CH₂)ₙ—O— | (CH₂)ₙ—O— |
| optionally substituted | optionally substituted | optionally substituted | optionally substituted |
| —NR²⁰C(O)(CH₂)ₙC₁₋₆ | —NR²⁰C(O)(CH₂)ₙC₁₋₆ | —NR²⁰C(O)(CH₂)ₙC₃₋₆ | —NR²⁰C(O)(CH₂)ₙC₃₋₆ |
| alkylene- | alkenylene- | cycloalkylene- | cycloalkenylene- |
| (CH₂)ₙ—C(O)— | (CH₂)ₙ—C(O)— | (CH₂)ₙ—O— | (CH₂)ₙ—O— |
| optionally substituted | optionally substituted | optionally substituted | |
| —(CH₂)ₙC₃₋₆ | —(CH₂)ₙC₃₋₆ | —(CH₂)ₙC₂₋₆ alkynylene | |
| heterocycloalkylene | heterocycloalkenylene | | |
| optionally substituted | optionally substituted | optionally substituted | |
| —(CH₂)ₙOC₃₋₆ | —(CH₂)ₙOC₃₋₆ | —(CH₂)ₙOC₂₋₆ alkynylene | |
| heterocycloalkylene | heterocycloalkenylene | | |
| optionally substituted | optionally substituted | optionally substituted | |
| —(CH₂)ₙC(O)C₃₋₆ | —(CH₂)ₙC(O)C₃₋₆ | —(CH₂)ₙC(O)C₂₋₆ | |
| heterocycloalkylene | heterocycloalkenylene | alkynylene | |
| optionally substituted | optionally substituted | optionally substituted | |
| —(CH₂)ₙC(O)OC₃₋₆ | —(CH₂)ₙC(O)OC₃₋₆ | —(CH₂)ₙC(O)OC₂₋₆ | |
| heterocycloalkylene | heterocycloalkenylene | alkynylene | |
| optionally substituted | optionally substituted | optionally substituted | |
| —(CH₂)ₙOC(O)C₃₋₆ | —(CH₂)ₙOC(O)C₃₋₆ | —(CH₂)ₙOC(O)C₂₋₆ | |
| heterocycloalkylene | heterocycloalkenylene | alkynylene | |
| optionally substituted | optionally substituted | optionally substituted | |
| —(CH₂)ₙNR²⁰C₃₋₆ | —(CH₂)ₙNR²⁰C₃₋₆ | —(CH₂)ₙNR²⁰C₂₋₆ | |
| heterocycloalkylene | heterocycloalkenylene | alkynylene | |
| optionally substituted | optionally substituted | optionally substituted | |
| —(CH₂)ₙNR²⁰C(O)—C₃₋₆ | —(CH₂)ₙNR²⁰C(O)—C₃₋₆ | —(CH₂)ₙNR²⁰C(O)—C₂₋₆ | |
| heterocycloalkylene | heterocycloalkenylene | alkynylene | |
| optionally substituted | optionally substituted | optionally substituted | |
| —(CH₂)ₙC(O)NR²⁰— | —(CH₂)ₙC(O)NR²⁰— | —(CH₂)ₙC(O)NR²⁰—C₂₋₆ | |
| optionally substituted | optionally substituted | alkynylene | |
| C₃₋₆ heterocycloalkylene | C₃₋₆ | | |
| | heterocycloalkenylene | | |
| optionally substituted | optionally substituted | optionally substituted | |
| —(CH₂)ₙ—S— | —(CH₂)ₙ—S— | —(CH₂)ₙ—S—C₂₋₆ | |
| C₃₋₆ heterocycloalkylene | C₃₋₆ | alkynylene | |
| | heterocycloalkenylene | | |
| optionally substituted | optionally substituted | optionally substituted | |
| —(CH₂)ₙC(O)(CH₂)ₙ—S— | —(CH₂)ₙC(O)(CH₂)ₙ—S— | —(CH₂)ₙC(O)(CH₂)ₙ—S— | |
| C₃₋₆ heterocycloalkylene | C₃₋₆ | C₂₋₆ alkynylene | |
| | heterocycloalkenylene | | |
| optionally substituted | optionally substituted | optionally substituted | |
| —(CH₂)ₙ—SO₂—C₃₋₆ | —(CH₂)ₙ—SO₂—C₃₋₆ | —(CH₂)ₙ—SO₂—C₂₋₆ | |
| heterocycloalkylene | heterocycloalkenylene | alkynylene | |
| optionally substituted | optionally substituted | optionally substituted | |
| —(CH₂)ₙC(O)(CH₂)ₙ— | —(CH₂)ₙC(O)(CH₂)ₙ— | —(CH₂)ₙC(O)(CH₂)ₙ— | |
| SO₂—C₃₋₆ | SO₂—C₃₋₆ | SO₂—C₂₋₆ alkynylene | |
| heterocycloalkylene | heterocycloalkenylene | | |
| optionally substituted | optionally substituted | optionally substituted | |
| —(CH₂)ₙ—SO—C₃₋₆ | —(CH₂)ₙ—SO—C₃₋₆ | —(CH₂)ₙ—SO—C₂₋₆ | |
| heterocycloalkylene | heterocycloalkenylene | alkynylene | |
| optionally substituted | optionally substituted | optionally substituted | |
| —(CH₂)ₙC(O)(CH₂)ₙ— | —(CH₂)ₙC(O)(CH₂)ₙ— | —(CH₂)ₙC(O)(CH₂)ₙ—SO— | |
| SO—C₃₋₆ | SO—C₃₋₆ | C₂₋₆ alkynylene | |
| heterocycloalkylene | heterocycloalkenylene | | |
| optionally substituted | optionally substituted | optionally substituted | |
| —(CH₂)ₙ—S—S—C₃₋₆ | —(CH₂)ₙ—S—S—C₃₋₆ | —(CH₂)ₙ—S—S—C₂₋₆ | |
| heterocycloalkylene | heterocycloalkenylene | alkynylene | |
| optionally substituted | optionally substituted | optionally substituted | |
| —(CH₂)ₙC(O)(CH₂)ₙ—S— | —(CH₂)ₙC(O)(CH₂)ₙ—S— | —(CH₂)ₙC(O)(CH₂)ₙ—S— | |
| S—C₃₋₆ | S—C₃₋₆ | S—C₂₋₆ alkynylene | |
| heterocycloalkylene | heterocycloalkenylene | | |
| optionally substituted | optionally substituted | optionally substituted | |
| —(CH₂)ₙC₃₋₆ | —(CH₂)ₙC₃₋₆ | —(CH₂)ₙC₂₋₆ alkynylene- | |
| heterocycloalkylene- | heterocycloalkenylene- | NR²¹— | |
| NR²¹— | NR²¹— | | |
| optionally substituted | optionally substituted | optionally substituted | |
| —(CH₂)ₙOC₃₋₆ | —(CH₂)ₙOC₃₋₆ | —(CH₂)ₙOC₂₋₆ alkynylene- | |
| heterocycloalkylene- | heterocycloalkenylene- | NR²¹— | |
| NR²¹— | NR²¹— | | |
| optionally substituted | optionally substituted | optionally substituted | |
| —(CH₂)ₙC(O)C₃₋₆ | —(CH₂)ₙC(O)C₃₋₆ | —(CH₂)ₙC(O)C₂₋₆ | |
| heterocycloalkylene- | heterocycloalkenylene- | alkynylene-NR²¹— | |
| NR²¹— | NR²¹— | | |
| optionally substituted | optionally substituted | optionally substituted | |
| —(CH₂)ₙC(O)O— | —(CH₂)ₙC(O)O— | —(CH₂)ₙC(O)O—C₂₋₆ | |
| C₃₋₆ | C₃₋₆ | alkynylene-NR²¹— | |
| heterocycloalkylene- | heterocycloalkenylene- | | |
| NR²¹— | NR²¹— | | |
| optionally substituted | optionally substituted | optionally substituted | |
| —(CH₂)ₙOC(O)— | —(CH₂)ₙOC(O)— | —(CH₂)ₙOC(O)—C₂₋₆ | |
| C₃₋₆ | C₃₋₆ | alkynylene-NR²¹— | |
| heterocycloalkylene- | heterocycloalkenylene- | | |
| NR²¹— | NR²¹— | | |
| optionally substituted | optionally substituted | optionally substituted | |
| —(CH₂)ₙNR²⁰C₃₋₆ | —(CH₂)ₙNR²⁰C₃₋₆ | —(CH₂)ₙNR²⁰C₂₋₆ | |
| heterocycloalkylene- | heterocycloalkenylene- | alkynylene-NR²¹— | |
| NR²¹— | NR²¹— | | |
| optionally substituted | optionally substituted | optionally substituted | |
| —(CH₂)ₙNR²⁰C(O)— | —(CH₂)ₙNR²⁰C(O)— | —(CH₂)ₙNR²⁰C(O)—C₂₋₆ | |
| C₃₋₆ | C₃₋₆ | alkynylene-NR²¹— | |
| heterocycloalkylene- | heterocycloalkenylene- | | |
| NR²¹— | NR²¹— | | |
| optionally substituted | optionally substituted | optionally substituted | |
| —(CH₂)ₙC(O)NR²⁰— | —(CH₂)ₙC(O)NR²⁰— | —(CH₂)ₙNR²⁰C(O)—C_{2.6} | |
| C₃₋₆ | C₃₋₆ | alkynylene-NR²¹— | |
| heterocycloalkylene- | heterocycloalkenylene- | | |
| NR²¹— | NR²¹— | | |
| optionally substituted | optionally substituted | optionally substituted | |
| —(CH₂)ₙ—S—C₃₋₆ | —(CH₂)ₙ—S—C₃₋₆ | —(CH₂)ₙ—S—C₃₋₆ | |
| heterocycloalkylene- | heterocycloalkenylene- | alkynylene | |
| NR²¹— | NR²¹— | | |
| optionally substituted | optionally substituted | optionally substituted | |
| —(CH₂)ₙC(O)(CH₂)ₙ—S— | —(CH₂)ₙC(O)(CH₂)ₙ—S— | —(CH₂)ₙC(O)(CH₂)ₙ—S— | |
| C₃₋₆ | C₃₋₆ | C₂₋₆ alkynylene-NR²¹— | |
| heterocycloalkylene- | heterocycloalkenylene- | | |
| NR²¹— | NR²¹— | | |
| optionally substituted | optionally substituted | optionally substituted | |
| —(CH₂)ₙ—SO₂—C₃₋₆ | —(CH₂)ₙ—SO₂—C₃₋₆ | —(CH₂)ₙ—SO₂—C₃₋₆ | |
| heterocycloalkylene- | heterocycloalkenylene- | alkynylene-NR²¹— | |
| NR²¹— | NR²¹— | | |
| optionally substituted | optionally substituted | optionally substituted | |
| —(CH₂)ₙC(O)(CH₂)ₙ | —(CH₂)ₙC(O)(CH₂)ₙ | —(CH₂)ₙC(O)(CH₂)ₙ | |
| SO₂—C₃₋₆ | SO₂—C₃₋₆ | SO₂—C₂₋₆ alkynylene- | |
| heterocycloalkylene- | heterocycloalkenylene- | NR²¹— | |
| NR²¹— | NR²¹— | | |
| optionally substituted | optionally substituted | optionally substituted | |
| —(CH₂)ₙ—SO—C₃₋₆ | —(CH₂)ₙ—SO—C₃₋₆ | —(CH₂)ₙ—SO—C₂₋₆ | |
| heterocycloalkylene- | heterocycloalkenylene- | alkynylene-NR²¹— | |
| NR²¹— | NR²¹— | | |
| optionally substituted | optionally substituted | optionally substituted | |
| —(CH₂)ₙC(O)(CH₂)ₙ— | —(CH₂)ₙC(O)(CH₂)ₙ— | —(CH₂)ₙC(O)(CH₂)ₙ—SO— | |
| SO—C₃.₆ | SO—C₃.₆ | C₂₋₆ alkynylene-NR²¹— | |
| heterocycloalkylene- | heterocycloalkenylene- | | |
| NR²¹— | NR²¹— | | |
| optionally substituted | optionally substituted | optionally substituted | |
| —(CH₂)ₙ—S—S—C₃₋₆ | —(CH₂)ₙ—S—S—C₃₋₆ | —(CH₂)ₙ—S—S—C₂₋₆ | |
| heterocycloalkylene- | heterocycloalkenylene- | alkynylene-NR²¹— | |
| NR²¹— | NR²¹— | | |
| optionally substituted | optionally substituted | optionally substituted | |
| —(CH₂)ₙC(O)(CH₂)ₙ—S— | —(CH₂)ₙC(O)(CH₂)ₙ—S— | —(CH₂)ₙC(O)(CH₂)ₙ—S— | |
| S—C₃₋₆ | S—C₃₋₆ | S—C₂₋₆ alkynylene-NR²¹— | |
| heterocycloalkylene- | heterocycloalkenylene- | | |
| NR²¹— | NR²¹— | | |
| optionally substituted | optionally substituted | optionally substituted | |
| —(CH₂)ₙC₃₋₆ | —(CH₂)ₙC₃₋₆ | —(CH₂)ₙC₂₋₆ alkynylene- | |
| heterocycloalkylene- | heterocycloalkenylene- | C(O)— | |
| C(O)— | C(O)— | | |
| optionally substituted | optionally substituted | optionally substituted | |
| —(CH₂)ₙOC₃₋₆ | —(CH₂)ₙOC₃₋₆ | —(CH₂)ₙOC₂₋₆ alkynylene- | |
| heterocycloalkylene- | heterocycloalkenylene- | C(O)— | |
| C(O)— | C(O)— | | |
| optionally substituted | optionally substituted | optionally substituted | |
| —(CH₂)ₙC(O)C₃₋₆ | —(CH₂)ₙC(O)C₃₋₆ | —(CH₂)ₙC(O)C₂₋₆ | |
| heterocycloalkylene- | heterocycloalkenylene- | alkynylene-C(O)— | |
| C(O)— | C(O)— | | |
| optionally substituted | optionally substituted | optionally substituted | |
| —(CH₂)ₙC(O)OC₃₋₆ | —(CH₂)ₙC(O)OC₃₋₆ | —(CH₂)ₙC(O)OC₂₋₆ | |
| heterocycloalkylene- | heterocycloalkenylene- | alkynylene-C(O)— | |
| C(O)— | C(O)— | | |
| optionally substituted | optionally substituted | optionally substituted | |
| —(CH₂)ₙOC(O)₃₋₆ | —(CH₂)ₙOC(O)₃₋₆ | —(CH₂)ₙOC(O)₂₋₆ | |
| heterocycloalkylene- | heterocycloalkenylene- | alkynylene-C(O)— | |
| C(O)— | C(O)— | | |
| optionally substituted | optionally substituted | optionally substituted | |
| —(CH₂)ₙNR²⁰C₃₋₆ | —(CH₂)ₙNR²⁰C₃₋₆ | —(CH₂)ₙNR²⁰C₂₋₆ | |
| heteroalkylene-C(O)— | heteroalkenylene- | alkynylene-C(O)— | |
| | C(O)— | | |
| optionally substituted | optionally substituted | optionally substituted | |
| —(CH₂)ₙNR²⁰C(O)C₃.₆ | —(CH₂)ₙNR²⁰C(O)C₃.₆ | —(CH₂)ₙNR²⁰C(O)C₂.₆ | |
| heterocycloalkylene- | heterocycloalkenylene- | alkynylene-C(O)— | |
| C(O)— | C(O)— | | |
| optionally substituted | optionally substituted | optionally substituted | |
| —(CH₂)ₙNR²⁰C₃₋₆ | —(CH₂)ₙNR²⁰C₃₋₆ | —(CH₂)ₙNR²⁰C₂₋₆ | |
| heterocycloalkylene- | heterocycloalkenylene- | alkynylene-C(O)— | |
| C(O)— | C(O)— | | |
| optionally substituted | optionally substituted | optionally substituted | |
| —(CH₂)ₙC(O)NR²⁰C₃₋₆ | —(CH₂)ₙC(O)NR²⁰C₃₋₆ | —(CH₂)ₙC(O)NR²⁰C₂₋₆ | |
| heterocycloalkylene- | heterocycloalkenylene- | alkynylene-C(O)— | |
| C(O)— | C(O)— | | |
| optionally substituted | optionally substituted | optionally substituted | |
| —(CH₂)ₙ—S—C₃₋₆ | —(CH₂)ₙ—S—C₃₋₆ | —(CH₂)ₙ—S—C₂₋₆ | |
| heterocycloalkylene- | heterocycloalkenylene- | alkynylene-C(O)— | |
| C(O)— | C(O)— | | |
| optionally substituted | optionally substituted | optionally substituted | |
| —(CH₂)ₙC(O)(CH₂)ₙ—S— | —(CH₂)ₙC(O)(CH₂)ₙ—S— | —(CH₂)ₙC(O)(CH₂)ₙ—S— | |
| C₃₋₆ | C₃₋₆ | C₂₋₆ alkynylene-C(O) | |
| heterocycloalkylene- | heterocycloalkenylene- | | |
| C(O) | C(O) | | |
| optionally substituted | optionally substituted | optionally substituted | |
| —(CH₂)ₙ—SO₂—C₃₋₆ | —(CH₂)ₙ—SO₂—C₃₋₆ | —(CH₂)ₙ—SO₂—C₂₋₆ | |
| heterocycloalkylene- | heterocycloalkenylene- | alkynylene-C(O)— | |
| C(O)— | C(O)— | | |
| optionally substituted | optionally substituted | optionally substituted | |
| —(CH₂)ₙC(O)(CH₂)ₙ— | —(CH₂)ₙC(O)(CH₂)ₙ— | —(CH₂)ₙC(O)(CH₂)ₙ— | |
| SO₂—C₃.₆ | SO₂—C₃.₆ | SO₂—C₂₋₆ alkynylene-C(O) | |
| heterocycloalkylene- | heterocycloalkenylene- | | |
| C(O) | C(O) | | |
| optionally substituted | optionally substituted | optionally substituted | |
| —(CH₂)ₙ—SO—C₃₋₆ | —(CH₂)ₙ—SO—C₃₋₆ | —(CH₂)ₙ—SO—C₂₋₆ | |
| heterocycloalkylene- | heterocycloalkenylene- | alkynylene-C(O)— | |
| C(O)— | C(O)— | | |
| optionally substituted | optionally substituted | optionally substituted | |
| —(CH₂)ₙC(O)(CH₂)ₙ—S— | —(CH₂)ₙC(O)(CH₂)ₙ—S— | —(CH₂)ₙC(O)(CH₂)ₙ—S— | |
| C₃₋₆ | C₃₋₆ | C₂₋₆ alkynylene-C(O) | |
| heterocycloalkylene- | heterocycloalkenylene- | | |
| C(O) | C(O) | | |
| optionally substituted | optionally substituted | optionally substituted | |
| —(CH₂)ₙ—S—S—C₃₋₆ | —(CH₂)ₙ—S—S—C₃₋₆ | —(CH₂)ₙ—S—S—C₂₋₆ | |
| heterocycloalkylene- | heterocycloalkenylene- | alkynylene-C(O)— | |
| C(O)— | C(O)— | | |
| optionally substituted | optionally substituted | optionally substituted | |
| —(CH₂)ₙC(O)(CH₂)ₙ—S— | —(CH₂)ₙC(O)(CH₂)ₙ—S— | —(CH₂)ₙC(O)(CH₂)ₙ—S— | |
| S—C₃₋₆ | S—C₃₋₆ | S—C₂₋₆ alkynylene-C(O) | |
| heterocycloalkylene- | heterocycloalkenylene- | | |
| C(O) | C(O) | | |
| optionally substituted | optionally substituted | optionally substituted | |
| —NR²⁰C(O)(CH₂)ₙO—C₃₋₆ | —NR²⁰C(O)(CH₂)ₙO—C₃₋₆ | —NR²⁰C(O)(CH₂)ₙO—C₂₋₆ | |
| heterocycloalkylene- | heterocycloalkenyl- | alkynylene-(CO) | |
| (CO) | ene-(CO) | | |
| optionally substituted | optionally substituted | optionally substituted | |
| NR²⁰C(O)(CH₂)ₙ—S—C₃₋₆ | NR²⁰C(O)(CH₂)ₙ—S—C₃₋₆ | NR²⁰C(O)(CH₂)ₙ—S—C₂₋₆ | |
| heterocycloalkylene- | heterocycloalkenyl- | alkynylene-(CO) | |
| (CO) | ene-(CO) | | |
| optionally substituted | optionally substituted | optionally substituted | |
| —NR²⁰C(O)(CH₂)ₙNR²¹— | —NR²⁰C(O)(CH₂)ₙNR²¹— | —NR²⁰C(O)(CH₂)ₙNR²¹— | |
| C₃₋₆ heterocycloalkylene- | C₃₋₆ heterocycloalkenyl- | C₂₋₆ alkynylene-(CO) | |
| (CO) | ene-(CO) | | |
| optionally substituted | optionally substituted | optionally substituted | |
| —C(O)NR²⁰(CH₂)ₙO—C₃₋₆ | —C(O)NR²⁰(CH₂)ₙO—C₃₋₆ | —C(O)NR²⁰(CH₂)ₙO—C₂₋₆ | |
| heterocycloalkylene- | heterocycloalkenyl | alkynylene-(CO) | |
| (CO) | ene-(CO) | | |
| optionally substituted | optionally substituted | optionally substituted | |
| —C(O)NR²⁰(CH₂)ₙ—S— | —C(O)NR²⁰(CH₂)ₙ—S— | —C(O)NR²⁰(CH₂)ₙ—S— | |
| C₃₋₆ heterocycloalkylene- | C₃₋₆ heterocycloalkenyl | C₂₋₆ alkynylene-(CO) | |
| (CO) | ene-(CO) | | |
| optionally substituted | optionally substituted | optionally substituted | |
| —C(O)NR²⁰(CH₂)ₙ—NR²¹— | C(O)NR²⁰(CH₂)ₙ—NR²¹— | C(O)NR²⁰(CH₂)ₙ—NR²¹— | |
| C₃₋₆ heterocycloalkylene- | C₃₋₆ heterocycloalkenyl- | C₂₋₆ alkynylene-(CO) | |
| (CO) | ene-(CO) | | |
| optionally substituted | optionally substituted | optionally substituted | |
| —C(O)(CH₂)ₙC₃₋₆ hetero- | —C(O)(CH₂)ₙC₃₋₆ | —C(O)(CH₂)ₙC₁₋₆ | |
| cycloalkylene- | heterocycloalkenyl- | alkynylene | |
| (CH₂)ₙ— | ene-(CH₂)ₙ— | (CH₂)ₙ— | |
| optionally substituted | optionally substituted | optionally substituted | |
| —C(O)O(CH₂)ₙ— | —C(O)O(CH₂)ₙ— | —C(O)O(CH₂)ₙ—C₁₋₆ | |
| C₃₋₆ | C₃₋₆ | alkynylene- | |
| heterocycloalkylene- | heterocycloalkenylene- | (CH₂)ₙ— | |
| (CH₂)ₙ | (CH₂)ₙ— | | |
| optionally substituted | optionally substituted | optionally substituted | |
| —C(O)(CH₂)ₙ— | —C(O)(CH₂)ₙ— | —C(O)(CH₂)ₙ—C₁₋₆ | |
| C₃₋₆ heterocycloalkylene- | C₃₋₆ heterocycloalkenyl | alkynylene-(CH₂)ₙ—O— | |
| (CH₂)ₙ—O— | ene-(CH₂)ₙ—O— | | |
| optionally substituted | optionally substituted | optionally substituted | |
| —C(O)O(CH₂)ₙ— | —C(O)O(CH₂)ₙ— | —C(O)O(CH₂)ₙ—C₁₋₆ | |
| C₃₋₆ heterocycloalkylene- | C₃₋₆ heterocycloalkenyl- | alkynylene- | |
| (CH₂)ₙ—O— | ene-(CH₂)ₙ—O— | (CH₂)ₙ—O— | |
| optionally substituted | optionally substituted | optionally substituted | |
| —C(O)(CH₂)ₙC₃₋₆ hetero- | —C(O)(CH₂)ₙC₃₋₆ | —C(O)(CH₂)ₙC₁₋₆ alkynylene | |
| cyclo- | heterocyclo- | (CH₂)ₙ—C(O)— | |
| alkylene-(CH₂)ₙ—C(O)— | alkenylene- | | |
| | (CH₂)ₙ—C(O)— | | |
| optionally substituted | optionally substituted | optionally substituted | |
| —C(O)(CH₂)ₙC₃₋₆ | —C(O)(CH₂)ₙC₃₋₆ | —C(O)(CH₂)ₙC₁₋₆ | |
| heterocyclo- | heterocyclo- | alkynylene- | |
| alkylene-(CH₂)ₙ—C(O)— | alkenylene-(CH₂)ₙ— | (CH₂)ₙ—C(O)— | |
| | C(O)— | | |
| optionally substituted | optionally substituted | optionally substituted | |
| —OC(O)(CH₂)ₙC₃₋₆ | —OC(O)(CH₂)ₙC₃₋₆ | —OC(O)(CH₂)ₙC₃₋₆ | |
| heterocyclo- | heterocycloalkenylene- | alkynylene- | |
| alkylene-(CH₂)ₙ | (CH₂)ₙ | (CH₂)ₙ | |
| optionally substituted | optionally substituted | optionally substituted | |
| —O(CH₂)ₙ—C₃₋₆ | —O(CH₂)ₙ—C₃₋₆ | —O(CH₂)ₙ—C₁₋₆ alkynylene- | |
| heterocyclo- | heterocyclo- | (CH₂)ₙ | |
| alkylene-(CH₂)ₙ | alkenylene-(CH₂)ₙ | | |
| optionally substituted | optionally substituted | optionally substituted | |
| —OC(O)(CH₂)ₙC₃₋₆ hetero- | —OC(O)(CH₂)ₙC₃₋₆ | —OC(O)(CH₂)ₙC₁₋₆ | |
| cyclo-alkylene-(CH₂)ₙ— | heterocyclo- | alkynylene- | |
| O— | alkenylene-(CH₂)ₙ—O— | (CH₂)ₙ—O— | |
| optionally substituted | optionally substituted | optionally substituted | |
| —O(CH₂)ₙC₃₋₆ | —O(CH₂)ₙC₃₋₆ | —O(CH₂)ₙC₃₋₆ alkynylene- | |
| heterocyclo- | heterocyclo- | (CH₂)ₙ—O— | |
| alkylene-(CH₂)ₙ—O— | alkenylene-(CH₂)ₙ—O— | | |
| optionally substituted | optionally substituted | optionally substituted | |
| —OC(O)(CH₂)ₙC₃₋₆ | —OC(O)(CH₂)ₙC₃₋₆ | —OC(O)(CH₂)ₙC₁₋₆ | |
| heterocyclo- | heterocyclo- | alkynylene- | |
| alkylene-(CH₂)ₙ—C(O)— | alkenylene-(CH₂)ₙ— | (CH₂)ₙ—C(O)— | |
| | C(O)— | | |
| optionally substituted | optionally substituted | optionally substituted | |
| —O(CH₂)ₙ— | —O(CH₂)ₙ— | —O(CH₂)ₙ—C₁₋₆ alkynylene- | |
| C₃₋₆ | C₃₋₆ | (CH₂)ₙ—C(O)— | |
| heterocycloalkylene- | heterocycloalkenylene | | |
| (CH₂)ₙ—C(O)— | (CH₂)ₙ—C(O)— | | |
| optionally substituted | optionally substituted | optionally substituted | |
| —C(O)NR²⁰(CH₂)ₙ—C₃₋₆ | —C(O)NR²⁰(CH₂)ₙ—C₃₋₆ | —C(O)NR²⁰(CH₂)ₙ—C₁₋₆ | |
| heterocycloalkylene- | C₃-e heterocycloalkenyl- | alkynylene- | |
| (CH₂)ₙ— | ene-(CH₂)ₙ— | (CH₂)ₙ— | |
| optionally substituted | optionally substituted | optionally substituted | |
| —NR²⁰C(O)—(CH₂)ₙ—C₃₋₆ | —NR²⁰C(O)—(CH₂)ₙ—C₃₋₆ | —NR²⁰C(O)—(CH₂)ₙ—C₁₋₆ | |
| heterocycloalkylene- | heterocycloalkenyl- | alkynylene- | |
| (CH₂)ₙ— | ene-(CH₂)ₙ— | (CH₂)ₙ— | |
| optionally substituted | optionally substituted | optionally substituted | |
| —C(O)NR²⁰(CH₂)ₙ—C₃₋₆ | —C(O)NR²⁰(CH₂)ₙ—C₃₋₆ | —C(O)NR²⁰(CH₂)ₙ—C₁₋₆ | |
| heterocycloalkylene- | heterocycloalkenyl | alkynylene- | |
| (CH₂)ₙ—O— | ene-(CH₂)ₙ—O— | (CH₂)ₙ—O— | |
| optionally substituted | optionally substituted | optionally substituted | |
| —NR²⁰C(O)(CH₂)ₙ—C₃₋₆ | —NR²⁰C(O)(CH₂)ₙ—C₃₋₆ | —NR²⁰C(O)(CH₂)ₙ—C₁₋₆ | |
| heterocycloalkylene- | heterocycloalkenyl | alkynylene- | |
| (CH₂)ₙ—O— | ene-(CH₂)ₙ—O— | (CH₂)ₙ—O— | |
| optionally substituted | optionally substituted | optionally substituted | |
| —C(O)NR²⁰(CH₂)ₙ—C₃₋₆ | —C(O)NR²⁰(CH₂)ₙ—C₃₋₆ | —C(O)NR²⁰(CH₂)ₙ—C₁₋₆ | |
| heterocycloalkylene- | heterocycloalkenyl | alkynylene- | |
| (CH₂)ₙ—C(O)— | ene-(CH₂)ₙ—C(O)— | (CH₂)ₙ—C(O)— | |
| optionally substituted | optionally substituted | optionally substituted | |
| —NR²⁰C(O)(CH₂)ₙ—C₃₋₆ | —NR²⁰C(O)(CH₂)ₙ—C₃₋₆ | —NR²⁰C(O)(CH₂)ₙ—C₁₋₆ | |
| heterocycloalkylene- | heterocycloalkenyl- | alkynylene- | |
| (CH₂)ₙ—C(O)— | ene-(CH₂)ₙ—C(O)— | (CH₂)ₙ—C(O)— | |

*Each R²⁰ and R²¹ is independently selected from the group consisting of hydrogen, hydroxy, OR²²,

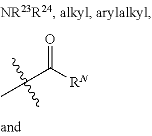

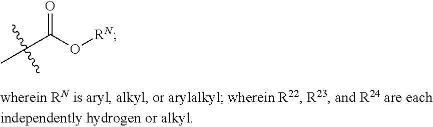

In another aspect, provided herein is a compound library that comprises a plurality of distinct tagged macrocyclic compounds according to any of the above. In certain embodiments, provided herein is a compound library that comprises at least about 10² distinct tagged macrocyclic compounds according to any of the above. In certain embodiments, provided herein is a compound library that comprises from about 10² to about 10¹⁰ distinct tagged macrocyclic compounds according to any of the above.

In a further aspect, provided herein is a method of making a library of tagged macrocyclic compounds as disclosed herein, the method comprising synthesizing a plurality of distinct tagged macrocyclic compounds according to any of the above.

In a still further aspect, provided herein is a method of making a tagged macrocyclic compound as disclosed herein, the method comprising operatively linking at least one oligonucleotide (D) to at least one of an FKBD, an effector domain, a first linking region, and a second linking region, and forming a macrocyclic ring comprising the FKBD, the effector domain, the first linking region, and the second linking region.

In certain embodiments, provided herein is a method of making a tagged macrocyclic compound as disclosed herein, the method comprising macrocyclic compound to at least one oligonucleotide (D), the macrocyclic compound comprising an FKBD, an effector domain, a first linking region, and a second linking region, wherein the FKBD, the effector domain, the first linking region, and the second linking region together form a macrocycle; and wherein the at least one oligonucleotide (D) can identify the structure of at least one of the FKBD, the effector domain, the first linking region, and the second linking region.

In yet a further aspect, the method of making a tagged macrocyclic compound comprises: operatively linking a compound of Formula (IV):

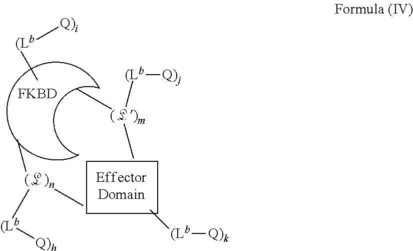

to a compound of Formula (V):

Q′—L^{c}—D Formula (V)

In some embodiments, [symbol] and [symbol] ′ are independently at each occurrence: a bond, —O—, —NR¹⁹—, —SO—, —SO₂—, —(CH₂)ₙ—,

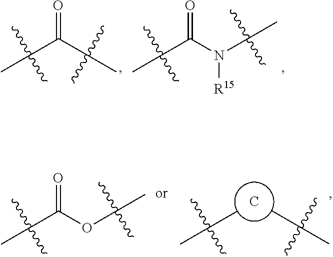

or a linking group selected from Table 1 wherein Ring C is a 5-6 membered heteroaryl, optionally substituted with 1-4 substituents, each of which is independently selected from the group consisting of hydrogen, hydroxy, halo, alkyl, alkoxy, haloalkyl, haloalkoxy, cyano, alkylthio, amino, alkylamino, dialkylamino; wherein R¹⁹ is selected from the group consisting of hydrogen, hydroxy, OR²², NR²³R²⁴, alkyl, arylalkyl,

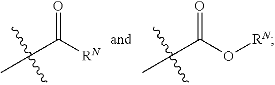

wherein R^{N} is aryl, alkyl, or arylalkyl; wherein R²², R²³, and R²⁴ are each independently hydrogen or alkyl; Q and Q′ are each independently selected from the group consisting of N₃, —C ≡CH, NR⁶R₇, —COOH, —ONH₂, —SH, —NH₂,

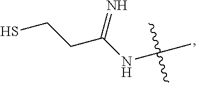 [SMILES: *NC(=N)CCS]

### —(C≡O)R′,

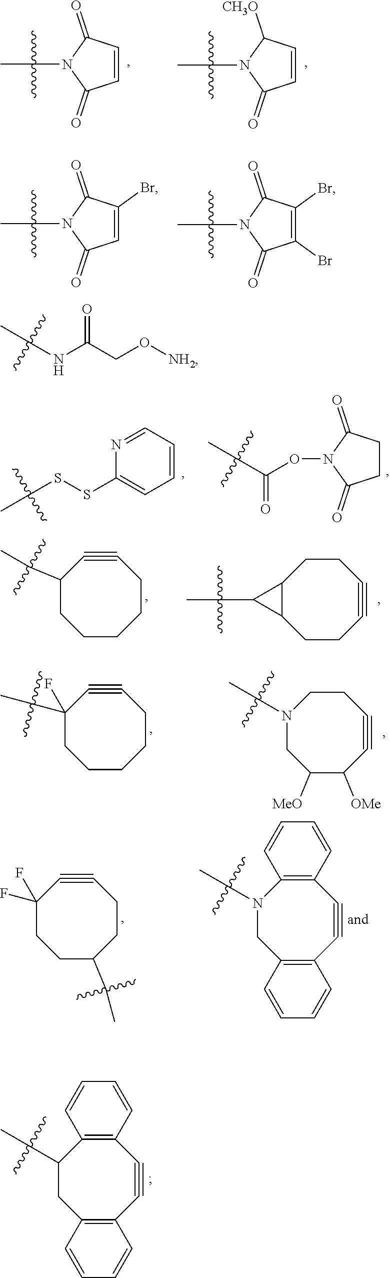

wherein R⁶ and R⁷ is each independently hydrogen, alkyl, arylalkyl,

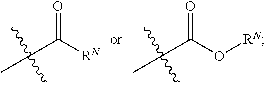

wherein R^{N} is aryl, alkyl, or arylalkyl; and R′ is hydrogen, alkyl, arylalkyl, or haloalkyl; L^{b} and L^{c} are independently at each occurrence selected from the group consisting of a bond, —O—, —S—, —OC(O)—, —C(O)O—, —(CH₂)ₙC(O)—, —(CH₂)ₙC(O)C(O)—, —(CH₂)ₙNR⁵C(O)C(O)—, —NR⁵(CH₂)ₙC(O)C(O)—, optionally substituted (CH₂)ₙC₁₋₆ alkylene-(CH₂)ₙ—, optionally substituted (CH₂)ₙC(O)C₁₋₆ alkylene-(CH₂)ₙ—, optionally substituted (CH₂)ₙNR⁵C₁₋₆ alkylene-(CH₂)ₙ—, optionally substituted (CH₂)ₙC(O)NR⁵C₁₋₆ alkylene-(CH₂)ₙ—, optionally substituted (CH₂)ₙNR⁵C(O)C₁₋₆ alkylene-(CH₂)ₙ—, optionally substituted (CH₂)ₙC(O)OC₁₋₆ alkylene-(CH₂)ₙ—, optionally substituted (CH₂)ₙOC(O)C₁₋₆ alkylene-(CH₂)ₙ—, optionally substituted (CH₂)ₙOC₁₋₆ alkylene-(CH₂)ₙ—, optionally substituted (CH₂)ₙNR⁵C₁₋₆ alkylene-(CH₂)ₙ—, optionally substituted (CH₂)ₙ—S—C₁₋₆ alkylene-(CH₂)ₙ—, and optionally substituted (CH₂CH₂O)ₙ; wherein each alkylene is optionally substituted with 1 or 2 groups independently selected from the group consisting of of halo, hydroxy, haloalkyl, haloalkoxy, alkyl, alkoxy, amino, carboxyl, cyano, nitro, NHFmoc; wherein each R⁵ is independently hydrogen, alkyl, arylalkyl,

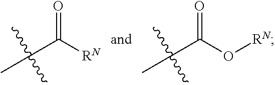

wherein R^{N}is aryl, alkyl, or arylalkyl;

D is an oligonucleotide; h, i, j, and k are each independently an integer from 0-20, provided that at least one of h, i, j, and k is not 0; n is an integer from 1-5; m is an integer from 1-5.

In another aspect, provided herein is a method of making a tagged macrocyclic compound, the method comprising operatively linking a compound of Formula (VI):

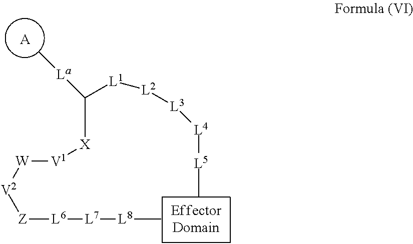

with a compound of Formula (V):

Ring A is a 5-10 membered aryl, cycloalkyl, heteroaryl or heterocycloalkyl, optionally substituted with 1-17 substituents, each of which is independently selected from the group consisting of hydrogen, hydroxy, halo, alkyl, alkoxy, cyano, haloalkyl, haloalkoxy, alkylthio, oxo, amino, alkylamino, dialkylamino,

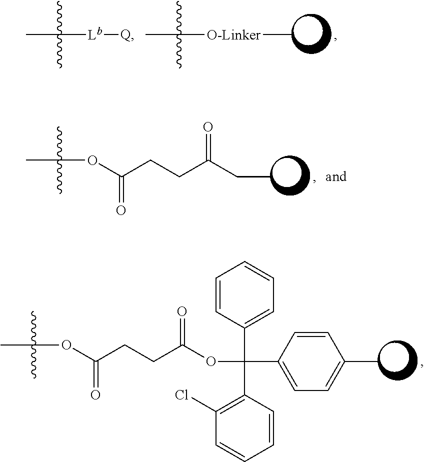

wherein [symbol] is a resin;

L^{b} and L^{c} are independently selected from the group consisting of a bond, —O—, —S—, —OC(O)—, —C(O)O—, —(CH₂)ₙC(O)—, —(CH₂)ₙC(O)C(O)—, —(CH₂)ₙ NR⁵C(O)C(O)—, —NR⁵(CH₂)ₙC(O)C(O)—, optionally substituted (CH₂)ₙC₁₋₆ alkylene-(CH₂)ₙ—, optionally substituted (CH₂)ₙC(O)C₁₋₆ alkylene-(CH₂)ₙ—, optionally substituted (CH₂)ₙNR⁵C₁₋₆ alkylene-(CH₂)ₙ—, optionally substituted (CH₂)ₙC(O)NR⁵C₁₋₆ alkylene-(CH₂)ₙ—, optionally substituted (CH₂)ₙNR⁵C(O)C₁₋₆ alkylene-(CH₂)ₙ—, optionally substituted (CH₂)ₙC(O)OC₁₋₆ alkylene-(CH₂)ₙ—, optionally substituted (CH₂)ₙOC(O)C₁₋₆ alkylene-(CH₂)ₙ—, optionally substituted (CH₂)ₙOC₁₋₆ alkylene-(CH₂)ₙ—, optionally substituted (CH₂)ₙNR⁵C₁₋₆ alkylene-(CH₂)ₙ—, optionally substituted (CH₂)ₙ—S—C₁₋₆ alkylene-(CH₂)ₙ—, and optionally substituted (CH₂CH₂O)ₙ; wherein each alkylene is optionally substituted with 1 or 2 groups independently selected from the group consisting of of halo, hydroxy, haloalkyl, haloalkoxy, alkyl, alkoxy, amino, carboxyl, cyano, nitro, NHFmoc; wherein each R⁵ is independently hydrogen, alkyl, arylalkyl,

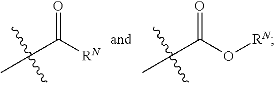

wherein R^{N}is aryl, alkyl, or arylalkyl;

Q and Q′ are independently selected from the group consisting of —N₃, —C≡CH, NR⁶R⁷, —COOH, —ONH₂, —SH, —NH₂,

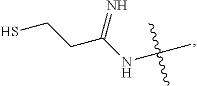 [SMILES: *NC(=N)CCS]

### —(C≡O)R′,

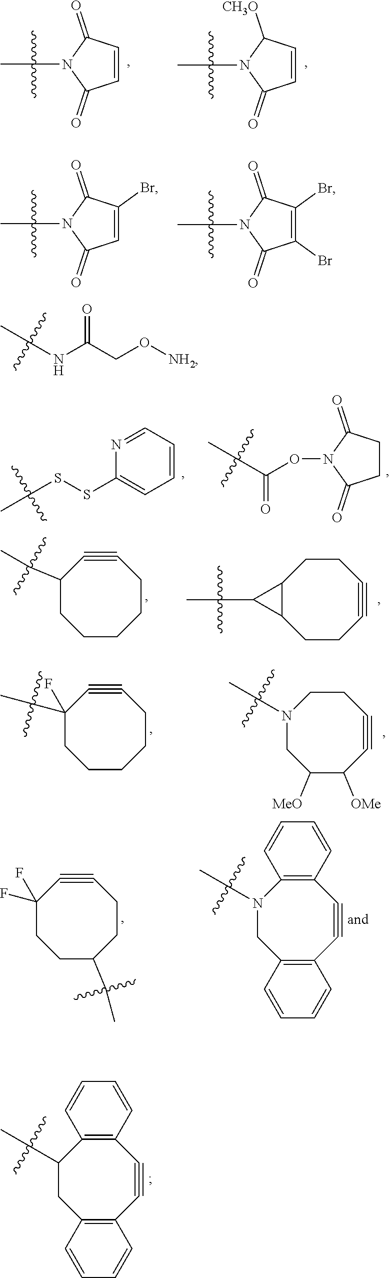

wherein R⁶ and R⁷ is each independently hydrogen, alkyl, arylalkyl,

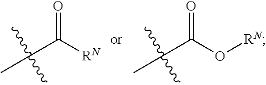

wherein R^{N} is aryl, alkyl, or arylalkyl; and R′ is hydrogen, alkyl, arylalkyl, or haloalkyl; X is O, S or NR⁸, wherein R⁸ is hydrogen, hydroxy, OR⁹, NR¹⁰R¹¹, alkyl, arylalkyl,

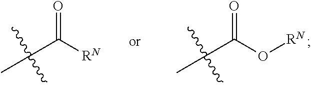

wherein R^{N} is aryl, alkyl, or arylalkyl; wherein R⁹, R¹⁰ and R¹¹ are each independently hydrogen or alkyl; V¹ and V² are each independently

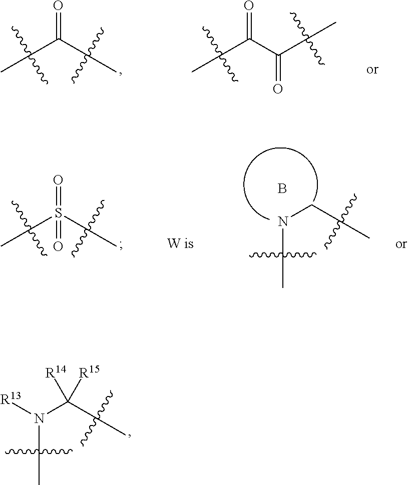

wherein Ring B is a 4-10 membered heterocycloalkyl, optionally substituted with 1-10 substituents, each of which is selected from the group consisting of hydrogen, hydroxy, halo, alkyl, alkoxy, cyano, haloalkyl, haloalkoxy, alkylthio, oxo, amino, alkylamino, dialkylamino, arylalkyl,

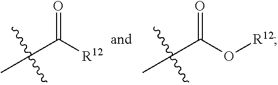

wherein R¹² is aryl, alkyl, or arylalkyl; wherein R¹³ is hydrogen, hydroxy, OR¹⁶, NR¹⁷R¹⁸, alkyl, arylalkyl,

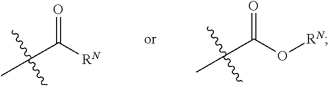

wherein R^{N} is aryl, alkyl, or arylalkyl; R¹⁴ and R¹⁵ is each independently hydrogen, hydroxy, halo, alkyl, alkoxy, haloalkyl, haloalkoxy, aryl, arylalkyl, or heteroaryl;

Z is bond,

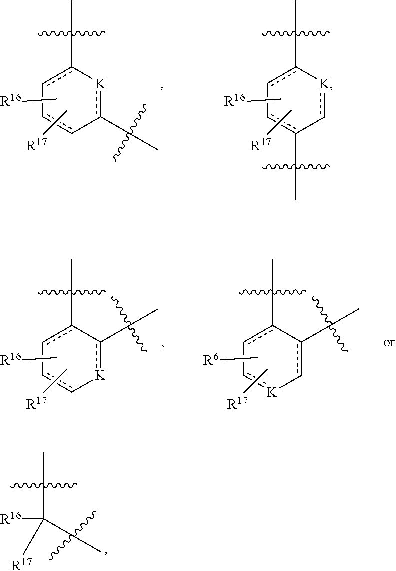

wherein R¹⁶ and R¹⁷ are each independently selected from the group consisting of of hydrogen, hydroxy, halo, alkyl, alkoxy, cycloalkyl, cyano, alkylthio, amino, alkylamino, and dialkylamino; K is O, CHR¹⁸, CR¹⁸, N, and NR¹⁸, wherein R¹⁸ is hydrogen or alkyl;

L^{a}, L¹, L², L³, L⁴, L⁵, L⁶, L⁷ and L⁸ are each independently a bond, —O—, —NR¹⁹—, —SO—,—SO₂—, —(CH₂)ₙ—,

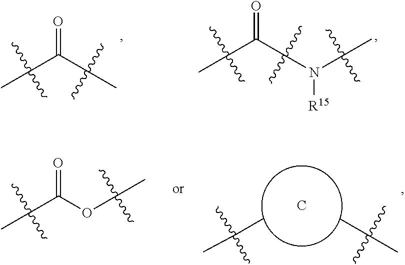

or a linking group selected from Table 1; wherein Ring C is a 5-6 membered heteroaryl, optionally substituted with 1-4 substituents, each of which is independently selected from the group consisting of hydrogen, hydroxy, halo, alkyl, alkoxy, haloalkyl, haloalkoxy, cyano, alkylthio, amino, alkylamino, dialkylamino and

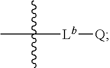

wherein R¹⁹ is selected from the group consisting of hydrogen, hydroxy, OR²², NR²³R²⁴, alkyl, arylalkyl,

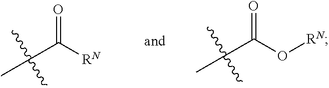

wherein R^{N} is aryl, alkyl, or arylalkyl; wherein R²², R²³, and R²⁴ are each independently hydrogen or alkyl;

n is 0, 1, 2, 3, 4, 5 or 6; wherein the Effector Domain has Formula (IIIa):

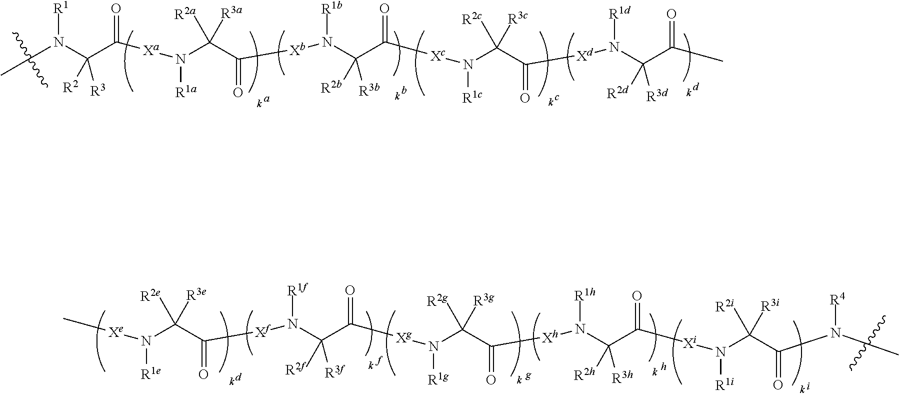

each k^{a}, k^{b}, k^{c}, k^{d}, k^{e}, k^{f}, k^{g}, k^{h}, and kⁱ is independently 0 or 1; each X^{a}, X^{b}, X^{c}, X^{d}, X^{e}, X^{f}, X^{g}, X^{h}, and Xⁱ is independently a bond, —S—, —S—S—, —S(O)—, —S(O)₂—, substituted or unsubstituted —(C₁-C₃) alkylene-, —(C₂-C₄) alkenylene-, —(C₂-C₄) alkynylene-, or

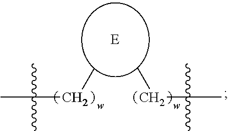

wherein Ring E is phenyl or a 5-6 heteroaryl or heterocycloalkyl; wherein each w is independently 0, 1, or 2; each R¹, R^{1a}, R^{1b}, R^{1c}, R^{1d}, R^{1e}, R^{1f}, R^{1g}, R^{1h}, R¹ⁱ, and R⁴ is independently hydrogen, alkyl, arylalkyl or NR²⁵ wherein R²⁵ is hydrogen, hydroxy, OR²⁶ N²⁷R²⁸, alkyl, arylalkyl,

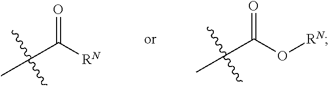

wherein R^{N} is aryl, alkyl, or arylalkyl; wherein R²⁶, R²7, and R²⁸ are each independently hydrogen or alkyl; each R², R³, R^{2a}, R^{3a}, R^{2b}, R^{3b}, R^{2c}, R^{3c}, R^{2d}, R^{3d}, R^{2e}, R^{3e}, R^{2f}, R^{3f}, R^{2g},R^{3g}, R^{2h}, R^{3h}, R²ⁱ, and R³ⁱ is independently selected from the group consisting of hydrogen, halo, amino, cyano, nitro, haloalkyl, optionally substituted alkyl, optionally substituted alkoxy, optionally substituted alkenyl, optionally substituted alkynyl, optionally substituted cycloalkyl, optionally substituted heterocycloalkyl, optionally substituted alkylamino, optionally substituted dialkylamino, optionally substituted aryl, optionally substituted heteroaryl, optionally substituted arylalkyl, optionally substituted heteroarylalkyl, and

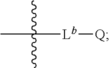

or wherein the Effector Domain has Formula (IIIb):

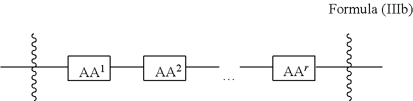

wherein each of AA¹, AA², . . . , and AA^{r} is an natural or unnatural amino acid residue; and r is 3, 4, 5, 6, 7, 8, 9, or 10;

or wherein the Effector Domain has Formula (IIIc):

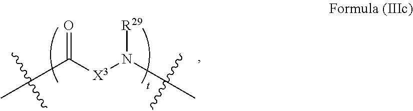

each t is independently an integer selected from 0, 1, 2, 3, 4, 5, 6, 7, 8, 9, and 10; R²⁹ is hydrogen, hydroxy, OR³⁰, NR³¹R³², alkyl, arylalkyl,

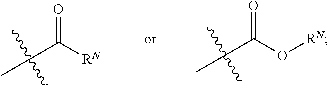

wherein R^{N} is aryl, alkyl, or arylalkyl; wherein R³⁰, R³¹, and R³² are each independently hydrogen or alkyl; X³ is substituted or unsubstituted —(C₁-C₆) alkylene-, —(C₂-C₆) alkenylene-, —(C₂-C₆) alkynylene-, or

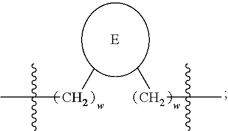

wherein Ring E is phenyl or a 5-6 heteroaryl or heterocycloalkyl; wherein each w is independently 0, 1, or 2;

or wherein the Effector Domain has Formula (IId):

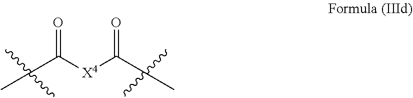

X⁴ is substituted or unsubstituted —(C₁-C₆) alkylene-, —(C₂-C₆) alkenylene-, —(C₂-C₆) alkynylene-, or

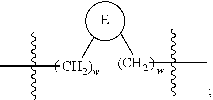

wherein Ring E is phenyl or a 5-6 heteroaryl or heterocycloalkyl; wherein each w is independently 0, 1, or 2;

or wherein the Effector Domain has Formula (IIIe):

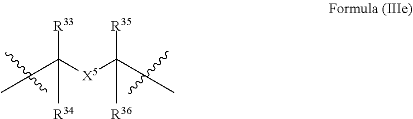

R³³, R³⁴, R³⁵ and R³⁶ are each hydrogen or alkyl; X⁵ is substituted or unsubstituted —(C₁-C₆) alkylene-, —(C₂-C₆) alkenylene-, —(C₂-C₆) alkynylene-, or

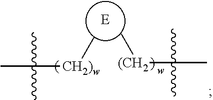

wherein Ring E is phenyl or a 5-6 heteroaryl or heterocycloalkyl; wherein each w is independently 0, 1, or 2;

or wherein the Effector Domain has Formula (IIIf):

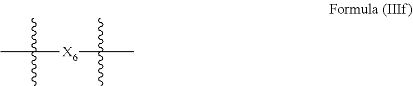

X⁶ is substituted or unsubstituted —(C₁-C₆) alkylene-, —(C₂-C₆) alkenylene-, —(C₂-C₆) alkynylene-, or

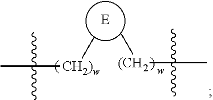

wherein Ring E is phenyl or a 5-6 heteroaryl or heterocycloalkyl; wherein each w is independently 0, 1, or 2; and provided that when Ring A is

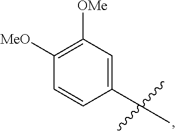 [SMILES: *c1ccc(OC)c(OC)c1]

L^{a} is ethylene, X is, O, W is

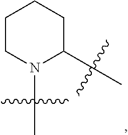 [SMILES: *C1CCCCN1*]

### V¹ is

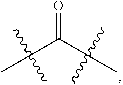 [SMILES: *C(*)=O]

### V² is

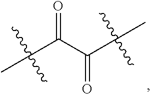 [SMILES: *C(=O)C(*)=O]

### Z is

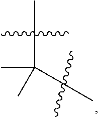 [SMILES: *C(*)(C)C]

L⁶—L⁷—L⁸— is

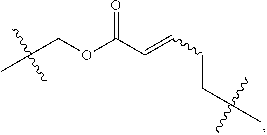 [SMILES: *CCC=CC(=O)OC*]

and —L¹—L²—L³—L⁴—L⁵— is not

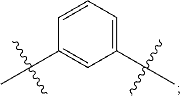 [SMILES: *c1cccc(*)c1]

D is an oligonucleotide; wherein Ring A is substituted with at least one

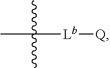

or at least one of R², R³, R^{2a}, R^{3a}, R^{2b}R^{3b}, R^{2c}, R^{3c}, R^{2d}, R^{3d}, R^{2e}, R^{3e}, R^{2f}, R^{3f}, R^{2g}, R³, R^{2h}, R^{3h}, R²ⁱ, and R³ⁱ is

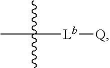

or at least one of L^{a}, L¹, L², L³, L⁴, L⁵, L⁶, L⁷ and L⁸ is Ring C substituted with at least one

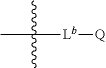

or wherein at least one of the linking groups selected from Table 1 is substituted with at least one

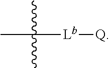

Also disclosed here is a compound according to Formula (VII) or an optically pure stereoisomer, pharmaceutically acceptable salt, or solvate thereof:

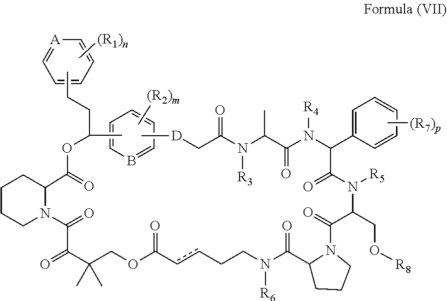

Each A and B can be independently CH or N. Each n, m, and p can be independently an integer selected from 0 to 4. “ [symbol] ” can be a single bond or double bond.

[symbol]

D can be selected from the group consisting of —O(CH₂)_{q}—, —S(CH₂)_{q}—, —COO(CH₂)_{q}—, —CONR₃(CH₂)_{q}—, and —NR₃(CH₂)_{q}—. q can be an integer selected from 0 to 4.

Each R₁, R₂, and R₇ can be independently selected from the group consisting of H, F, Br, C₁, CF₃, CN, N₃, NH₂, NO₂, OH, OCH₃, methyl, ethyl, and propyl.

Each R₃, R₄, R₅, and R₆ can be independently selected from the group consisting of H, methyl, ethyl, propyl, and isopropyl.

R₈ can be selected from the group consisting of H, methyl, ethyl, n-propyl, isopropyl, n-butyl, isobutyl, sec-butyl, and tert-butyl.

In some aspects, the carbon connected to

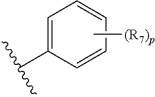

can adopt R or S stereochemistry.

In yet another aspect, provided herein is a method for identifying one or more compounds that bind to a biological target the method comprising: (a) incubating the biological target with at least a portion of the plurality of distinct tagged macrocyclic compounds of the compound library of claim 2 to make at least one bound compound and at least one unbound compound of the plurality of distinct tagged macrocyclic compounds; (b) removing the at least one unbound compound; and (c) sequencing each of the oligonucleotides (D) of the at least one bound compound.

In certain embodiments, the DNA-encoded library can be a single pharmacophore library, wherein only one chemical moiety can be attached to a single strand of DNA, as described in, e.g., Neri & Lerner, Annu. Rev. Biochem. (2018) 87:5.1-5.24, which is hereby incorporated by reference in its entirety. In certain embodiments, the DNA-encoded library can be a dual pharmacophore library, wherein two independent molecules can be attached to the double strands of DNA, as described in, e.g., Id; Mannocci et al., Chem. Commun. (2011) 47:12747-53, which is hereby incorporated by reference in its entirety.

In a further aspect, provided herein is a method of making a library of tagged macrocyclic compounds, the method comprising synthesizing a plurality of distinct tagged macrocyclic compounds. In certain embodiments, each tagged macrocyclic compound of the plurality of distinct tagged macrocyclic compounds comprising a macrocyclic compound operatively linked to at least one oligonucleotide (D). In certain embodiments, each compound of the plurality of distinct tagged macrocyclic compounds of the compound library comprises a macrocyclic compound operatively linked to at least one oligonucleotide (D). In certain embodiments, the macrocyclic compound comprising an FKBD, an effector domain, a first linking region, and a second linking region. In certain embodiments, the FKBD, the effector domain, the first linking region, and the second linking region together form a macrocycle. In certain embodiments, each of the at least one oligonucleotide (D) can identify at least one of the FKBD, the effector domain, the first linking region, and the second linking region of each of the plurality of distinct tagged macrocyclic compounds. In certain embodiments, each compound of the plurality of distinct tagged macrocyclic compounds of the compound library comprises a compound of Formula (A) (as above-defined). In certain embodiments, each compound of the plurality of distinct tagged macrocyclic compounds of the compound library comprises a compound of Formula (I) (as above-defined herein). In certain embodiments, each compound of the plurality of distinct tagged macrocyclic compounds of the compound library can be a reaction product of operatively linking a compound of Formula (B) (as above-defined herein) with a compound of Formula (C) (as above-defined herein). In certain embodiments, each compound of the plurality of distinct tagged macrocyclic compounds of the compound library can be a reaction product of operatively linking a compound of Formula (B′) (as above-defined herein) with a compound of Formula (C) (as above-defined herein).

In certain embodiments, the method of synthesizing a library of compounds can be selected from the group consisting of the split-and-pool method, DNA-templated library synthesis (DTS), encoded self-assembling chemical (ESAC) library synthesis, DNA-recorded library synthesis, DNA-directed library synthesis, DNA-routing, and 3-D proximity-based library synthesis (YoctoReactor). As a person of ordinary skill in the art would be aware, various techniques for synthesizing the library of tagged macrocyclic compounds are described in, e.g., Neri & Lerner, Annu. Rev. Biochem. (2018) 87:5.1-5.24; Roman et al., SLASDiscov. (2018) 23(5):387-396; Lim, C&EN, (2017) 95 (29):10-10; Halford, C&EN, (2017) 95(25): 28-33; Estevez, Tetrahedron: Asymmetry. (2017) 28:837-842; Neri, Chembiochem. (2017) 4;18(9):827-828; Yuen & Franzini, Chembiochem. (2017) 4;18(9):829-836; Skopic et al., Chem Sci. (2017) 1;8(5):3356-3361; Shi et al.; BioorgMed Chem Lett. (2017) 1;27(3):361-69; Zimmermann & Neri, Drug Discov Today. (2016) 21(11):1828-1834; Satz et al., Bioconjug Chem. (2015) 19;26(8):1623-32; Ding et al., ACS Comb Sci. (2016) 10;18(10):625-629; Arico-Muendel, MedChemComm, (2016) 7(10): 1898-1909; Skopic, MedChemComm, (2016) 7(10): 1957-1965; Satz, CS Comb. Sci. (2016) 18 (7):415-424; Tian et al., MedChemComm, (2016) 7(7): 1316-1322; Salamon et al., ACS Chem Biol. (2016) 19;11(2):296-307; Satz et al., Bioconjug Chem. (2015) 19;26(8):1623-32; Connors et al., Curr Opin Chem Biol. (2015) 26:42-7; Blakskjaer et al., Curr Opin Chem Biol. (2015) 26:62-71; Scheuermann & Neri, Curr Opin Chem Biol. (2015) 26:99-103; Franzini et al., Angew Chem IntEd Engl. (2015) 23;54(13):3927-31; Franzini et al., Bioconjug Chem. (2014) 20;25(8):1453-61; Franzini, Neri & Scheuermann, Acc Chem Res. (2014) 15;47(4):1247-55; Mannocci et al., Chem. Commun. (2011) 47:12747-53; Kleiner et al., Chem Soc Rev. (2011) 40(12): 5707-17; Clark, Curr Opin Chem Biol. (2010) 14(3):396-403; Mannocci et al., Proc Nat! Acad Sci USA. (2008) 18;105(46):17670-75; Buller et al., BioorgMed Chem Lett. (2008) 18(22):5926-31; Scheuermann et al., Bioconjugate Chem. (2008) 19:778-85; Zimmerman et al., ChemBioChem (2017) 18(9):853-57, and Cuozzo et al., ChemBioChem (2017), 18(9):864-71, each of which is hereby incorporated by reference in its entirety.

In some embodiments, the method of synthesizing a library of tagged macrocyclic compounds comprises DNA-recorded library synthesis, in which encoding and library synthesis take place separately, as described in, e.g. Shi et al., Bioorg Med Chem Lett. (2017) 1;27(3):361-369; Kleiner et al., Chem Soc Rev. (2011) 40(12): 5707-17. In certain embodiments, the DNA-recorded library synthesis c comprises split-and-pool methods, which are described in, e.g., Krall, Scheuermann & Neri, Angew Chem. Int. Ed Engl. (2013) 28;52(5):1384-402; Mannocci et al., Chem. Commun. (2011) 47:12747-53; and U.S. Pat. No. 7,989,395 to Morgan et al., each of which is hereby incorporated by reference in its entirety. In certain embodiments, the split-and-pool method comprises successive chemical ligation of oligonucleotide tags to an initial oligonucleotide (or headpiece), which can be covalently linked to a chemically generated entity by successive split-and-pool steps. In certain embodiments, during each split step, a chemical synthesis step can be performed along with an oligonucleotide ligation step.

In some embodiments, the library can be synthesized by a sequence of split-and-pool cycles, wherein an initial oligonucleotide (or headpiece) can be reacted with a first set of building blocks (e.g., a plurality of FKBD building blocks). For each building block of the first set of building blocks (e.g., each FKBD building block), an oligonucleotide (D) can be appended to the initial oligonucleotide (or headpiece) and the resulting product can be pooled (or mixed), and subsequently split into separate reactions. Subsequently, in certain embodiments, a second set of building blocks (e.g., a plurality of effector domain building blocks) can be added, and an oligonucleotide (D) can be appended to each building block of the second set of building blocks. In certain embodiments, each oligonucleotide (D) identifies a distinct building block.

In some embodiments, the method of synthesizing a library of tagged macrocyclic compounds comprises DNA-directed library synthesis, in which DNA both encodes and templates library synthesis as described in, e.g. Kleiner et al., Bioconjugate Chem. (2010) 21, 1836-41; and Shi et. al, Bioorg Med Chem Lett. (2017) 1;27(3):361-369, each of which is hereby incorporated by reference in its entirety. In certain embodiments, the DNA-directed library synthesis comprises the DNA-templated synthesis (DTS) method as described in, e.g., Mannocci et al., Chem. Commun. (2011) 47:12747-53, Franzini, Neri & Scheuermann, Acc Chem Res. (2014) 15;47(4):1247-55; and Mannocci et al., Chem. Commun. (2011) 47:12747-53, each of which are hereby incorporated by reference in its entirety. In certain embodiments, the DTS method comprises DNA oligonucleotides that not only encode but also direct the construction of the library. See Buller et al., Bioconjugate Chem. (2010) 21, 1571-80, which is hereby incorporated by reference in its entirety. In certain embodiments different building blocks can be incorporated into molecules using DNA-linked reagents that can be forced into proximity by base pairing between their DNA tags. See Gartner et al., Science (2004) 305:1601-05, which is hereby incorporated by reference in its entirety. In certain embodiments, a library of long oligonucleotides can be synthesized first as a template for the DNA-encoded library. In certain embodiments, the oligonucleotides can be subjected to sequence-specific chemical reactions through immobilization on resin tagged with complementary DNA sequences. See Wrenn & Harbury, Annu. Rev. Biochem. (2007) 76:331-49, which is hereby incorporated by reference in its entirety.

In certain embodiments, the DNA-directed library synthesis comprises 3-D proximity-based library synthesis, also known as YoctoReactor technology, which is described in, e.g., Blakskjaer et al., Curr Opin Chem Biol. (2015) 26:62-7, which is hereby incorporated by reference in its entirety.

In certain embodiments, the method of synthesizing a library of tagged macrocyclic compounds comprises encoded self-assembling chemical (ESAC) library synthesis, also known as double-pharmacophore DNA-encoded chemical libraries, as described in, e.g., Mannocci et al., Chem. Commun. (2011) 47:12747-53; Melkko et al., Nat. Biotechnol. (2004) 22(5):568-74; Scheuermann et al., Bioconjugate Chem. (2008) 19:778-85; and U.S. Pat. No. 8,642,215 to Neri et al. each of which is hereby incorporated by reference in its entirety. In certain embodiments, synthesizing a library of tagged macrocyclic compounds by ESAC synthesis comprises, for example, non-covalent combinatorial assembly of complementary oligonucleotide sub-libraries, in which each sub-library can include a first oligonucleotide appended to a first building block, wherein the first oligonucleotide comprises a coding domain that identifies the first building block, and a hybridization domain, which self-assembles to a second oligonucleotide appended to a second building block, second oligonucleotide comprising a coding domain that identifies the second building block, and a hybridization domain that self-assembles to the first oligonucleotide.

In some embodiments, the method of synthesizing a library of tagged macrocyclic compounds comprises DNA-routing, as described in, e.g. Clark, Curr Opin Chem Biol. (2010) 14(3):396-403, which is hereby incorporated by reference in its entirety.

In certain embodiments, oligonucleotide ligation can utilize one of several methods that would be appreciated be a person of ordinary skill in the art, described, for example, in Zimmermann & Neri, Drug Discov. Today. (2016) 21(11):1828-1834; and Keefe et al., Curr Opin Chem Biol. (2015) 26:80-88, each of which are hereby incorporated by reference in its entirety. In certain embodiments, the oligonucleotide ligation can be an enzymatic ligation. In certain embodiments, the oligonucleotide ligation can be a chemical ligation.

In certain embodiments, the ligation comprises base-pairing a short, complementary “adapter” oligonucleotide to single-stranded oligonucleotides to either end of the ligation site, allowing ligation of single-stranded DNA tags in each cycle. See Clark et al., Nat. Chem. Biol. (2009) 5:647-54, which is hereby incorporated by reference in its entirety. In certain embodiments, the oligonucleotide ligation comprises utilizing 2-base overhangs at the 3′ end of the headpiece and of each building block's DNA tag to form sticky ends for ligation. In certain embodiments, the sequences of the overhangs can depend on the cycle but not on the building block, so that any DNA tag can be ligated to any DNA tag from the previous cycle, but not to a truncated sequence. See id. In certain embodiments, the oligonucleotide ligation step can utilize oligonucleotides of opposite sense for subsequent cycles, with a small region of overlap in which the two oligonucleotides are complementary. In certain embodiments, in lieu of ligation, DNA polymerase can be used to fill in the rest of the complementary sequences, creating a double-strand oligonucleotide comprising both tags. In certain embodiments, the oligonucleotide ligation can be chemical. While not wishing to be bound by theory, it is thought that chemical ligation may permit greater flexibility with regard to solution conditions and may reduce the buffer exchange steps necessary. See Keefe et al., Curr Opin Chem Biol. (2015) 26:80-88, which is hereby incorporated by reference in its entirety.

In certain embodiments, provided herein is a method for identifying one or more compounds that bind to a biological target, the method comprising: (a) incubating the biological target with at least a portion of a plurality of distinct tagged macrocyclic compounds of a compound library to make at least one bound compound and at least one unbound compound of the plurality of distinct tagged macrocyclic compounds; (b) removing the at least one unbound compound; (c) sequencing each of the at least one oligonucleotide (D) of the at least one bound compound. In certain embodiments, each compound of the plurality of distinct tagged macrocyclic compounds of the compound library comprises a macrocyclic compound operatively linked to at least one oligonucleotide (D). In certain embodiments, the macrocyclic compound comprises an FKBD, an effector domain, a first linking region, and a second linking region. In certain embodiments, the FKBD, the effector domain, the first linking region, and the second linking region together form a macrocycle. In certain embodiments, each at least one oligonucleotide (D) can identify at least one of the FKBD, the effector domain, the first linking region, and the second linking region of each of the plurality of distinct tagged macrocyclic compounds. In certain embodiments, each compound of the plurality of distinct tagged macrocyclic compounds of the compound library comprises a compound of Formula (A) (as above-defined). In certain embodiments, each compound of the plurality of distinct tagged macrocyclic compounds of the compound library comprises a compound of Formula (I) (as above-defined). As a person of ordinary skill in the art would be aware, various techniques for synthesizing the library of tagged macrocyclic compounds are described in, e.g., Kuai et al., SLASDiscov. (2018) 23(5):405-416; Brown et al., Annu. Rev. Biochem. (2018) 87:5.1-5.24; Roman et al., SLAS Discov. (2018) 23(5):387-396; Amigo et al., SLAS Discov. (2018) 23(5):397-404; Shi et al., Bioconjug Chem. (2017) 20;28(9):2293-2301; Machutta et al., Nat Commun. (2017) 8:16081; Li et al., Chembiochem. (2017) 4;18(9):848-852; Satz et al., ACS Comb Sci. (2017) 10;19(4):234-238; Denton & Krusemark, MedChemComm, (2016) 7(10): 2020-2027; Eidam & Satz, MedChemComm, (2016) 7(7): 1323-1331; Bao et al., Anal. Chem., (2016) 88 (10):5498-5506; Decurtins et al., Nat Protoc. (2016) 11(4):764-80; Harris et al., J. Med. Chem. (2016) 59 (5):2163-78; Satz, ACS Chem Biol. (2016) 16;10(10):2237-45; Chan et al., Curr Opin Chem Biol. (2015) 26:55-61; Franzini et al., Chem Commun. (2015) 11;51(38):8014-16; and Buller et al., BioorgMed Chem Lett. (2010) 15;20(14):4188-92. each of which is hereby incorporated by reference in its entirety.

In certain embodiments, the incubating step can be performed under conditions suitable for at least one of the plurality of distinct tagged macrocyclic compounds of the compound library to bind to the biological target. A person of ordinary skill in the art would understand what conditions would be considered suitable for at least one of the plurality of distinct tagged macrocyclic compounds of the compound library to bind to the biological target.

In certain embodiments, the identifying one or more compounds that bind to a biological target comprises a bind-wash-elute procedure for molecule selection as described in, e.g., Ding et al., ACS Med. Chem. Lett. (2015) 7;6(8):888-93, which is hereby incorporated by reference in its entirety. In certain embodiments, the incubating step (a comprises contacting the plurality of tagged compounds in the compound library with a target protein, wherein the target protein can be immobilized on a substrate (e.g., resin). In certain embodiments, the removing step (b) comprises washing the substrate to remove the at least one unbound compound. In certain embodiments, the sequencing step (c) comprises sequencing the at least one oligonucleotide (D) to identify which of the plurality of tagged compounds bound to the target protein.

In certain embodiments, the identifying one or more compounds that bind to a biological target comprises utilizing unmodified, non-immobilized target protein. Such methods, which can utilize a a ligate-crosslink-purify strategy are described in, e.g., Shi et al., Bioconjug. Chem. (2017) 20;28(9):2293-2301, which is hereby incorporated by reference in its entirety. In certain embodiments, other methods for identifying the one or more compounds that bind to the biological target can be utilized. Such methods would be apparently to a person of ordinary skill in the art, and examples of such methods are described in, e.g., Machutta et al., Nat. Commun. (2017) 8:16081; Chan et al., Curr. Opin. Chem. Biol. (2015) 26:55-61; Lim, C&EN, (2017) 95 (29):10; Amigo et al., SLASDiscov. (2018) 23(5):397-404; Tian et al., MedChemComm. (2016) 7(7): 1316-1322; See Satz, CS Comb. Sci. (2016) 18 (7):415-424 each of which is hereby incorporated by reference in its entirety.

The following explanations of terms and methods are provided to better describe the present disclosure and to guide those of ordinary skill in the art in the practice of the present disclosure. The singular terms “a,” “an,” and “the” include plural referents unless context clearly indicates otherwise. Similarly, the word “or” is intended to include “and” unless the context clearly indicates otherwise. The term “comprises” means “includes.” Thus, “comprising A or B,” means “including A, B, or A and B,” without excluding additional elements. The term “about” will be understood by persons of ordinary skill in the art. Whether the term “about” is used explicitly or not, every quantity given herein refers to the actual given value, and it is also meant to refer to the approximation to such given value that would be reasonably inferred based on the ordinary skill in the art.

It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of this disclosure, suitable methods and materials are described below.

Unless otherwise explained, all technical and scientific terms used herein have the same meaning as commonly understood by one of ordinary skill in the art to which this disclosure belongs. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

Unless indicated otherwise, the nomenclature of substituents that are not explicitly defined herein are arrived at by naming the terminal portion of the functionality followed by the adjacent functionality toward the point of attachment. A person of ordinary skill in the art would recognize that the above definitions are not intended to include impermissible substitution patterns (e.g., methyl substituted with 5 different groups, pentavalent carbon, and the like). Such impermissible substitution patterns are easily recognized by a person of ordinary skill in the art. All publications, patent applications, patents, and other references mentioned herein are incorporated by reference in their entirety. All sequences provided in the disclosed Genbank Accession numbers are incorporated herein by reference as available on Aug. 11, 2011. In case of conflict, the present specification, including explanations of terms, will control. In addition, the materials, methods, and examples are illustrative only and not intended to be limiting.

Alkyl groups refer to univalent groups derived from alkanes by removal of a hydrogen atom from any carbon atom, which include straight chain and branched chain with from 1 to 12 carbon atoms, and typically from 1 to about 10 carbons or in some embodiments, from 1 to about 6 carbon atoms, or in other embodiments having 1, 2, 3 or 4 carbon atoms. Examples of straight chain alkyl groups include, but are not limited to, methyl, ethyl, n-propyl, n-butyl, n-pentyl, and n-hexyl groups. Examples of branched chain alkyl groups include, but are not limited to isopropyl, isobutyl, sec-butyl and tert-butyl groups. Alkyl groups may be substituted or unsubstituted. Representative substituted alkyl groups may be mono-substituted or substituted more than once, such as, but not limited to, mono-, di-, or tri-substituted. As used herein, the term alkyl, unless otherwise stated, refers to both cyclic and noncyclic groups.

The terms “cyclic alkyl” or “cycloalkyl” refer to univalent groups derived from cycloalkanes by removal of a hydrogen atom from a ring carbon atom. Cycloalkyl groups are saturated or partially saturated non-aromatic structures with a single ring or multiple rings including isolated, fused, bridged, and spiro ring systems, having 3 to 14 carbon atoms, or in some embodiments, from 3 to 12, or 3 to 10, or 3 to 8, or 3, 4, 5, 6 or 7 carbon atoms. Cycloalkyl groups may be substituted or unsubstituted. Representative substituted cycloalkyl groups may be mono-substituted or substituted more than once, such as, but not limited to, mono-, di-, or tri-substituted. Examples of monocyclic cycloalkyl groups include, but are not limited to cyclopropyl, cyclobutyl, cyclopentyl, and cyclohexyl groups. Examples of multi-cyclic ring systems include, but are not limited to, bicycle[4.4.0]decane, bicycle[2.2.1]heptane, spiro[2.2]pentane, and the like. (Cycloalkyl)oxy refers to —O-cycloalkyl. (Cycloalkyl)thio refers to —S-cycloalkyl. This term also encompasses oxidized forms of sulfur, such as —S(O)-cycloalkyl, or —S(O)₂-cycloalkyl.

Alkenyl groups refer to straight and branched chain and cycloalkyl groups as defined above, with one or more double bonds between two carbon atoms. Alkenyl groups may have 2 to about 12 carbon atoms, or in some embodiment from 1 to about 10 carbons or in other embodiments, from 1 to about 6 carbon atoms, or 1, 2, 3 or 4 carbon atoms in other embodiments. Alkenyl groups may be substituted or unsubstituted. Representative substituted alkenyl groups may be mono-substituted or substituted more than once, such as, but not limited to, mono-, di-, or tri-substituted. Examples of alkenyl groups include, but are not limited to, vinyl, allyl, —CH═CH(CH₃), —CH═C(CH₃)₂, —C(CH₃)═CH₂, cyclopentenyl, cyclohexenyl, butadienyl, pentadienyl, and hexadienyl, among others.

Alkynyl groups refer to straight and branched chain and cycloalkyl groups as defined above, with one or more triple bonds between two carbon atoms. Alkynyl groups may have 2 to about 12 carbon atoms, or in some embodiment from 1 to about 10 carbons or in other embodiments, from 1 to about 6 carbon atoms, or 1, 2, 3 or 4 carbon atoms in other embodiments. Alkynyl groups may be substituted or unsubstituted. Representative substituted alkynyl groups may be mono-substituted or substituted more than once, such as, but not limited to, mono-, di-, or tri-substituted. Exemplary alkynyl groups include, but are not limited to, ethynyl, propargyl, and —C≡C(CH₃), among others.

Aryl groups are cyclic aromatic hydrocarbons that include single and multiple ring compounds, including multiple ring compounds that contain separate and/or fused aryl groups. Aryl groups may contain from 6 to about 18 ring carbons, or in some embodiments from 6 to 14 ring carbons or even 6 to 10 ring carbons in other embodiments. Aryl group also includes heteroaryl groups, which are aromatic ring compounds containing 5 or more ring members, one or more ring carbon atoms of which are replaced with heteroatom such as, but not limited to, N, O, and S. Aryl groups may be substituted or unsubstituted. Representative substituted aryl groups may be mono-substituted or substituted more than once, such as, but not limited to, mono-, di-, or tri-substituted. Aryl groups include, but are not limited to, phenyl, biphenylenyl, triphenylenyl, naphthyl, anthryl, and pyrenyl groups. Aryloxy refers to —O-aryl. Arylthio refers to —S-aryl, wherein aryl is as defined herein. This term also encompasses oxidized forms of sulfur, such as —S(O)-aryl, or —S(O)₂-aryl. Heteroaryloxy refers to —O-heteroaryl. Heteroarylthio refers to —S-heteroaryl. This term also encompasses oxidized forms of sulfur, such as —S(O)-heteroaryl, or —S(O)₂-heteoaryl.

Suitable heterocyclyl groups include cyclic groups with atoms of at least two different elements as members of its rings, of which one or more is a heteroatom such as, but not limited to, N, O, or S. Heterocyclyl groups may include 3 to about 20 ring members, or 3 to 18 in some embodiments, or about 3 to 15, 3 to 12, 3 to 10, or 3 to 6 ring members. The ring systems in heterocyclyl groups may be unsaturated, partially saturated, and/or saturated. Heterocyclyl groups may be substituted or unsubstituted. Representative substituted heterocyclyl groups may be mono-substituted or substituted more than once, such as, but not limited to, mono-, di-, or tri-substituted. Exemplary heterocyclyl groups include, but are not limited to, pyrrolidinyl, tetrahydrofuryl, dihydrofuryl, tetrahydrothienyl, tetrahydrothiopyranyl, piperidyl, morpholinyl, thiomorpholinyl, thioxanyl, piperazinyl, azetidinyl, aziridinyl, imidazolidinyl, pyrazolidinyl, thiazolidinyl, tetrahydrothiophenyl, tetrahydrofuranyl, dioxolyl, furanyl, thiophenyl, pyrrolyl, imidazolyl, pyrazolyl, pyrazolinyl, triazolyl, tetrazolyl, oxazolyl, isoxazolyl, thiazolyl, thiazolinyl, oxetanyl, thietanyl, homopiperidyl, oxepanyl, thiepanyl, oxazepinyl, diazepinyl, thiazepinyl, 1,2,3,6-tetrahydropyridyl, indolinyl, 2H-pyranyl, 4H-pyranyl, dioxolanyl, dioxanyl, purinyl, quinolizinyl, cinnolinyl, phthalazinyl, pteridinyl, and benzothiazolyl groups. Heterocyclyloxy refers to —O— heterocycyl. Heterocyclylthio refers to —S-heterocycyl. This term also encompasses oxidized forms of sulfur, such as —S(O)-heterocyclyl, or —S(O)₂-heterocyclyl.

Polycyclic or polycyclyl groups refer to two or more rings in which two or more carbons are common to the two adjoining rings, wherein the rings are “fused rings”; if the rings are joined by one common carbon atom, these are “spiro” ring systems. Rings that are joined through non-adjacent atoms are “bridged” rings. Polycyclic groups may be substituted or unsubstituted. Representative polycyclic groups may be substituted one or more times.

Halogen groups include F, C₁, Br, and I; nitro group refers to —NO₂; cyano group refers to —CN; isocyano group refers to —N≡C; epoxy groups encompass structures in which an oxygen atom is directly attached to two adjacent or non-adjacent carbon atoms of a carbon chain or ring system, which is essentially a cyclic ether structure. An epoxide is a cyclic ether with a three-atom ring.

An alkoxy group is a substituted or unsubstituted alkyl group, as defined above, singular bonded to oxygen. Alkoxy groups may be substituted or unsubstituted. Representative substituted alkoxy groups may be substituted one or more times. Exemplary alkoxy groups include, but are not limited to, methoxy, ethoxy, propoxy, butoxy, pentoxy, hexoxy, isopropoxy, sec-butoxy, tert-butoxy, cyclopropyloxy, cyclobutyloxy, cyclopentyloxy, and cyclohexyloxy groups.

Thiol refers to —SH. Thiocarbonyl refers to (═S). Sulfonyl refers to —SO₂-alkyl, —SO₂-substituted alkyl, —SO₂-cycloalkyl, —SO₂-substituted cycloalkyl, —SO₂-aryl, —SO₂-substituted aryl, —SO₂-heteroaryl, —SO₂-substituted heteroaryl, —SO₂-heterocyclyl, and —SO₂-substituted heterocyclyl. Sulfonylamino refers to —NRaSO₂alkyl, —NRaSO₂-substituted alkyl, —NRaSO₂cycloalkyl, —NR^{a}SO₂substituted cycloalkyl, —NR^{a}SO₂aryl, —NR^{a}SO₂substituted aryl, -—NR^{a}SO₂heteroaryl, —NR^{a}SO₂ substituted heteroaryl, —NR^{a}SO₂heterocyclyl, —NR^{a}SO₂ substituted heterocyclyl, wherein each R^{a} independently is as defined herein.

Carboxyl refers to —COOH or salts thereof. Carboxyester refers to —C(O)O-alkyl, —C(O)O— substituted alkyl, —C(O)O-aryl, —C(O)O-substituted aryl, —C(O)β-cycloalkyl, —C(O)O-substituted cycloalkyl, —C(O)O-heteroaryl, —C(O)O-substituted heteroaryl, —C(O)O-heterocyclyl, and —C(O)O— substituted heterocyclyl. (Carboxyester)amino refers to —N α—C(O)O-alkyl, —NR^{a}—C(O)O— substituted alkyl, —NR^{a}—C(O)O-aryl, —NR^{a}—C(O)O-substituted aryl, —NR^{a}—C(O)β-cycloalkyl, —NR^{a}C(O)O-substituted cycloalkyl, —NR^{a}—C(O)O-heteroaryl, —NR^{a}C(O)O-substituted heteroaryl, —NR^{a}C(O)O-heterocyclyl, and —NR^{a}—C(O)O-substituted heterocyclyl, wherein R^{a} is as recited herein. (Carboxyester)oxy refers to —O—C(O)O-alkyl, —O—C(O)O— substituted alkyl, —O—C(O)O-aryl, —O—C(O)O-substituted aryl, —O—C(O)β-cycloalkyl, —O—C(O)O-substituted cycloalkyl, —O—C(O)O— heteroaryl, —O—C(O)O-substituted heteroaryl, —O—C(O)O-heterocyclyl, and —O—C(O)O-substituted heterocyclyl. Oxo refers to (═O).

The terms “amine” and “amino” refer to derivatives of ammonia, wherein one of more hydrogen atoms have been replaced by a substituent which include, but are not limited to alkyl, alkenyl, aryl, and heterocyclyl groups. Carbamate groups refers to —O(C≡O)NR₁R₂, where R₁ and R₂ are independently hydrogen, aliphatic groups, aryl groups, or heterocyclyl groups.

Aminocarbonyl refers to —C(O)N(R)₂, wherein each R^{b} independently is selected from hydrogen, alkyl, substituted alkyl, aryl, substituted aryl, cycloalkyl, substituted cycloalkyl, heteroaryl, substituted heteroaryl, heterocyclyl, substituted heterocyclyl. Also, each R^{b} may optionally be joined together with the nitrogen bound thereto to form a heterocyclyl or substituted heterocyclyl group, provided that both R^{b} are not both hydrogen. Aminocarbonylalkyl refers to-alkylC(O)N(R)₂, wherein each R^{b} independently is selected from hydrogen, alkyl, substituted alkyl, aryl, substituted aryl, cycloalkyl, substituted cycloalkyl, heteroaryl, substituted heteroaryl, heterocyclyl, substituted heterocyclyl. Also, each R^{b} may optionally be joined together with the nitrogen bound thereto to form a heterocyclyl or substituted heterocyclyl group, provided that both R^{b} are not both hydrogen. Aminocarbonylamino refers to —NR^{a}C(O)N(R)₂, wherein R^{a} and each R^{b}are as defined herein. Aminodicarbonylamino refers to —NR^{a}C(O)C(O)N(R^{b})₂, wherein R^{a} and each R^{b} are as defined herein. Aminocarbonyloxy refers to —O—C(O)N(R^{b})₂, wherein each R^{b}independently is as defined herein. Aminosulfonyl refers to —SO₂N(R^{b})₂, wherein each R^{b}independently is as defined herein.

Imino refers to —N═R^{c} wherein R^{c} may be selected from hydrogen, aminocarbonylalkyloxy, substituted aminocarbonylalkyloxy, aminocarbonylalkylamino, and substituted aminocarbonylalkylamino.

The term “optionally substituted” means the anteceding group may be substituted or unsubstituted. When substituted, the substituents of an “optionally substituted” group may include, without limitation, one or more substituents independently selected from the following groups or a particular designated set of groups, alone or in combination: lower alkyl, lower alkenyl, lower alkynyl, lower alkanoyl, lower heteroalkyl, lower heterocycloalkyl, lower haloalkyl, lower haloalkenyl, lower haloalkynyl, lower perhaloalkyl, lower perhaloalkoxy, lower cycloalkyl, phenyl, aryl, aryloxy, lower alkoxy, lower haloalkoxy, oxo, lower acyloxy, carbonyl, carboxyl, lower alkylcarbonyl, lower carboxyester, lower carboxamido, cyano, hydrogen, halogen, hydroxy, amino, lower alkylamino, arylamino, amido, nitro, thiol, lower alkylthio, lower haloalkylthio, lower perhaloalkylthio, arylthio, sulfonate, sulfonic acid, trisubstituted silyl, N₃, SH, SCH₃, C(O)CH₃, CO₂CH₃, CO₂H, pyridinyl, thiophene, furanyl, lower carbamate, and lower urea. Two substituents may be joined together to form a fused five-, six-, or seven-membered carbocyclic or heterocyclic ring consisting of zero to three heteroatoms, for example forming methylenedioxy or ethylenedioxy. An optionally substituted group may be unsubstituted (e.g., —CH₂CH₃), fully substituted (e.g., —CF₂CF₃), monosubstituted (e.g., —CH₂CH₂F) or substituted at a level anywhere in-between fully substituted and monosubstituted (e.g., —CH₂CF₃). Where substituents are recited without qualification as to substitution, both substituted and unsubstituted forms are encompassed. Where a substituent is qualified as “substituted,” the substituted form is specifically intended. Additionally, different sets of optional substituents to a particular moiety may be defined as needed; in these cases, the optional substitution will be as defined, often immediately following the phrase, “optionally substituted with.”

The term “bioconjugation linking moiety,” as used herein, is defined as the chemical product of a chemically co-reactive pair. A bioconjugation linking moiety can comprise a number of various moieties that would be appreciated by a person of ordinary skill in the art.

The term “biological target,” as used herein, is defined a protein, peptide, or nucleic acid with activity that can be modulated by some other entity, e.g., like an endogenous ligand or drug.

The term “building block,” as used herein, is defined as a structural unit of a chemical entity, where the unit is directly linked to other chemical structural units or indirectly linked through the scaffold. When the chemical entity is polymeric or oligomeric, the building blocks are the monomeric units of the polymer or oligomer. Building blocks can have one or more diversity nodes that allow for the addition of one or more other building blocks or scaffolds. In most cases, each diversity node is a functional group capable of reacting with one or more building blocks or scaffolds to form a chemical entity. Generally, the building blocks have at least two diversity nodes (or reactive functional groups), but some building blocks may have one diversity node (or reactive functional group). Alternatively, the encoded chemical or binding steps may include several chemical components (e.g., multi-component condensation reactions or multi-step processes). Reactive groups on two different building blocks should be complementary, i.e., capable of reacting together to form a covalent or a non-covalent bond.

The term “chemically co-reactive pair,” as used herein is defined as a pair of reactive groups that participates in a modular reaction with high yield and a high thermodynamic gain, thus producing a spacer. Exemplary reactions and chemically co-reactive pairs include a Huisgen 1,3-dipolar cycloaddition reaction with a pair of an optionally substituted alkynyl group and an optionally substituted azido group; a Diels-Alder reaction with a pair of an optionally substituted diene having a 4.pi. electron system and an optionally substituted dienophile or an optionally substituted heterodienophile having a 2.pi. electron system; a ring opening reaction with a nucleophile and a strained heterocyclyl electrophile; a splint ligation reaction with a phosphorothioate group and an iodo group; and a reductive amination reaction with an aldehyde group and an amino group, as described herein.

“Covalent” or “covalent bond,” as used herein, is defined as a chemical bond that involves the sharing of electron pairs between atoms. These electron pairs are known as shared pairs or bonding pairs and the stable balance of attractive and repulsive forces between atoms when they share electrons is known as covalent bonding. For many molecules, the sharing of electrons allows each atom to attain the equivalent of a full outer shell, corresponding to a stable electronic configuration. Covalent bonding includes many kinds of interactions, including α-bonding, 71-bonding, metal-to-metal bonding, agostic interactions, bent bonds, and three-center two-electron bonds.

Pharmaceutically acceptable salts of compounds described herein include conventional nontoxic salts or quaternary ammonium salts of a compound, e.g., from non-toxic organic or inorganic acids. For example, such conventional nontoxic salts include those derived from inorganic acids such as hydrochloride, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2- acetoxybenzoic, fumaric, toluenesulfonic, methanesulfonic, ethane disulfonic, oxalic, isothionic, and the like. In other cases, described compounds may contain one or more acidic functional groups and, thus, are capable of forming pharmaceutically acceptable salts with pharmaceutically acceptable bases. These salts can likewise be prepared in situ in the administration vehicle or the dosage form manufacturing process, or by separately reacting the purified compound in its free acid form with a suitable base, such as the hydroxide, carbonate or bicarbonate of a pharmaceutically acceptable metal cation, with ammonia, or with a pharmaceutically acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like.

The term “oligonucleotide,” as used herein, is defined as a polymer of nucleotides having a 5′-terminus, a 3′-terminus, and one or more nucleotides at the internal position between the 5′- and 3′-termini. The oligonucleotide may include DNA, RNA, or any derivative thereof known in the art that can be synthesized and used for base-pair recognition. The oligonucleotide does not have to have contiguous bases but can be interspersed with linker moieties. The oligonucleotide polymer and nucleotide (e.g., modified DNA or RNA) may include natural bases (e.g., adenosine, thymidine, guanosine, cytidine, uridine, deoxyadenosine, deoxythymidine, deoxyguanosine, deoxycytidine, inosine, or diamino purine), base analogs (e.g., 2-aminoadenosine, 2-thiothymidine, inosine, pyrrolo-pyrimidine, 3-methyl adenosine, C5-propynylcytidine, C5-propynyluridine, C5-bromouridine, C5-fluorouridine, C5-iodouridine, C5-methylcytidine, 7-deazaadenosine, 7-deazaguanosine, 8-oxoadenosine, 8-oxoguanosine, (6)-methylguanine, and 2-thiocytidine), modified bases (e.g., 2′-substituted nucleotides, such as 2′-methylated bases and 2′-fluoro bases), intercalated bases, modified sugars (e.g., 2′-fluororibose; ribose; 2′-deoxyribose; arabinose; hexose; anhydrohexitol; altritol; mannitol; cyclohexanyl; cyclohexenyl; morpholino that also has a phosphoramidate backbone; locked nucleic acids (LNA, e.g., where the 2′-hydroxyl of the ribose is connected by a alkylene or heteroalkylene bridge to the 4′-carbon of the same ribose sugar, where exemplary bridges included methylene, propylene, ether, or amino bridges); glycol nucleic acid (GNA, e.g., R-GNA or S-GNA, where ribose is replaced by glycol units attached to phosphodiester bonds); threose nucleic acid (TNA, where ribose is replace with α—L-threofuranosyl-(3′→2′)); and/or replacement of the oxygen in ribose (e.g., with S, Se, or alkylene, such as methylene or ethylene)), modified backbones (e.g., peptide nucleic acid (PNA), where 2-amino-ethyl-glycine linkages replace the ribose and phosphodiester backbone), and/or modified phosphate groups (e.g., phosphorothioates, 5′—N-phosphoramidites, phosphoroselenates, boranophosphates, boranophosphate esters, hydrogen phosphonates, phosphoramidates, phosphorodiamidates, alkyl or aryl phosphonates, phosphotriesters, bridged phosphoramidates, bridged phosphorothioates, and bridged methylene-phosphonates). The oligonucleotide can be single-stranded (e.g., hairpin), double-stranded, or possess other secondary or tertiary structures (e.g., stem-loop structures, double helixes, triplexes, quadruplexes, etc.). Oligonucleotides may also contain one or more 3′-3′ or 5′-5′ linkages, or one or more inverted nucleotides. This may mean that they contain two 3′-termini or two 5′-termini. Oligonucleotides may also branch one or more times, wherein they may contain more than two termini. Oligonucleotides may also be circularized, wherein they may contain less than two termini and may contain no termini at all.

The terms “operatively linked” or “operative linkage,” as used herein, is defined as two or more chemical structures that are directly or indirectly linked together in such a way as to remain linked through the various manipulations they are expected to undergo.

The term “treatment” is used interchangeably herein with the term “therapeutic method” and refers to both 1) therapeutic treatments or measures that cure, slow down, lessen symptoms of, and/or halt progression of a diagnosed pathologic conditions, disease or disorder, and 2) and prophylactic/preventative measures. Those in need of treatment may include individuals already having a particular medical disease or disorder as well as those who may ultimately acquire the disorder (i.e., those needing preventive measures).

The term “subject” as used herein refers to any individual or patient to which the subject methods are performed. Generally, the subject is human, although as will be appreciated by those in the art, the subject may be an animal.

The terms “therapeutically effective amount”, “effective dose”, “therapeutically effective dose”, “effective amount,” or the like refer to the amount of a subject compound that will elicit the biological or medical response in a tissue, system, animal or human that is being sought by administering said compound. Generally, the response is either amelioration of symptoms in a patient or a desired biological outcome. Such amount should be sufficient to inhibit MIF activity.

Also disclosed herein are pharmaceutical compositions including compounds with the structures of Formula (I). The term “pharmaceutically acceptable carrier” refers to a non-toxic carrier that may be administered to a patient, together with a compound of this disclosure, and which does not destroy the pharmacological activity thereof. Pharmaceutically acceptable carriers that may be used in these compositions include, but are not limited to, ion exchangers, alumina, aluminum stearate, lecithin, serum proteins such as human serum albumin, buffer substances such as phosphates, glycine, sorbic acid, potassium sorbate, partial glyceride mixtures of saturated vegetable fatty acids, water, salts or electrolytes such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Pharmaceutically acceptable carriers that may be used in the pharmaceutical compositions of this disclosure include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, wool fat and self-emulsifying drug delivery systems (SEDDS) such as α-tocopherol, polyethyleneglycol 1000 succinate, or other similar polymeric delivery matrices.

In pharmaceutical composition comprising only the compounds described herein as the active component, methods for administering these compositions may additionally comprise the step of administering to the subject an additional agent or therapy. Such therapies include, but are not limited to, an anemia therapy, a diabetes therapy, a hypertension therapy, a cholesterol therapy, neuropharmacologic drugs, drugs modulating cardiovascular function, drugs modulating inflammation, immune function, production of blood cells; hormones and antagonists, drugs affecting gastrointestinal function, chemotherapeutics of microbial diseases, and/or chemotherapeutics of neoplastic disease. Other pharmacological therapies can include any other drug or biologic found in any drug class. For example, other drug classes can comprise allergy/cold/ENT therapies, analgesics, anesthetics, anti-inflammatories, antimicrobials, antivirals, asthma/pulmonary therapies, cardiovascular therapies, dermatology therapies, endocrine/metabolic therapies, gastrointestinal therapies, cancer therapies, immunology therapies, neurologic therapies, ophthalmic therapies, psychiatric therapies or rheumatologic therapies. Other examples of agents or therapies that can be administered with the compounds described herein include a matrix metalloprotease inhibitor, a lipoxygenase inhibitor, a cytokine antagonist, an immunosuppressant, a cytokine, a growth factor, an immunomodulator, a prostaglandin or an anti-vascular hyperproliferation compound.

The term “therapeutically effective amount” as used herein refers to the amount of active compound or pharmaceutical agent that elicits the biological or medicinal response in a tissue, system, animal, individual or human that is being sought by a researcher, veterinarian, medical doctor or other clinician, which includes one or more of the following: (1) Preventing the disease; for example, preventing a disease, condition or disorder in an individual that may be predisposed to the disease, condition or disorder but does not yet experience or display the pathology or symptomatology of the disease, (2) Inhibiting the disease; for example, inhibiting a disease, condition or disorder in an individual that is experiencing or displaying the pathology or symptomatology of the disease, condition or disorder (i.e., arresting further development of the pathology and/or symptomatology), and (3) Ameliorating the disease; for example, ameliorating a disease, condition or disorder in an individual that is experiencing or displaying the pathology or symptomatology of the disease, condition or disorder (i.e., reversing the pathology and/or symptomatology).

The compounds of this disclosure may be employed in a conventional manner for controlling the disease described herein, including, but not limited to, myocardial infarction, renal ischemia, stroke, ischemia, Alzheimer's disease, Parkinson's disease, Lewy Body Dementia, Multi-Systems Atrophy, Gehrig's disease (Amyotrophic Lateral Sclerosis), Huntington's disease, Multiple Sclerosis, senile dementia, subcortical dementia, arteriosclerotic dementia, AIDS-associated dementia, other dementias, cerebral vasculitis, epilepsy, Tourette's syndrome, Guillain Bane Syndrome, Wilson's disease, Pick's disease, encephalitis, encephalomyelitis, meningitis, prion diseases, cerebellar ataxias, cerebellar degeneration, spinocerebellar degeneration syndromes, Friedrich's ataxia, ataxia telangiectasia, spinal dysmyotrophy, progressive supranuclear palsy, dystonia, muscle spasticity, tremor, retinitis pigmentosa, striatonigral degeneration, mitochondrial encephalomyopathies and neuronal ceroid lipofuscinosis.. Such methods of treatment, their dosage levels and requirements may be selected by those of ordinary skill in the art from available methods and techniques.

Alternatively, the compounds of this disclosure may be used in compositions and methods for treating or protecting individuals against the diseases described herein, including but not limited to a myocardial infarction, renal ischemia stroke, ischemia, Alzheimer's disease, Parkinson's disease, Lewy Body Dementia, Multi-Systems Atrophy, Gehrig's disease (Amyotrophic Lateral Sclerosis), Huntington's disease, Multiple Sclerosis, senile dementia, subcortical dementia, arteriosclerotic dementia, AIDS-associated dementia, other dementias, cerebral vasculitis, epilepsy, Tourette's syndrome, Guillain Bane Syndrome, Wilson's disease, Pick's disease, encephalitis, encephalomyelitis, meningitis, prion diseases, cerebellar ataxias, cerebellar degeneration, spinocerebellar degeneration syndromes, Friedrich's ataxia, ataxia telangiectasia, spinal dysmyotrophy, progressive supranuclear palsy, dystonia, muscle spasticity, tremor, retinitis pigmentosa, striatonigral degeneration, mitochondrial encephalomyopathies and neuronal ceroid lipofuscinosis, over extended periods of time. The compounds may be employed in such compositions either alone or together with other compounds of this disclosure in a manner consistent with the conventional utilization of such compounds in pharmaceutical compositions. For example, a compound of this disclosure may be combined with pharmaceutically acceptable adjuvants conventionally employed in vaccines and administered in prophylactically effective amounts to protect individuals over an extended period of time against the diseases described herein.

As used herein, the terms “combination,” “combined,” and related terms refer to the simultaneous or sequential administration of therapeutic agents in accordance with this disclosure. For example, a described compound may be administered with another therapeutic agent simultaneously or sequentially in separate unit dosage forms or together in a single unit dosage form. Accordingly, the present disclosure provides a single unit dosage form comprising a described compound, an additional therapeutic agent, and a pharmaceutically acceptable carrier, adjuvant, or vehicle. Two or more agents are typically considered to be administered “in combination” when a patient or individual is simultaneously exposed to both agents. In many embodiments, two or more agents are considered to be administered “in combination” when a patient or individual simultaneously shows therapeutically relevant levels of the agents in a particular target tissue or sample (e.g., in brain, in serum, etc.).

When the compounds of this disclosure are administered in combination therapies with other agents, they may be administered sequentially or concurrently to the patient. Alternatively, pharmaceutical or prophylactic compositions according to this disclosure comprise a combination of ivermectin, or any other compound described herein, and another therapeutic or prophylactic agent. Additional therapeutic agents that are normally administered to treat a particular disease or condition may be referred to as “agents appropriate for the disease, or condition, being treated.”

The compounds utilized in the compositions and methods of this disclosure may also be modified by appending appropriate functionalities to enhance selective biological properties. Such modifications are known in the art and include those, which increase biological penetration into a given biological system (e.g., blood, lymphatic system, or central nervous system), increase oral availability, increase solubility to allow administration by injection, alter metabolism and/or alter rate of excretion.

According to a preferred embodiment, the compositions of this disclosure are formulated for pharmaceutical administration to a subject or patient, e.g., a mammal, preferably a human being. Such pharmaceutical compositions are used to ameliorate, treat or prevent any of the diseases described herein in a subject.

Agents of the disclosure are often administered as pharmaceutical compositions comprising an active therapeutic agent, i.e., and a variety of other pharmaceutically acceptable components. See Remington's Pharmaceutical Science (15th ed., Mack Publishing Company, Easton, Pa., 1980). The preferred form depends on the intended mode of administration and therapeutic application. The compositions can also include, depending on the formulation desired, pharmaceutically acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, physiological phosphate-buffered saline, Ringer's solutions, dextrose solution, and Hank's solution. In addition, the pharmaceutical composition or formulation may also include other carriers, adjuvants, or nontoxic, nontherapeutic, nonimmunogenic stabilizers and the like.

In some embodiments, the present disclosure provides pharmaceutically acceptable compositions comprising a therapeutically effective amount of one or more of a described compound, formulated together with one or more pharmaceutically acceptable carriers (additives) and/or diluents for use in treating the diseases described herein, including, but not limited to stroke, ischemia, Alzheimer's, ankylosing spondylitis, arthritis, osteoarthritis, rheumatoid arthritis, psoriatic arthritis, asthma atherosclerosis, Crohn's disease, colitis, dermatitis diverticulitis, fibromyalgia, hepatitis, irritable bowel syndrome, systemic lupus erythematous, nephritis, ulcerative colitis and Parkinson's disease. While it is possible for a described compound to be administered alone, it is preferable to administer a described compound as a pharmaceutical formulation (composition) as described herein. Described compounds may be formulated for administration in any convenient way for use in human or veterinary medicine, by analogy with other pharmaceuticals.

As described in detail, pharmaceutical compositions of the present disclosure may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream or foam; sublingually; ocularly; transdermally; or nasally, pulmonary and to other mucosal surfaces.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, alpha-tocopherol, and the like; and metal chelating agents, such as citric acid, ethylenediamine tetraacetic acid (EDTA), sorbitol, tartaric acid, phosphoric acid, and the like.

Formulations for use in accordance with the present disclosure include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient, which can be combined with a carrier material, to produce a single dosage form will vary depending upon the host being treated, and the particular mode of administration. The amount of active ingredient that can be combined with a carrier material to produce a single dosage form will generally be that amount of the compound, which produces a therapeutic effect. Generally, this amount will range from about 1% to about 99% of active ingredient. In some embodiments, this amount will range from about 5% to about 70%, from about 10% to about 50%, or from about 20% to about 40%.

In certain embodiments, a formulation as described herein comprises an excipient selected from the group consisting of cyclodextrins, liposomes, micelle forming agents, e.g., bile acids, and polymeric carriers, e.g., polyesters and polyanhydrides; and a compound of the present disclosure. In certain embodiments, an aforementioned formulation renders orally bioavailable a described compound of the present disclosure.

Methods of preparing formulations or compositions comprising described compounds include a step of bringing into association a compound of the present disclosure with the carrier and, optionally, one or more accessory ingredients. In general, formulations may be prepared by uniformly and intimately bringing into association a compound of the present disclosure with liquid carriers, or finely divided solid carriers, or both, and then, if necessary, shaping the product.

The pharmaceutical compositions may be in the form of a sterile injectable preparation, for example, as a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long-chain alcohol diluent or dispersant, such as those described in Pharmacopeia Helvetica, or a similar alcohol. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

In some cases, in order to prolong the effect of a drug, it may be desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which in turn, may depend upon crystal size and crystalline form. Alternatively, delayed absorption of a parenterally administered drug form is accomplished by dissolving or suspending the drug in an oil vehicle.

Injectable depot forms are made by forming microencapsule matrices of the described compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions, which are compatible with body tissue.

The pharmaceutical compositions of this disclosure may be orally administered in any orally acceptable dosage form including, but not limited to, capsules, tablets, and aqueous suspensions and solutions. In the case of tablets for oral use, carriers, which are commonly used include lactose and corn starch. Lubricating agents, such as magnesium stearate, are also typically added. For oral administration in a capsule form, useful diluents include lactose and dried cornstarch. When aqueous suspensions and solutions and propylene glycol are administered orally, the active ingredient is combined with emulsifying and suspending agents. If desired, certain sweetening and/or flavoring and/or coloring agents may be added.

Formulations described herein suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present disclosure as an active ingredient. Compounds described herein may also be administered as a bolus, electuary or paste.

In solid dosage forms for oral administration (capsules, tablets, pills, dragees, powders, granules and the like), an active ingredient is mixed with one or more pharmaceutically-acceptable carriers, such as sodium citrate or dicalcium phosphate, and/or any of the following: fillers or extenders, such as starches, lactose, sucrose, glucose, mannitol, and/or silicic acid; binders, such as, for example, carboxymethylcellulose, alginates, gelatin, polyvinyl pyrrolidone, sucrose and/or acacia; humectants, such as glycerol; disintegrating agents, such as agar-agar, calcium carbonate, potato or tapioca starch, alginic acid, certain silicates, and sodium carbonate; solution retarding agents, such as paraffin; absorption accelerators, such as quaternary ammonium compounds; wetting agents, such as, for example, cetyl alcohol, glycerol monostearate, and non-ionic surfactants; absorbents, such as kaolin and bentonite clay; lubricants, such as talc, calcium stearate, magnesium stearate, solid polyethylene glycols, sodium lauryl sulfate, and mixtures thereof; and coloring agents. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

Tablets may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made in a suitable machine in which a mixture of the powdered compound is moistened with an inert liquid diluent. If a solid carrier is used, the preparation can be in tablet form, placed in a hard gelatin capsule in powder or pellet form, or in the form of a troche or lozenge. The amount of solid carrier will vary, e.g., from about 25 to 800 mg, preferably about 25 mg to 400 mg. When a liquid carrier is used, the preparation can be, e.g., in the form of a syrup, emulsion, soft gelatin capsule, sterile injectable liquid such as an ampule or nonaqueous liquid suspension. Where the composition is in the form of a capsule, any routine encapsulation is suitable, for example, using the aforementioned carriers in a hard gelatin capsule shell.

Tablets and other solid dosage forms, such as dragees, capsules, pills and granules, may optionally be scored or prepared with coatings and shells, such as enteric coatings and other coatings well known in the pharmaceutical-formulating art. They may alternatively or additionally be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze- dried. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms for oral administration of compounds of the disclosure include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3- butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, oral compositions can also include adjuvants such as wetting agents, emulsifying and suspending agents, sweetening, flavoring, coloring, perfuming and preservative agents.

Suspensions, in addition to active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

The pharmaceutical compositions of this disclosure may also be administered in the form of suppositories for rectal administration. These compositions can be prepared by mixing a compound of this disclosure with a suitable non-irritating excipient, which is solid at room temperature but liquid at the rectal temperature and therefore will melt in the rectum to release the active components. Such materials include, but are not limited to, cocoa butter, beeswax and polyethylene glycols.

Topical administration of the pharmaceutical compositions of this disclosure is especially useful when the desired treatment involves areas or organs readily accessible by topical application. For application topically to the skin, the pharmaceutical composition should be formulated with a suitable ointment containing the active components suspended or dissolved in a carrier. Carriers for topical administration of the compounds of this disclosure include, but are not limited to, mineral oil, liquid petroleum, white petroleum, propylene glycol, polyoxyethylene polyoxypropylene compound, emulsifying wax and water. Alternatively, the pharmaceutical composition can be formulated with a suitable lotion or cream containing the active compound suspended or dissolved in a carrier. Suitable carriers include, but are not limited to, mineral oil, sorbitan monostearate, polysorbate 60, cetyl esters wax, cetearyl alcohol, 2-octyldodecanol, benzyl alcohol and water. The pharmaceutical compositions of this disclosure may also be topically applied to the lower intestinal tract by rectal suppository formulation or in a suitable enema formulation. Topically-administered transdermal patches are also included in this disclosure.

The pharmaceutical compositions of this disclosure may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

For ophthalmic use, the pharmaceutical compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutical compositions may be formulated in an ointment such as petrolatum.

Transdermal patches have the added advantage of providing controlled delivery of a compound of the present disclosure to the body. Dissolving or dispersing the compound in the proper medium can make such dosage forms. Absorption enhancers can also be used to increase the flux of the compound across the skin. Either providing a rate controlling membrane or dispersing the compound in a polymer matrix or gel can control the rate of such flux.

Examples of suitable aqueous and nonaqueous carriers, which may be employed in the pharmaceutical compositions of the disclosure, include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

Such compositions may also contain adjuvants such as preservatives, wetting agents, emulsifying agents and dispersing agents. Inclusion of one or more antibacterial and/orantifungal agents, for example, paraben, chlorobutanol, phenol sorbic acid, and the like, may be desirable in certain embodiments. It may alternatively or additionally be desirable to include isotonic agents, such as sugars, sodium chloride, and the like into the compositions. In addition, prolonged absorption of the injectable pharmaceutical form may be brought about by the inclusion of agents, which delay absorption such as aluminum monostearate and gelatin.

In certain embodiments, a described compound or pharmaceutical preparation is administered orally. In other embodiments, a described compound or pharmaceutical preparation is administered intravenously. Alternative routes of administration include sublingual, intramuscular, and transdermal administrations.

When compounds described herein are administered as pharmaceuticals, to humans and animals, they can be given per se or as a pharmaceutical composition containing, for example, 0.1% to 99.5% (more preferably, 0.5% to 90%) of active ingredient in combination with a pharmaceutically acceptable carrier.

Preparations described herein may be given orally, parenterally, topically, or rectally. They are of course given in forms suitable for the relevant administration route. For example, they are administered in tablets or capsule form, by injection, inhalation, eye lotion, ointment, suppository, etc. administration by injection, infusion or inhalation; topical by lotion or ointment; and rectal by suppositories. Oral administrations are preferred.

Such compounds may be administered to humans and other animals for therapy by any suitable route of administration, including orally, nasally, as by, for example, a spray, rectally, intravaginally, parenterally, intracisternally and topically, as by powders, ointments or drops, including buccally and sublingually.

Regardless of the route of administration selected, compounds described herein which may be used in a suitable hydrated form, and/or the pharmaceutical compositions of the present disclosure, are formulated into pharmaceutically-acceptable dosage forms by conventional methods known to those of skill in the art.

Actual dosage levels of the active ingredients in the pharmaceutical compositions of the disclosure may be varied so as to obtain an amount of the active ingredient that is effective to achieve the desired therapeutic response for a particular patient, composition, and mode of administration, without being toxic to the patient.

The terms “administration of” and or “administering” should be understood to mean providing a pharmaceutical composition in a therapeutically effective amount to the subject in need of treatment. Administration routes can be enteral, topical or parenteral. As such, administration routes include but are not limited to intracutaneous, subcutaneous, intravenous, intraperitoneal, intraarterial, intrathecal, intracapsular, intraorbital, intracardiac, intradermal, transdermal, transtracheal, subcuticular, intraarticulare, subcapsular, subarachnoid, intraspinal and intrasternal, oral, sublingual buccal, rectal, vaginal, nasal ocular administrations, as well infusion, inhalation, and nebulization.

In treatment, the dose of agent optionally ranges from about 0.0001 mg/kg to about 100 mg/kg, about 0.01 mg/kg to about 5 mg/kg, about 0.15 mg/kg to about 3 mg/kg, 0.5 mg/kg to about 2 mg/kg and about 1 mg/kg to about 2 mg/kg of the subject's body weight. In other embodiments the dose ranges from about 100 mg/kg to about 5 g/kg, about 500 mg/kg to about 2 mg/kg and about 750 mg/kg to about 1.5 g/kg of the subject's body weight. For example, depending on the type and severity of the disease, about 1 μg/kg to 15 mg/kg (e.g., 0.1-20 mg/kg) of agent is a candidate dosage for administration to the patient, whether, for example, by one or more separate administrations, or by continuous infusion. A typical daily dosage is in the range from about 1 μg/kg to 100 mg/kg or more, depending on the factors mentioned above. For repeated administrations over several days or longer, depending on the condition, the treatment is sustained until a desired suppression of disease symptoms occurs. However, other dosage regimens may be useful. Unit doses can be in the range, for instance of about 5 mg to 500 mg, such as 50 mg, 100 mg, 150 mg, 200 mg, 250 mg and 300 mg. The progress of therapy is monitored by conventional techniques and assays.

In some embodiments, an agent is administered to a human patient at an effective amount (or dose) of less than about 1 μg/kg, for instance, about 0.35 to about 0.75 μg/kg or about 0.40 to about 0.60 μg/kg. In some embodiments, the dose of an agent is about 0.35 μg/kg, or about 0.40 μg/kg, or about 0.45 μg/kg, or about 0.50 μg/kg, or about 0.55 μg/kg, or about 0.60 μg/kg, or about 0.65 μg/kg, or about 0.70 μg/kg, or about 0.75 μg/kg, or about 0.80 μg/kg, or about 0.85 μg/kg, or about 0.90 μg/kg, or about 0.95 μg/kg or about 1 μg/kg. In various embodiments, the absolute dose of an agent is about 2 μg/subject to about 45 μg/subject, or about 5 to about 40, or about 10 to about 30, or about 15 to about 25 μg/subject. In some embodiments, the absolute dose of an agent is about 20 μg, or about 30 μg, or about 40 μg.

In various embodiments, the dose of an agent may be determined by the human patient's body weight. For example, an absolute dose of an agent of about 2 μg for a pediatric human patient of about 0 to about 5 kg (e.g. about 0, or about 1, or about 2, or about 3, or about 4, or about 5 kg); or about 3 μg for a pediatric human patient of about 6 to about 8 kg (e.g. about 6, or about 7, or about 8 kg), or about 5 μg for a pediatric human patient of about 9 to about 13 kg (e.g. 9, or about 10, or about 11, or about 12, or about 13 kg); or about 8 μg for a pediatric human patient of about 14 to about 20 kg (e.g. about 14, or about 16, or about 18, or about 20 kg), or about 12 μg for a pediatric human patient of about 21 to about 30 kg (e.g. about 21, or about 23, or about 25, or about 27, or about 30 kg), or about 13 μg for a pediatric human patient of about 31 to about 33 kg (e.g. about 31, or about 32, or about 33 kg), or about 20 μg for an adult human patient of about 34 to about 50 kg (e.g. about 34, or about 36, or about 38, or about 40, or about 42, or about 44, or about 46, or about 48, or about 50 kg), or about 30 μg for an adult human patient of about 51 to about 75 kg (e.g. about 51, or about 55, or about 60, or about 65, or about 70, or about 75 kg), or about 45 μg for an adult human patient of greater than about 114 kg (e.g. about 114, or about 120, or about 130, or about 140, or about 150 kg).

In certain embodiments, an agent in accordance with the methods provided herein is administered subcutaneously (s.c.), intraveneously (i.v.), intramuscularly (i.m.), intranasally or topically. Administration of an agent described herein can, independently, be one to four times daily or one to four times per month or one to six times per year or once every two, three, four or five years. Administration can be for the duration of one day or one month, two months, three months, six months, one year, two years, three years, and may even be for the life of the human patient. The dosage may be administered as a single dose or divided into multiple doses. In some embodiments, an agent is administered about 1 to about 3 times (e.g. 1, or 2 or 3 times).

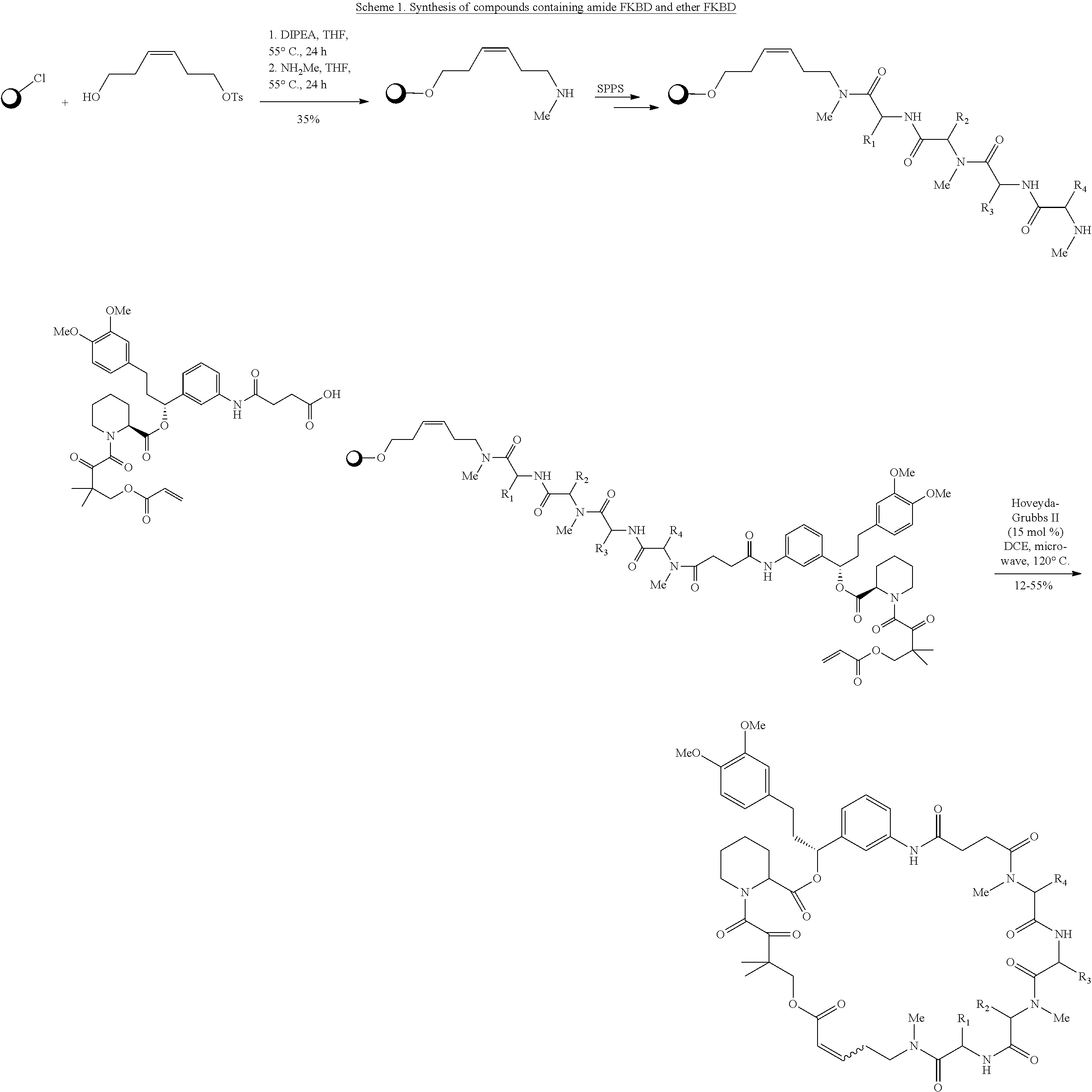

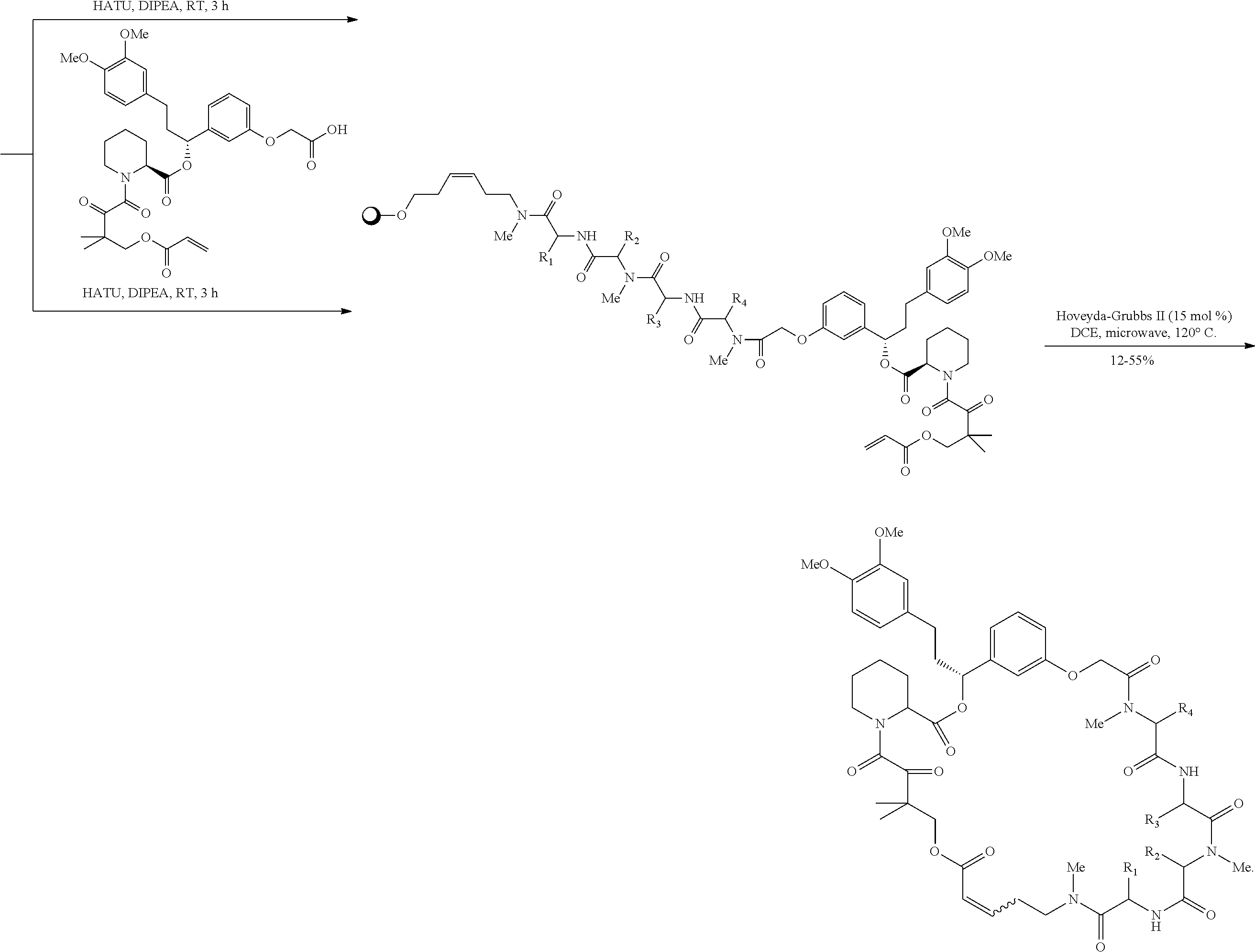

Scheme 1 shows synthesis of compounds containing amide FKBD and ether FKBD. The synthesis of the ether FKBD is shown in Scheme 2. More information regarding synthesis of the macrocyclic compounds in the present disclosure can be found in Guo et al. (2018) Nat. Chem. 11:254-63, which is incorporated herein by reference in its entirety.

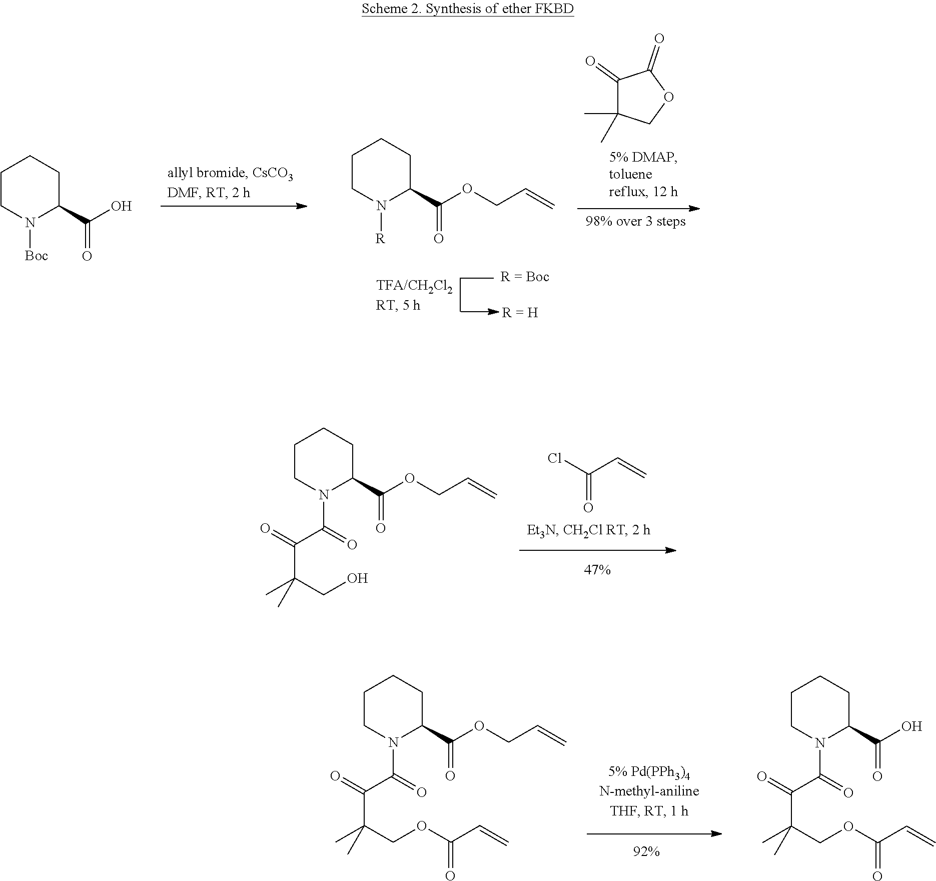

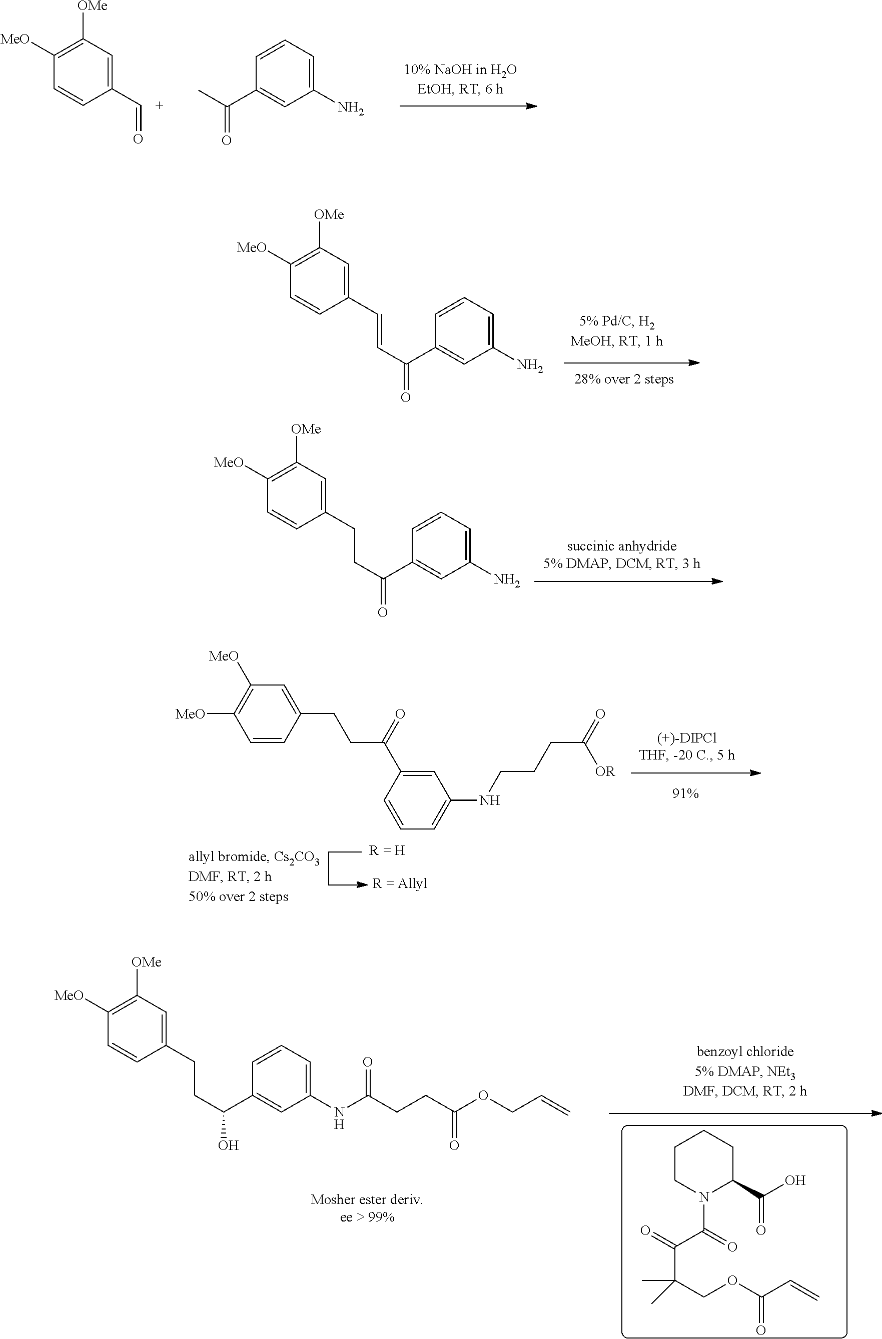

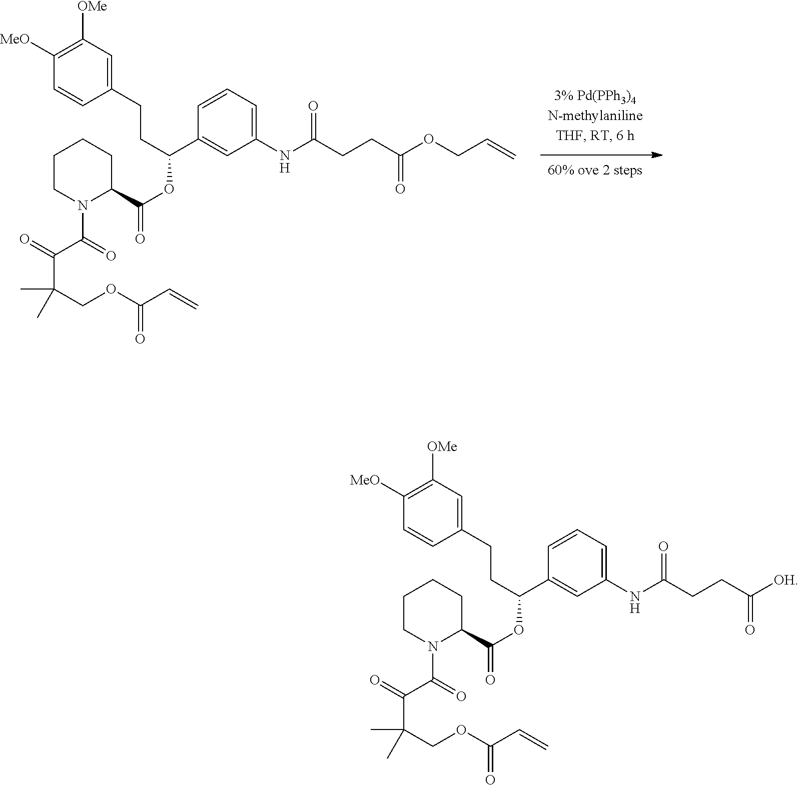

The synthesis of the amide FKBD is shown in Scheme 3.

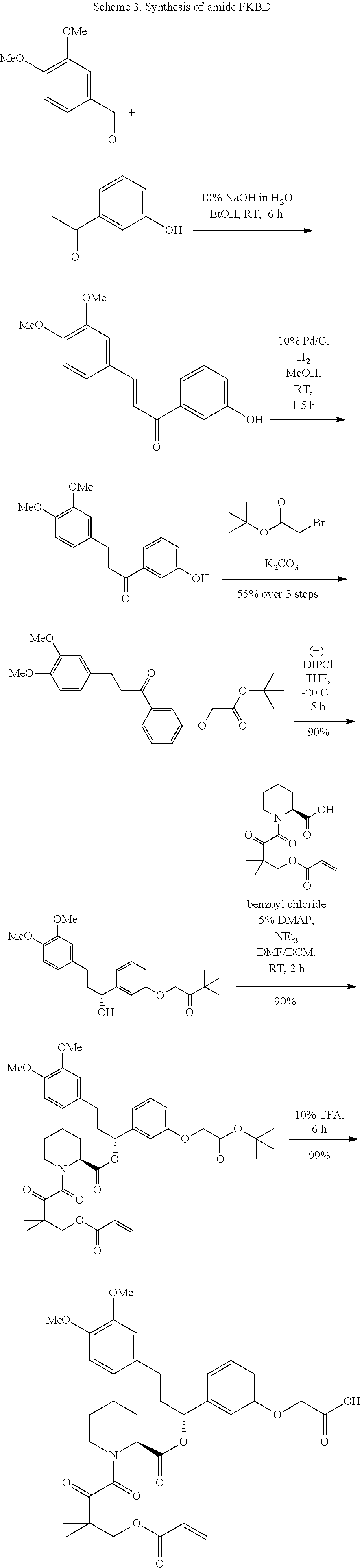

Table 2 below illustrates all the compounds synthesized and characterized in the instant disclosure.

TABLE 2
The compounds in the instant disclosure.

| Compound No. | Exact Mass | Chemical Structure |
|---|---|---|
| 1 | 1076.6 | 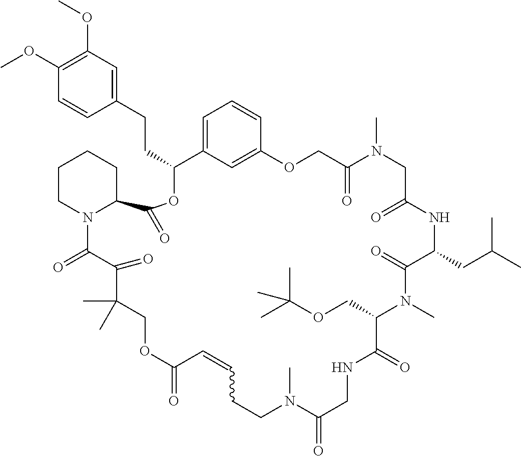 [SMILES: COc1ccc(CC[C@H]2OC(=O)[C@@H]3CCCCN3C(=O)C(=O)C(C)(C)COC(=O)C=CCCN(C)C(=O)CNC(=O)[C@H](COC(C)(C)C)N(C)C(=O)[C@@H](CC(C)C)NC(=O)CN(C)C(=O)COc3cccc2c3)cc1OC] |
| 2 | 1090.6 | 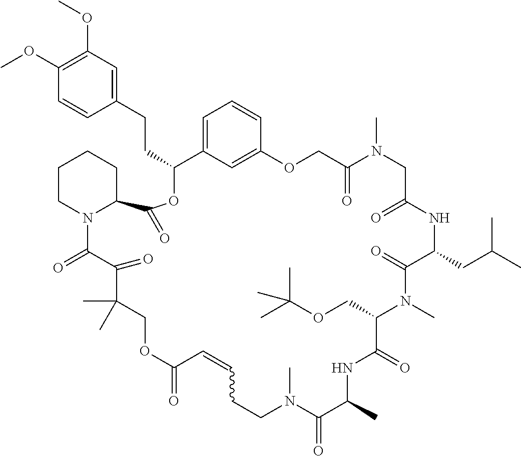 [SMILES: COc1ccc(CC[C@H]2OC(=O)[C@@H]3CCCCN3C(=O)C(=O)C(C)(C)COC(=O)C=CCCN(C)C(=O)[C@H](C)NC(=O)[C@H](COC(C)(C)C)N(C)C(=O)[C@@H](CC(C)C)NC(=O)CN(C)C(=O)COc3cccc2c3)cc1OC] |
| 3 | 1090.6 | 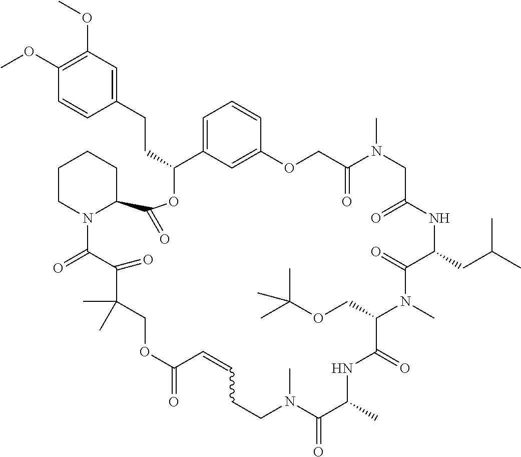 [SMILES: COc1ccc(CC[C@H]2OC(=O)[C@@H]3CCCCN3C(=O)C(=O)C(C)(C)COC(=O)C=CCCN(C)C(=O)[C@@H](C)NC(=O)[C@H](COC(C)(C)C)N(C)C(=O)[C@@H](CC(C)C)NC(=O)CN(C)C(=O)COc3cccc2c3)cc1OC] |
| 4 | 1118.6 | 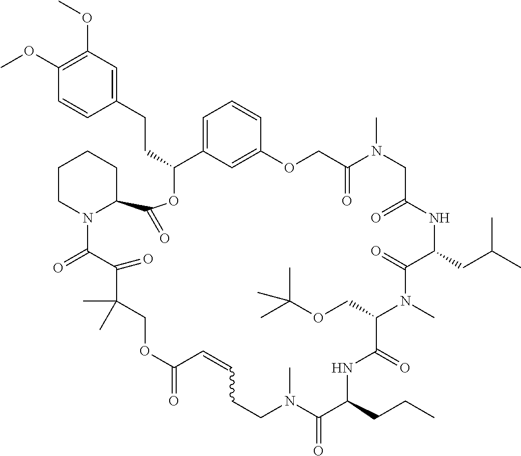 [SMILES: CCC[C@@H]1NC(=O)[C@H](COC(C)(C)C)N(C)C(=O)[C@@H](CC(C)C)NC(=O)CN(C)C(=O)COc2cccc(c2)[C@@H](CCc2ccc(OC)c(OC)c2)OC(=O)[C@@H]2CCCCN2C(=O)C(=O)C(C)(C)COC(=O)C=CCCN(C)C1=O] |
| 5 | 1134.6 | 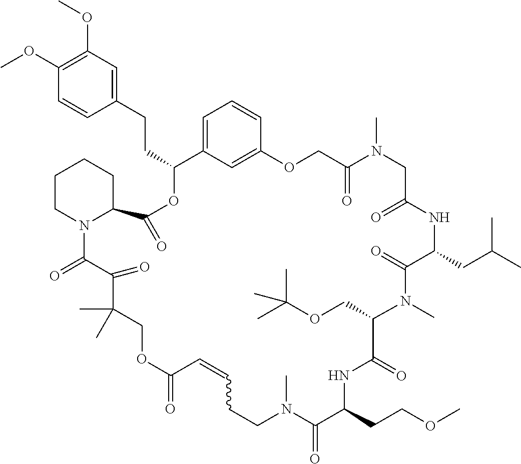 [SMILES: COCC[C@@H]1NC(=O)[C@H](COC(C)(C)C)N(C)C(=O)[C@@H](CC(C)C)NC(=O)CN(C)C(=O)COc2cccc(c2)[C@@H](CCc2ccc(OC)c(OC)c2)OC(=O)[C@@H]2CCCCN2C(=O)C(=O)C(C)(C)COC(=O)C=CCCN(C)C1=O] |
| 6 | 1132.6 | 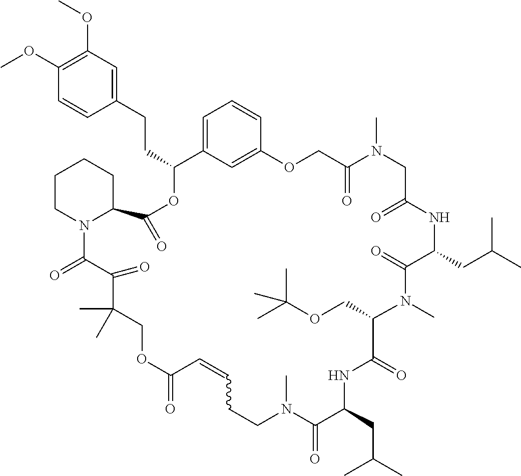 [SMILES: COc1ccc(CC[C@H]2OC(=O)[C@@H]3CCCCN3C(=O)C(=O)C(C)(C)COC(=O)C=CCCN(C)C(=O)[C@H](CC(C)C)NC(=O)[C@H](COC(C)(C)C)N(C)C(=O)[C@@H](CC(C)C)NC(=O)CN(C)C(=O)COc3cccc2c3)cc1OC] |
| 7 | 1132.6 | 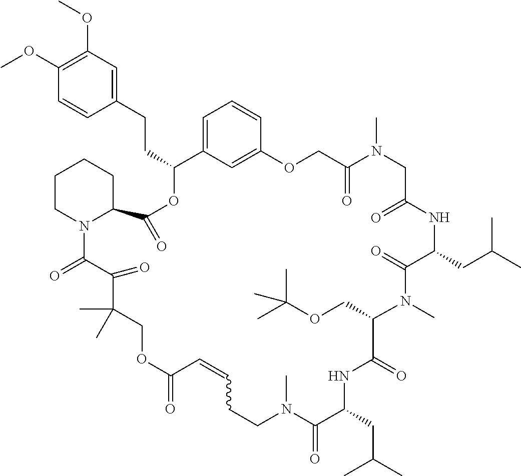 [SMILES: COc1ccc(CC[C@H]2OC(=O)[C@@H]3CCCCN3C(=O)C(=O)C(C)(C)COC(=O)C=CCCN(C)C(=O)[C@@H](CC(C)C)NC(=O)[C@H](COC(C)(C)C)N(C)C(=O)[C@@H](CC(C)C)NC(=O)CN(C)C(=O)COc3cccc2c3)cc1OC] |
| 8 | 1166.6 | 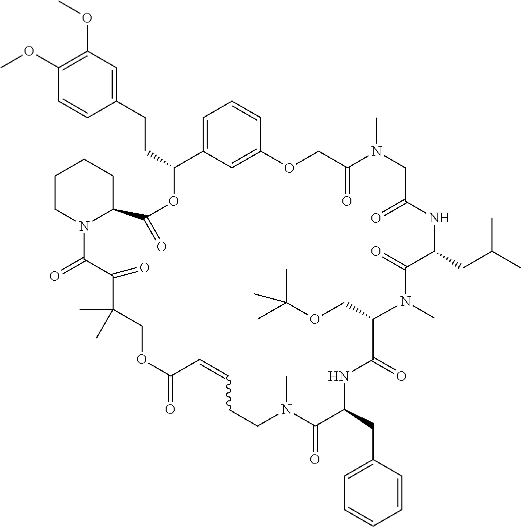 [SMILES: COc1ccc(CC[C@H]2OC(=O)[C@@H]3CCCCN3C(=O)C(=O)C(C)(C)COC(=O)C=CCCN(C)C(=O)[C@H](Cc3ccccc3)NC(=O)[C@H](COC(C)(C)C)N(C)C(=O)[C@@H](CC(C)C)NC(=O)CN(C)C(=O)COc3cccc2c3)cc1OC] |
| 9 | 1166.6 | 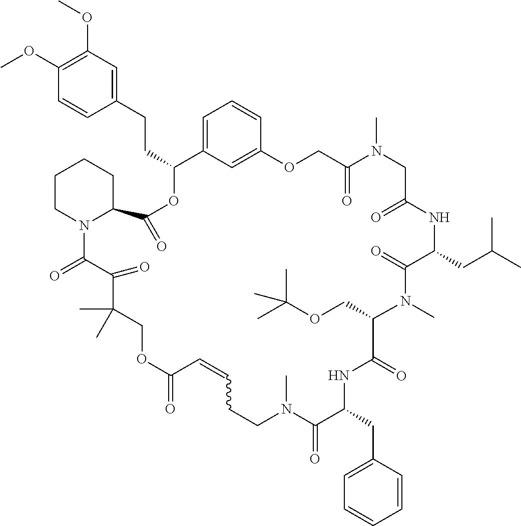 [SMILES: COc1ccc(CC[C@H]2OC(=O)[C@@H]3CCCCN3C(=O)C(=O)C(C)(C)COC(=O)C=CCCN(C)C(=O)[C@@H](Cc3ccccc3)NC(=O)[C@H](COC(C)(C)C)N(C)C(=O)[C@@H](CC(C)C)NC(=O)CN(C)C(=O)COc3cccc2c3)cc1OC] |
| 10 | 1184.6 | 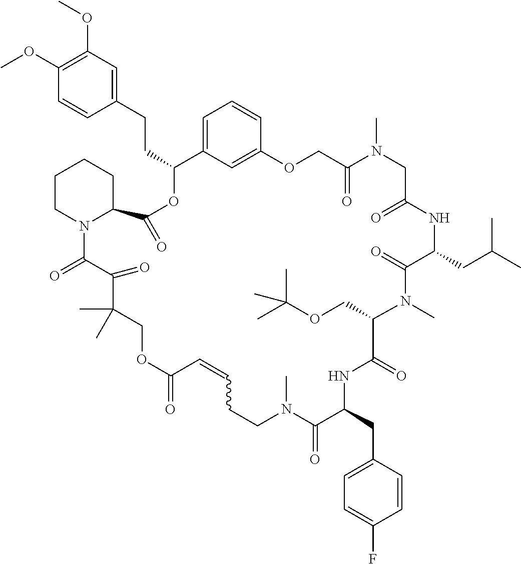 [SMILES: COc1ccc(CC[C@H]2OC(=O)[C@@H]3CCCCN3C(=O)C(=O)C(C)(C)COC(=O)C=CCCN(C)C(=O)[C@H](Cc3ccc(F)cc3)NC(=O)[C@H](COC(C)(C)C)N(C)C(=O)[C@@H](CC(C)C)NC(=O)CN(C)C(=O)COc3cccc2c3)cc1OC] |
| 11 | 1116.6 | 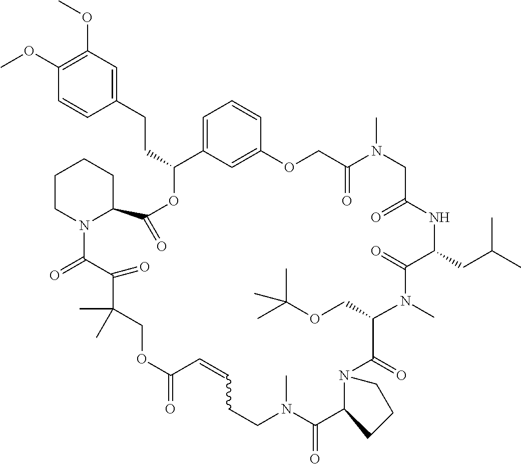 [SMILES: COc1ccc(CC[C@H]2OC(=O)[C@@H]3CCCCN3C(=O)C(=O)C(C)(C)COC(=O)C=CCCN(C)C(=O)[C@@H]3CCCN3C(=O)[C@H](COC(C)(C)C)N(C)C(=O)[C@@H](CC(C)C)NC(=O)CN(C)C(=O)COc3cccc2c3)cc1OC] |
| 12 | 1172.7 | 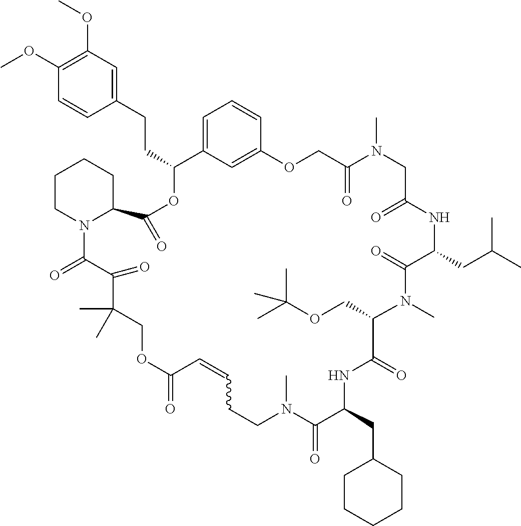 [SMILES: COc1ccc(CC[C@H]2OC(=O)[C@@H]3CCCCN3C(=O)C(=O)C(C)(C)COC(=O)C=CCCN(C)C(=O)[C@H](CC3CCCCC3)NC(=O)[C@H](COC(C)(C)C)N(C)C(=O)[C@@H](CC(C)C)NC(=O)CN(C)C(=O)COc3cccc2c3)cc1OC] |
| 13 | 1216.6 | 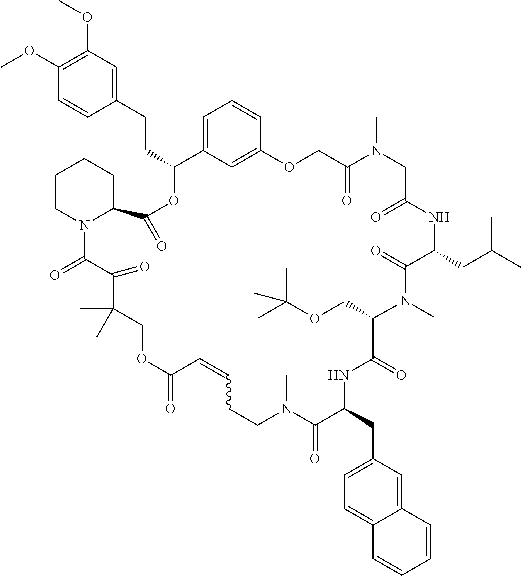 [SMILES: COc1ccc(CC[C@H]2OC(=O)[C@@H]3CCCCN3C(=O)C(=O)C(C)(C)COC(=O)C=CCCN(C)C(=O)[C@H](Cc3ccc4ccccc4c3)NC(=O)[C@H](COC(C)(C)C)N(C)C(=O)[C@@H](CC(C)C)NC(=O)CN(C)C(=O)COc3cccc2c3)cc1OC] |
| 14 | 1118.6 | 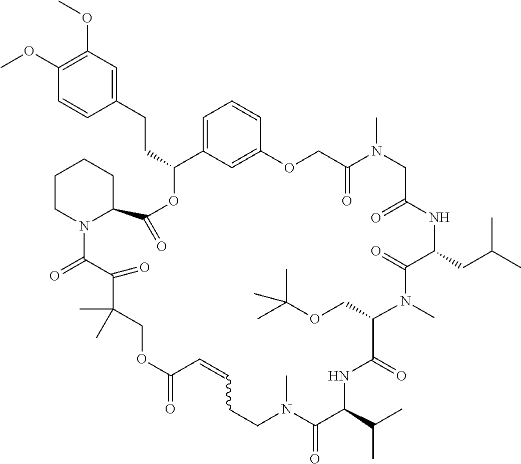 [SMILES: COc1ccc(CC[C@H]2OC(=O)[C@@H]3CCCCN3C(=O)C(=O)C(C)(C)COC(=O)C=CCCN(C)C(=O)[C@H](C(C)C)NC(=O)[C@H](COC(C)(C)C)N(C)C(=O)[C@@H](CC(C)C)NC(=O)CN(C)C(=O)COc3cccc2c3)cc1OC] |
| 15 | 1152.6 | 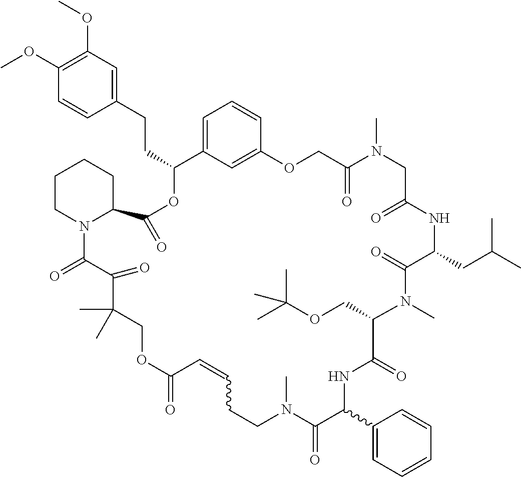 [SMILES: COc1ccc(CC[C@H]2OC(=O)[C@@H]3CCCCN3C(=O)C(=O)C(C)(C)COC(=O)C=CCCN(C)C(=O)C(c3ccccc3)NC(=O)[C@H](COC(C)(C)C)N(C)C(=O)[C@@H](CC(C)C)NC(=O)CN(C)C(=O)COc3cccc2c3)cc1OC] |
| 16 | 1160.6 | 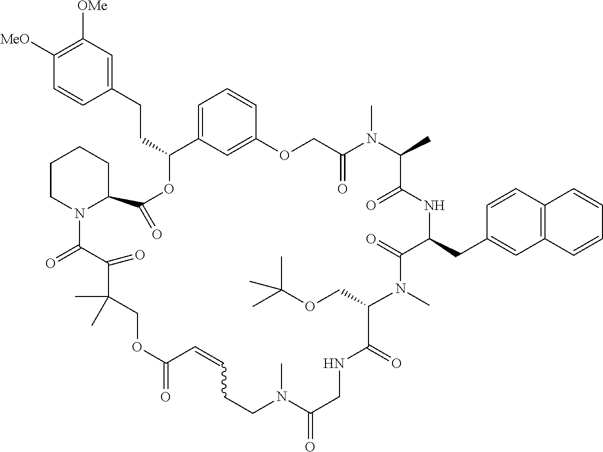 [SMILES: COc1ccc(CC[C@H]2OC(=O)[C@@H]3CCCCN3C(=O)C(=O)C(C)(C)COC(=O)C=CCCN(C)C(=O)CNC(=O)[C@H](COC(C)(C)C)N(C)C(=O)[C@H](Cc3ccc4ccccc4c3)NC(=O)[C@H](C)N(C)C(=O)COc3cccc2c3)cc1OC] |
| 17 | 1188.6 | 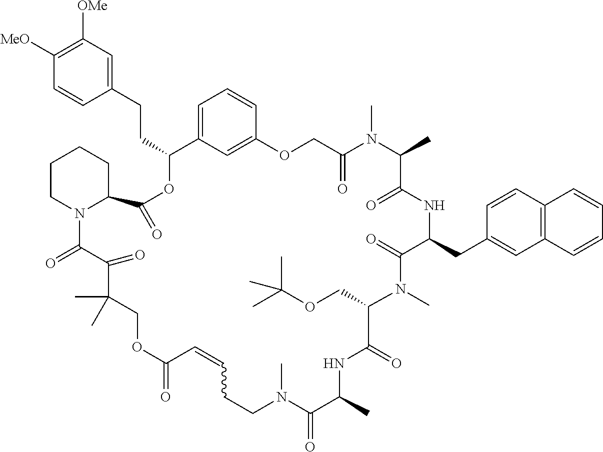 [SMILES: COc1ccc(CC[C@H]2OC(=O)[C@@H]3CCCCN3C(=O)C(=O)C(C)(C)COC(=O)C=CCCN(C)C(=O)[C@H](C)NC(=O)[C@H](COC(C)(C)C)N(C)C(=O)[C@H](Cc3ccc4ccccc4c3)NC(=O)[C@H](C)N(C)C(=O)COc3cccc2c3)cc1OC] |
| 18 | 1188.6 | 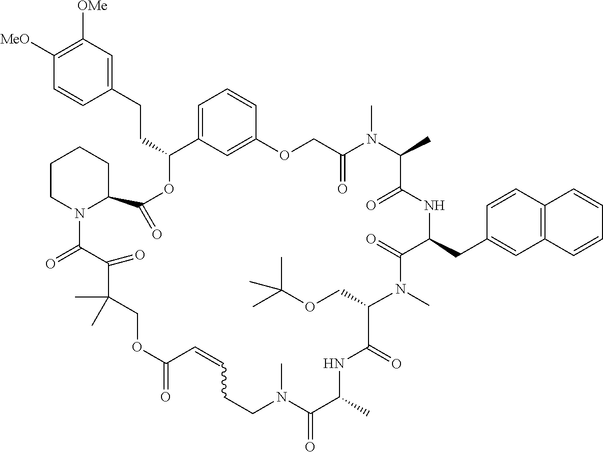 [SMILES: COc1ccc(CC[C@H]2OC(=O)[C@@H]3CCCCN3C(=O)C(=O)C(C)(C)COC(=O)C=CCCN(C)C(=O)[C@@H](C)NC(=O)[C@H](COC(C)(C)C)N(C)C(=O)[C@H](Cc3ccc4ccccc4c3)NC(=O)[C@H](C)N(C)C(=O)COc3cccc2c3)cc1OC] |
| 19 | 1216.6 | 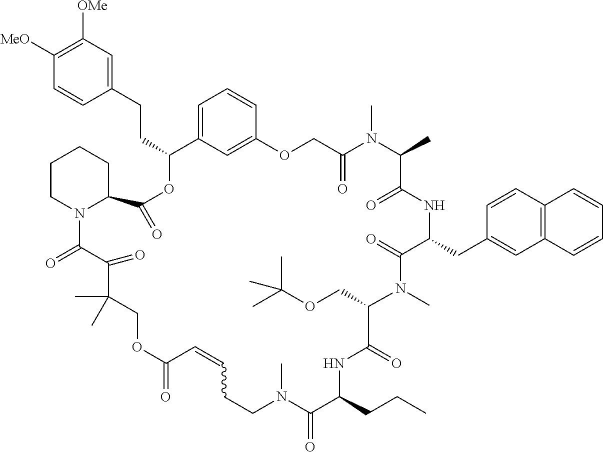 [SMILES: CCC[C@@H]1NC(=O)[C@H](COC(C)(C)C)N(C)C(=O)[C@@H](Cc2ccc3ccccc3c2)NC(=O)[C@H](C)N(C)C(=O)COc2cccc(c2)[C@@H](CCc2ccc(OC)c(OC)c2)OC(=O)[C@@H]2CCCCN2C(=O)C(=O)C(C)(C)COC(=O)C=CCCN(C)C1=O] |
| 20 | 1232.6 | 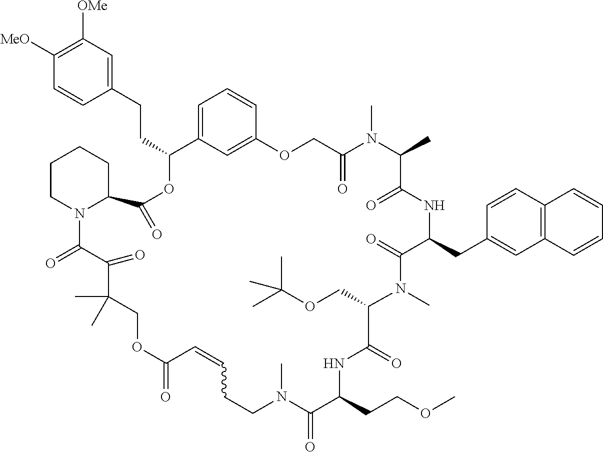 [SMILES: COCC[C@@H]1NC(=O)[C@H](COC(C)(C)C)N(C)C(=O)[C@H](Cc2ccc3ccccc3c2)NC(=O)[C@H](C)N(C)C(=O)COc2cccc(c2)[C@@H](CCc2ccc(OC)c(OC)c2)OC(=O)[C@@H]2CCCCN2C(=O)C(=O)C(C)(C)COC(=O)C=CCCN(C)C1=O] |
| 21 | 1230.6 | 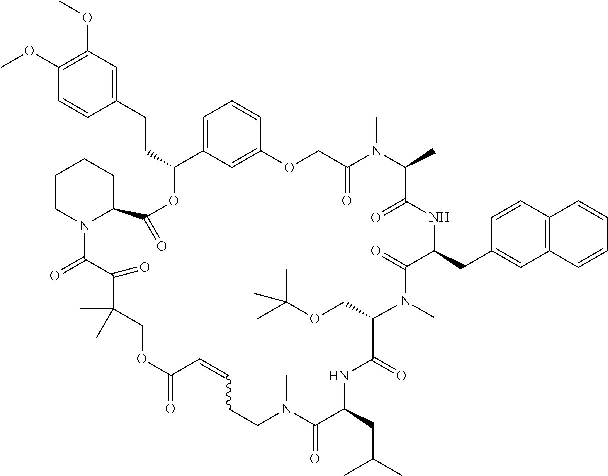 [SMILES: COc1ccc(CC[C@H]2OC(=O)[C@@H]3CCCCN3C(=O)C(=O)C(C)(C)COC(=O)C=CCCN(C)C(=O)[C@H](CC(C)C)NC(=O)[C@H](COC(C)(C)C)N(C)C(=O)[C@H](Cc3ccc4ccccc4c3)NC(=O)[C@H](C)N(C)C(=O)COc3cccc2c3)cc1OC] |
| 22 | 1230.6 | 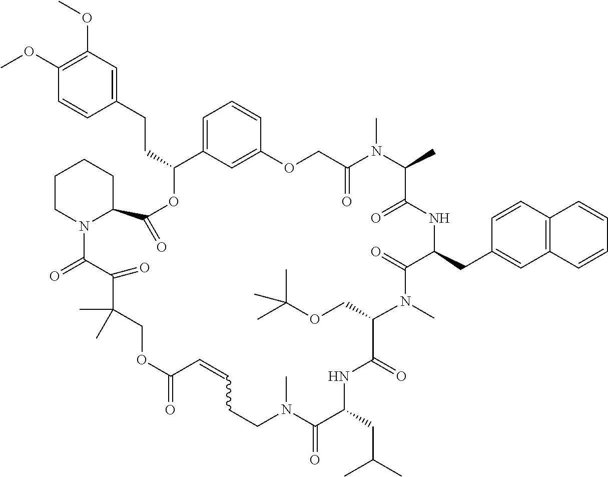 [SMILES: COc1ccc(CC[C@H]2OC(=O)[C@@H]3CCCCN3C(=O)C(=O)C(C)(C)COC(=O)C=CCCN(C)C(=O)[C@@H](CC(C)C)NC(=O)[C@H](COC(C)(C)C)N(C)C(=O)[C@H](Cc3ccc4ccccc4c3)NC(=O)[C@H](C)N(C)C(=O)COc3cccc2c3)cc1OC] |
| 23 | 1264.6 | 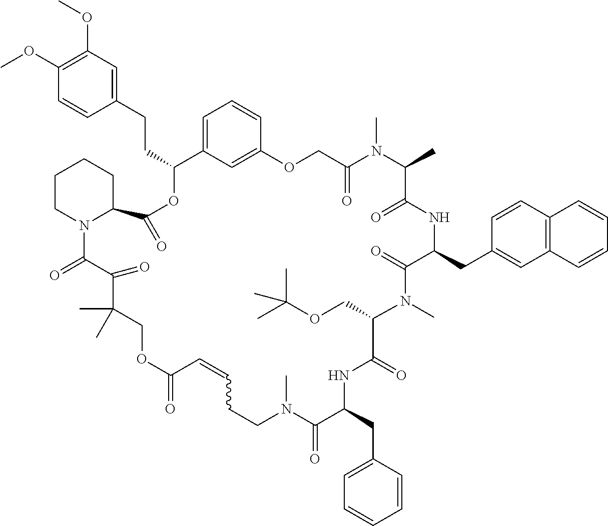 [SMILES: COc1ccc(CC[C@H]2OC(=O)[C@@H]3CCCCN3C(=O)C(=O)C(C)(C)COC(=O)C=CCCN(C)C(=O)[C@H](Cc3ccccc3)NC(=O)[C@H](COC(C)(C)C)N(C)C(=O)[C@H](Cc3ccc4ccccc4c3)NC(=O)[C@H](C)N(C)C(=O)COc3cccc2c3)cc1OC] |
| 24 | 1264.6 | 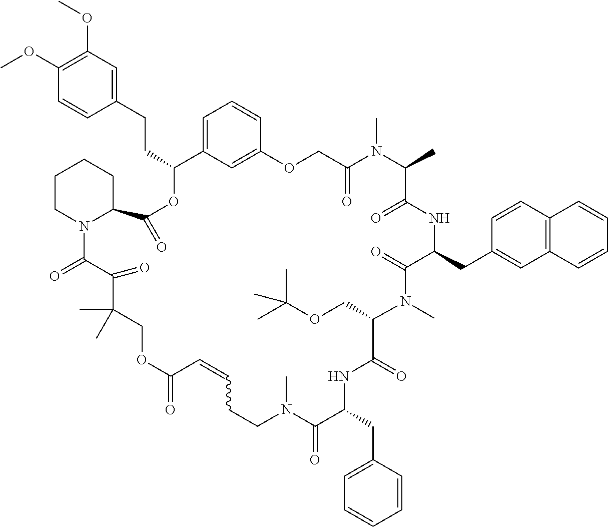 [SMILES: COc1ccc(CC[C@H]2OC(=O)[C@@H]3CCCCN3C(=O)C(=O)C(C)(C)COC(=O)C=CCCN(C)C(=O)[C@@H](Cc3ccccc3)NC(=O)[C@H](COC(C)(C)C)N(C)C(=O)[C@H](Cc3ccc4ccccc4c3)NC(=O)[C@H](C)N(C)C(=O)COc3cccc2c3)cc1OC] |
| 25 | 1282.6 | 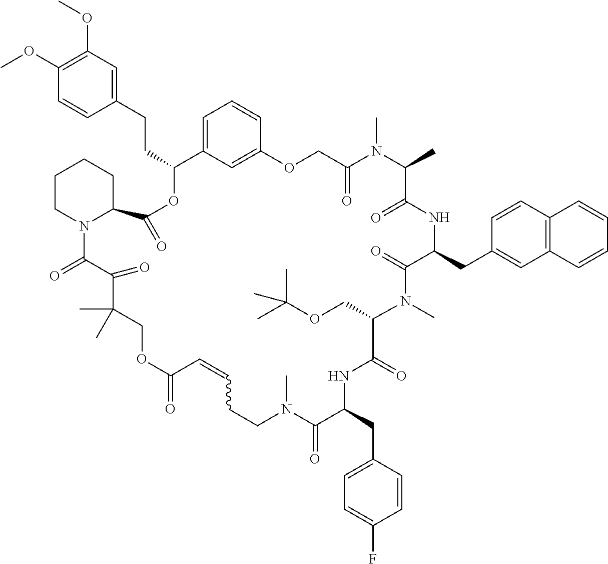 [SMILES: COc1ccc(CC[C@H]2OC(=O)[C@@H]3CCCCN3C(=O)C(=O)C(C)(C)COC(=O)C=CCCN(C)C(=O)[C@H](Cc3ccc(F)cc3)NC(=O)[C@H](COC(C)(C)C)N(C)C(=O)[C@H](Cc3ccc4ccccc4c3)NC(=O)[C@H](C)N(C)C(=O)COc3cccc2c3)cc1OC] |
| 26 | 1214.6 | 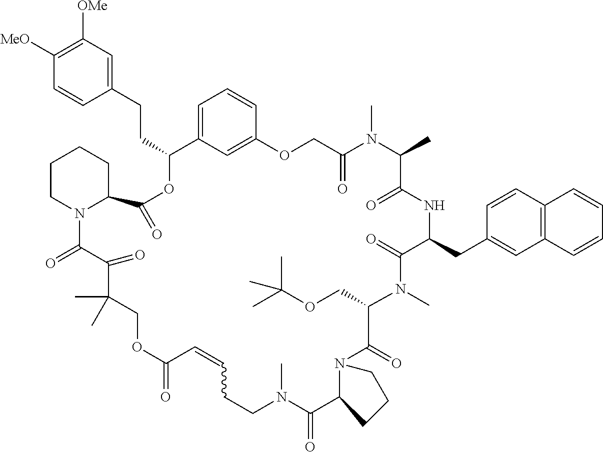 [SMILES: COc1ccc(CC[C@H]2OC(=O)[C@@H]3CCCCN3C(=O)C(=O)C(C)(C)COC(=O)C=CCCN(C)C(=O)[C@@H]3CCCN3C(=O)[C@H](COC(C)(C)C)N(C)C(=O)[C@H](Cc3ccc4ccccc4c3)NC(=O)[C@H](C)N(C)C(=O)COc3cccc2c3)cc1OC] |
| 27 | 1270.7 | 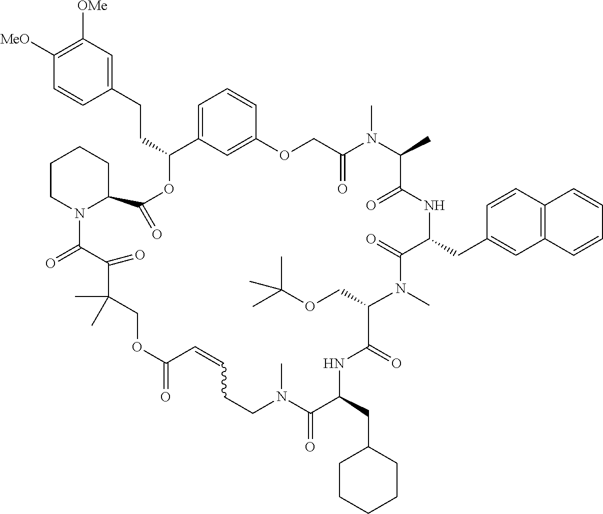 [SMILES: COc1ccc(CC[C@H]2OC(=O)[C@@H]3CCCCN3C(=O)C(=O)C(C)(C)COC(=O)C=CCCN(C)C(=O)[C@H](CC3CCCCC3)NC(=O)[C@H](COC(C)(C)C)N(C)C(=O)[C@@H](Cc3ccc4ccccc4c3)NC(=O)[C@H](C)N(C)C(=O)COc3cccc2c3)cc1OC] |
| 28 | 1314.6 | 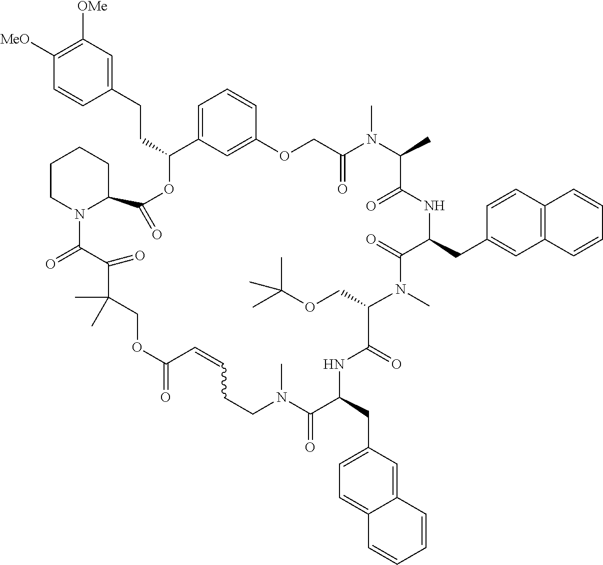 [SMILES: COc1ccc(CC[C@H]2OC(=O)[C@@H]3CCCCN3C(=O)C(=O)C(C)(C)COC(=O)C=CCCN(C)C(=O)[C@H](Cc3ccc4ccccc4c3)NC(=O)[C@H](COC(C)(C)C)N(C)C(=O)[C@H](Cc3ccc4ccccc4c3)NC(=O)[C@H](C)N(C)C(=O)COc3cccc2c3)cc1OC] |
| 29 | 1216.6 | 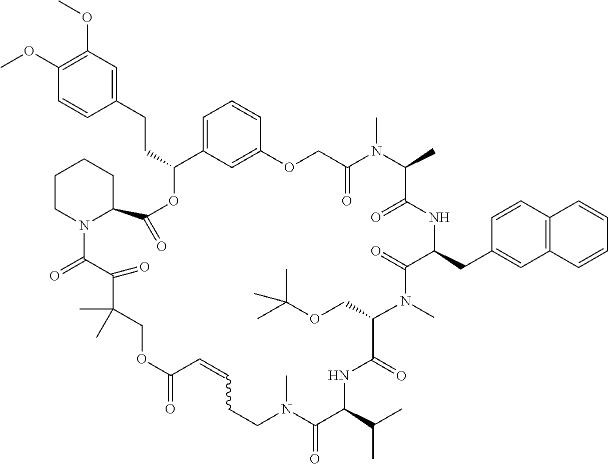 [SMILES: COc1ccc(CC[C@H]2OC(=O)[C@@H]3CCCCN3C(=O)C(=O)C(C)(C)COC(=O)C=CCCN(C)C(=O)[C@H](C(C)C)NC(=O)[C@H](COC(C)(C)C)N(C)C(=O)[C@H](Cc3ccc4ccccc4c3)NC(=O)[C@H](C)N(C)C(=O)COc3cccc2c3)cc1OC] |
| 30 | 1250.6 | 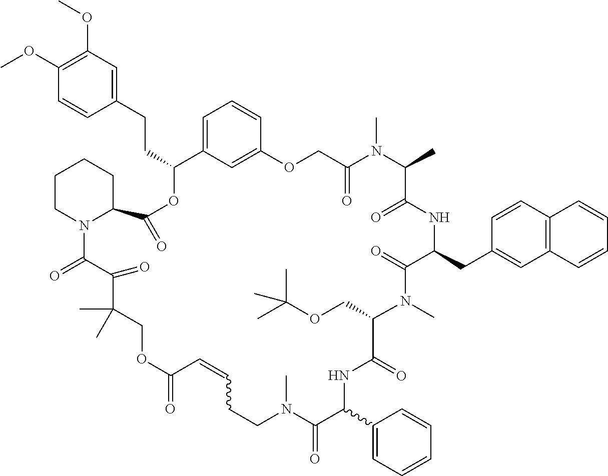 [SMILES: COc1ccc(CC[C@H]2OC(=O)[C@@H]3CCCCN3C(=O)C(=O)C(C)(C)COC(=O)C=CCCN(C)C(=O)C(c3ccccc3)NC(=O)[C@H](COC(C)(C)C)N(C)C(=O)[C@H](Cc3ccc4ccccc4c3)NC(=O)[C@H](C)N(C)C(=O)COc3cccc2c3)cc1OC] |
| 31 | 1096.5 | 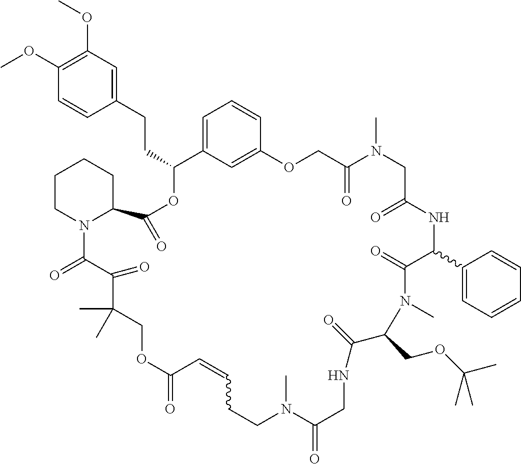 [SMILES: COc1ccc(CC[C@H]2OC(=O)[C@@H]3CCCCN3C(=O)C(=O)C(C)(C)COC(=O)C=CCCN(C)C(=O)CNC(=O)[C@H](COC(C)(C)C)N(C)C(=O)C(c3ccccc3)NC(=O)CN(C)C(=O)COc3cccc2c3)cc1OC] |
| 32 | 1110.5 | 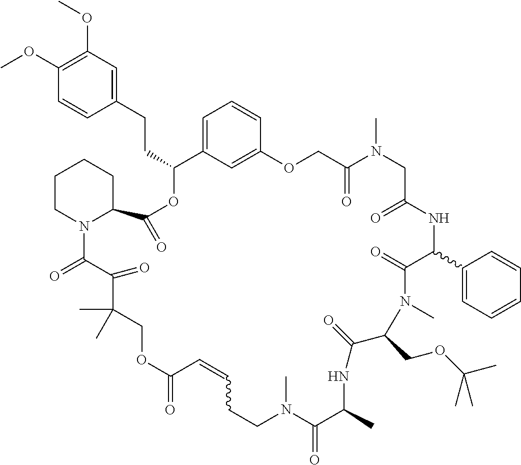 [SMILES: COc1ccc(CC[C@H]2OC(=O)[C@@H]3CCCCN3C(=O)C(=O)C(C)(C)COC(=O)C=CCCN(C)C(=O)[C@H](C)NC(=O)[C@H](COC(C)(C)C)N(C)C(=O)C(c3ccccc3)NC(=O)CN(C)C(=O)COc3cccc2c3)cc1OC] |
| 33 | 1110.5 | 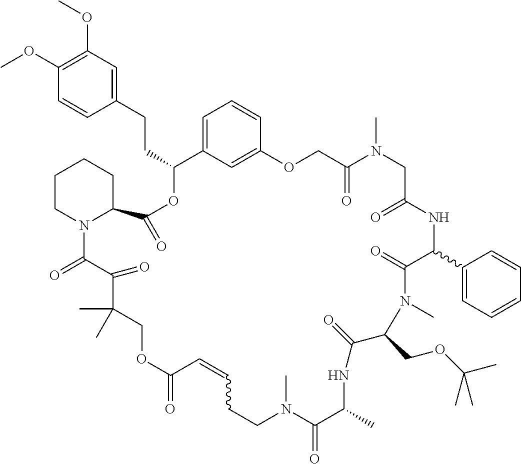 [SMILES: COc1ccc(CC[C@H]2OC(=O)[C@@H]3CCCCN3C(=O)C(=O)C(C)(C)COC(=O)C=CCCN(C)C(=O)[C@@H](C)NC(=O)[C@H](COC(C)(C)C)N(C)C(=O)C(c3ccccc3)NC(=O)CN(C)C(=O)COc3cccc2c3)cc1OC] |
| 34 | 1138.6 | 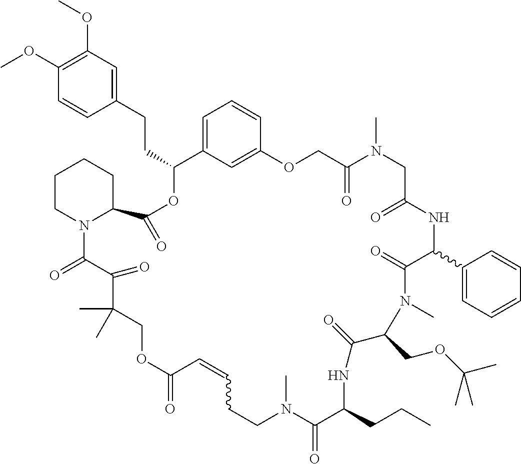 [SMILES: CCC[C@@H]1NC(=O)[C@H](COC(C)(C)C)N(C)C(=O)C(c2ccccc2)NC(=O)CN(C)C(=O)COc2cccc(c2)[C@@H](CCc2ccc(OC)c(OC)c2)OC(=O)[C@@H]2CCCCN2C(=O)C(=O)C(C)(C)COC(=O)C=CCCN(C)C1=O] |
| 35 | 1154.5 | 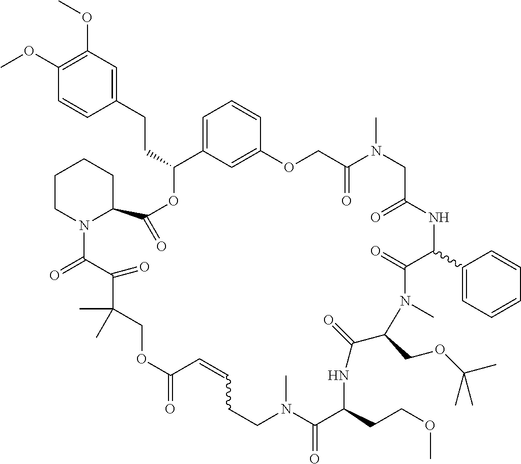 [SMILES: COCC[C@@H]1NC(=O)[C@H](COC(C)(C)C)N(C)C(=O)C(c2ccccc2)NC(=O)CN(C)C(=O)COc2cccc(c2)[C@@H](CCc2ccc(OC)c(OC)c2)OC(=O)[C@@H]2CCCCN2C(=O)C(=O)C(C)(C)COC(=O)C=CCCN(C)C1=O] |
| 36 | 1152.6 | 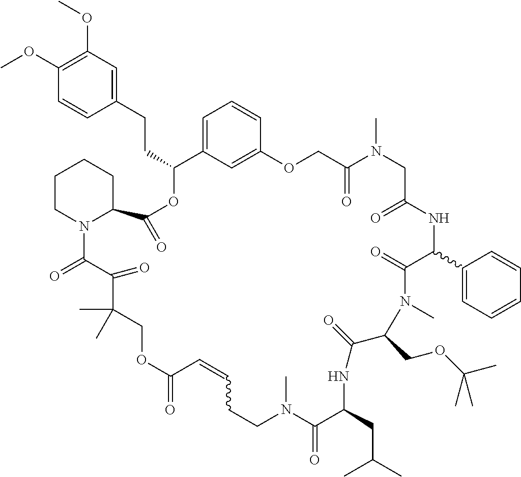 [SMILES: COc1ccc(CC[C@H]2OC(=O)[C@@H]3CCCCN3C(=O)C(=O)C(C)(C)COC(=O)C=CCCN(C)C(=O)[C@H](CC(C)C)NC(=O)[C@H](COC(C)(C)C)N(C)C(=O)C(c3ccccc3)NC(=O)CN(C)C(=O)COc3cccc2c3)cc1OC] |
| 37 | 1152.6 | 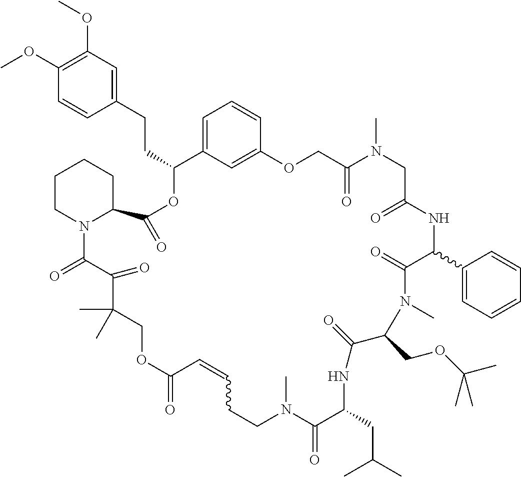 [SMILES: COc1ccc(CC[C@H]2OC(=O)[C@@H]3CCCCN3C(=O)C(=O)C(C)(C)COC(=O)C=CCCN(C)C(=O)[C@@H](CC(C)C)NC(=O)[C@H](COC(C)(C)C)N(C)C(=O)C(c3ccccc3)NC(=O)CN(C)C(=O)COc3cccc2c3)cc1OC] |
| 38 | 1186.6 | 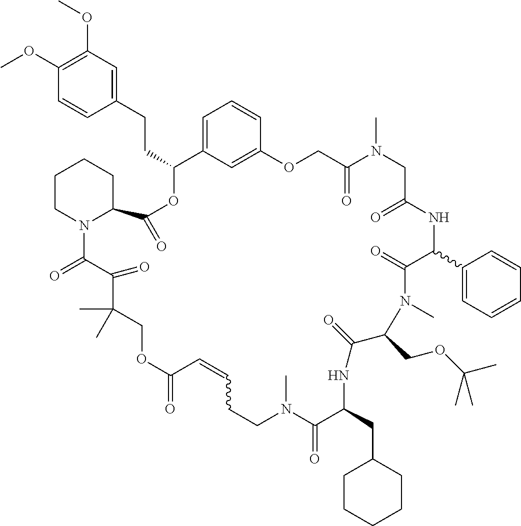 [SMILES: COc1ccc(CC[C@H]2OC(=O)[C@@H]3CCCCN3C(=O)C(=O)C(C)(C)COC(=O)C=CCCN(C)C(=O)[C@H](CC3CCCCC3)NC(=O)[C@H](COC(C)(C)C)N(C)C(=O)C(c3ccccc3)NC(=O)CN(C)C(=O)COc3cccc2c3)cc1OC] |
| 39 | 1186.6 | 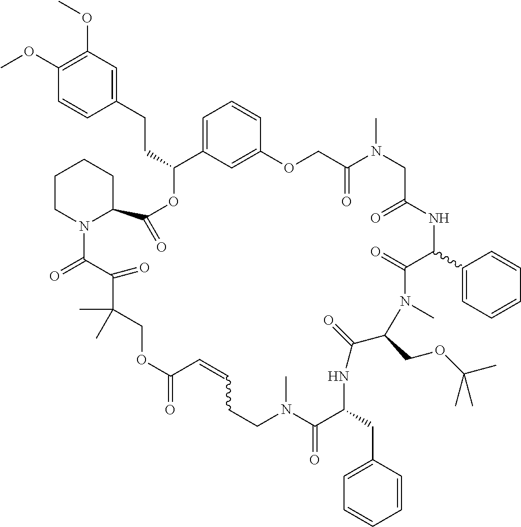 [SMILES: COc1ccc(CC[C@H]2OC(=O)[C@@H]3CCCCN3C(=O)C(=O)C(C)(C)COC(=O)C=CCCN(C)C(=O)[C@@H](Cc3ccccc3)NC(=O)[C@H](COC(C)(C)C)N(C)C(=O)C(c3ccccc3)NC(=O)CN(C)C(=O)COc3cccc2c3)cc1OC] |
| 40 | 1204.6 | 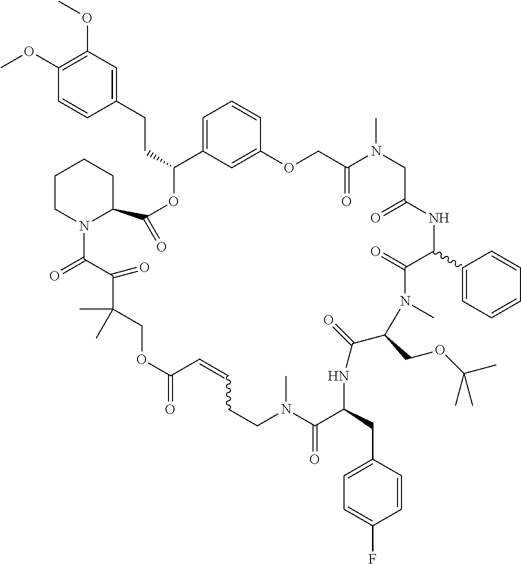 [SMILES: COc1ccc(CC[C@H]2OC(=O)[C@@H]3CCCCN3C(=O)C(=O)C(C)(C)COC(=O)C=CCCN(C)C(=O)[C@H](Cc3ccc(F)cc3)NC(=O)[C@H](COC(C)(C)C)N(C)C(=O)C(c3ccccc3)NC(=O)CN(C)C(=O)COc3cccc2c3)cc1OC] |
| 41 | 1136.6 | 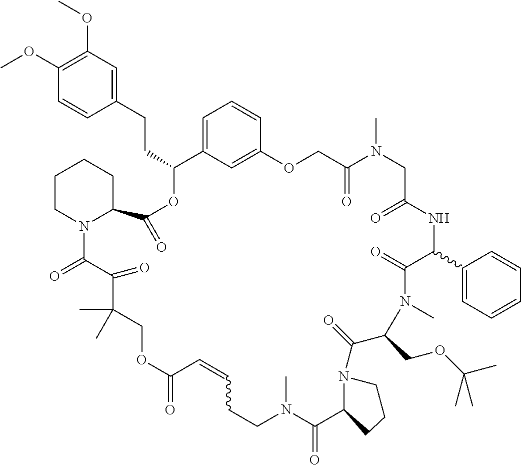 [SMILES: COc1ccc(CC[C@H]2OC(=O)[C@@H]3CCCCN3C(=O)C(=O)C(C)(C)COC(=O)C=CCCN(C)C(=O)[C@@H]3CCCN3C(=O)[C@H](COC(C)(C)C)N(C)C(=O)C(c3ccccc3)NC(=O)CN(C)C(=O)COc3cccc2c3)cc1OC] |
| 42 | 1192.6 | 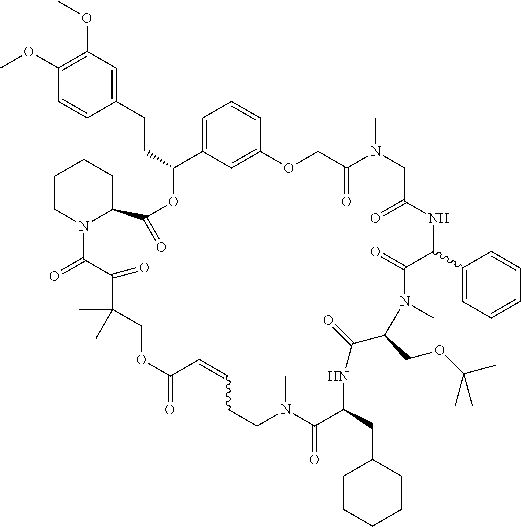 [SMILES: COc1ccc(CC[C@H]2OC(=O)[C@@H]3CCCCN3C(=O)C(=O)C(C)(C)COC(=O)C=CCCN(C)C(=O)[C@H](CC3CCCCC3)NC(=O)[C@H](COC(C)(C)C)N(C)C(=O)C(c3ccccc3)NC(=O)CN(C)C(=O)COc3cccc2c3)cc1OC] |
| 43 | 1236.6 | 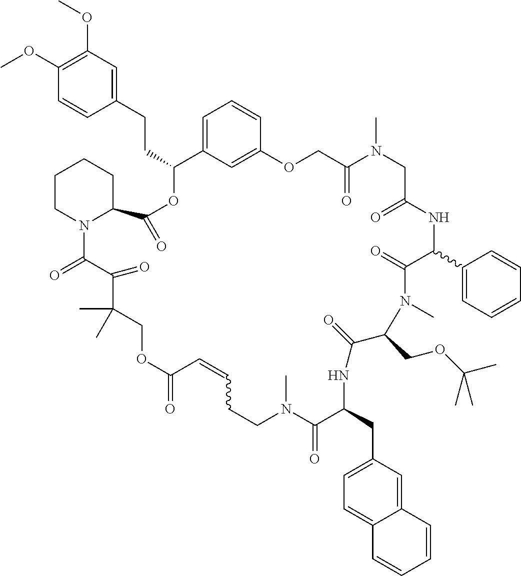 [SMILES: COc1ccc(CC[C@H]2OC(=O)[C@@H]3CCCCN3C(=O)C(=O)C(C)(C)COC(=O)C=CCCN(C)C(=O)[C@H](Cc3ccc4ccccc4c3)NC(=O)[C@H](COC(C)(C)C)N(C)C(=O)C(c3ccccc3)NC(=O)CN(C)C(=O)COc3cccc2c3)cc1OC] |
| 44 | 1138.6 | 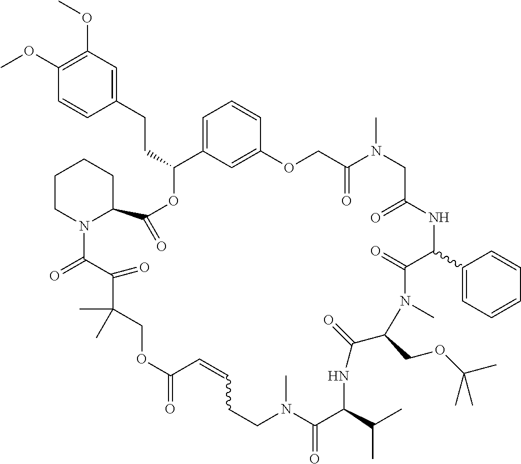 [SMILES: COc1ccc(CC[C@H]2OC(=O)[C@@H]3CCCCN3C(=O)C(=O)C(C)(C)COC(=O)C=CCCN(C)C(=O)[C@H](C(C)C)NC(=O)[C@H](COC(C)(C)C)N(C)C(=O)C(c3ccccc3)NC(=O)CN(C)C(=O)COc3cccc2c3)cc1OC] |
| 45 | 1172.6 | 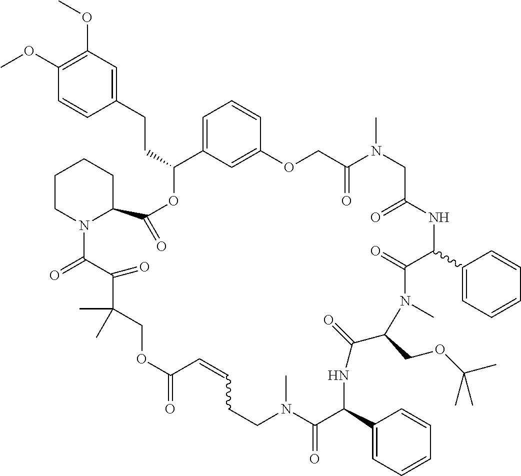 [SMILES: COc1ccc(CC[C@H]2OC(=O)[C@@H]3CCCCN3C(=O)C(=O)C(C)(C)COC(=O)C=CCCN(C)C(=O)[C@H](c3ccccc3)NC(=O)[C@H](COC(C)(C)C)N(C)C(=O)C(c3ccccc3)NC(=O)CN(C)C(=O)COc3cccc2c3)cc1OC] |
| 46 | 1110.5 | 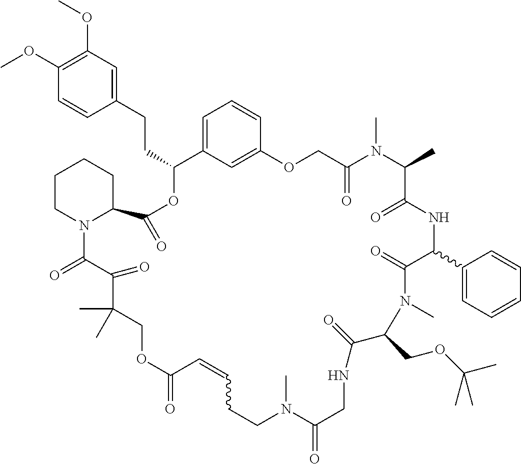 [SMILES: COc1ccc(CC[C@H]2OC(=O)[C@@H]3CCCCN3C(=O)C(=O)C(C)(C)COC(=O)C=CCCN(C)C(=O)CNC(=O)[C@H](COC(C)(C)C)N(C)C(=O)C(c3ccccc3)NC(=O)[C@H](C)N(C)C(=O)COc3cccc2c3)cc1OC] |
| 47 | 1124.6 | 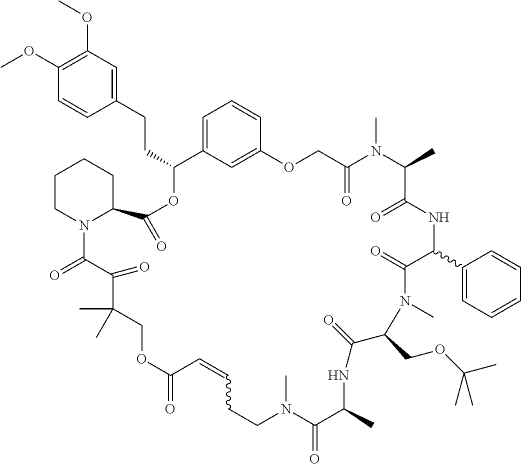 [SMILES: COc1ccc(CC[C@H]2OC(=O)[C@@H]3CCCCN3C(=O)C(=O)C(C)(C)COC(=O)C=CCCN(C)C(=O)[C@H](C)NC(=O)[C@H](COC(C)(C)C)N(C)C(=O)C(c3ccccc3)NC(=O)[C@H](C)N(C)C(=O)COc3cccc2c3)cc1OC] |
| 48 | 1124.6 | 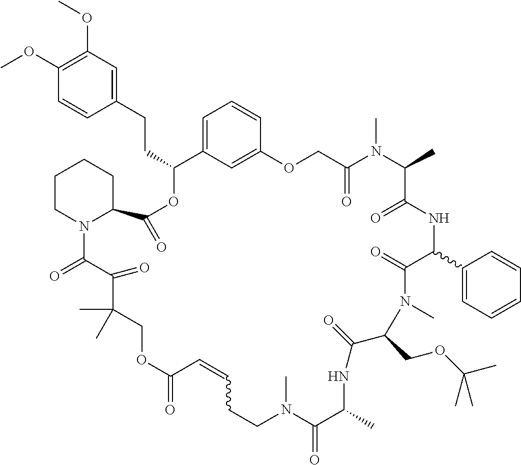 [SMILES: COc1ccc(CC[C@H]2OC(=O)[C@@H]3CCCCN3C(=O)C(=O)C(C)(C)COC(=O)C=CCCN(C)C(=O)[C@@H](C)NC(=O)[C@H](COC(C)(C)C)N(C)C(=O)C(c3ccccc3)NC(=O)[C@H](C)N(C)C(=O)COc3cccc2c3)cc1OC] |
| 49 | 1152.6 | 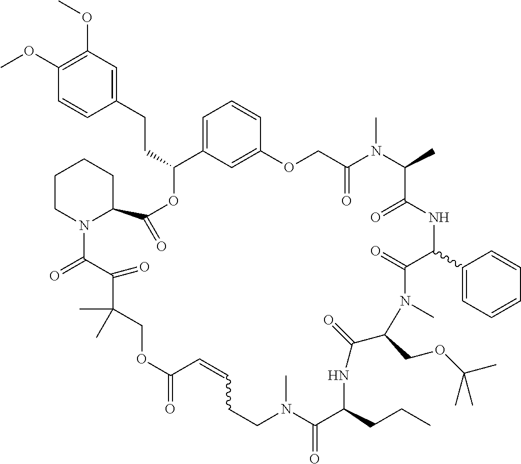 [SMILES: CCC[C@@H]1NC(=O)[C@H](COC(C)(C)C)N(C)C(=O)C(c2ccccc2)NC(=O)[C@H](C)N(C)C(=O)COc2cccc(c2)[C@@H](CCc2ccc(OC)c(OC)c2)OC(=O)[C@@H]2CCCCN2C(=O)C(=O)C(C)(C)COC(=O)C=CCCN(C)C1=O] |
| 50 | 1168.5 | 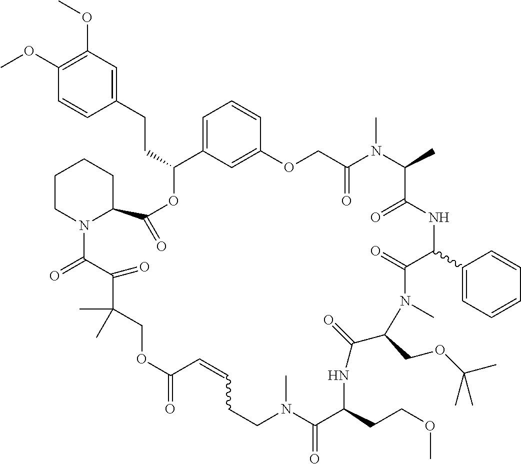 [SMILES: COCC[C@@H]1NC(=O)[C@H](COC(C)(C)C)N(C)C(=O)C(c2ccccc2)NC(=O)[C@H](C)N(C)C(=O)COc2cccc(c2)[C@@H](CCc2ccc(OC)c(OC)c2)OC(=O)[C@@H]2CCCCN2C(=O)C(=O)C(C)(C)COC(=O)C=CCCN(C)C1=O] |
| 51 | 1166.6 | 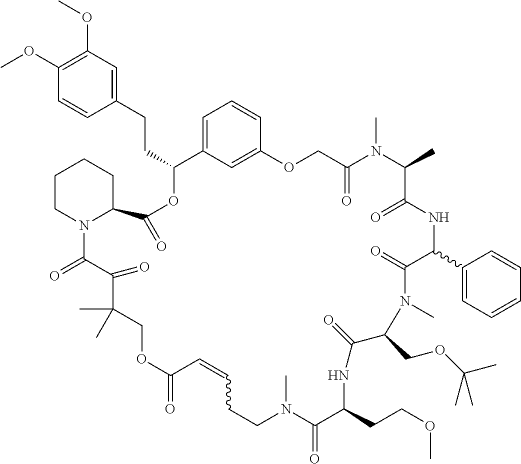 [SMILES: COCC[C@@H]1NC(=O)[C@H](COC(C)(C)C)N(C)C(=O)C(c2ccccc2)NC(=O)[C@H](C)N(C)C(=O)COc2cccc(c2)[C@@H](CCc2ccc(OC)c(OC)c2)OC(=O)[C@@H]2CCCCN2C(=O)C(=O)C(C)(C)COC(=O)C=CCCN(C)C1=O] |
| 52 | 1166.6 | 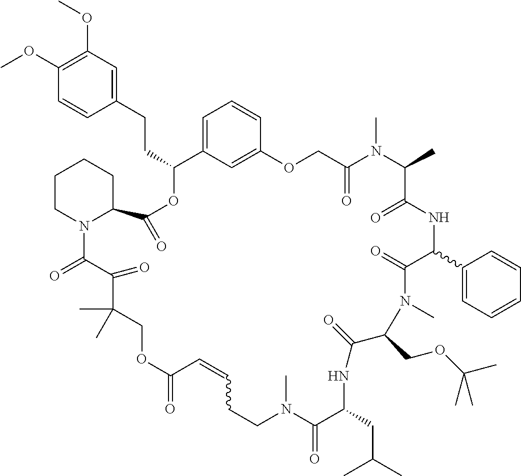 [SMILES: COc1ccc(CC[C@H]2OC(=O)[C@@H]3CCCCN3C(=O)C(=O)C(C)(C)COC(=O)C=CCCN(C)C(=O)[C@@H](CC(C)C)NC(=O)[C@H](COC(C)(C)C)N(C)C(=O)C(c3ccccc3)NC(=O)[C@H](C)N(C)C(=O)COc3cccc2c3)cc1OC] |
| 53 | 1200.6 | 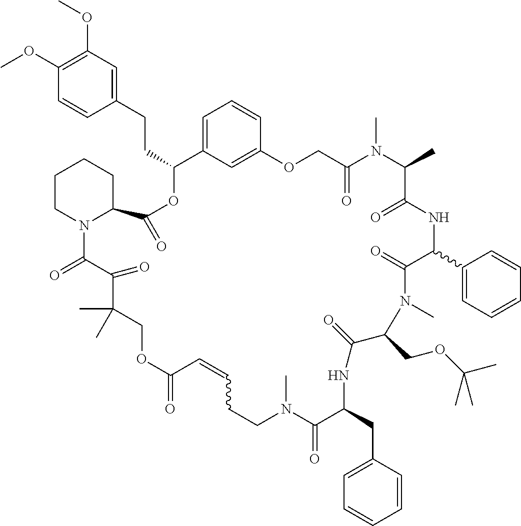 [SMILES: COc1ccc(CC[C@H]2OC(=O)[C@@H]3CCCCN3C(=O)C(=O)C(C)(C)COC(=O)C=CCCN(C)C(=O)[C@H](Cc3ccccc3)NC(=O)[C@H](COC(C)(C)C)N(C)C(=O)C(c3ccccc3)NC(=O)[C@H](C)N(C)C(=O)COc3cccc2c3)cc1OC] |
| 54 | 1200.6 | 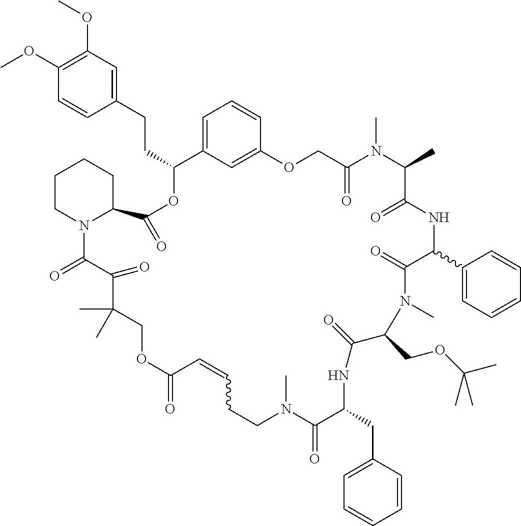 [SMILES: COc1ccc(CC[C@H]2OC(=O)[C@@H]3CCCCN3C(=O)C(=O)C(C)(C)COC(=O)C=CCCN(C)C(=O)[C@@H](Cc3ccccc3)NC(=O)[C@H](COC(C)(C)C)N(C)C(=O)C(c3ccccc3)NC(=O)[C@H](C)N(C)C(=O)COc3cccc2c3)cc1OC] |
| 55 | 1218.6 | 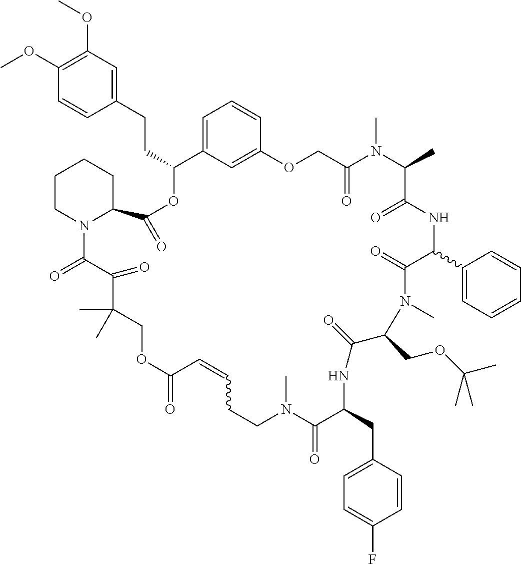 [SMILES: COc1ccc(CC[C@H]2OC(=O)[C@@H]3CCCCN3C(=O)C(=O)C(C)(C)COC(=O)C=CCCN(C)C(=O)[C@H](Cc3ccc(F)cc3)NC(=O)[C@H](COC(C)(C)C)N(C)C(=O)C(c3ccccc3)NC(=O)[C@H](C)N(C)C(=O)COc3cccc2c3)cc1OC] |
| 56 | 1150.6 | 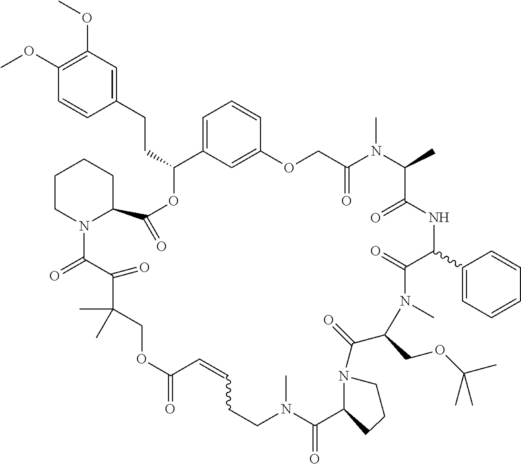 [SMILES: COc1ccc(CC[C@H]2OC(=O)[C@@H]3CCCCN3C(=O)C(=O)C(C)(C)COC(=O)C=CCCN(C)C(=O)[C@@H]3CCCN3C(=O)[C@H](COC(C)(C)C)N(C)C(=O)C(c3ccccc3)NC(=O)[C@H](C)N(C)C(=O)COc3cccc2c3)cc1OC] |
| 57 | 1206.6 | 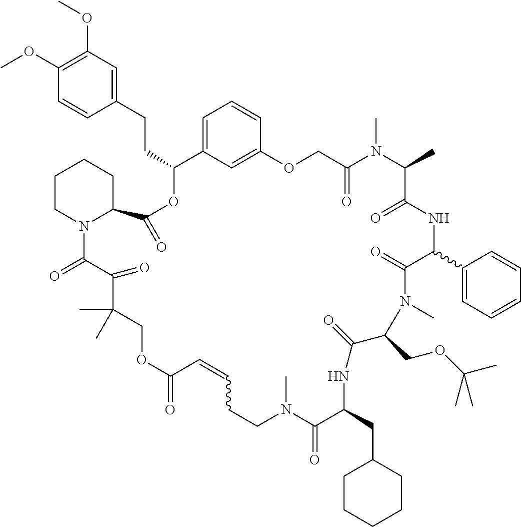 [SMILES: COc1ccc(CC[C@H]2OC(=O)[C@@H]3CCCCN3C(=O)C(=O)C(C)(C)COC(=O)C=CCCN(C)C(=O)[C@H](CC3CCCCC3)NC(=O)[C@H](COC(C)(C)C)N(C)C(=O)C(c3ccccc3)NC(=O)[C@H](C)N(C)C(=O)COc3cccc2c3)cc1OC] |
| 58 | 1250.6 | 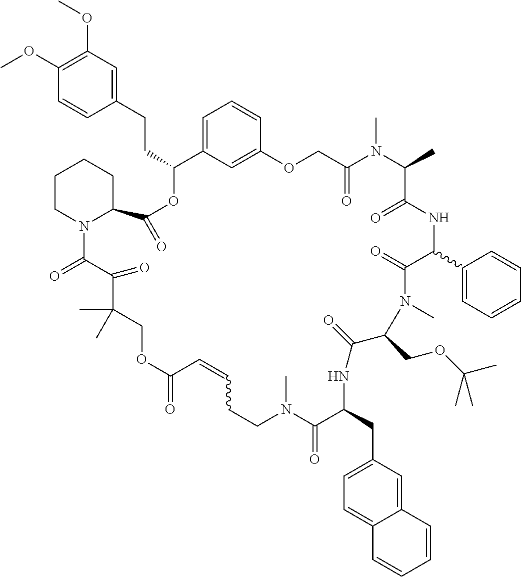 [SMILES: COc1ccc(CC[C@H]2OC(=O)[C@@H]3CCCCN3C(=O)C(=O)C(C)(C)COC(=O)C=CCCN(C)C(=O)[C@H](Cc3ccc4ccccc4c3)NC(=O)[C@H](COC(C)(C)C)N(C)C(=O)C(c3ccccc3)NC(=O)[C@H](C)N(C)C(=O)COc3cccc2c3)cc1OC] |
| 59 | 1152.6 | 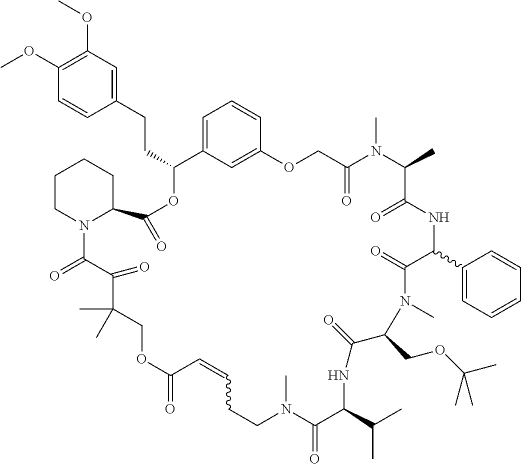 [SMILES: COc1ccc(CC[C@H]2OC(=O)[C@@H]3CCCCN3C(=O)C(=O)C(C)(C)COC(=O)C=CCCN(C)C(=O)[C@H](C(C)C)NC(=O)[C@H](COC(C)(C)C)N(C)C(=O)C(c3ccccc3)NC(=O)[C@H](C)N(C)C(=O)COc3cccc2c3)cc1OC] |
| 60 | 1186.6 | 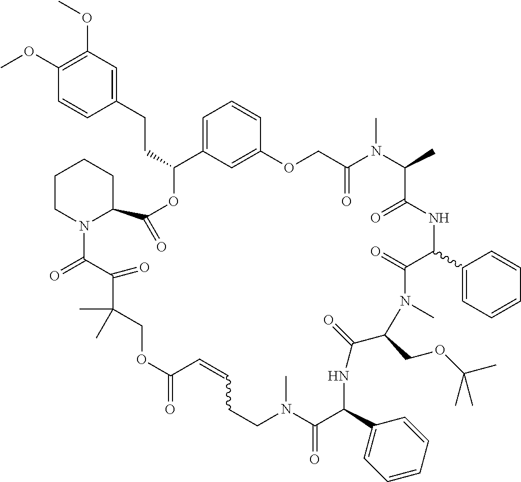 [SMILES: COc1ccc(CC[C@H]2OC(=O)[C@@H]3CCCCN3C(=O)C(=O)C(C)(C)COC(=O)C=CCCN(C)C(=O)[C@H](c3ccccc3)NC(=O)[C@H](COC(C)(C)C)N(C)C(=O)C(c3ccccc3)NC(=O)[C@H](C)N(C)C(=O)COc3cccc2c3)cc1OC] |
| 61 | 1038.5 | 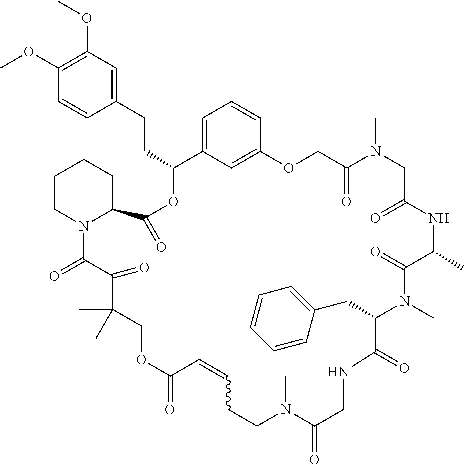 |
| 62 | 1052.5 | 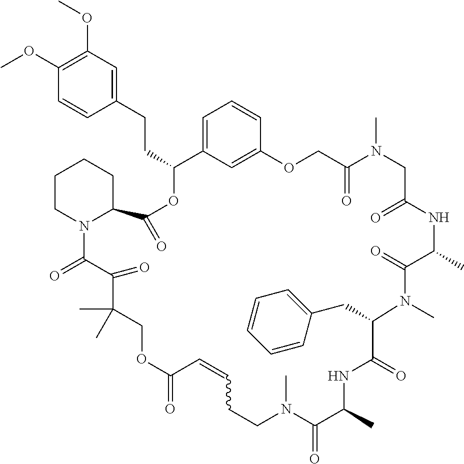 |
| 63 | 1052.5 | 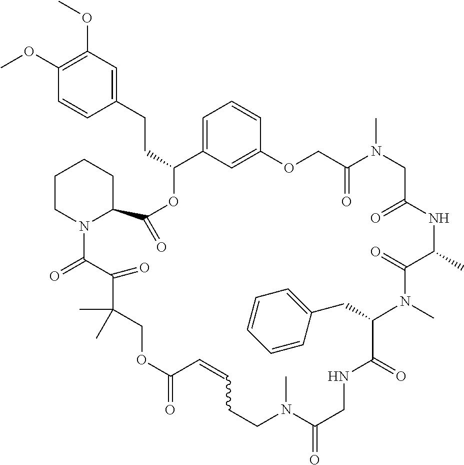 |
| 64 | 1080.5 | 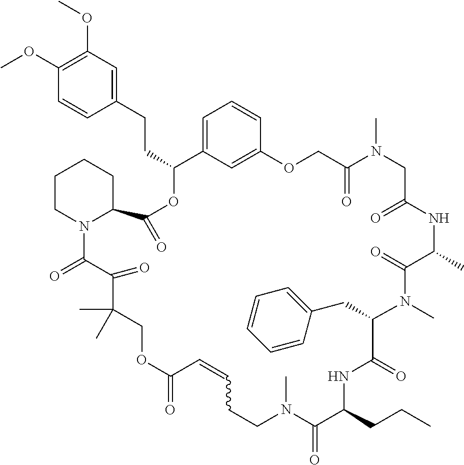 |
| 65 | 1096.5 | 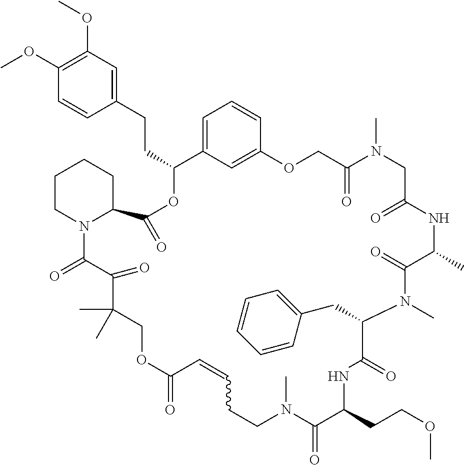 |
| 66 | 1094.5 | 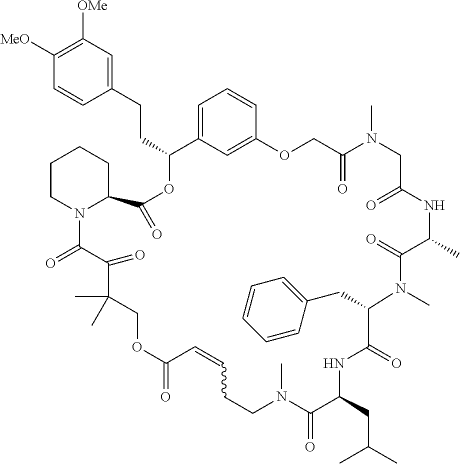 [SMILES: COc1ccc(CC[C@H]2OC(=O)[C@@H]3CCCCN3C(=O)C(=O)C(C)(C)COC(=O)C=CCCN(C)C(=O)[C@H](CC(C)C)NC(=O)[C@H](Cc3ccccc3)N(C)C(=O)[C@@H](C)NC(=O)CN(C)C(=O)COc3cccc2c3)cc1OC] |
| 67 | 1094.5 | 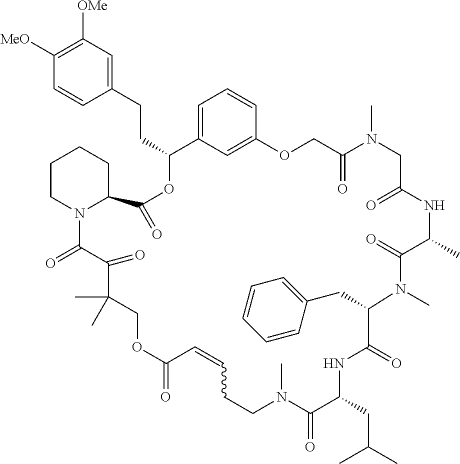 [SMILES: COc1ccc(CC[C@H]2OC(=O)[C@@H]3CCCCN3C(=O)C(=O)C(C)(C)COC(=O)C=CCCN(C)C(=O)[C@@H](CC(C)C)NC(=O)[C@H](Cc3ccccc3)N(C)C(=O)[C@@H](C)NC(=O)CN(C)C(=O)COc3cccc2c3)cc1OC] |
| 68 | 1128.5 | 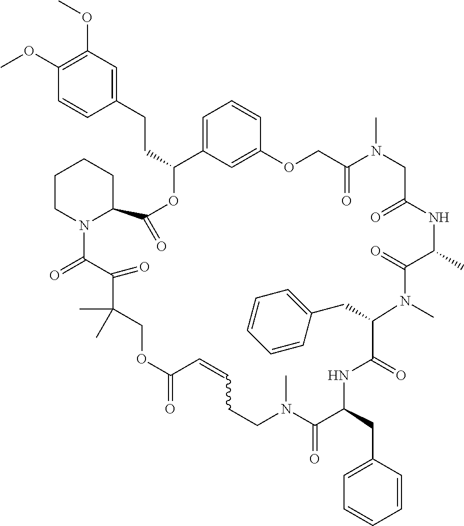 |
| 69 | 1128.5 | 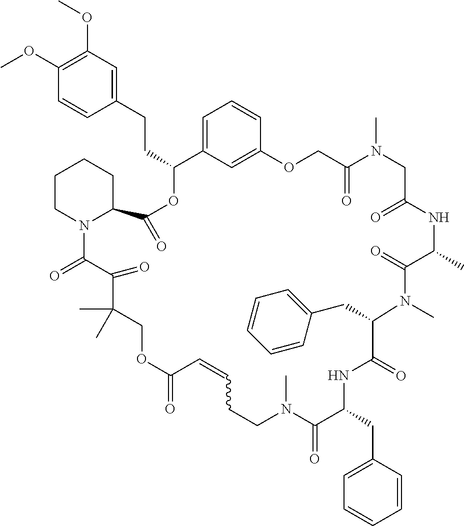 |
| 70 | 1146.5 | 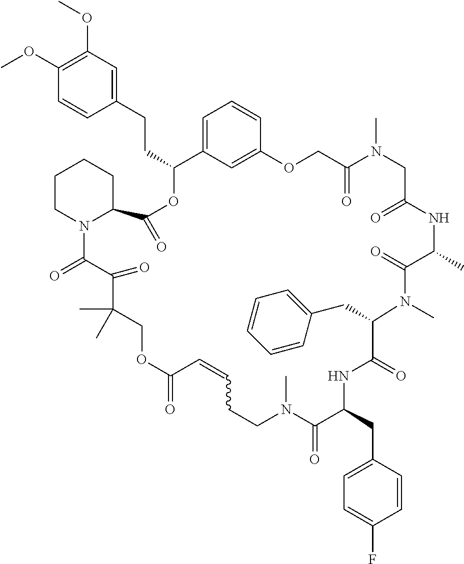 |
| 71 | 1078.5 | 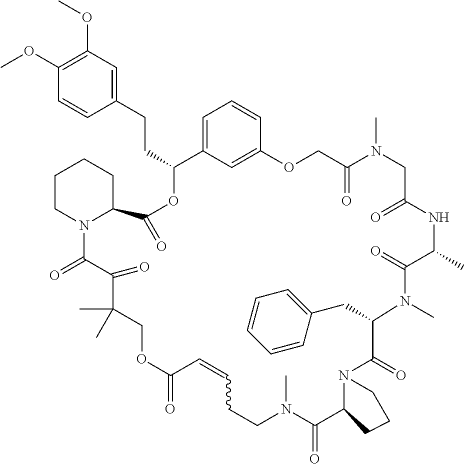 |
| 72 | 1134.6 | 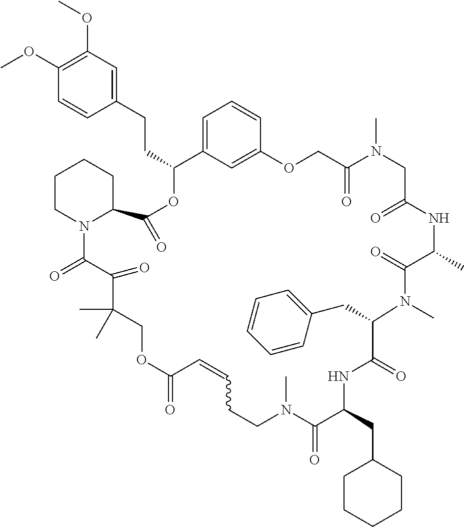 |
| 73 | 1178.5 | 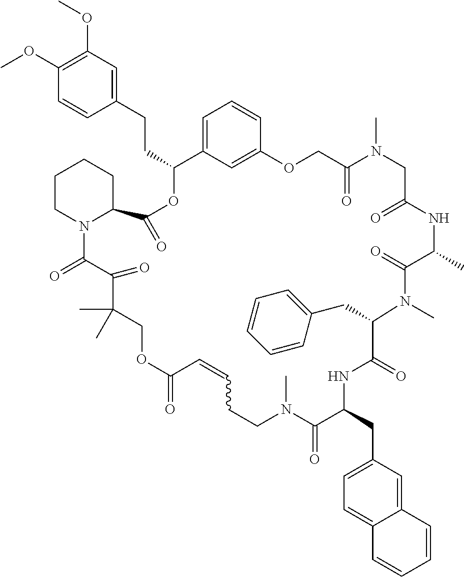 |
| 74 | 1080.5 | 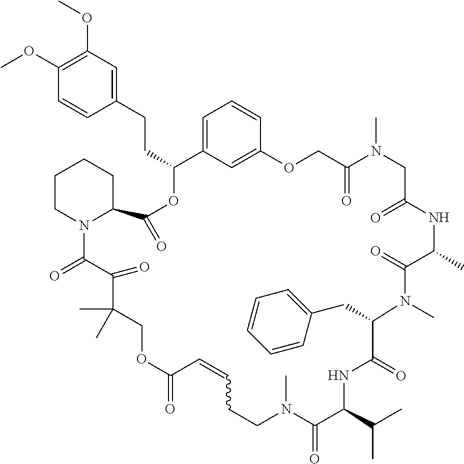 |
| 75 | 1114.5 | 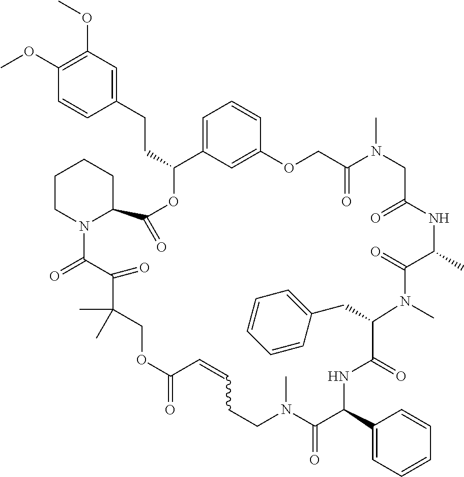 |
| 76 | 1163.6 | 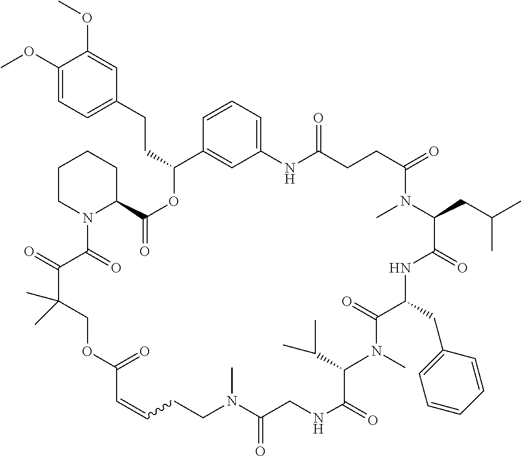 [SMILES: COc1ccc(CC[C@H]2OC(=O)[C@@H]3CCCCN3C(=O)C(=O)C(C)(C)COC(=O)C=CCCN(C)C(=O)CNC(=O)[C@H](C(C)C)N(C)C(=O)[C@@H](Cc3ccccc3)NC(=O)[C@H](CC(C)C)N(C)C(=O)CCC(=O)Nc3cccc2c3)cc1OC] |
| 77 | 1177.6 | 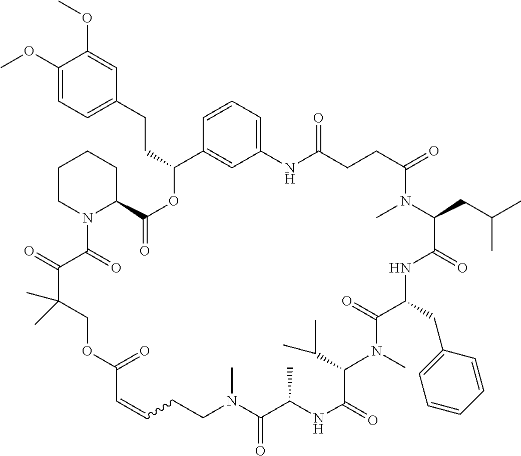 [SMILES: COc1ccc(CC[C@H]2OC(=O)[C@@H]3CCCCN3C(=O)C(=O)C(C)(C)COC(=O)C=CCCN(C)C(=O)[C@H](C)NC(=O)[C@H](C(C)C)N(C)C(=O)[C@@H](Cc3ccccc3)NC(=O)[C@H](CC(C)C)N(C)C(=O)CCC(=O)Nc3cccc2c3)cc1OC] |
| 78 | 1177.6 | 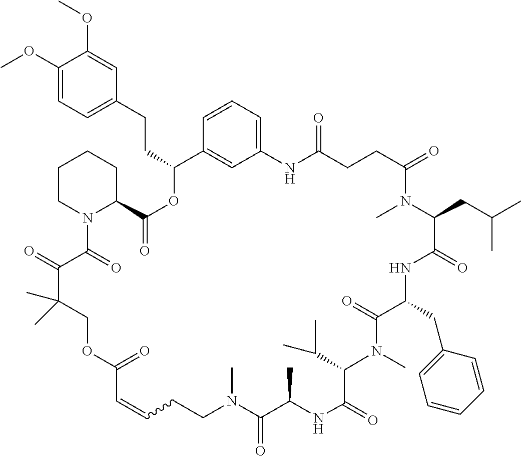 [SMILES: COc1ccc(CC[C@H]2OC(=O)[C@@H]3CCCCN3C(=O)C(=O)C(C)(C)COC(=O)C=CCCN(C)C(=O)[C@@H](C)NC(=O)[C@H](C(C)C)N(C)C(=O)[C@@H](Cc3ccccc3)NC(=O)[C@H](CC(C)C)N(C)C(=O)CCC(=O)Nc3cccc2c3)cc1OC] |
| 79 | 1205.6 | 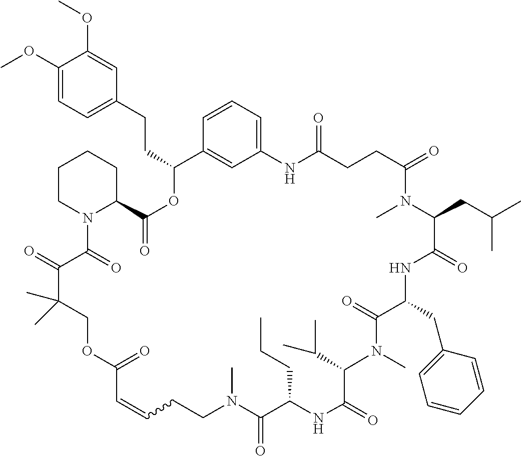 [SMILES: CCC[C@@H]1NC(=O)[C@H](C(C)C)N(C)C(=O)[C@@H](Cc2ccccc2)NC(=O)[C@H](CC(C)C)N(C)C(=O)CCC(=O)Nc2cccc(c2)[C@@H](CCc2ccc(OC)c(OC)c2)OC(=O)[C@@H]2CCCCN2C(=O)C(=O)C(C)(C)COC(=O)C=CCCN(C)C1=O] |
| 80 | 1221.6 | 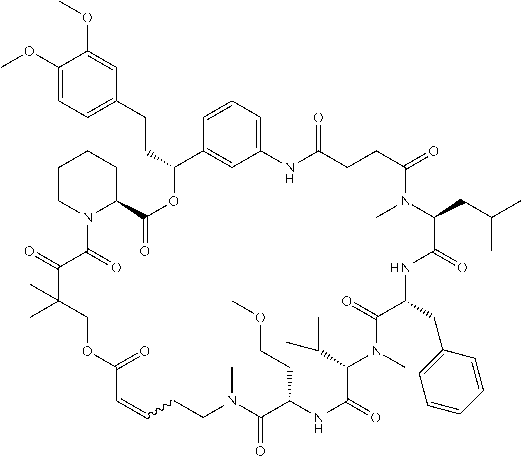 [SMILES: COCC[C@@H]1NC(=O)[C@H](C(C)C)N(C)C(=O)[C@@H](Cc2ccccc2)NC(=O)[C@H](CC(C)C)N(C)C(=O)CCC(=O)Nc2cccc(c2)[C@@H](CCc2ccc(OC)c(OC)c2)OC(=O)[C@@H]2CCCCN2C(=O)C(=O)C(C)(C)COC(=O)C=CCCN(C)C1=O] |
| 81 | 1219.7 | 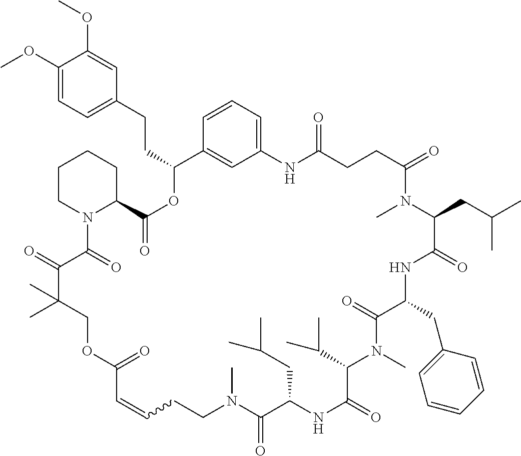 [SMILES: COc1ccc(CC[C@H]2OC(=O)[C@@H]3CCCCN3C(=O)C(=O)C(C)(C)COC(=O)C=CCCN(C)C(=O)[C@H](CC(C)C)NC(=O)[C@H](C(C)C)N(C)C(=O)[C@@H](Cc3ccccc3)NC(=O)[C@H](CC(C)C)N(C)C(=O)CCC(=O)Nc3cccc2c3)cc1OC] |
| 82 | 1219.7 | 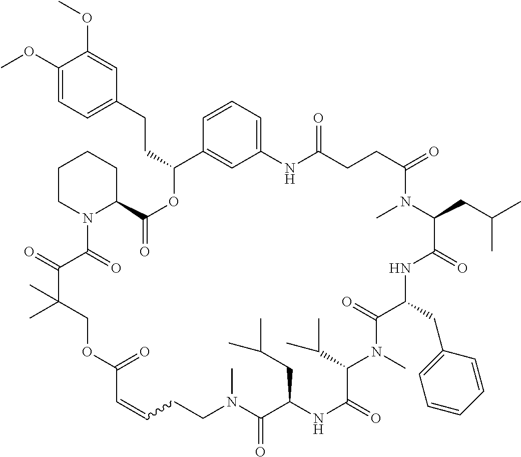 [SMILES: COc1ccc(CC[C@H]2OC(=O)[C@@H]3CCCCN3C(=O)C(=O)C(C)(C)COC(=O)C=CCCN(C)C(=O)[C@@H](CC(C)C)NC(=O)[C@H](C(C)C)N(C)C(=O)[C@@H](Cc3ccccc3)NC(=O)[C@H](CC(C)C)N(C)C(=O)CCC(=O)Nc3cccc2c3)cc1OC] |
| 83 | 1253.6 | 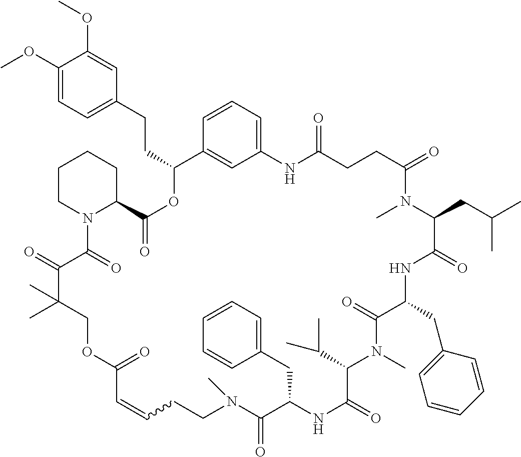 [SMILES: COc1ccc(CC[C@H]2OC(=O)[C@@H]3CCCCN3C(=O)C(=O)C(C)(C)COC(=O)C=CCCN(C)C(=O)[C@H](Cc3ccccc3)NC(=O)[C@H](C(C)C)N(C)C(=O)[C@@H](Cc3ccccc3)NC(=O)[C@H](CC(C)C)N(C)C(=O)CCC(=O)Nc3cccc2c3)cc1OC] |
| 84 | 1253.6 | 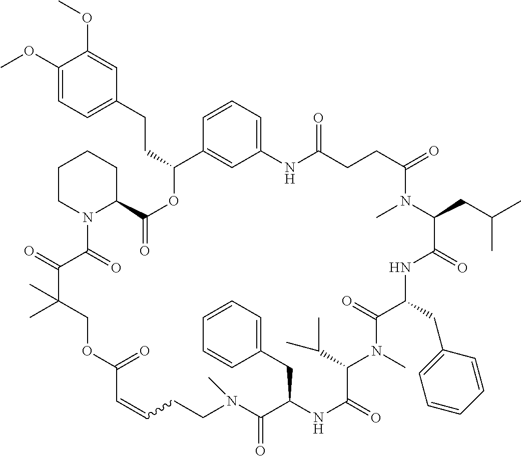 [SMILES: COc1ccc(CC[C@H]2OC(=O)[C@@H]3CCCCN3C(=O)C(=O)C(C)(C)COC(=O)C=CCCN(C)C(=O)[C@@H](Cc3ccccc3)NC(=O)[C@H](C(C)C)N(C)C(=O)[C@@H](Cc3ccccc3)NC(=O)[C@H](CC(C)C)N(C)C(=O)CCC(=O)Nc3cccc2c3)cc1OC] |
| 85 | 1271.6 | 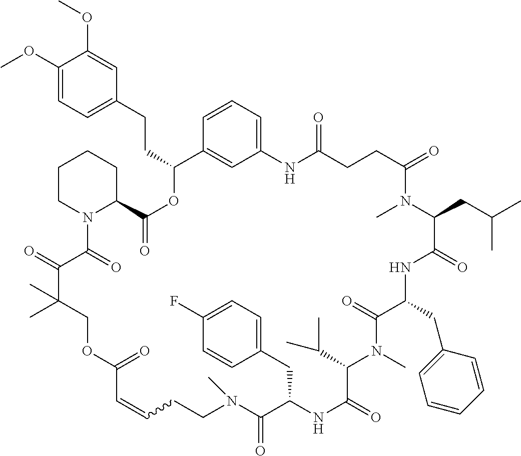 [SMILES: COc1ccc(CC[C@H]2OC(=O)[C@@H]3CCCCN3C(=O)C(=O)C(C)(C)COC(=O)C=CCCN(C)C(=O)[C@H](Cc3ccc(F)cc3)NC(=O)[C@H](C(C)C)N(C)C(=O)[C@@H](Cc3ccccc3)NC(=O)[C@H](CC(C)C)N(C)C(=O)CCC(=O)Nc3cccc2c3)cc1OC] |
| 86 | 1203.6 | 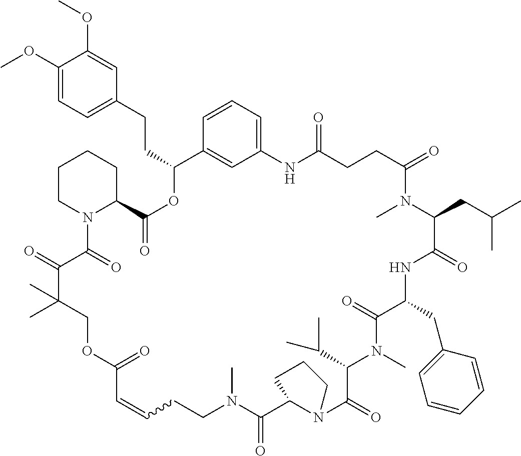 [SMILES: COc1ccc(CC[C@H]2OC(=O)[C@@H]3CCCCN3C(=O)C(=O)C(C)(C)COC(=O)C=CCCN(C)C(=O)[C@@H]3CCCN3C(=O)[C@H](C(C)C)N(C)C(=O)[C@@H](Cc3ccccc3)NC(=O)[C@H](CC(C)C)N(C)C(=O)CCC(=O)Nc3cccc2c3)cc1OC] |
| 87 | 1259.7 | 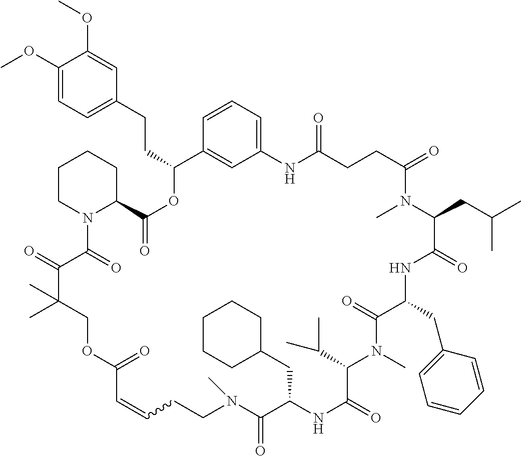 [SMILES: COc1ccc(CC[C@H]2OC(=O)[C@@H]3CCCCN3C(=O)C(=O)C(C)(C)COC(=O)C=CCCN(C)C(=O)[C@H](CC3CCCCC3)NC(=O)[C@H](C(C)C)N(C)C(=O)[C@@H](Cc3ccccc3)NC(=O)[C@H](CC(C)C)N(C)C(=O)CCC(=O)Nc3cccc2c3)cc1OC] |
| 88 | 1303.7 | 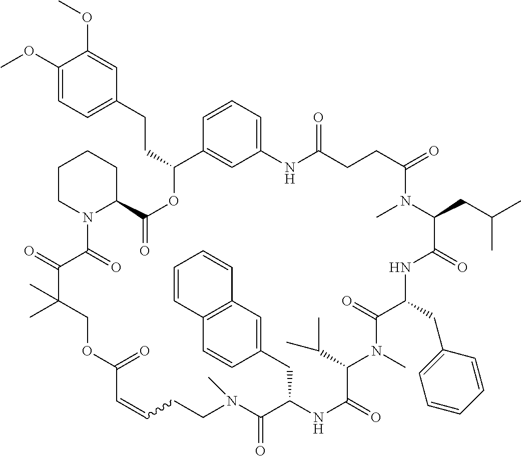 [SMILES: COc1ccc(CC[C@H]2OC(=O)[C@@H]3CCCCN3C(=O)C(=O)C(C)(C)COC(=O)C=CCCN(C)C(=O)[C@H](Cc3ccc4ccccc4c3)NC(=O)[C@H](C(C)C)N(C)C(=O)[C@@H](Cc3ccccc3)NC(=O)[C@H](CC(C)C)N(C)C(=O)CCC(=O)Nc3cccc2c3)cc1OC] |
| 89 | 1205.6 | 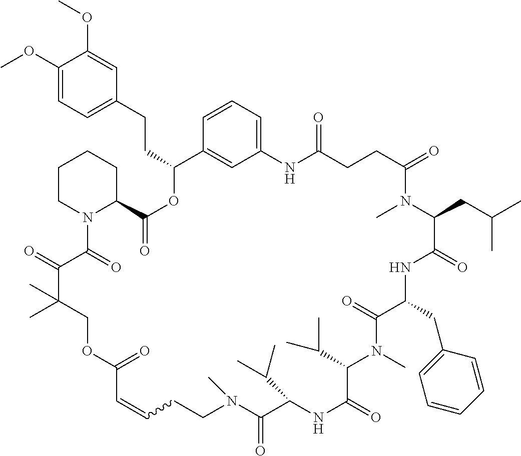 [SMILES: COc1ccc(CC[C@H]2OC(=O)[C@@H]3CCCCN3C(=O)C(=O)C(C)(C)COC(=O)C=CCCN(C)C(=O)[C@H](C(C)C)NC(=O)[C@H](C(C)C)N(C)C(=O)[C@@H](Cc3ccccc3)NC(=O)[C@H](CC(C)C)N(C)C(=O)CCC(=O)Nc3cccc2c3)cc1OC] |
| 90 | 1239.6 | 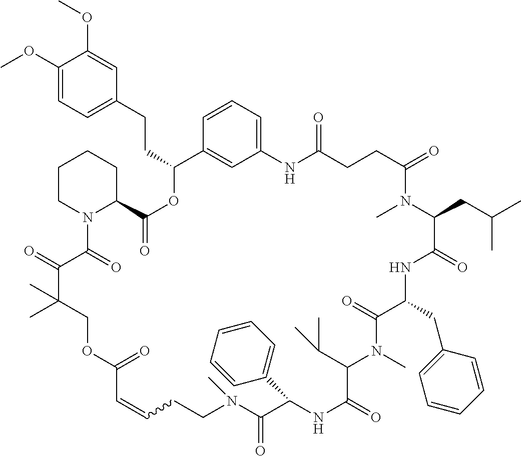 [SMILES: COc1ccc(CC[C@H]2OC(=O)[C@@H]3CCCCN3C(=O)C(=O)C(C)(C)COC(=O)C=CCCN(C)C(=O)[C@H](c3ccccc3)NC(=O)C(C(C)C)N(C)C(=O)[C@@H](Cc3ccccc3)NC(=O)[C@H](CC(C)C)N(C)C(=O)CCC(=O)Nc3cccc2c3)cc1OC] |
| 91 | 1150.6 | 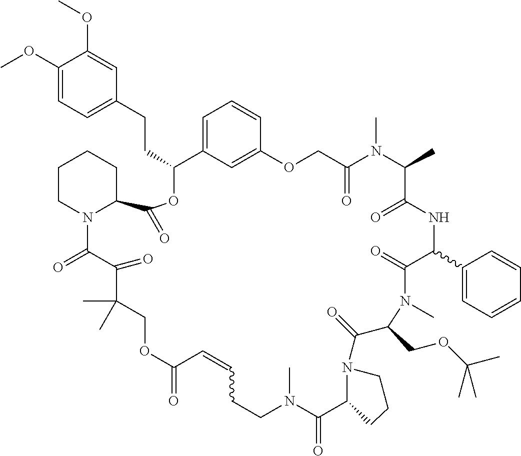 [SMILES: COc1ccc(CC[C@H]2OC(=O)[C@@H]3CCCCN3C(=O)C(=O)C(C)(C)COC(=O)C=CCCN(C)C(=O)[C@H]3CCCN3C(=O)[C@H](COC(C)(C)C)N(C)C(=O)C(c3ccccc3)NC(=O)[C@H](C)N(C)C(=O)COc3cccc2c3)cc1OC] |
| 92 | 1164.6 | 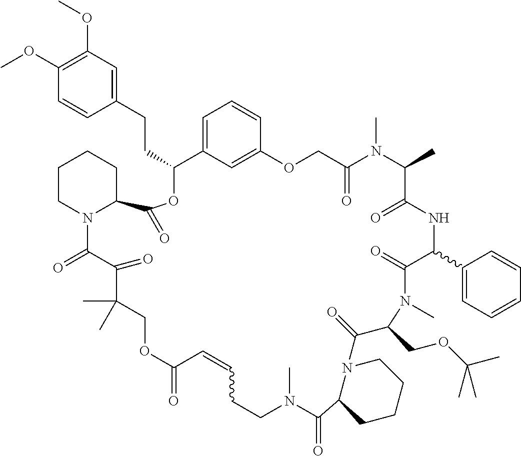 [SMILES: COc1ccc(CC[C@H]2OC(=O)[C@@H]3CCCCN3C(=O)C(=O)C(C)(C)COC(=O)C=CCCN(C)C(=O)[C@@H]3CCCCN3C(=O)[C@H](COC(C)(C)C)N(C)C(=O)C(c3ccccc3)NC(=O)[C@H](C)N(C)C(=O)COc3cccc2c3)cc1OC] |
| 93 | 1166.6 | 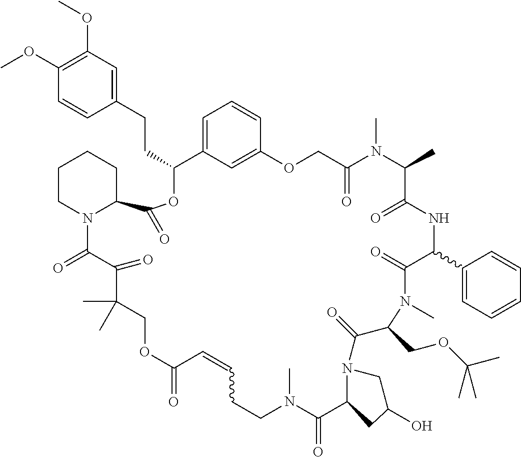 |
| 94 | 1138.6 | 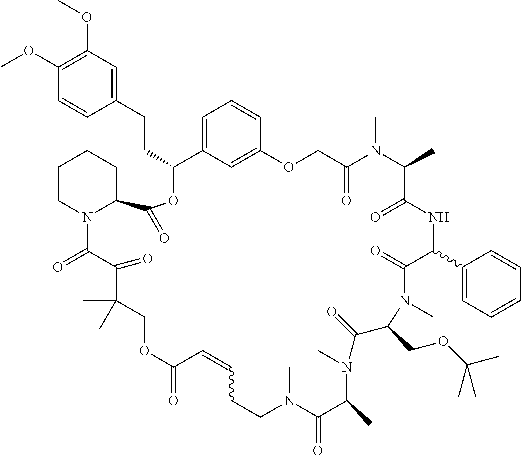 [SMILES: COc1ccc(CC[C@H]2OC(=O)[C@@H]3CCCCN3C(=O)C(=O)C(C)(C)COC(=O)C=CCCN(C)C(=O)[C@H](C)N(C)C(=O)[C@H](COC(C)(C)C)N(C)C(=O)C(c3ccccc3)NC(=O)[C@H](C)N(C)C(=O)COc3cccc2c3)cc1OC] |
| 95 | 1178.5 | 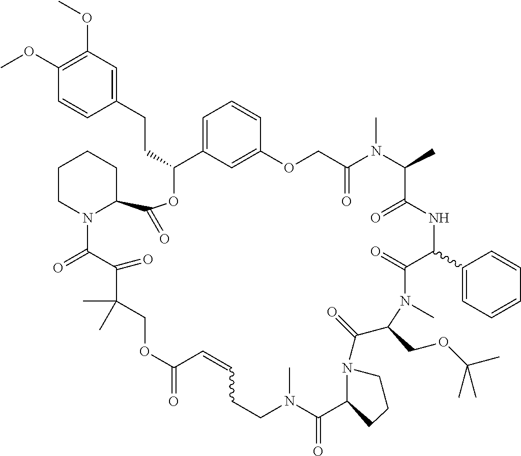 [SMILES: COc1ccc(CC[C@H]2OC(=O)[C@@H]3CCCCN3C(=O)C(=O)C(C)(C)COC(=O)C=CCCN(C)C(=O)[C@@H]3CCCN3C(=O)[C@H](COC(C)(C)C)N(C)C(=O)C(c3ccccc3)NC(=O)[C@H](C)N(C)C(=O)COc3cccc2c3)cc1OC] |
| 96 | 1184.6 | 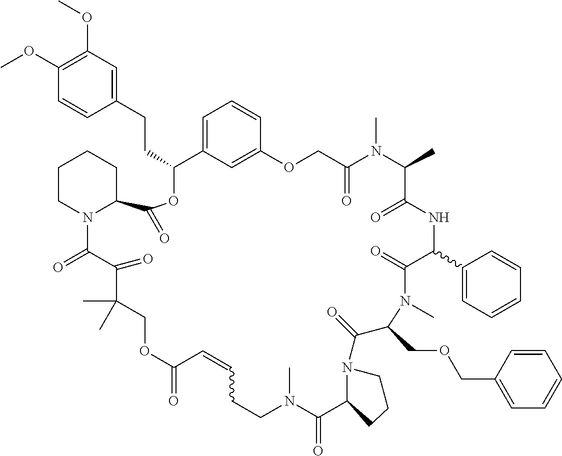 [SMILES: COc1ccc(CC[C@H]2OC(=O)[C@@H]3CCCCN3C(=O)C(=O)C(C)(C)COC(=O)C=CCCN(C)C(=O)[C@@H]3CCCN3C(=O)[C@H](COCc3ccccc3)N(C)C(=O)C(c3ccccc3)NC(=O)[C@H](C)N(C)C(=O)COc3cccc2c3)cc1OC] |
| 97 | 1094.5 | 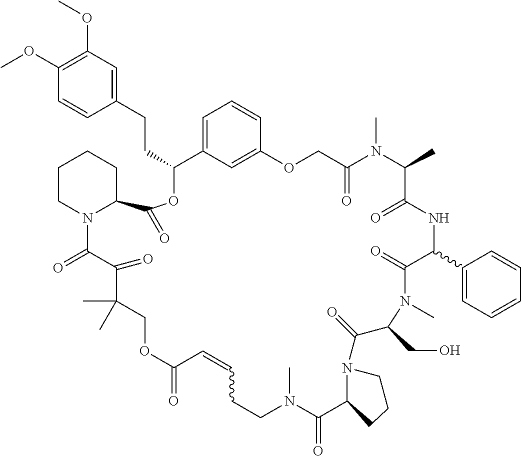 [SMILES: COc1ccc(CC[C@H]2OC(=O)[C@@H]3CCCCN3C(=O)C(=O)C(C)(C)COC(=O)C=CCCN(C)C(=O)[C@@H]3CCCN3C(=O)[C@H](CO)N(C)C(=O)C(c3ccccc3)NC(=O)[C@H](C)N(C)C(=O)COc3cccc2c3)cc1OC] |
| 98 | 1226.6 | 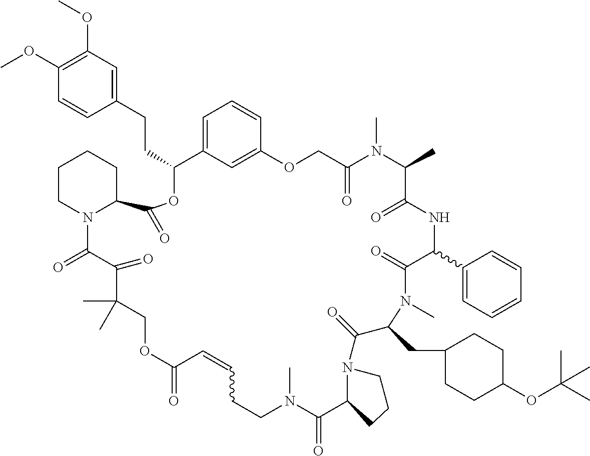 [SMILES: COc1ccc(CC[C@H]2OC(=O)[C@@H]3CCCCN3C(=O)C(=O)C(C)(C)COC(=O)C=CCCN(C)C(=O)[C@@H]3CCCN3C(=O)[C@H](CC3CCC(OC(C)(C)C)CC3)N(C)C(=O)C(c3ccccc3)NC(=O)[C@H](C)N(C)C(=O)COc3cccc2c3)cc1OC] |
| 99 | 1164.6 | 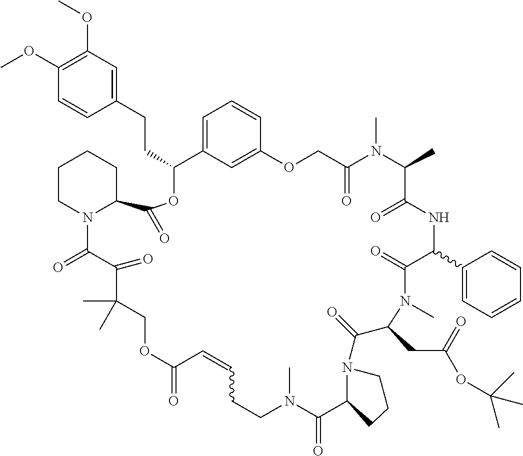 [SMILES: COc1ccc(CC[C@H]2OC(=O)[C@@H]3CCCCN3C(=O)C(=O)C(C)(C)COC(=O)C=CCCN(C)C(=O)[C@@H]3CCCN3C(=O)[C@H](CC(=O)OC(C)(C)C)N(C)C(=O)C(c3ccccc3)NC(=O)[C@H](C)N(C)C(=O)COc3cccc2c3)cc1OC] |
| 100 | 1088.6 | 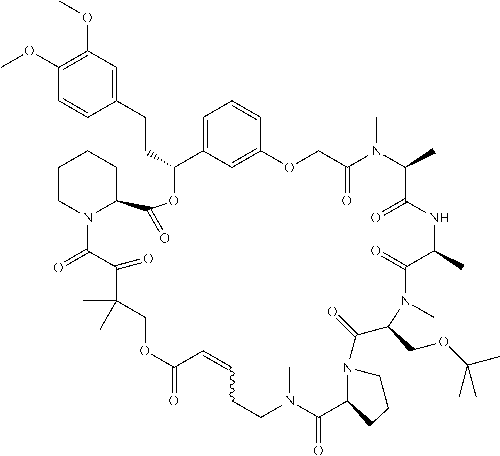 [SMILES: COc1ccc(CC[C@H]2OC(=O)[C@@H]3CCCCN3C(=O)C(=O)C(C)(C)COC(=O)C=CCCN(C)C(=O)[C@@H]3CCCN3C(=O)[C@H](COC(C)(C)C)N(C)C(=O)[C@H](C)NC(=O)[C@H](C)N(C)C(=O)COc3cccc2c3)cc1OC] |
| 101 | 1164.6 | 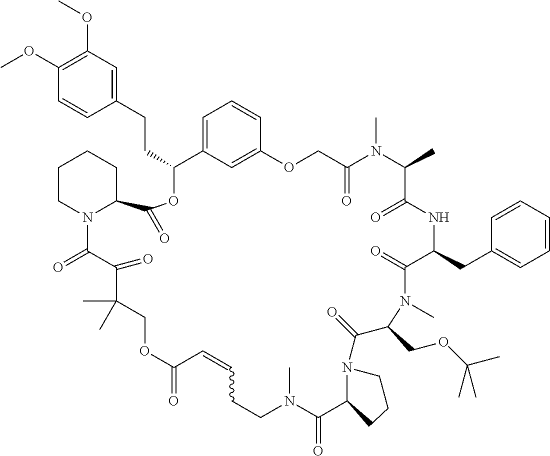 [SMILES: COc1ccc(CC[C@H]2OC(=O)[C@@H]3CCCCN3C(=O)C(=O)C(C)(C)COC(=O)C=CCCN(C)C(=O)[C@@H]3CCCN3C(=O)[C@H](COC(C)(C)C)N(C)C(=O)[C@H](Cc3ccccc3)NC(=O)[C@H](C)N(C)C(=O)COc3cccc2c3)cc1OC] |
| 102 | 1150.6 | 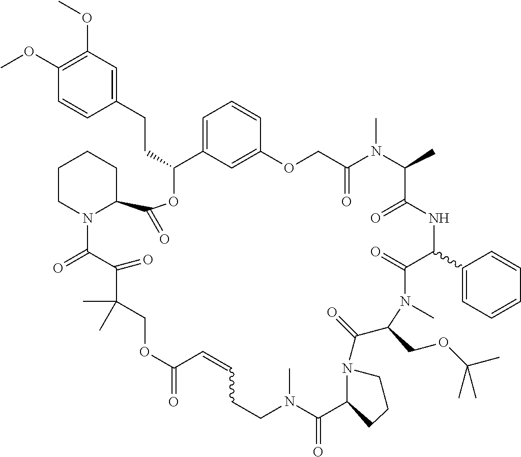 [SMILES: COc1ccc(CC[C@H]2OC(=O)[C@@H]3CCCCN3C(=O)C(=O)C(C)(C)COC(=O)C=CCCN(C)C(=O)[C@@H]3CCCN3C(=O)[C@H](COC(C)(C)C)N(C)C(=O)C(c3ccccc3)NC(=O)[C@H](C)N(C)C(=O)COc3cccc2c3)cc1OC] |
| 103 | 1182.6 | 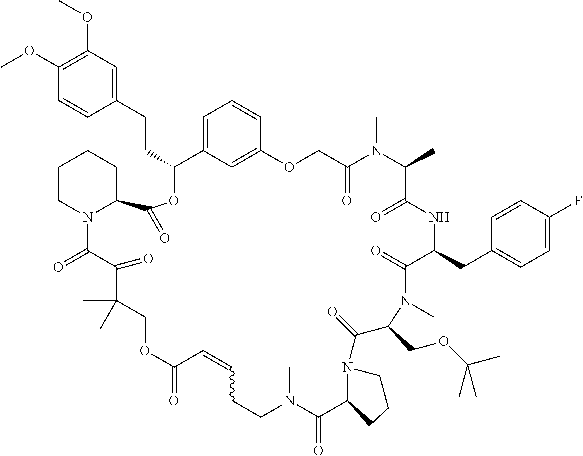 [SMILES: COc1ccc(CC[C@H]2OC(=O)[C@@H]3CCCCN3C(=O)C(=O)C(C)(C)COC(=O)C=CCCN(C)C(=O)[C@@H]3CCCN3C(=O)[C@H](COC(C)(C)C)N(C)C(=O)[C@H](Cc3ccc(F)cc3)NC(=O)[C@H](C)N(C)C(=O)COc3cccc2c3)cc1OC] |
| 104 | 1165.6 | 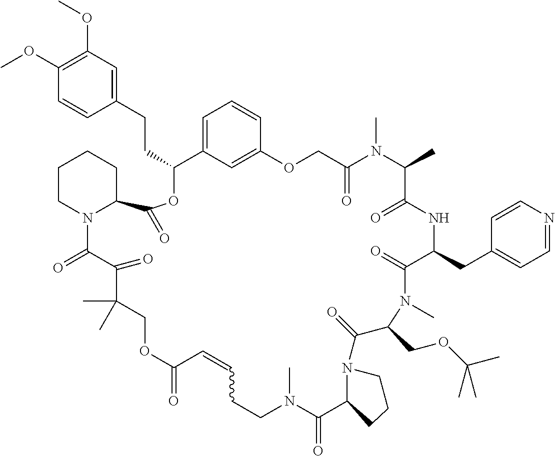 [SMILES: COc1ccc(CC[C@H]2OC(=O)[C@@H]3CCCCN3C(=O)C(=O)C(C)(C)COC(=O)C=CCCN(C)C(=O)[C@@H]3CCCN3C(=O)[C@H](COC(C)(C)C)N(C)C(=O)[C@H](Cc3ccncc3)NC(=O)[C@H](C)N(C)C(=O)COc3cccc2c3)cc1OC] |
| 105 | 1136.6 | 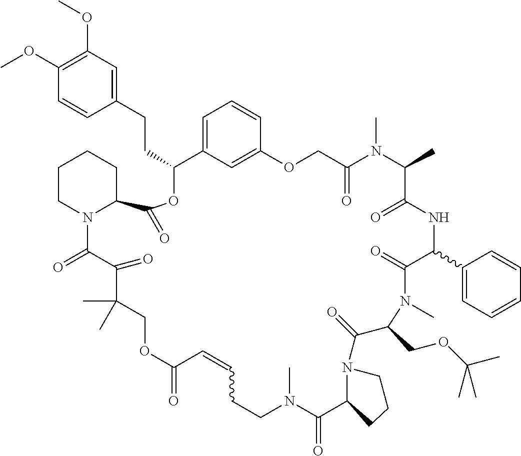 [SMILES: COc1ccc(CC[C@H]2OC(=O)[C@@H]3CCCCN3C(=O)C(=O)C(C)(C)COC(=O)C=CCCN(C)C(=O)[C@@H]3CCCN3C(=O)[C@H](COC(C)(C)C)N(C)C(=O)C(c3ccccc3)NC(=O)[C@H](C)N(C)C(=O)COc3cccc2c3)cc1OC] |
| 106 | 1150.6 | 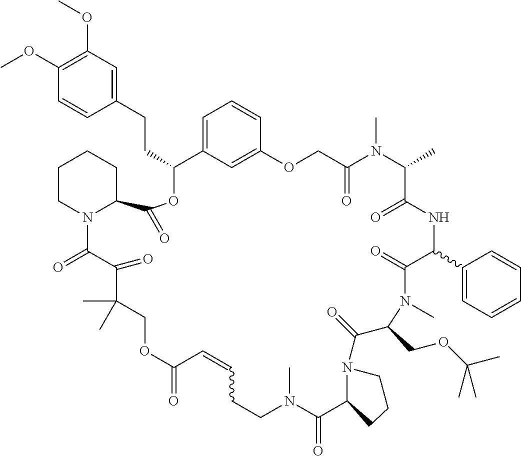 [SMILES: COc1ccc(CC[C@H]2OC(=O)[C@@H]3CCCCN3C(=O)C(=O)C(C)(C)COC(=O)C=CCCN(C)C(=O)[C@@H]3CCCN3C(=O)[C@H](COC(C)(C)C)N(C)C(=O)C(c3ccccc3)NC(=O)[C@@H](C)N(C)C(=O)COc3cccc2c3)cc1OC] |
| 107 | 1136.6 | 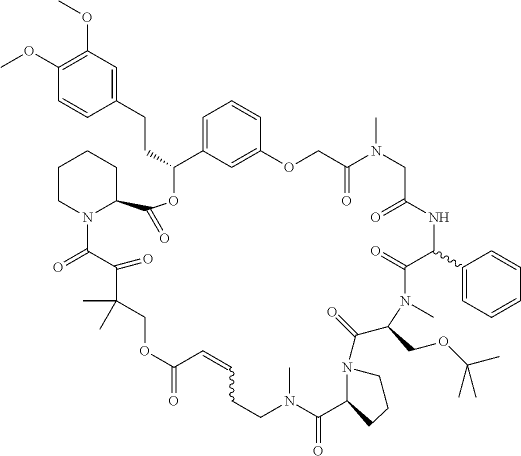 [SMILES: COc1ccc(CC[C@H]2OC(=O)[C@@H]3CCCCN3C(=O)C(=O)C(C)(C)COC(=O)C=CCCN(C)C(=O)[C@@H]3CCCN3C(=O)[C@H](COC(C)(C)C)N(C)C(=O)C(c3ccccc3)NC(=O)CN(C)C(=O)COc3cccc2c3)cc1OC] |
| 108 | 1110.5 | 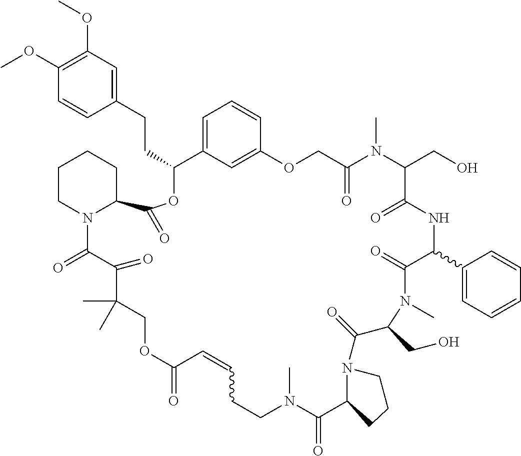 [SMILES: COc1ccc(CC[C@H]2OC(=O)[C@@H]3CCCCN3C(=O)C(=O)C(C)(C)COC(=O)C=CCCN(C)C(=O)[C@@H]3CCCN3C(=O)[C@H](CO)N(C)C(=O)C(c3ccccc3)NC(=O)C(CO)N(C)C(=O)COc3cccc2c3)cc1OC] |
| 109 | 1169.5 | 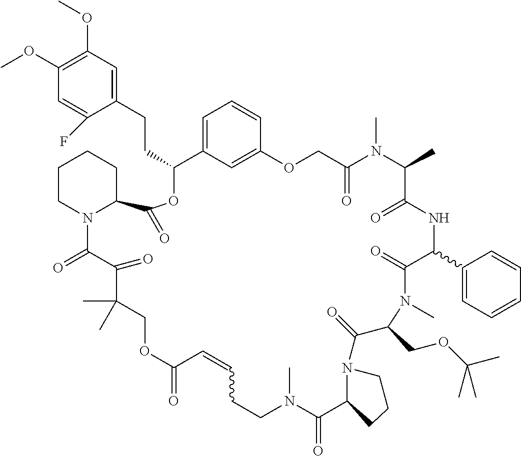 [SMILES: COc1cc(F)c(CC[C@H]2OC(=O)[C@@H]3CCCCN3C(=O)C(=O)C(C)(C)COC(=O)C=CCCN(C)C(=O)[C@@H]3CCCN3C(=O)[C@H](COC(C)(C)C)N(C)C(=O)C(c3ccccc3)NC(=O)[C@H](C)N(C)C(=O)COc3cccc2c3)cc1OC] |
| 110 | 1169.5 | 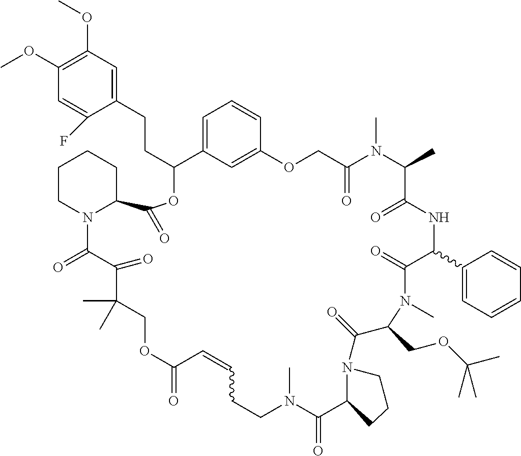 [SMILES: COc1cc(F)c(CCC2OC(=O)[C@@H]3CCCCN3C(=O)C(=O)C(C)(C)COC(=O)C=CCCN(C)C(=O)[C@@H]3CCCN3C(=O)[C@H](COC(C)(C)C)N(C)C(=O)C(c3ccccc3)NC(=O)[C@H](C)N(C)C(=O)COc3cccc2c3)cc1OC] |
| 111 | 1169.5 | 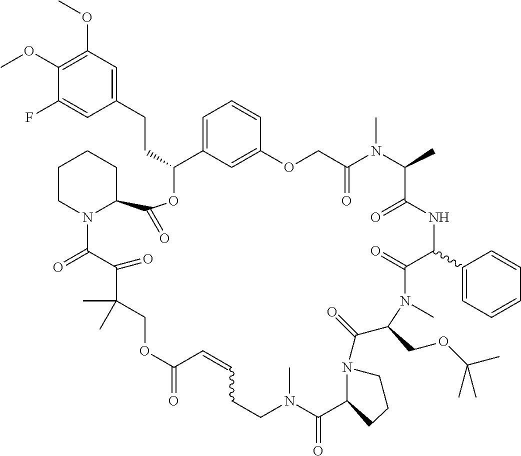 [SMILES: COc1cc(CC[C@H]2OC(=O)[C@@H]3CCCCN3C(=O)C(=O)C(C)(C)COC(=O)C=CCCN(C)C(=O)[C@@H]3CCCN3C(=O)[C@H](COC(C)(C)C)N(C)C(=O)C(c3ccccc3)NC(=O)[C@H](C)N(C)C(=O)COc3cccc2c3)cc(F)c1OC] |
| 112 | 1169.5 | 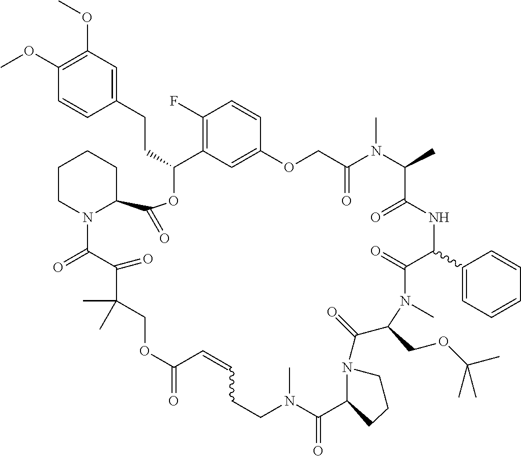 [SMILES: COc1ccc(CC[C@H]2OC(=O)[C@@H]3CCCCN3C(=O)C(=O)C(C)(C)COC(=O)C=CCCN(C)C(=O)[C@@H]3CCCN3C(=O)[C@H](COC(C)(C)C)N(C)C(=O)C(c3ccccc3)NC(=O)[C@H](C)N(C)C(=O)COc3ccc(F)c2c3)cc1OC] |
| 113 | 1181.5 | 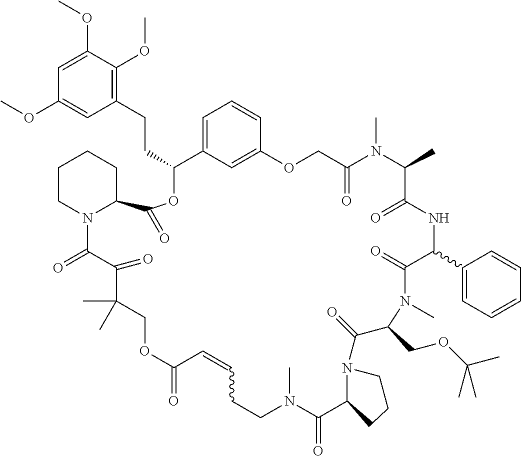 [SMILES: COc1cc(CC[C@H]2OC(=O)[C@@H]3CCCCN3C(=O)C(=O)C(C)(C)COC(=O)C=CCCN(C)C(=O)[C@@H]3CCCN3C(=O)[C@H](COC(C)(C)C)N(C)C(=O)C(c3ccccc3)NC(=O)[C@H](C)N(C)C(=O)COc3cccc2c3)c(OC)c(OC)c1] |
| 114 | 1169.5 | 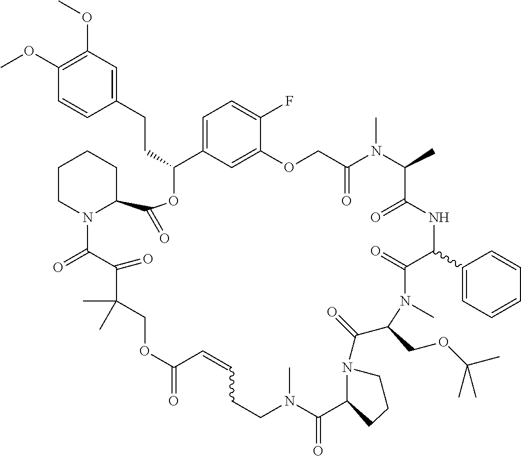 [SMILES: COc1ccc(CC[C@H]2OC(=O)[C@@H]3CCCCN3C(=O)C(=O)C(C)(C)COC(=O)C=CCCN(C)C(=O)[C@@H]3CCCN3C(=O)[C@H](COC(C)(C)C)N(C)C(=O)C(c3ccccc3)NC(=O)[C@H](C)N(C)C(=O)COc3cc2ccc3F)cc1OC] |
| 115 | 1181.5 | 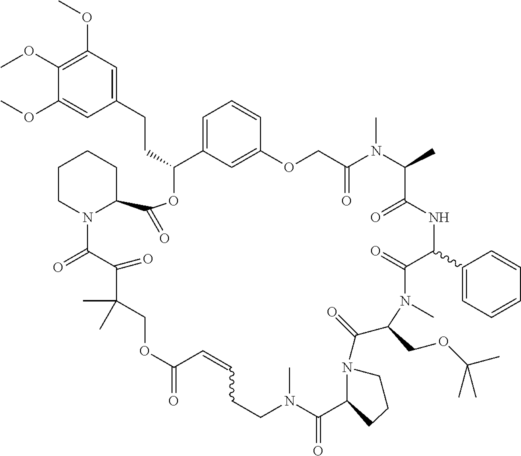 [SMILES: COc1cc(CC[C@H]2OC(=O)[C@@H]3CCCCN3C(=O)C(=O)C(C)(C)COC(=O)C=CCCN(C)C(=O)[C@@H]3CCCN3C(=O)[C@H](COC(C)(C)C)N(C)C(=O)C(c3ccccc3)NC(=O)[C@H](C)N(C)C(=O)COc3cccc2c3)cc(OC)c1OC] |
| 116 | 1166.6 | 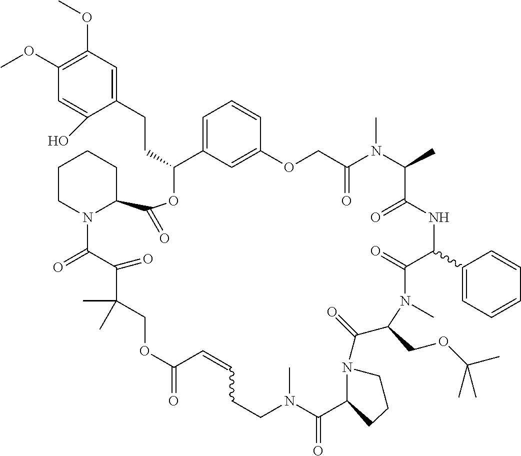 [SMILES: COc1cc(O)c(CC[C@H]2OC(=O)[C@@H]3CCCCN3C(=O)C(=O)C(C)(C)COC(=O)C=CCCN(C)C(=O)[C@@H]3CCCN3C(=O)[C@H](COC(C)(C)C)N(C)C(=O)C(c3ccccc3)NC(=O)[C@H](C)N(C)C(=O)COc3cccc2c3)cc1OC] |
| 117 | 1169.5 | 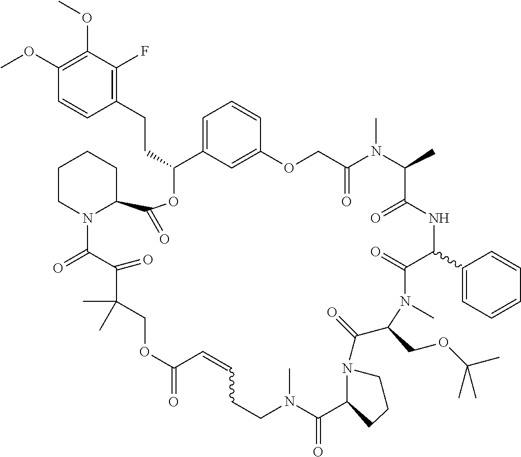 [SMILES: COc1ccc(CC[C@H]2OC(=O)[C@@H]3CCCCN3C(=O)C(=O)C(C)(C)COC(=O)C=CCCN(C)C(=O)[C@@H]3CCCN3C(=O)[C@H](COC(C)(C)C)N(C)C(=O)C(c3ccccc3)NC(=O)[C@H](C)N(C)C(=O)COc3cccc2c3)c(F)c1OC] |
| 118 | 1166.5 | 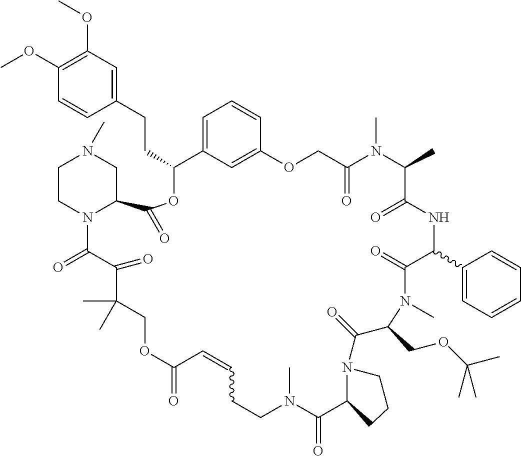 [SMILES: COc1ccc(CC[C@H]2OC(=O)[C@@H]3CN(C)CCN3C(=O)C(=O)C(C)(C)COC(=O)C=CCCN(C)C(=O)[C@@H]3CCCN3C(=O)[C@H](COC(C)(C)C)N(C)C(=O)C(c3ccccc3)NC(=O)[C@H](C)N(C)C(=O)COc3cccc2c3)cc1OC] |
| 119 | 1167.5 | 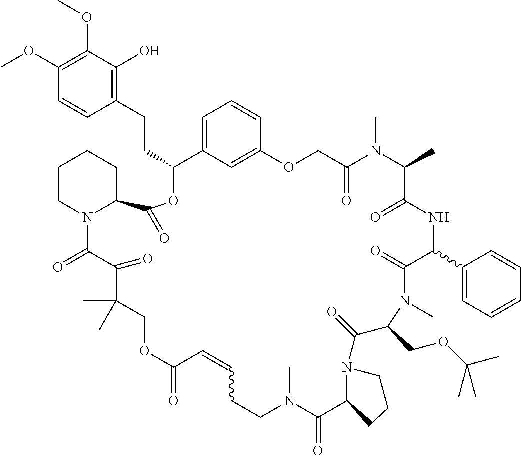 [SMILES: COc1ccc(CC[C@H]2OC(=O)[C@@H]3CCCCN3C(=O)C(=O)C(C)(C)COC(=O)C=CCCN(C)C(=O)[C@@H]3CCCN3C(=O)[C@H](COC(C)(C)C)N(C)C(=O)C(c3ccccc3)NC(=O)[C@H](C)N(C)C(=O)COc3cccc2c3)c(O)c1OC] |
| 120 | 1167.5 | 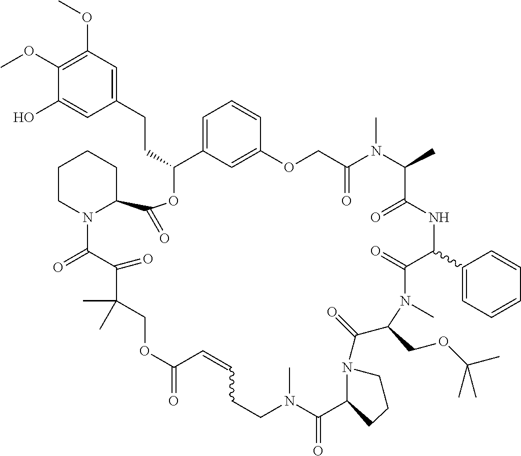 [SMILES: COc1cc(CC[C@H]2OC(=O)[C@@H]3CCCCN3C(=O)C(=O)C(C)(C)COC(=O)C=CCCN(C)C(=O)[C@@H]3CCCN3C(=O)[C@H](COC(C)(C)C)N(C)C(=O)C(c3ccccc3)NC(=O)[C@H](C)N(C)C(=O)COc3cccc2c3)cc(O)c1OC] |
| 121 | 1169.5 | 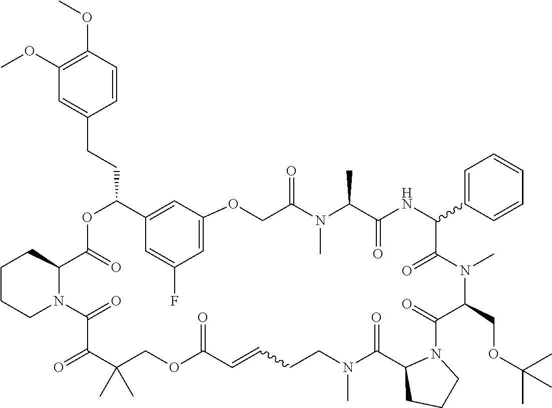 [SMILES: COc1ccc(CC[C@H]2OC(=O)[C@@H]3CCCCN3C(=O)C(=O)C(C)(C)COC(=O)C=CCCN(C)C(=O)[C@@H]3CCCN3C(=O)[C@H](COC(C)(C)C)N(C)C(=O)C(c3ccccc3)NC(=O)[C@H](C)N(C)C(=O)COc3cc(F)cc2c3)cc1OC] |
| 122 | 1181.5 | 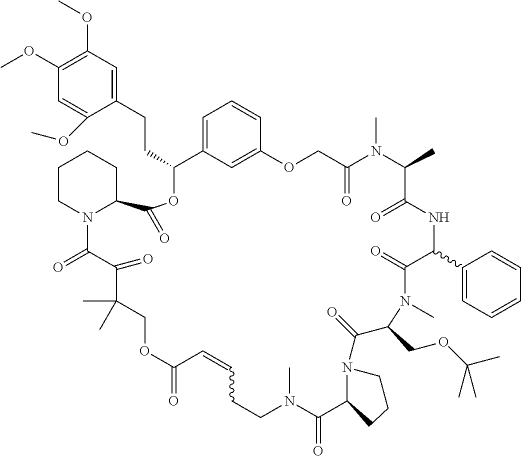 [SMILES: COc1cc(OC)c(OC)cc1CC[C@H]1OC(=O)[C@@H]2CCCCN2C(=O)C(=O)C(C)(C)COC(=O)C=CCCN(C)C(=O)[C@@H]2CCCN2C(=O)[C@H](COC(C)(C)C)N(C)C(=O)C(c2ccccc2)NC(=O)[C@H](C)N(C)C(=O)COc2cccc1c2] |
| 123 | 1181.5 | 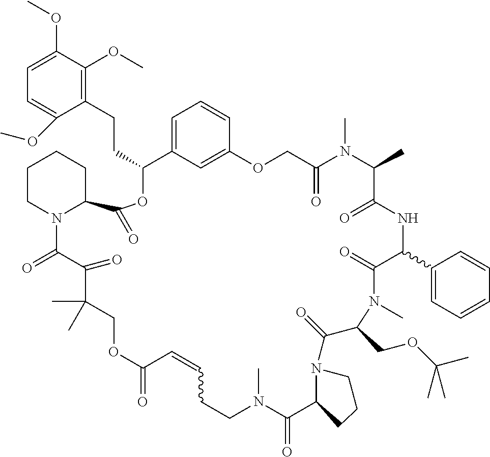 [SMILES: COc1ccc(OC)c(OC)c1CC[C@H]1OC(=O)[C@@H]2CCCCN2C(=O)C(=O)C(C)(C)COC(=O)C=CCCN(C)C(=O)[C@@H]2CCCN2C(=O)[C@H](COC(C)(C)C)N(C)C(=O)C(c2ccccc2)NC(=O)[C@H](C)N(C)C(=O)COc2cccc1c2] |
| 124 | 1181.5 | 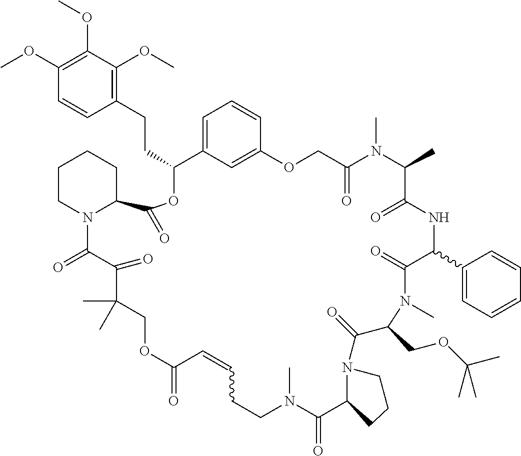 [SMILES: COc1ccc(CC[C@H]2OC(=O)[C@@H]3CCCCN3C(=O)C(=O)C(C)(C)COC(=O)C=CCCN(C)C(=O)[C@@H]3CCCN3C(=O)[C@H](COC(C)(C)C)N(C)C(=O)C(c3ccccc3)NC(=O)[C@H](C)N(C)C(=O)COc3cccc2c3)c(OC)c1OC] |
| 125 | 1169.5 | 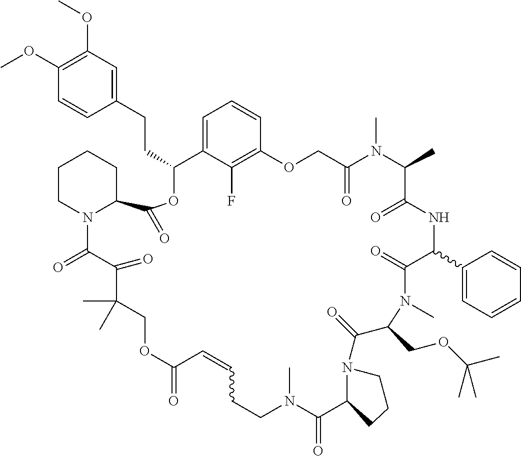 [SMILES: COc1ccc(CC[C@H]2OC(=O)[C@@H]3CCCCN3C(=O)C(=O)C(C)(C)COC(=O)C=CCCN(C)C(=O)[C@@H]3CCCN3C(=O)[C@H](COC(C)(C)C)N(C)C(=O)C(c3ccccc3)NC(=O)[C@H](C)N(C)C(=O)COc3cccc2c3F)cc1OC] |
| 126 | 1152.5 | 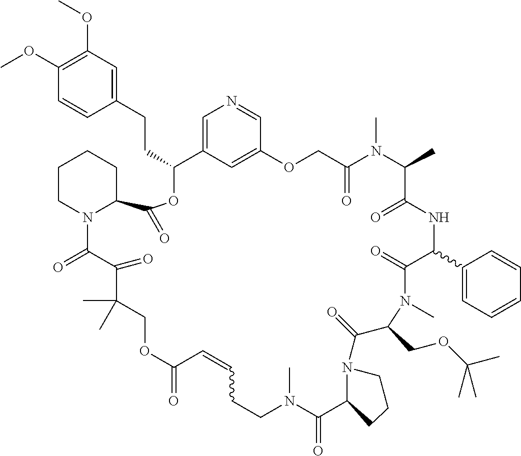 [SMILES: COc1ccc(CC[C@H]2OC(=O)[C@@H]3CCCCN3C(=O)C(=O)C(C)(C)COC(=O)C=CCCN(C)C(=O)[C@@H]3CCCN3C(=O)[C@H](COC(C)(C)C)N(C)C(=O)C(c3ccccc3)NC(=O)[C@H](C)N(C)C(=O)COc3cncc2c3)cc1OC] |
| 127 | 1374.6 | 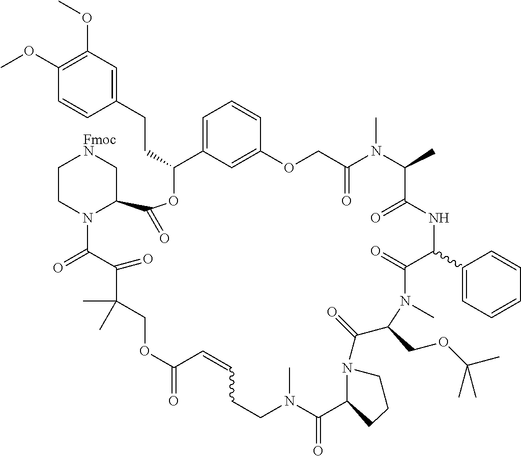 |
| 128 | 1152.5 | 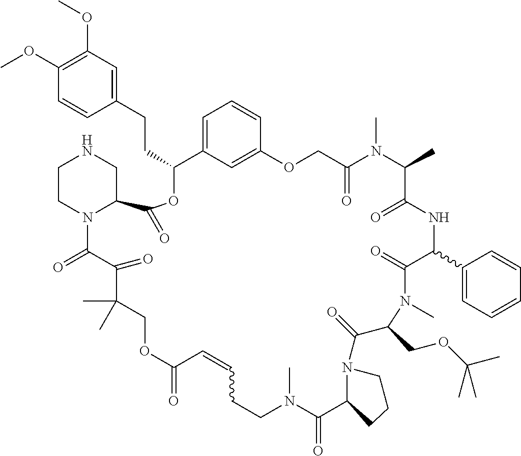 [SMILES: COc1ccc(CC[C@H]2OC(=O)[C@@H]3CNCCN3C(=O)C(=O)C(C)(C)COC(=O)C=CCCN(C)C(=O)[C@@H]3CCCN3C(=O)[C@H](COC(C)(C)C)N(C)C(=O)C(c3ccccc3)NC(=O)[C@H](C)N(C)C(=O)COc3cccc2c3)cc1OC] |
| 129 | 1224.5 | 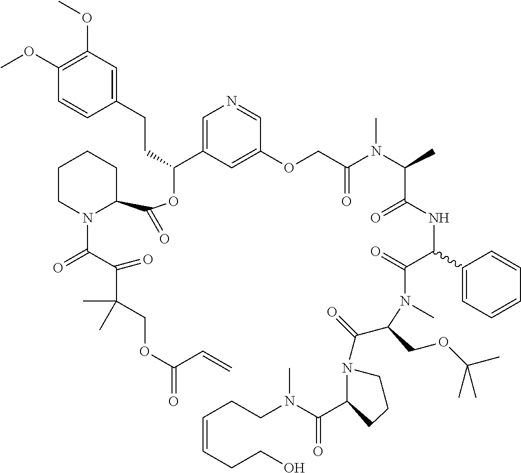 [SMILES: C=CC(=O)OCC(C)(C)C(=O)C(=O)N1CCCC[C@H]1C(=O)O[C@H](CCc1ccc(OC)c(OC)c1)c1cncc(OCC(=O)N(C)[C@@H](C)C(=O)NC(C(=O)N(C)[C@@H](COC(C)(C)C)C(=O)N2CCC[C@H]2C(=O)N(C)CC/C=C\CCO)c2ccccc2)c1] |
| 130 | 1595.7 | 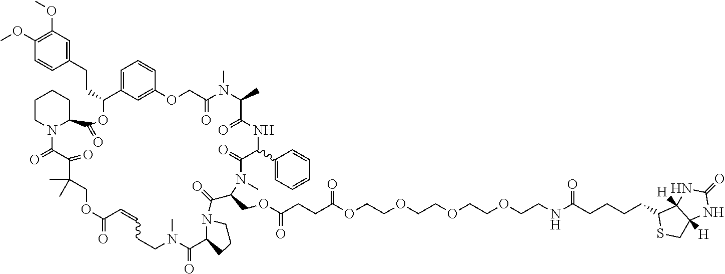 |
| 131 | 1122.5 | 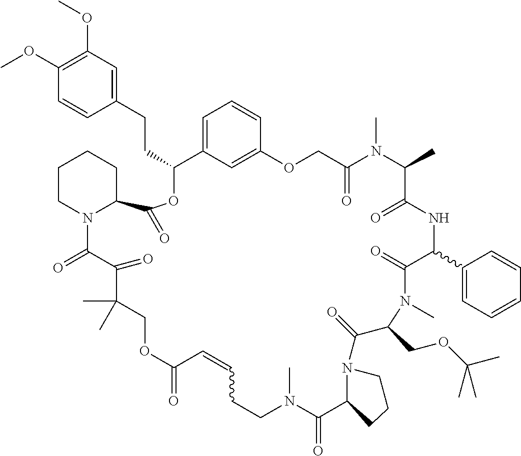 [SMILES: COc1ccc(CC[C@H]2OC(=O)[C@@H]3CCCCN3C(=O)C(=O)C(C)(C)COC(=O)C=CCCN(C)C(=O)[C@@H]3CCCN3C(=O)[C@H](COC(C)(C)C)N(C)C(=O)C(c3ccccc3)NC(=O)[C@H](C)N(C)C(=O)COc3cccc2c3)cc1OC] |
| 132 | 1150.7 | 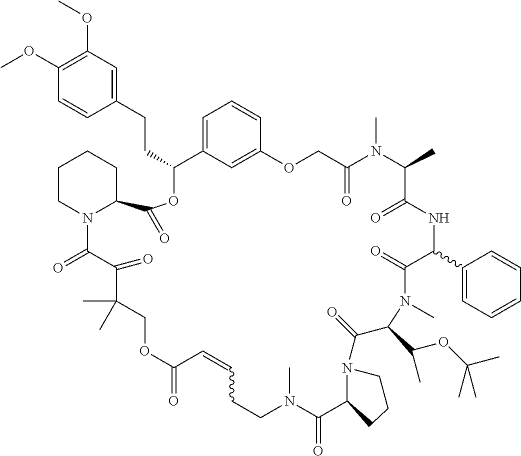 [SMILES: COc1ccc(CC[C@H]2OC(=O)[C@@H]3CCCCN3C(=O)C(=O)C(C)(C)COC(=O)C=CCCN(C)C(=O)[C@@H]3CCCN3C(=O)[C@H](C(C)OC(C)(C)C)N(C)C(=O)C(c3ccccc3)NC(=O)[C@H](C)N(C)C(=O)COc3cccc2c3)cc1OC] |
| 133 | 1106.5 | 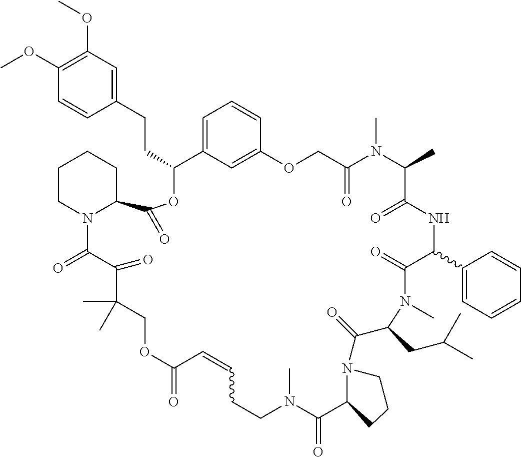 [SMILES: COc1ccc(CC[C@H]2OC(=O)[C@@H]3CCCCN3C(=O)C(=O)C(C)(C)COC(=O)C=CCCN(C)C(=O)[C@@H]3CCCN3C(=O)[C@H](CC(C)C)N(C)C(=O)C(c3ccccc3)NC(=O)[C@H](C)N(C)C(=O)COc3cccc2c3)cc1OC] |
| 134 | 1106.5 | 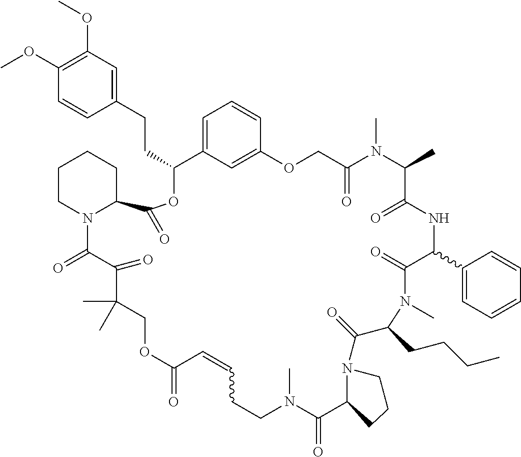 [SMILES: CCCC[C@H]1C(=O)N2CCC[C@H]2C(=O)N(C)CCC=CC(=O)OCC(C)(C)C(=O)C(=O)N2CCCC[C@H]2C(=O)O[C@H](CCc2ccc(OC)c(OC)c2)c2cccc(c2)OCC(=O)N(C)[C@@H](C)C(=O)NC(c2ccccc2)C(=O)N1C] |
| 135 | 1148.5 | 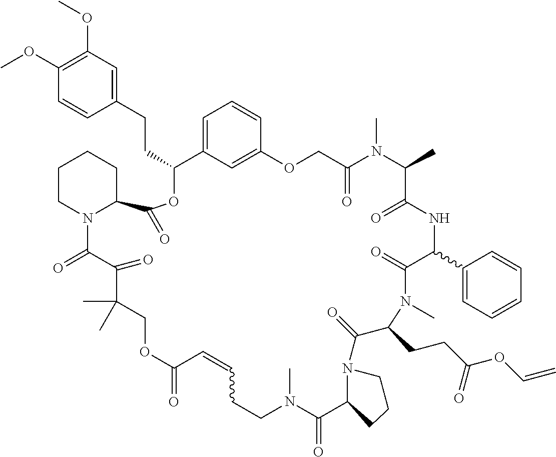 [SMILES: C=COC(=O)CC[C@H]1C(=O)N2CCC[C@H]2C(=O)N(C)CCC=CC(=O)OCC(C)(C)C(=O)C(=O)N2CCCC[C@H]2C(=O)O[C@H](CCc2ccc(OC)c(OC)c2)c2cccc(c2)OCC(=O)N(C)[C@@H](C)C(=O)NC(c2ccccc2)C(=O)N1C] |
| 136 | 1144.5 | 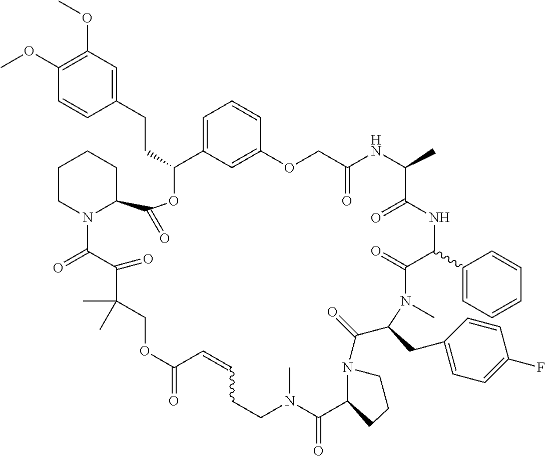 [SMILES: COc1ccc(CC[C@H]2OC(=O)[C@@H]3CCCCN3C(=O)C(=O)C(C)(C)COC(=O)C=CCCN(C)C(=O)[C@@H]3CCCN3C(=O)[C@H](Cc3ccc(F)cc3)N(C)C(=O)C(c3ccccc3)NC(=O)[C@H](C)NC(=O)COc3cccc2c3)cc1OC] |
| 137 | 1150.6 | 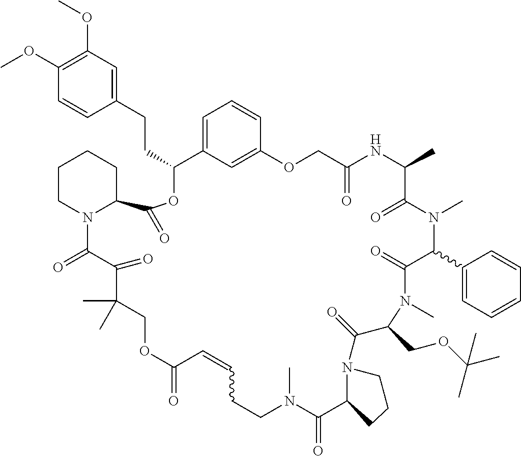 [SMILES: COc1ccc(CC[C@H]2OC(=O)[C@@H]3CCCCN3C(=O)C(=O)C(C)(C)COC(=O)C=CCCN(C)C(=O)[C@@H]3CCCN3C(=O)[C@H](COC(C)(C)C)N(C)C(=O)C(c3ccccc3)N(C)C(=O)[C@H](C)NC(=O)COc3cccc2c3)cc1OC] |
| 138 | 1164.6 | 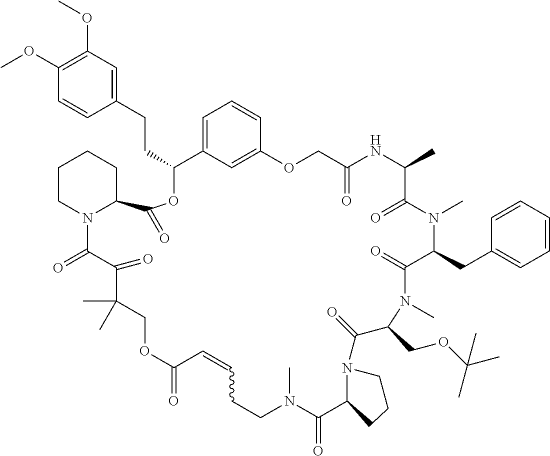 [SMILES: COc1ccc(CC[C@H]2OC(=O)[C@@H]3CCCCN3C(=O)C(=O)C(C)(C)COC(=O)C=CCCN(C)C(=O)[C@@H]3CCCN3C(=O)[C@H](COC(C)(C)C)N(C)C(=O)[C@H](Cc3ccccc3)N(C)C(=O)[C@H](C)NC(=O)COc3cccc2c3)cc1OC] |
| 139 | 1074.5 | 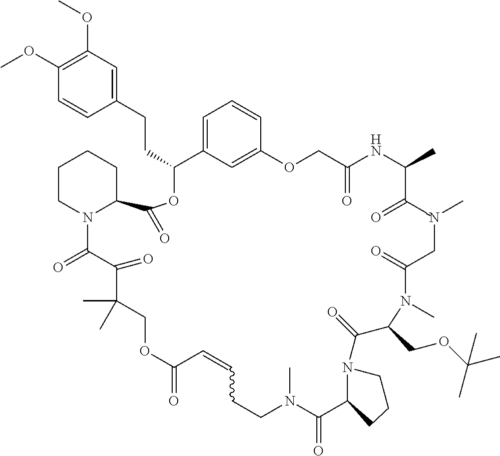 |
| 140 | 1160.6 | 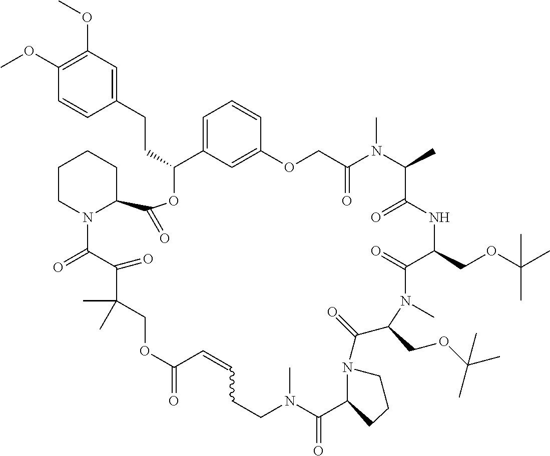 [SMILES: COc1ccc(CC[C@H]2OC(=O)[C@@H]3CCCCN3C(=O)C(=O)C(C)(C)COC(=O)C=CCCN(C)C(=O)[C@@H]3CCCN3C(=O)[C@H](COC(C)(C)C)N(C)C(=O)[C@H](COC(C)(C)C)NC(=O)[C@H](C)N(C)C(=O)COc3cccc2c3)cc1OC] |
| 141 | 1180.6 | 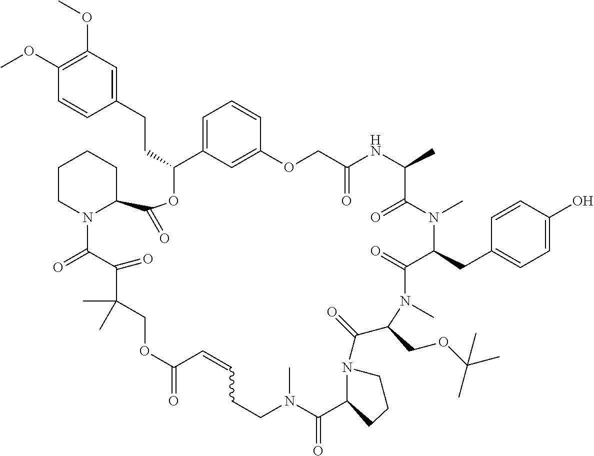 [SMILES: COc1ccc(CC[C@H]2OC(=O)[C@@H]3CCCCN3C(=O)C(=O)C(C)(C)COC(=O)C=CCCN(C)C(=O)[C@@H]3CCCN3C(=O)[C@H](COC(C)(C)C)N(C)C(=O)[C@H](Cc3ccc(O)cc3)N(C)C(=O)[C@H](C)NC(=O)COc3cccc2c3)cc1OC] |
| 142 | 1166.6 | 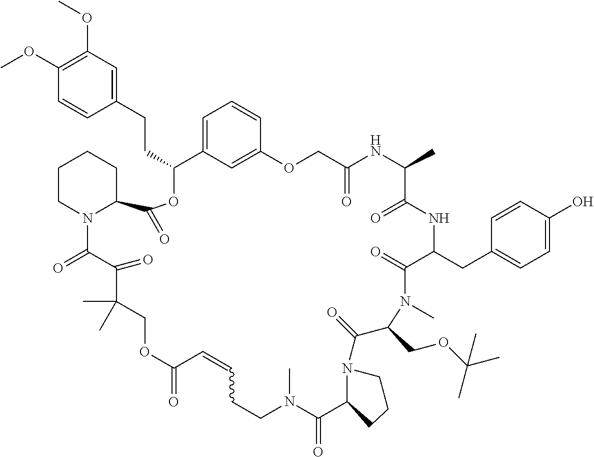 [SMILES: COc1ccc(CC[C@H]2OC(=O)[C@@H]3CCCCN3C(=O)C(=O)C(C)(C)COC(=O)C=CCCN(C)C(=O)[C@@H]3CCCN3C(=O)[C@H](COC(C)(C)C)N(C)C(=O)C(Cc3ccc(O)cc3)NC(=O)[C@H](C)NC(=O)COc3cccc2c3)cc1OC] |
| 143 | 1175.6 | 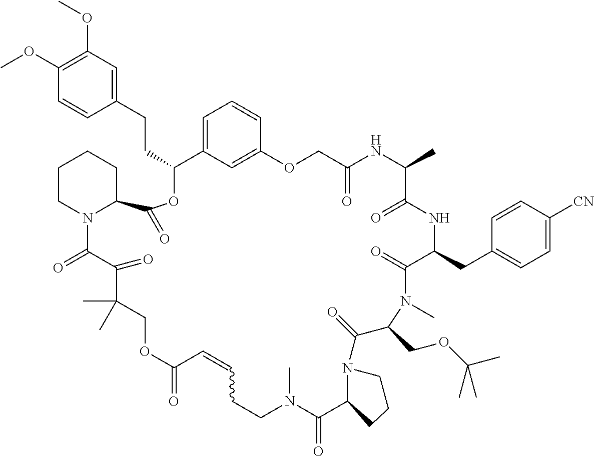 [SMILES: COc1ccc(CC[C@H]2OC(=O)[C@@H]3CCCCN3C(=O)C(=O)C(C)(C)COC(=O)C=CCCN(C)C(=O)[C@@H]3CCCN3C(=O)[C@H](COC(C)(C)C)N(C)C(=O)[C@H](Cc3ccc(C#N)cc3)NC(=O)[C@H](C)NC(=O)COc3cccc2c3)cc1OC] |
| 144 | 1212.6 | 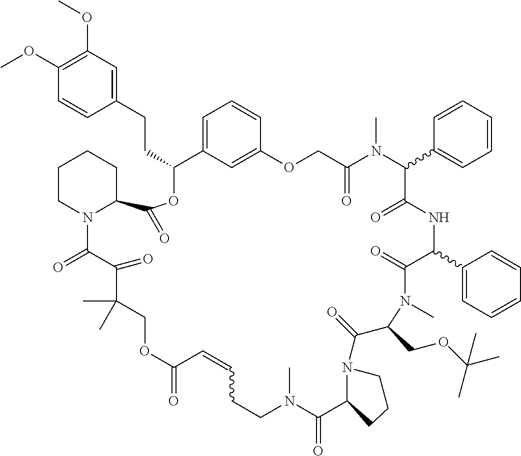 [SMILES: COc1ccc(CC[C@H]2OC(=O)[C@@H]3CCCCN3C(=O)C(=O)C(C)(C)COC(=O)C=CCCN(C)C(=O)[C@@H]3CCCN3C(=O)[C@H](COC(C)(C)C)N(C)C(=O)C(c3ccccc3)NC(=O)C(c3ccccc3)N(C)C(=O)COc3cccc2c3)cc1OC] |
| 145 | 1226.6 | 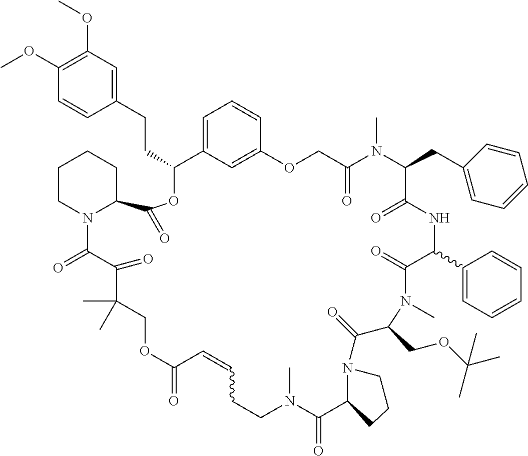 [SMILES: COc1ccc(CC[C@H]2OC(=O)[C@@H]3CCCCN3C(=O)C(=O)C(C)(C)COC(=O)C=CCCN(C)C(=O)[C@@H]3CCCN3C(=O)[C@H](COC(C)(C)C)N(C)C(=O)C(c3ccccc3)NC(=O)[C@H](Cc3ccccc3)N(C)C(=O)COc3cccc2c3)cc1OC] |
| 146 | 1222.6 | 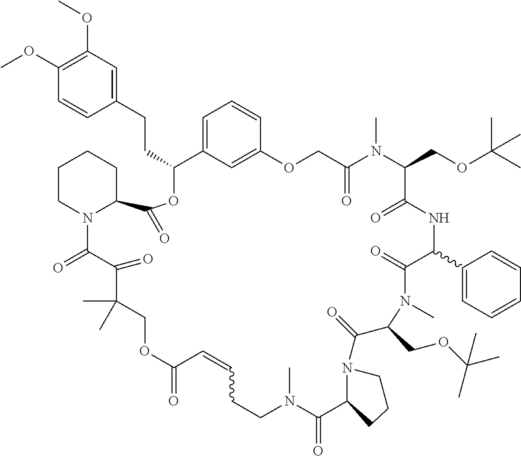 [SMILES: COc1ccc(CC[C@H]2OC(=O)[C@@H]3CCCCN3C(=O)C(=O)C(C)(C)COC(=O)C=CCCN(C)C(=O)[C@@H]3CCCN3C(=O)[C@H](COC(C)(C)C)N(C)C(=O)C(c3ccccc3)NC(=O)[C@H](COC(C)(C)C)N(C)C(=O)COc3cccc2c3)cc1OC] |
| 147 | 1236.7 | 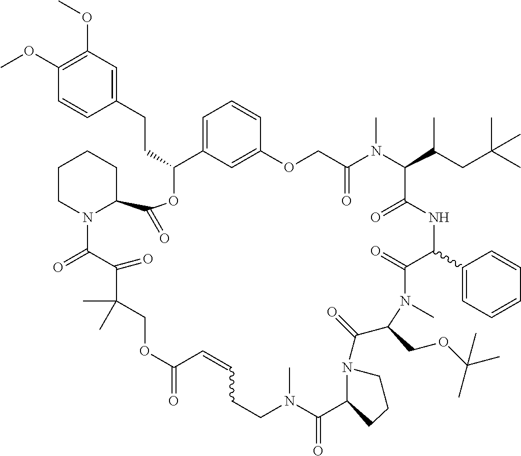 [SMILES: COc1ccc(CC[C@H]2OC(=O)[C@@H]3CCCCN3C(=O)C(=O)C(C)(C)COC(=O)C=CCCN(C)C(=O)[C@@H]3CCCN3C(=O)[C@H](COC(C)(C)C)N(C)C(=O)C(c3ccccc3)NC(=O)[C@H](C(C)CC(C)(C)C)N(C)C(=O)COc3cccc2c3)cc1OC] |
| 148 | 1242.6 | 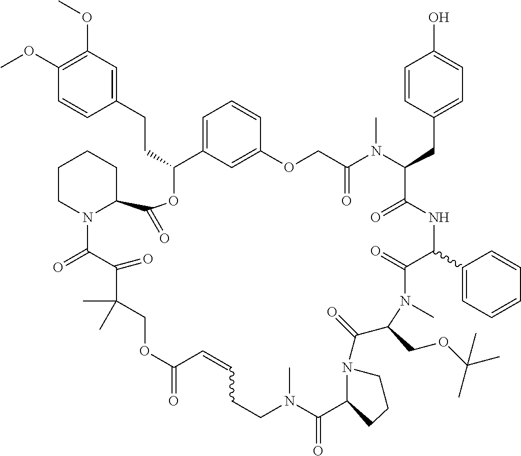 [SMILES: COc1ccc(CC[C@H]2OC(=O)[C@@H]3CCCCN3C(=O)C(=O)C(C)(C)COC(=O)C=CCCN(C)C(=O)[C@@H]3CCCN3C(=O)[C@H](COC(C)(C)C)N(C)C(=O)C(c3ccccc3)NC(=O)[C@H](Cc3ccc(O)cc3)N(C)C(=O)COc3cccc2c3)cc1OC] |
| 149 | 1090.6 | 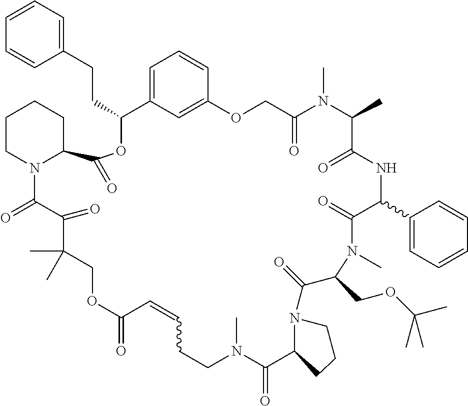 [SMILES: C[C@H]1C(=O)NC(c2ccccc2)C(=O)N(C)[C@@H](COC(C)(C)C)C(=O)N2CCC[C@H]2C(=O)N(C)CCC=CC(=O)OCC(C)(C)C(=O)C(=O)N2CCCC[C@H]2C(=O)O[C@H](CCc2ccccc2)c2cccc(c2)OCC(=O)N1C] |
| 150 | 1120.6 | 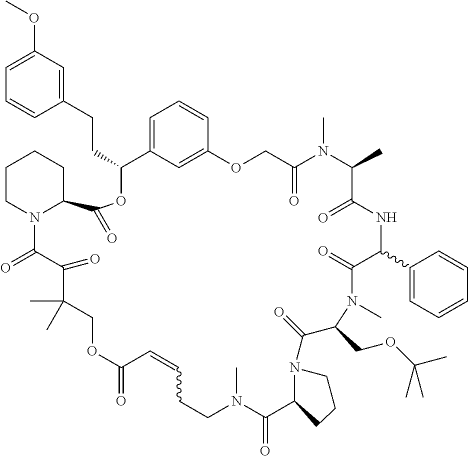 [SMILES: COc1cccc(CC[C@H]2OC(=O)[C@@H]3CCCCN3C(=O)C(=O)C(C)(C)COC(=O)C=CCCN(C)C(=O)[C@@H]3CCCN3C(=O)[C@H](COC(C)(C)C)N(C)C(=O)C(c3ccccc3)NC(=O)[C@H](C)N(C)C(=O)COc3cccc2c3)c1] |
| 151 | 1120.6 | 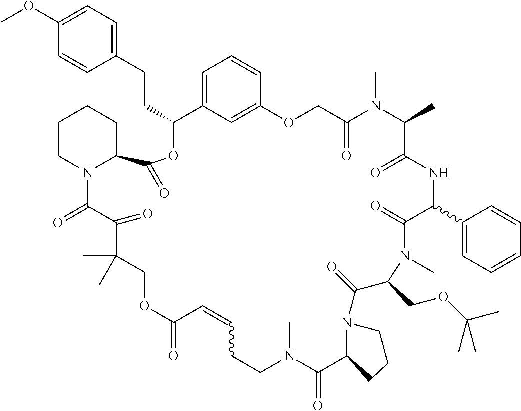 [SMILES: COc1ccc(CC[C@H]2OC(=O)[C@@H]3CCCCN3C(=O)C(=O)C(C)(C)COC(=O)C=CCCN(C)C(=O)[C@@H]3CCCN3C(=O)[C@H](COC(C)(C)C)N(C)C(=O)C(c3ccccc3)NC(=O)[C@H](C)N(C)C(=O)COc3cccc2c3)cc1] |
| 152 | 1152.6 | 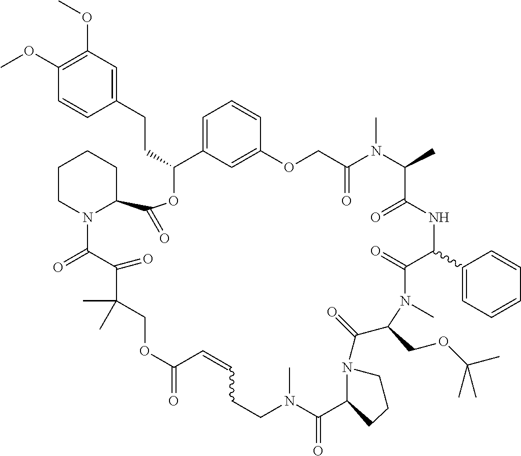 [SMILES: COc1ccc(CC[C@H]2OC(=O)[C@@H]3CCCCN3C(=O)C(=O)C(C)(C)COC(=O)C=CCCN(C)C(=O)[C@@H]3CCCN3C(=O)[C@H](COC(C)(C)C)N(C)C(=O)C(c3ccccc3)NC(=O)[C@H](C)N(C)C(=O)COc3cccc2c3)cc1OC] |
| 153 | 1169.5 | 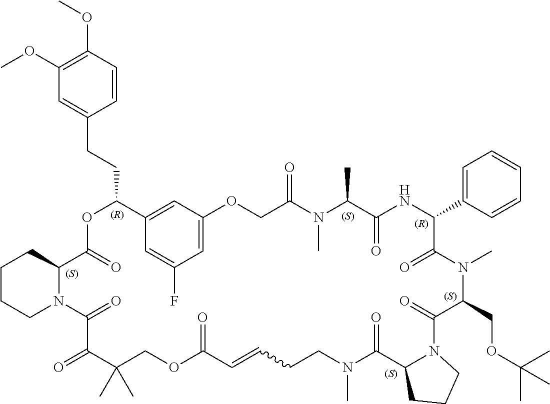 |
| 154 | 1169.5 | 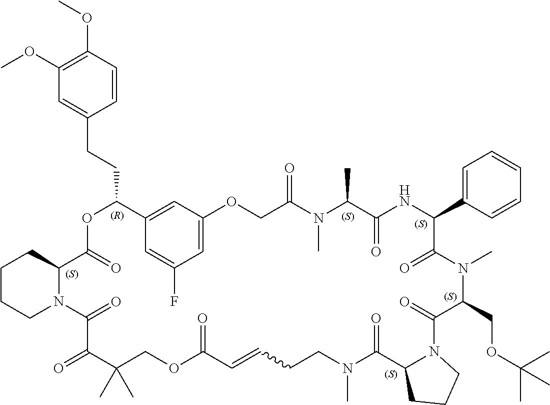 |

Compound 121 is a racemic mixture of compound 153 (R-enantiomer) and compound 154 (S-enantiomer).

In various embodiments, the compounds of the disclosure are useful as neuroprotective agents and treatment of disease.

In one embodiment, the disclosure provides a method of inducing a neuroprotective response in a cell. The method includes administering to the subject a therapeutically effective amount of a compound of the disclosure, thereby inducing a neuroprotective response in the cell. In some embodiments, the compound has the structure of Formula (I). In one embodiment, the compound is selected from those compounds set forth in Table 2.

In one embodiment, the disclosure provides a method of treating a neurodegenerative disease in a subject. The method includes administering to the subject a therapeutically effective amount of a compound of the disclosure, thereby treating the disease. In some embodiments, the compound has the structure of Formula (I). In one embodiment, the compound is selected from those compounds set forth in Table 2.

Recent studies highlight the importance of parthanatos, in pathologic α-synuclein (α-syn)-mediated neurodegeneration in Parkinson's disease (PD). Parthanatos Associated AIF (apoptosis-inducing factor) Nuclease (PAAN), also known as macrophage migration inhibitor factor (MIF) is a member of the PD-D/E(X)K superfamily of nucleases where it acts as the final executioner in parthanatic cell death through its nuclease activity. The role of PAAN/MIF in PD is not known. The present disclosure shows that pathologic α-syn induces neurodegeneration via the nuclease activity of PAAN/MIF. We identified a synthetic macrocycle compound 121 as a specific and potent inhibitor of PAAN, which blocks a critical step in parthanatos, by inhibiting PAAN/MIF's nuclease activity. Genetic depletion of PAAN/MIF, a PAAN/MIF mutant lacking nuclease activity and pharmacological inhibition of PAAN by compound 121 prevent the loss of dopaminergic neurons and behavioral deficits in the α-syn preformed fibril (α-syn PFF) mouse model of sporadic PD. Our findings suggest that inhibition of PAAN could be a potential disease modifying therapy with broad relevance in human pathologies where parthanatos plays a role.

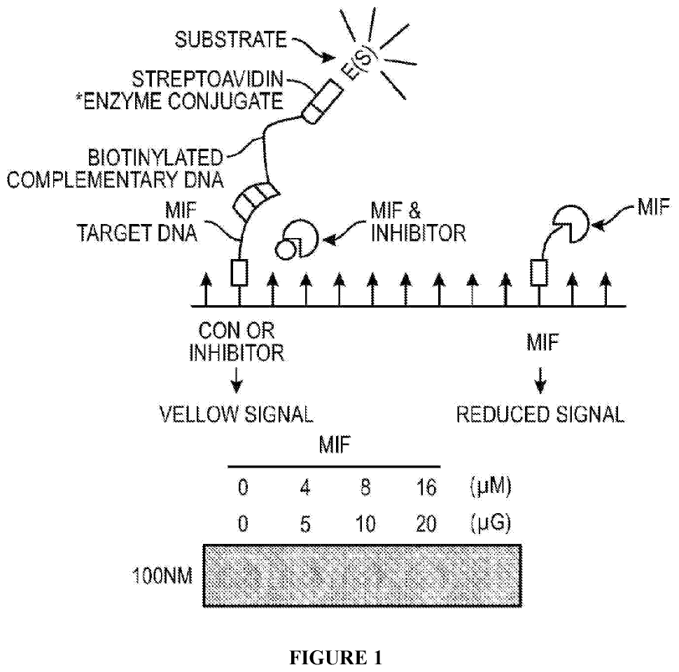

FIG. 1 illustrates the schematic representation of macrocyclic screening of PAAN/MIF inhibitors based on PAAN/MIF nuclease DNA cleavage assay. Single strand amino-modified oligonucleotide (RF substrate) was immobilized on DNA-BIND plates and incubated in PAAN/MIF nuclease with or without inhibitors. After PAAN/MIF's cleavage, the fragments were hybridized with biotin-labeled complementary oligonucleotides and detected by monitoring absorbance at 450 nm.

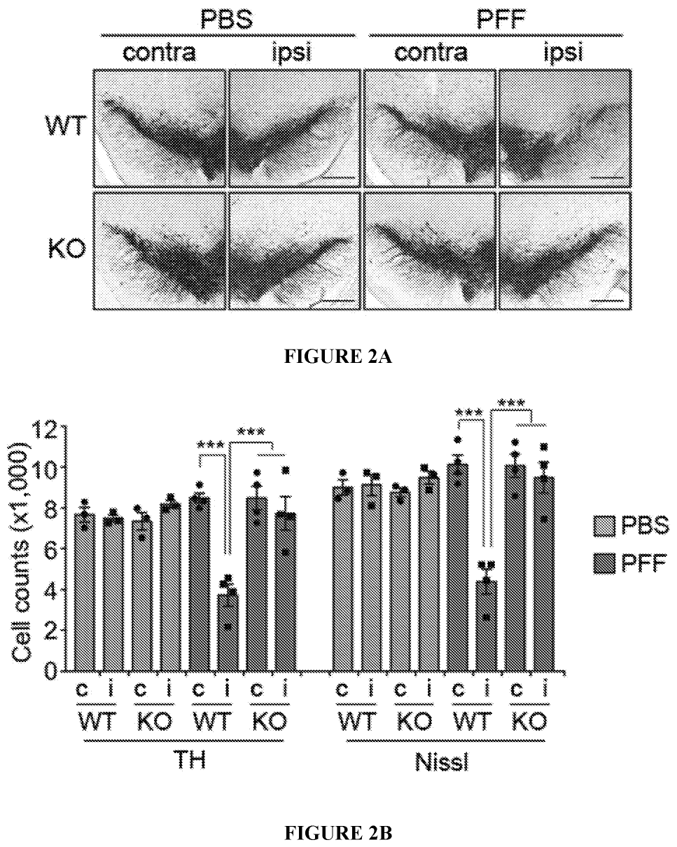

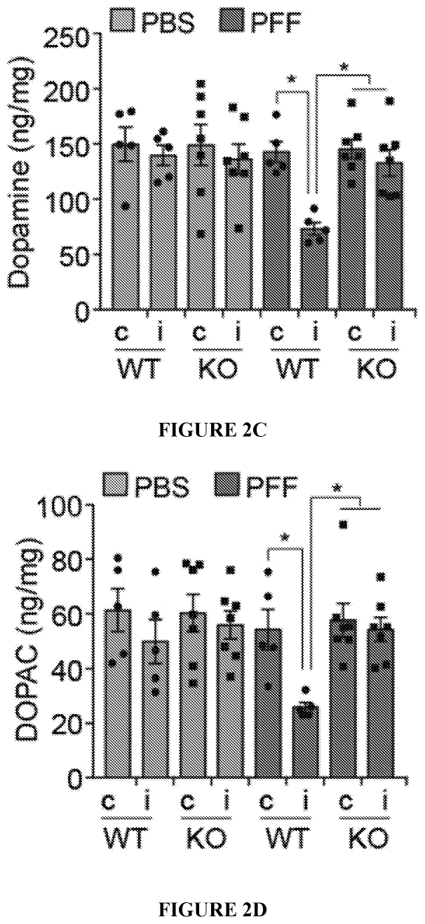

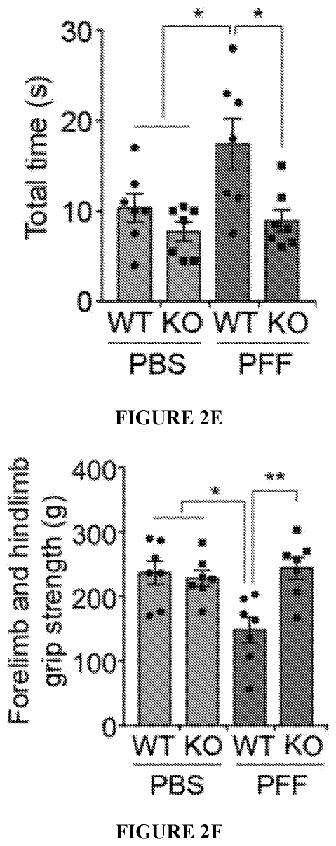

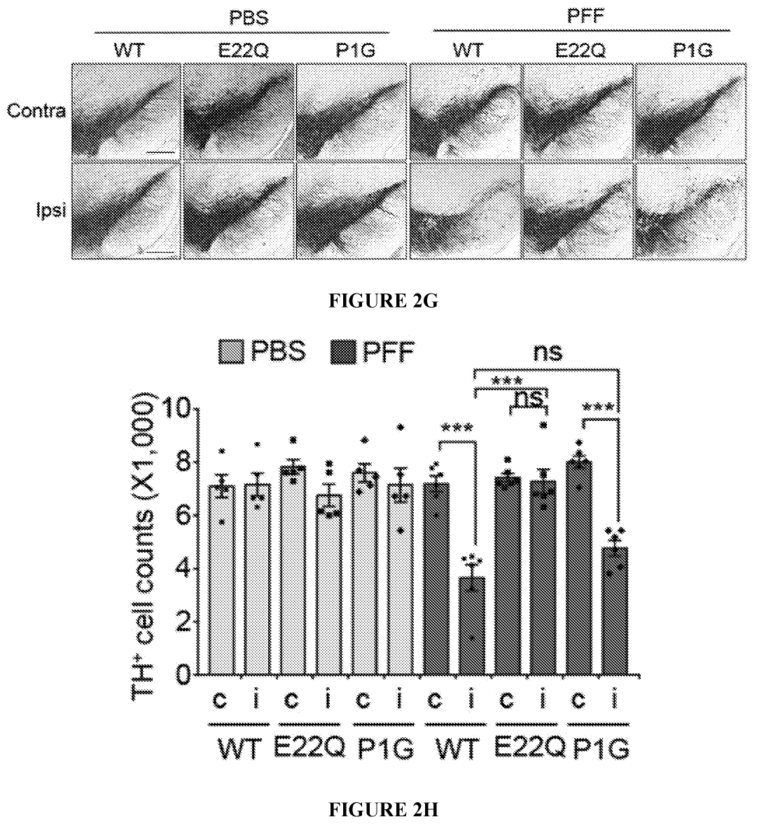

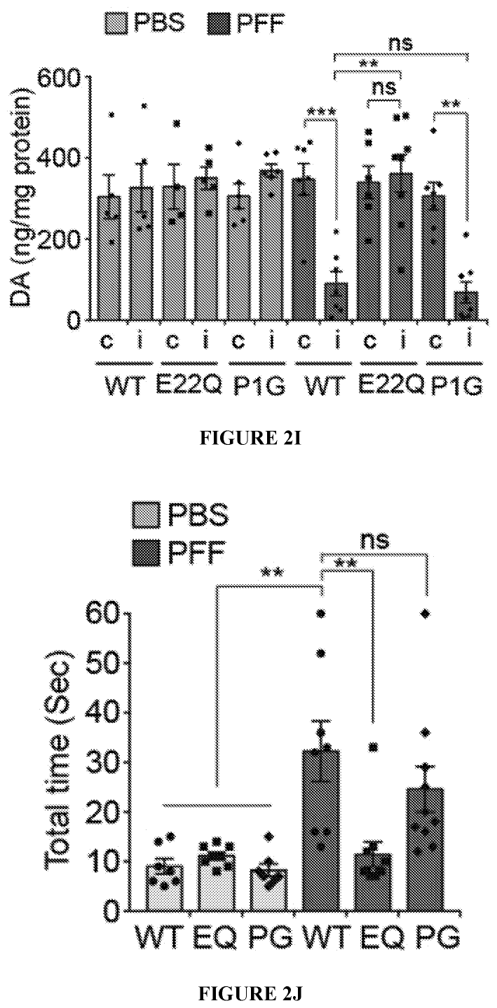

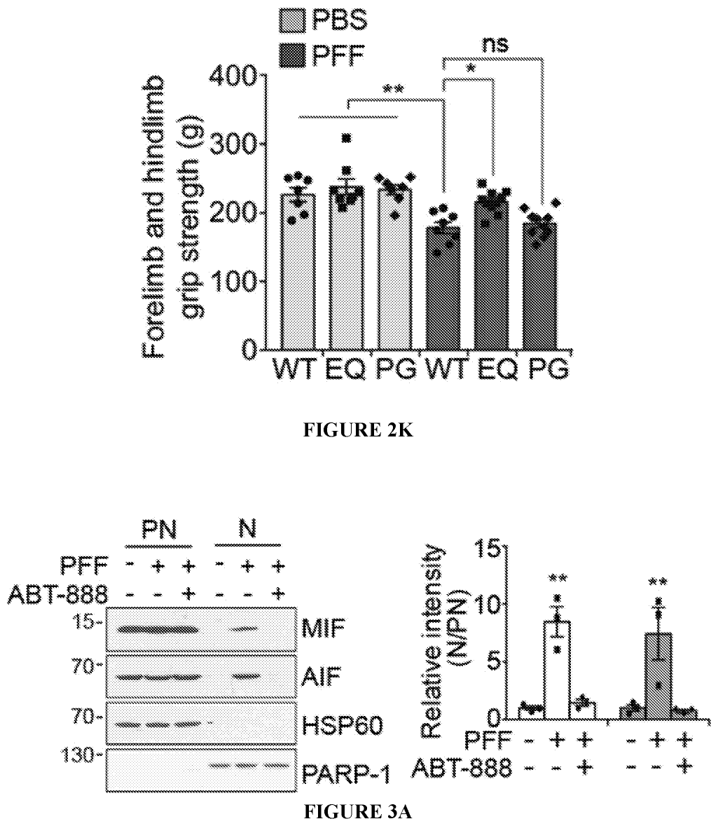

FIGS. 2A-2K show α-syn PFF-induced pathology is reduced by deletion of PAAN/MIF or deletion of PAAN/MIF's nuclease activity in vivo. FIG. 2A is an imaging illustrating representative TH and Nissl staining of SNpc DA neurons of α-syn PFF injected WT and PAAN/MIF KO at 6 months after α-syn PFF or PBS injection. Scale bars, 400 μM. FIG. 2B shows stereological counts. Data are mean±s.e.m. ***P<0.0005, two-way ANOVA followed by Tukey's post hoc test (n=3 to 4 mice per group). FIGS. 2C and 2D show dopamine and DOPAC concentrations in the striatum of WT and PAAN/MIF KO at 6 months after α-syn PFF or PBS injection measured by HPLC. Bars represent mean±s.e.m. *P<0.05, two-way ANOVA followed by Tukey's post hoc test (n=5 to 7 mice per group). FIGS. 2E and 2F show pole test and grip strength test results 180 days after intrastriatal α-syn PBS or PFF injection, performed in WT or PAAN/MIF KO. Data are the mean±s.e.m. *P<0.05, **P<0.005, two-way ANOVA followed by Tukey's post hoc test (n=7 mice per group). FIG. 2G shows representative TH and Nissl staining of SNpc DA neurons of WT, E22Q and P1G knock-in mice at 6 months after intrastriatal α-syn PFF or PBS injection. Scale bars, 400 μM. FIG. 2H shows stereological counts of TH cells. Data are mean±s.e.m. ***P<0.0005, two-way ANOVA followed by Tukey's post hoc test (n=5 to 6 mice per group). FIG. 2I shows dopamine concentrations in the striatum of PAAN/MIF WT, E22Q and P1G knock-in mice at 6 months after α-syn PFF or PBS injection measured by HPLC. Bars represent mean±s.e.m. **P<0.005, ***P<0.0005, two-way ANOVA followed by Tukey's post hoc test (n=5 to 8 mice per group). FIGS. 2J and 2K show pole test and grip strength test result 180 days after intrastriatal PBS or α-syn PFF injection in PAAN/MIF WT, E22Q and P1G knock-in mice. Data are the mean±s.e.m. *P<0.05, **P<0.005, two-way ANOVA followed by Tukey's post hoc test (n=7 to 10 mice per group).

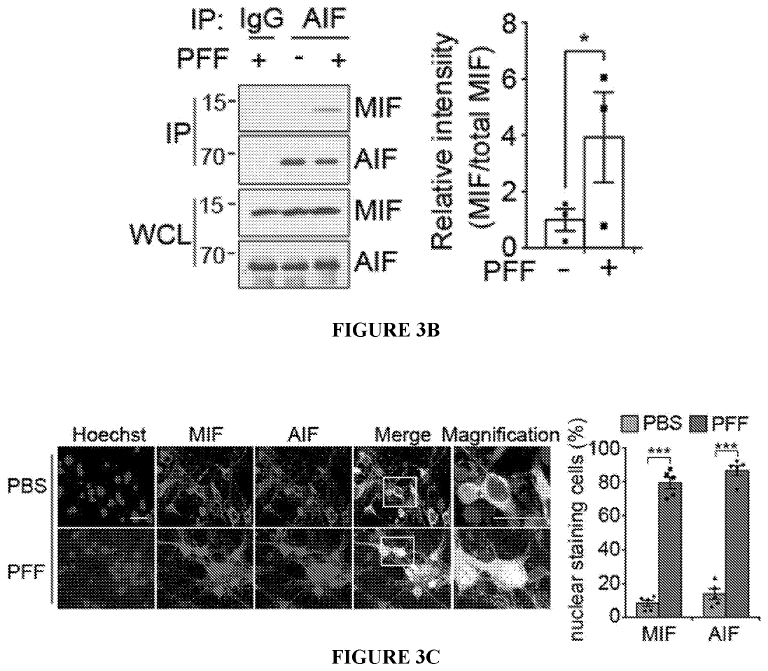

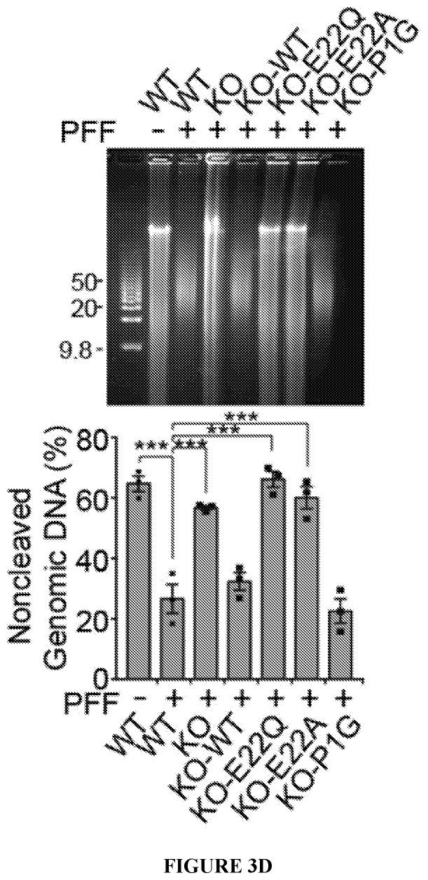

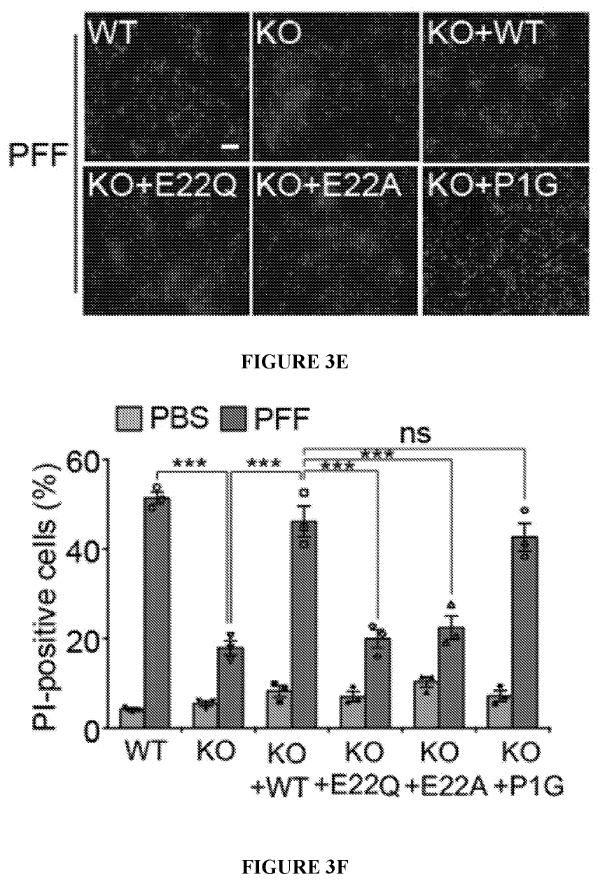

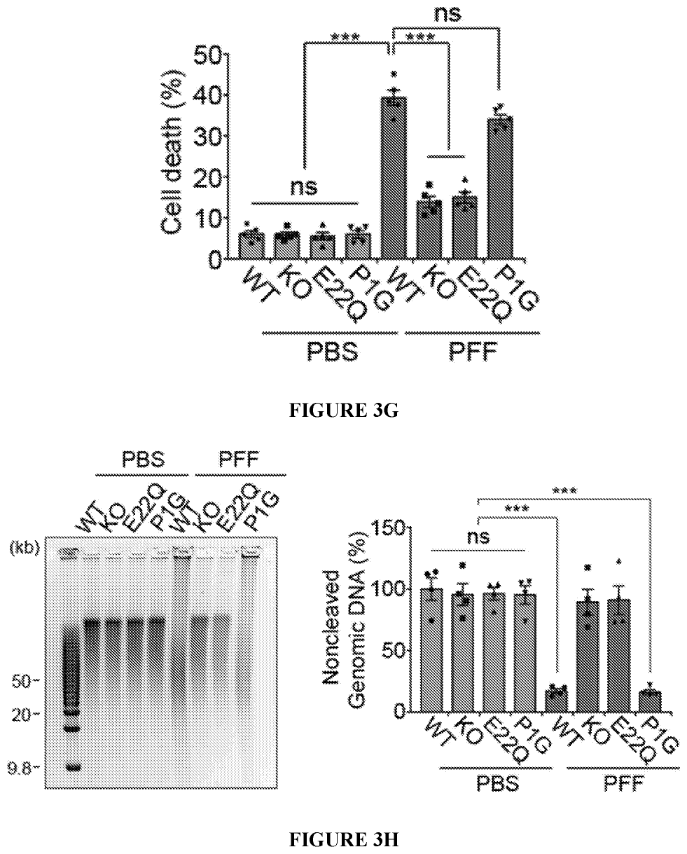

FIGS. 3A-3H show PAAN/MIF nuclease activity is required for prevention of α-syn PFF-induced neurotoxicity in neurons. FIG. 3A shows nuclear translocation of AIF and PAAN/MIF after α-syn PFF treatment in the presence of the PARP inhibitor, ABT-888 in cortical neurons. See Donawho, et al. (2007) Clin. Cancer Res. 13:2728-37. Intensity of PAAN/MIF and AIF signal is shown in the graph. **P<0.005 versus the PBS control group in the nuclear (N) fraction, two-way ANOVA followed by Tukey's post hoc test. FIG. 3B shows immunoprecipitation (IP) of PAAN/MIF and AIF in PBS or α-syn PFF-treated cortical neurons. Intensity of AIF-bound PAAN/MIF is shown in the graph. *P<0.05, student's t-test. FIG. 3C shows images of nuclear translocation of PAAN/MIF (green) and AIF (red) after α-syn PFF treatment in primary cortical neurons. Scale bars, 20 μm. The white color in the merged images indicates the overlay of AIF, PAAN/MIF and Hoechst dye in the nucleus. The percentage of cells with nuclear localization of PAAN/MIF and AIF is shown in the graph. ***P<0.0005, student's t-test. FIG. 3D shows pulsed-field gel electrophoresis of α-syn PFF-induced DNA damage in PAAN/MIF WT and KO neurons and KO neurons expressing PAAN/MIF WT, E22Q, E22A or P1G. Intensity of noncleaved genomic DNA is shown in the graph. ***P<0.0005, two-way ANOVA. FIG. 3E shows representative images of Hoechst and PI staining from primary cortical neurons transduced with AAV containing PAAN/MIF WT, E22Q, E22A or P1G and further incubated with α-syn PFF. Scale bar, 20 μm. FIG. 3F shows quantification of cell death. Bars represent mean±s.e.m. ***P<0.0005, two-way ANOVA followed by Tukey's post hoc test (n=3). FIG. 3G shows quantification of cell death from Hoechst and PI staining of primary cortical neurons from PAAN/MIF WT, KO, E22Q and P1G KI mice. Bars represent mean±s.e.m. ***P<0.0005, two-way ANOVA followed by Tukey's post hoc test (n=5). FIG. 3H shows pulsed-field gel electrophoresis of α-syn PFF-induced DNA damage in PAAN/MIF WT, KO, E22Q and P1G KI neurons treated with PBS or α-syn PFF. Intensity of noncleaved genomic DNA is shown in the graph. ***P<0.0005, two-way ANOVA followed by Tukey's post hoc test.

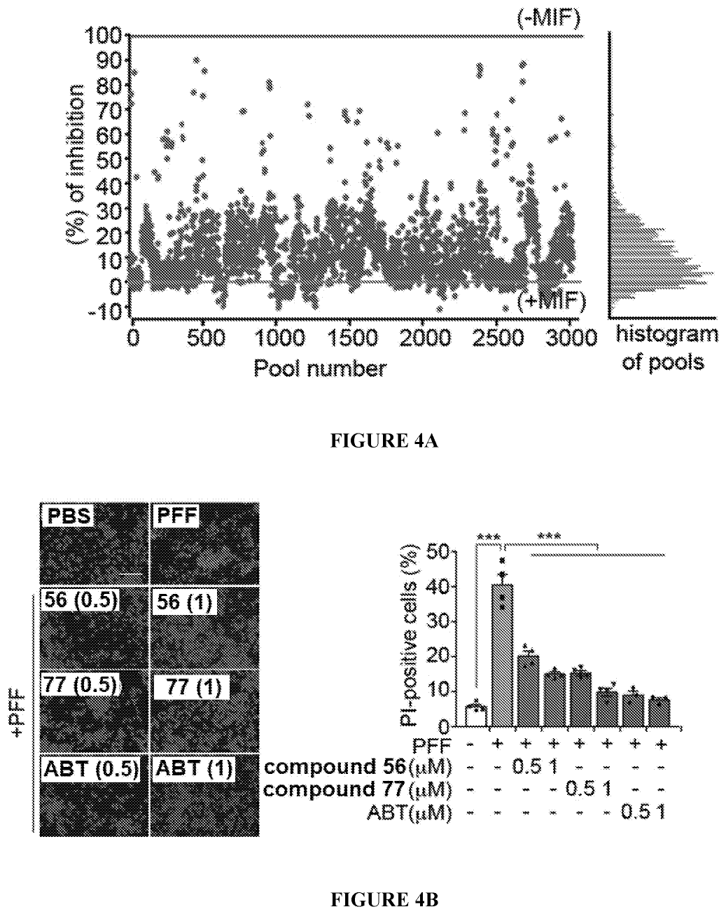

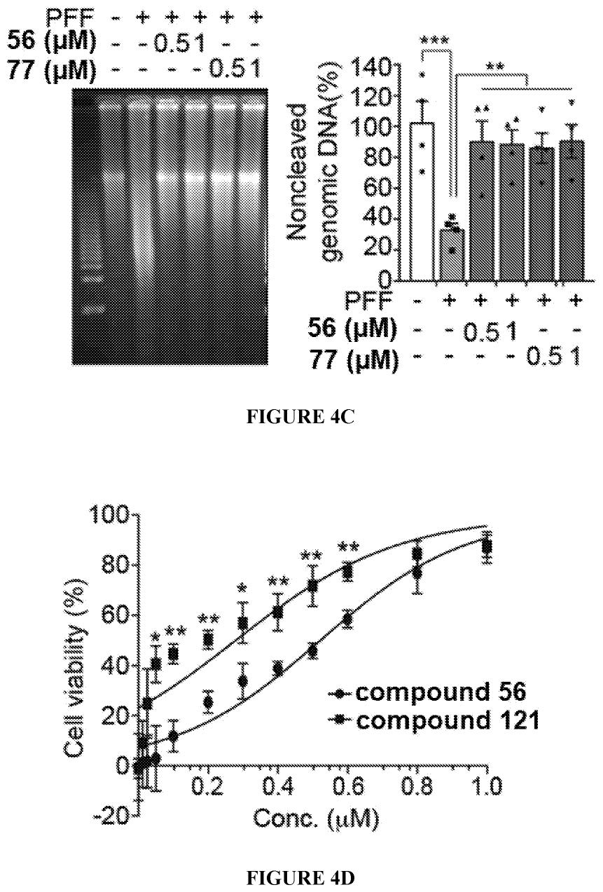

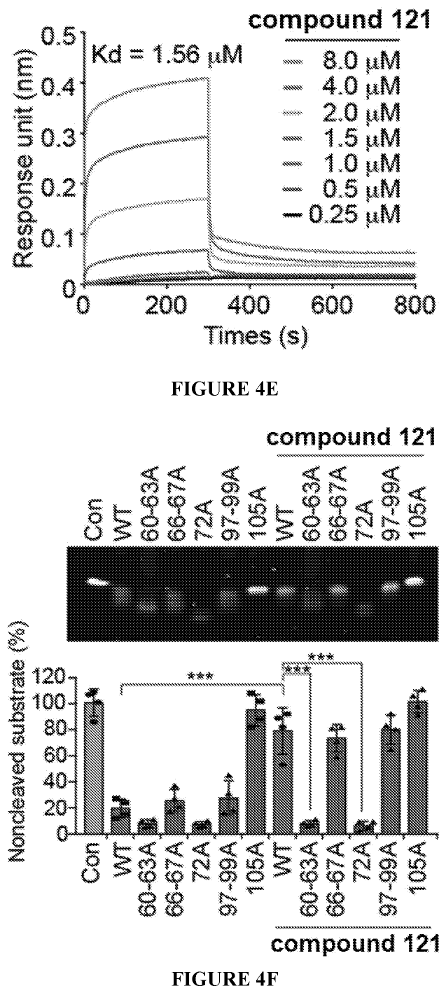

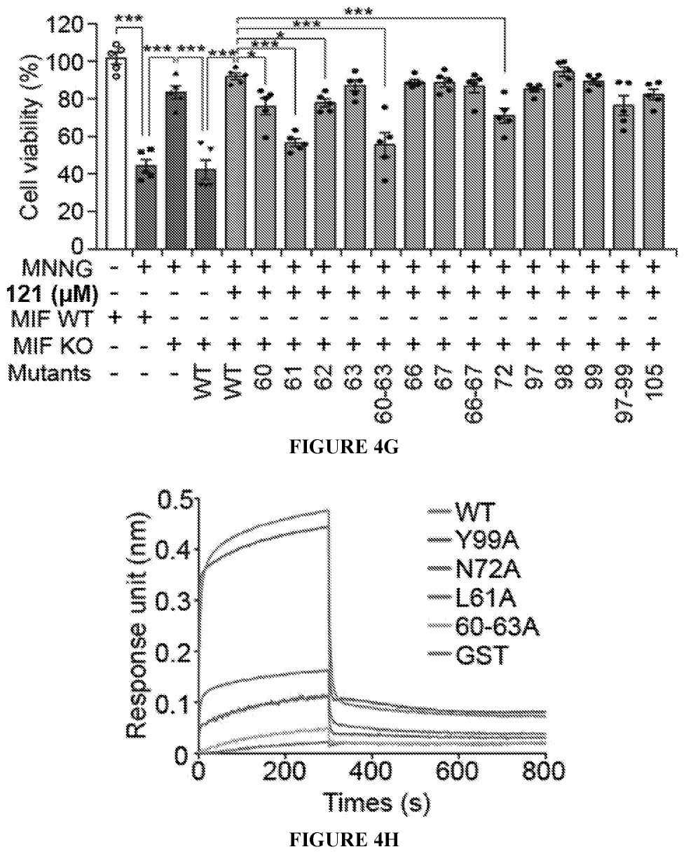

FIGS. 4A-4H shows of compound 121 is identified as a PAAN/MIF nuclease inhibitor. FIG. 4A shows scatter plot of percentage inhibition of PAAN/MIF cleavage from 45,000 compounds with 3,000 pools in 38 plates of the macrocyclic library. The top line (blue) is the incubation without PAAN/MIF and bottom line (green) is the incubation with PAAN/MIF. Right graph represents the histogram of the compounds tested. FIG. 4B shows representative images of Hoechst and PI staining from human cortical neurons pre-incubated with compounds 56, 77, or ABT-888 for 1 h and further incubated with α-syn PFF for 14 days. Scale bar, 20 μm. Quantification of cell death. Bars represent mean±s.e.m. ***P<0.0005, two-way ANOVA followed by Tukey's post hoc test (n=4). FIG. 4C shows pulsed-field gel electrophoresis of α-syn PFF-induced DNA damage in human cortical neurons treated with compound 56 or 77. Intensity of noncleaved genomic DNA is shown in the graph. **P<0.005, ***P<0.0005, two-way ANOVA. FIG. 4D shows SH-SY5Y cells were pre-incubated compound 56 or 121 with concentrations as indicated for 1 h, followed by 50 μM MNNG for 15 min. After 24 h, cell viability was measured by Alamar blue. Data represent mean±s.e.m. (n=3). *P<0.05, **P<0.005, student's t-test. The half maximal inhibitory concentration (IC₅₀) of compound 56 is 0.52 μM and compound 121 is 0.28 μM. FIG. 4E shows binding affinities of PAAN/MIF WT for compound 121 determined by biolayer interferometry (ForteBio Octet) assay. Data are representative of three independent experiments. FIG. 4F shows in vitro PAAN/MIF's nuclease assay using PAAN/MIF or PAAN/MIF mutants with compound 121. Quantification of noncleaved substrate DNA. Bars represent mean±s.e.m. ***P<0.0005, two-way ANOVA followed by Tukey's post hoc test (n=4). FIG. 4G shows WT or PAAN/MIF KO SH-SY5Y cells expressing Flag-PAAN/MIF WT or mutants were pre-incubated 1 μM of compound 121 for 1 h, followed by 50 μM MNNG for 15 min. After 24 h, cell viability was measured by Alamar blue. Data represent mean±s.e.m. *P<0.05, ***P<0.0005, two-way ANOVA followed by Tukey's post hoc test (n=5). FIG. 4H shows binding of PAAN/MIF WT or mutants for compound 121 determined by biolayer interferometry (ForteBio Octet) assay. Data are representative of three independent experiments.

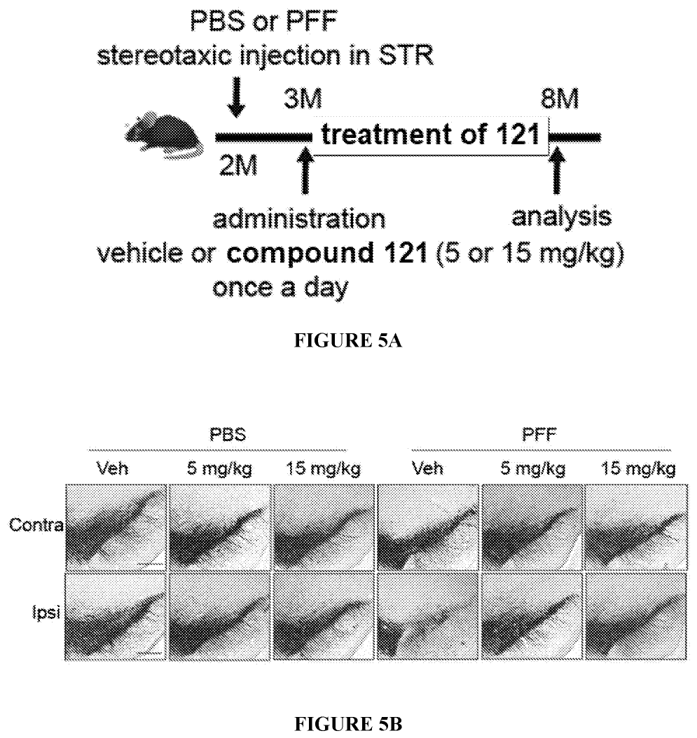

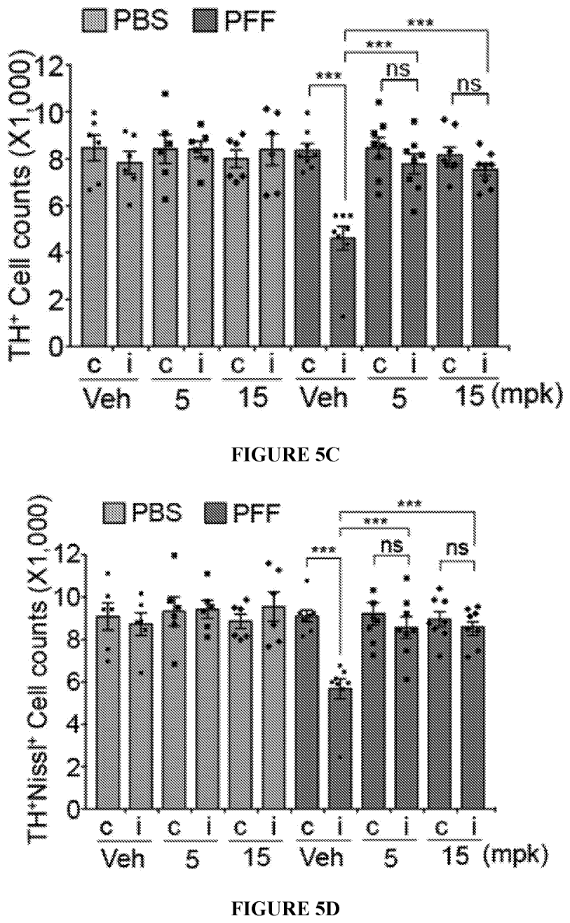

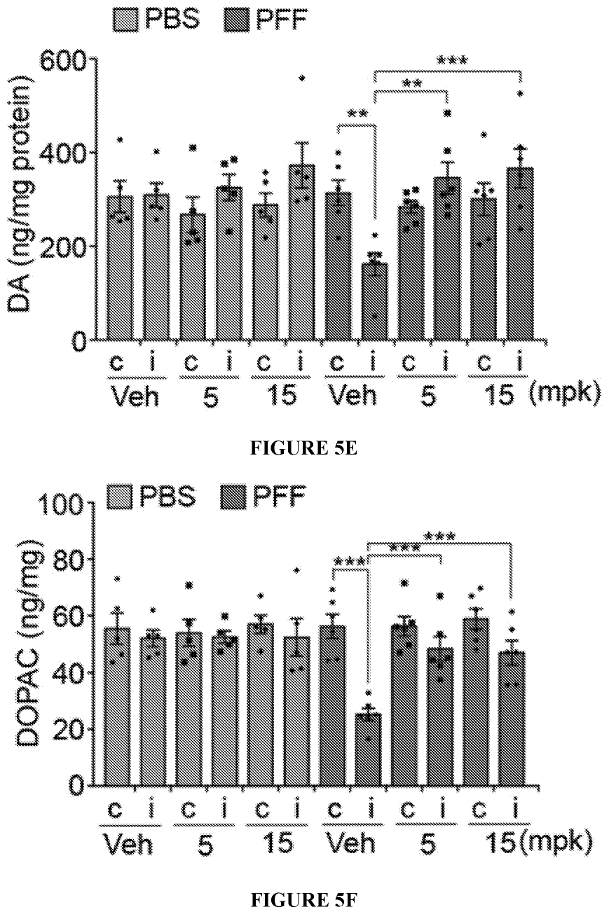

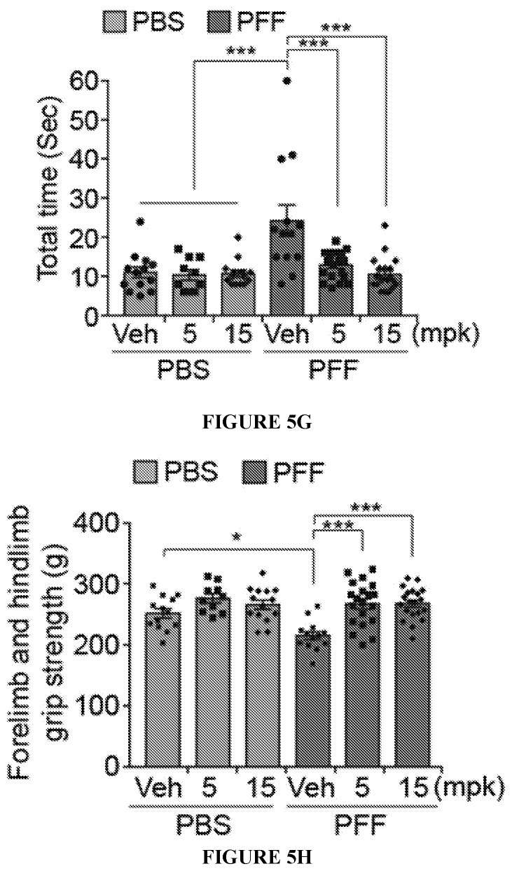

FIGS. 5A-5I demonstrate compound 121 protects against α-syn PFF-induced pathology in vivo. FIG. 5A shows schematic diagram of the experimental design. PBS or α-syn PFF were injected into the striatum of 2 month-old WT mice. After 1 month, compound 121 or vehicle was delivered by oral administration with two different doses (5 or 15 mg/kg) for 5 months. FIG. 5B shows representative TH and Nissl staining of SNpc DA neurons of PBS or α-syn PFF injected WT mice treated with vehicle or compound 121. Scale bars, 400 μM. FIGS. 5C and 5D show stereological counts of TH-positive cells and Nissl-positive cells. Data are mean±s.e.m. ***P<0.0005, two-way ANOVA followed by Tukey's post hoc test (n=6 to 8 mice per group). FIGS. 5E and 5F show dopamine and DOPAC concentrations in the striatum of PBS or α-syn PFF injected WT mice treated with vehicle of PAANIB-1 as assessed by HPLC. Bars represent mean±s.e.m. *P<0.05, two-way ANOVA followed by Tukey's post hoc test (n=5 to 6 mice per group). FIGS. 5G and 5H show pole test and grip strength test results 180 days after intrastriatal α-syn PFF or PBS injection in WT mice treated with vehicle or compound 121. Data are the mean±s.e.m. *P<0.05, ***P<0.0005, two-way ANOVA. FIG. 5I shows DNA fragmentation determined by pulsed-field gel electrophoresis in PBS or α-syn PFF injected WT mice treated with vehicle or compound 121. Intensity of noncleaved genomic DNA is shown in the graph. ***P<0.0005, two-way ANOVA followed by Tukey's post hoc test (n=3 to 5 mice per group).

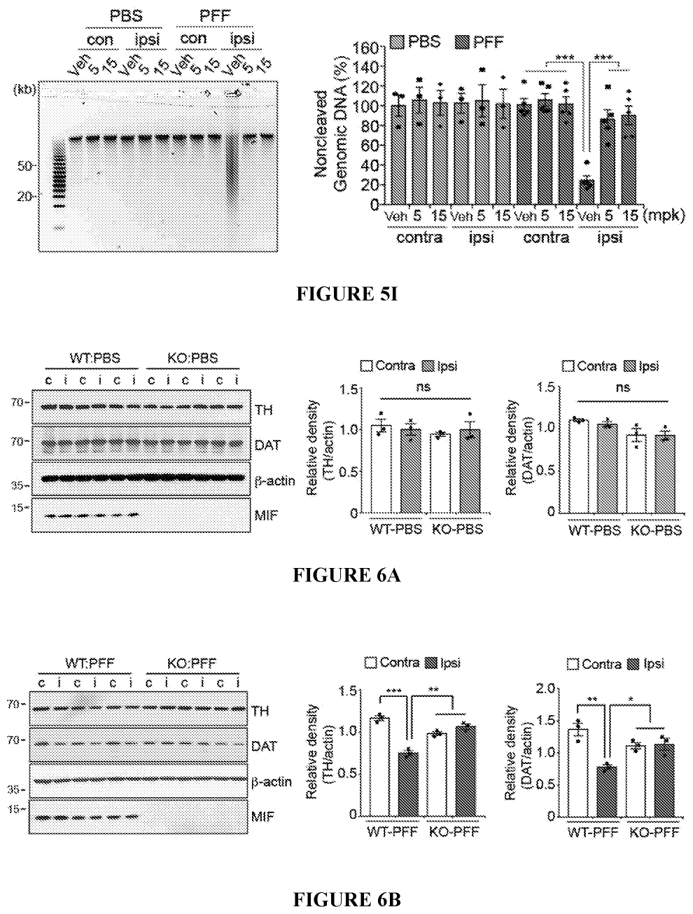

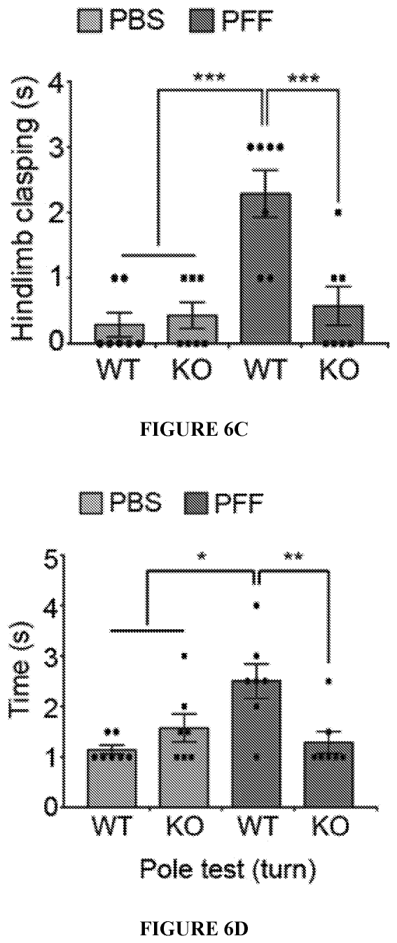

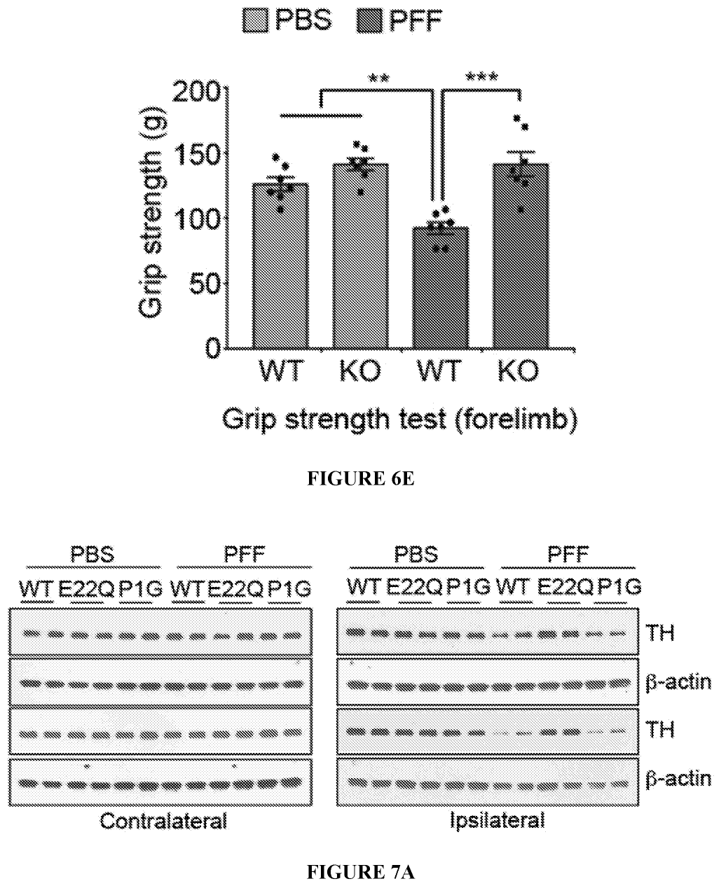

FIGS. 6A-6E demonstrate α-syn PFF-induced pathology is reduced by deletion of MIF/PAAN in vivo. FIGS. 6A and 6B show representative immunoblots and quantification of TH and DAT levels in the (c) contralateral and (i) ipsilateral striatum of (6A) PBS— and (6B) α-syn PFF-injected WT or MIF/PAAN KO mice. Bars represent the mean±s.e.m. *P<0.05, **P<0.005, ***P<0.0005, two-way ANOVA followed by Tukey's post hoc test (n=3). FIGS. 6C-6E show behavioral abnormalities of PBS and α-syn PFF-injected mice at 6 months as measured by (6C) clasping test, (6D) pole test and (6E) grip strength test. Bars are the means±s.e.m. *P<0.05, **P <0.005, ***P<0.0005, two-way ANOVA followed by Tukey's post hoc test.

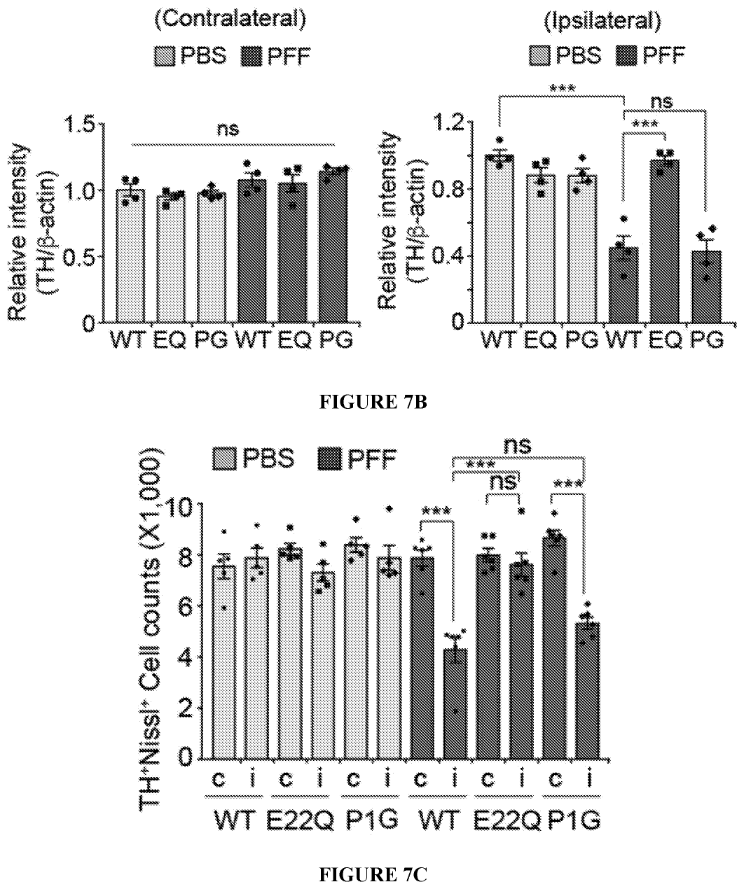

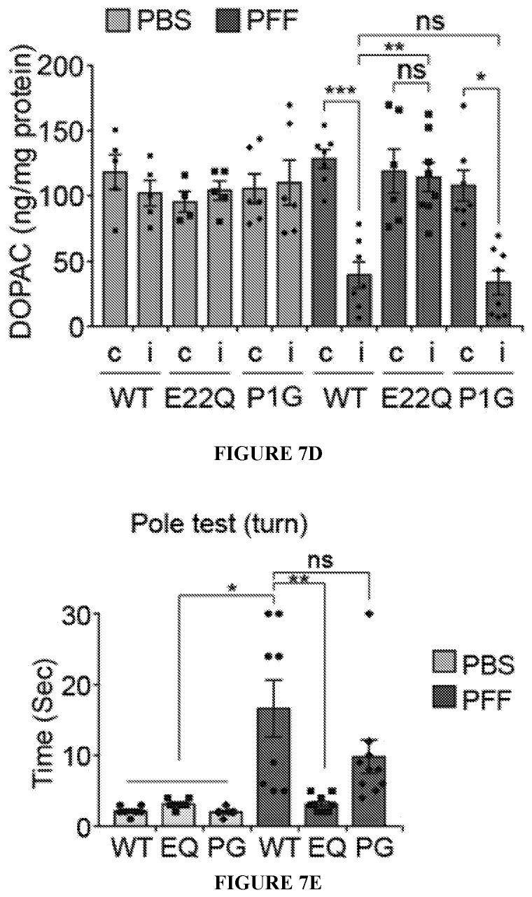

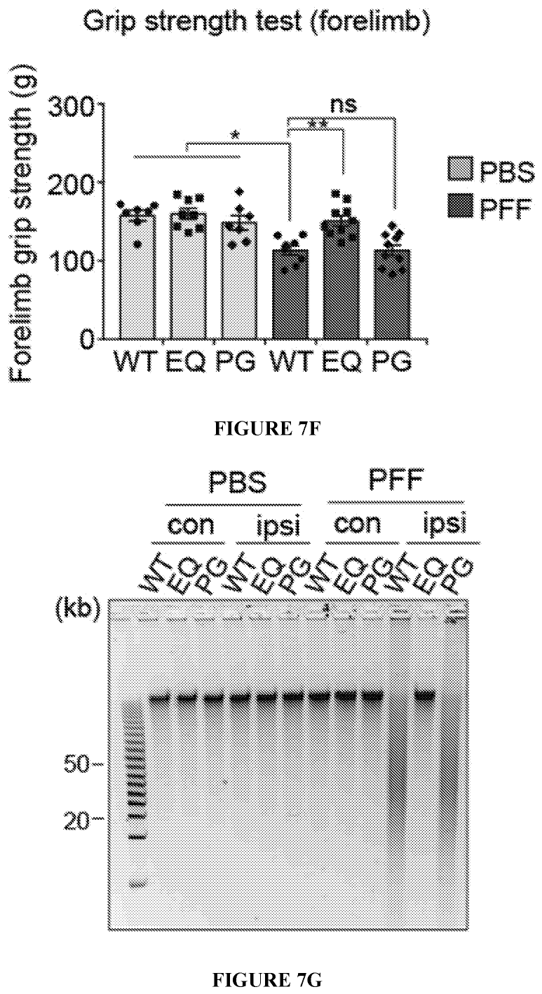

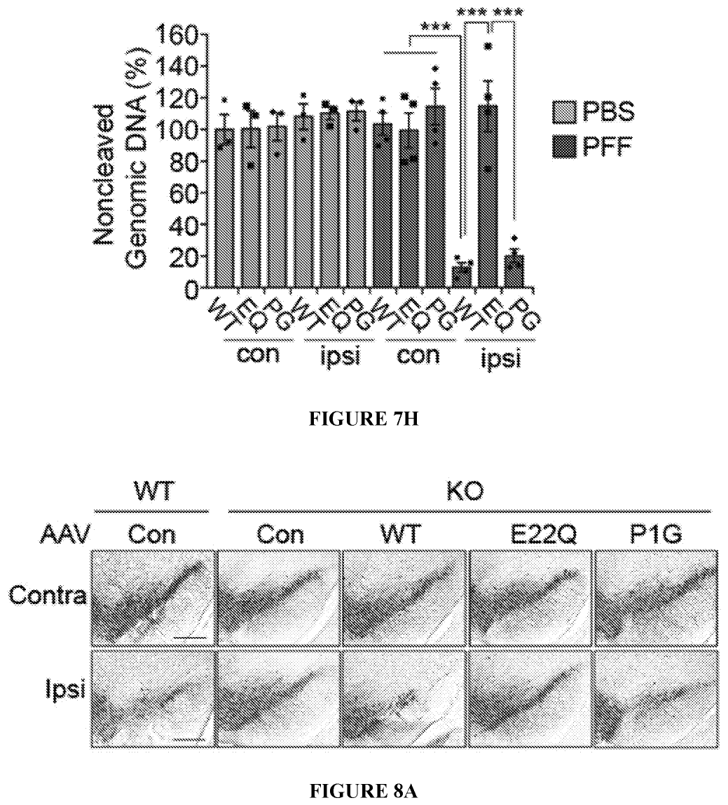

FIGS. 7A-7H demonstrate α-syn PFF-induced pathology is reduced by defection of MIF/PAAN's nuclease activity in vivo. FIG. 7A shows representative immunoblots of TH and 0-actin in the contralateral and ipsilateral striatum of PBS and α-syn PFF-injected MIF/PAAN WT, MIF/PAAN E22Q KI or MIF P1G KI mice at 6 months. FIG. 7B shows quantification of TH levels in the striatum normalized to β-actin. Bars represent the mean±s.e.m. ***P<0.0005, two-way ANOVA followed by Tukey's post hoc test (n=4). FIG. 7C shows stereological counts of TH+Nissl⁺ cells. Data are mean±s.e.m. ***P<0.0005, two-way ANOVA followed by Tukey's post hoc test (n=5 to 6 mice per group). FIG. 7D shows DOPAC concentrations in the striatum of WT, E22Q and P1G knock-in mice at 6 months after α-syn PFF or PBS injection measured by HPLC. Bars represent mean±s.e.m. *P<0.05, **P<0.005, ***P<0.0005, two-way ANOVA followed by Tukey's post hoc test (n=5 to 8 mice per group). FIGS. 7E and 7F show behavioral abnormalities of PBS and α-syn PFF-injected MIF/PAAN WT, MIF/PAAN E22Q or MIF P1G KI mice at 6 months measured by (7E) pole test and (7F) grip strength test. Bars are the mean±s.e.m. *P<0.05, **P<0.005, two-way ANOVA followed by Tukey's post hoc test. FIG. 7G shows pulsed-field gel electrophoresis in PBS or α-syn PFF injected MIF/PAAN WT, MIF/PAAN E22Q KI or MIF P1G KI mice at 6 months. FIG. 7H shows intensity of noncleaved genomic DNA. P<0.0005, two-way ANOVA followed by Tukey's post hoc test (n=3 to 4 mice per group).

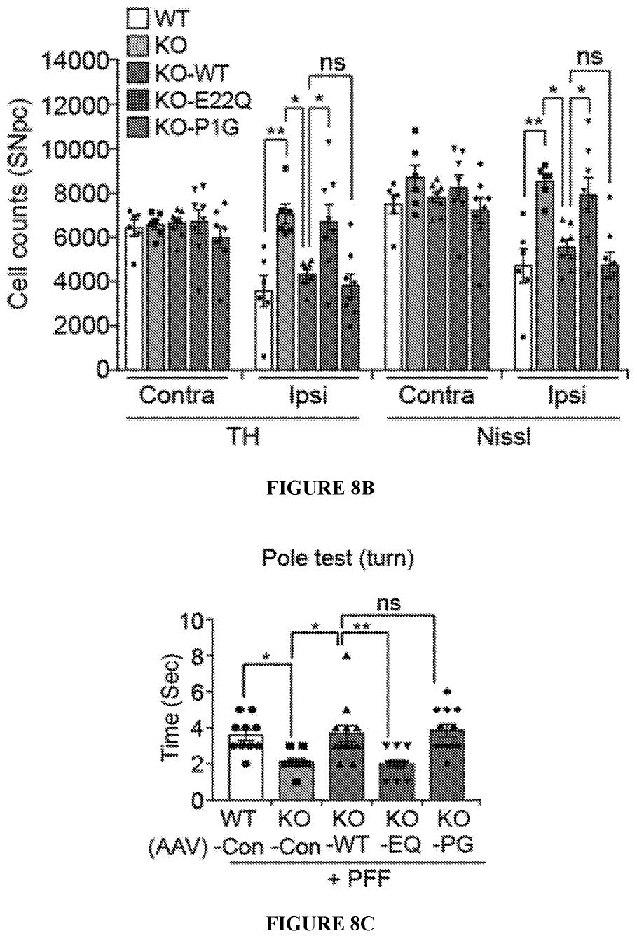

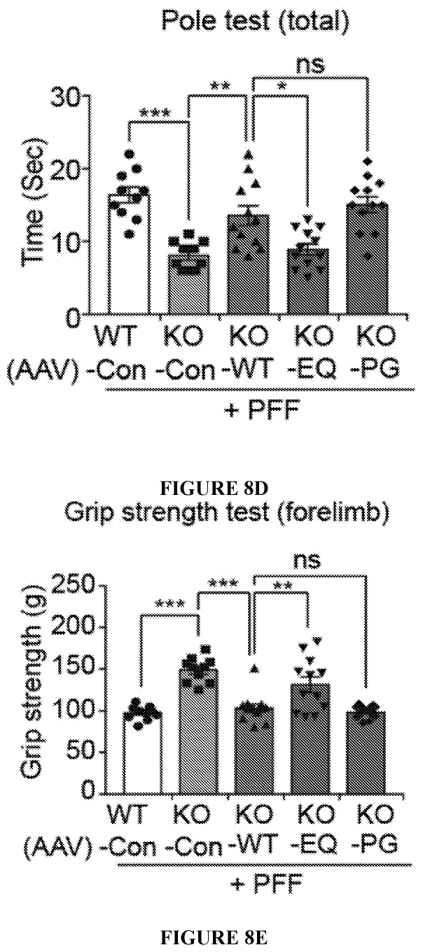

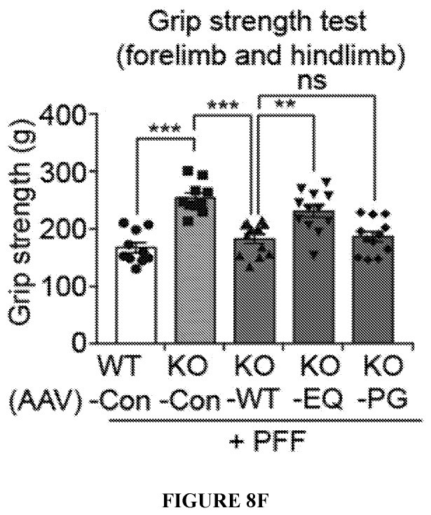

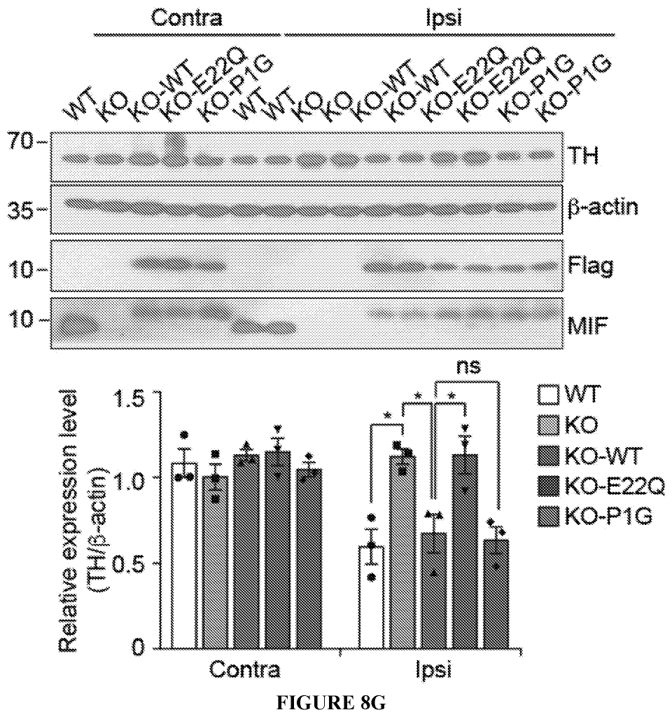

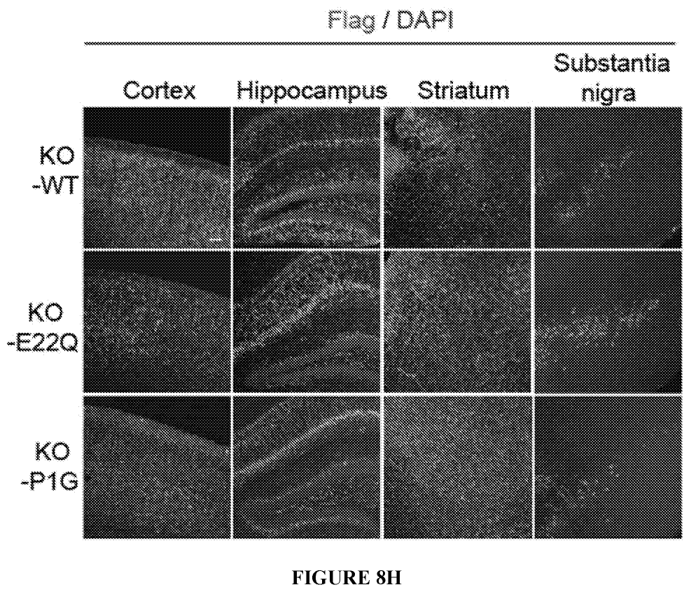

FIGS. 8A-8H demonstrate MIF/PAAN nuclease activity is critical for α-syn PFF-induced pathology. FIG. 8A shows representative TH and Nissl staining of SNpc DA neurons of MIF/PAAN WT, MIF/PAAN KO and MIF/PAAN KO mice injected with AAV2-Flag-MIF/PAAN WT, E22Q or P1G at 6 months after α-syn PFF or PBS injection. Scale bars, 400 μM. FIG. 8B shows stereological counts. Data are mean±s.e.m. *P<0.05, **P<0.005, two-way ANOVA followed by Tukey's post hoc test. FIGS. 8C-8F show 180 days after intrastiriatal α-syn PBS or PFF injection, (8C, time to turn and bottom; 8D, time to turn) pole test and (8E, forelimb; 8F, forelimb and hindlimb;) grip strength test were performed in WT, MIF/PAAN KO and MIF/PAAN KO mice injected with AAV2-MIF/PAAN WT, E22Q or P1G-Flag. *P<0.05, **P<0.005, ***P<0.0005, two-way ANOVA. FIG. 8G shows representative immunoblots of TH, Flag, MIF/PAAN and j-actin in the contralateral and ipsilateral striatum of PBS and α-syn PFF-injected MIF/PAAN WT, MIF/PAAN KO and MIF/PAAN KO mice injected with AAV2-Flag-MIF/PAAN WT, E22Q or P 1G at 6 months. Quantification of TH levels in the striatum normalized to β-actin. Bars represent the mean±s.e.m. *P<0.05, two-way ANOVA followed by Tukey's post hoc test (n=3). FIG. 8H shows representative immunostaining images of expression of AAV2-Flag-MIF/PAAN WT, MIF/PAAN-E22Q and MIF P1G in cortex, hippocampus, striatum and substantia nigra 6 month after injection. Scale bar, 100 μm.

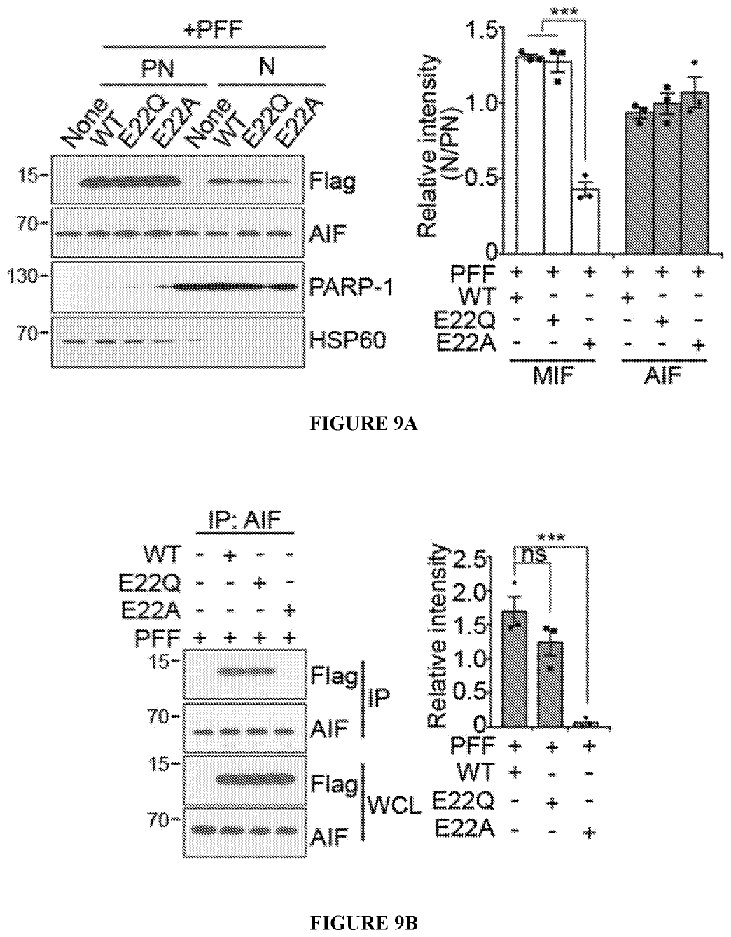

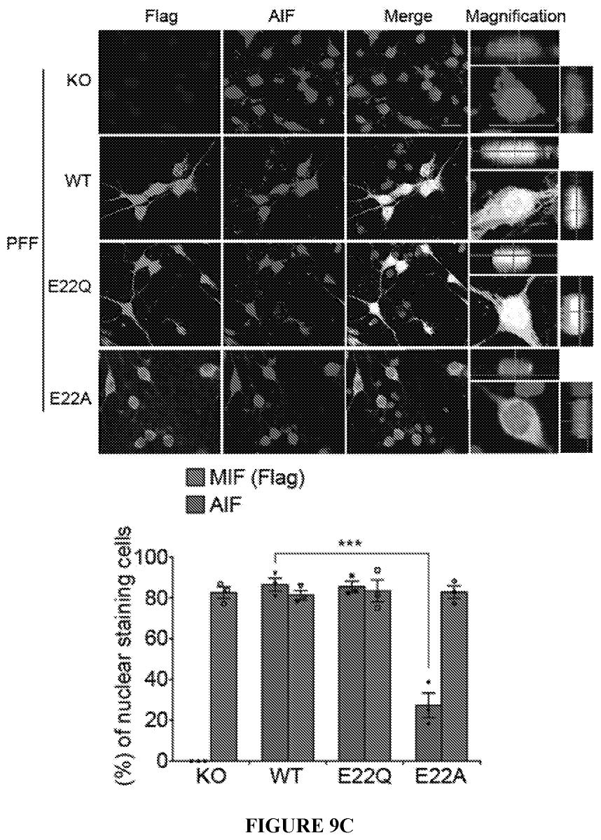

FIGS. 9A-9C demonstrate MIF/PAAN E22A mutant prevent AIF's recruitment of MIF/PAAN to the nucleus in α-syn PFF-induced toxicity. In FIG. 9A, MIF/PAAN KO cortical neurons were transduced with AAV containing Flag-tagged MIF/PAAN WT or MIF/PAAN mutants (E22Q and E22A), followed by incubation with α-syn PFF. Nuclear translocation of AIF and MIF/PAAN variants were determined by western blot analysis from post nuclear (PN) and nuclear (N) fraction. Relative levels of MIF/PAAN and AIF in the nuclear fraction (N) is shown in the graph. Data are the means±s.e.m. ***P<0.0005, two-way ANOVA. PARP-1 and HSP60 are used for nuclear and mitochondrial marker, respectively. FIG. 9B shows co-immunoprecipitation (IP) of Flag-tagged MIF/PAAN variants and AIF in cortical neurons after α-syn PFF treatment. Intensity of Flag is shown in the graph. ***P<0.0005, one-way ANOVA. FIG. 9C shows images of nuclear translocation of AIF and Flag-tagged MIF/PAAN variants after α-syn PFF treatment in MIF/PAAN KO cortical neurons. Scale bar, 20 μm. White color indicates the overlay of AIF, MIF/PAAN and DAPI, showing the nuclear translocation of AIF and MIF. Purple color indicates the overlay of AIF and DAPI, showing the nuclear translocation of AIF. Z stacks illustrating the x,z and y,z axis are provided to demarcate the nucleus. Quantification of the percentage of cells with nuclear translocation of MIF/PAAN and AIF after treatment of α-syn PFF is shown in the graph. Data are the means±s.e.m. ***P<0.0005, two-way ANOVA.

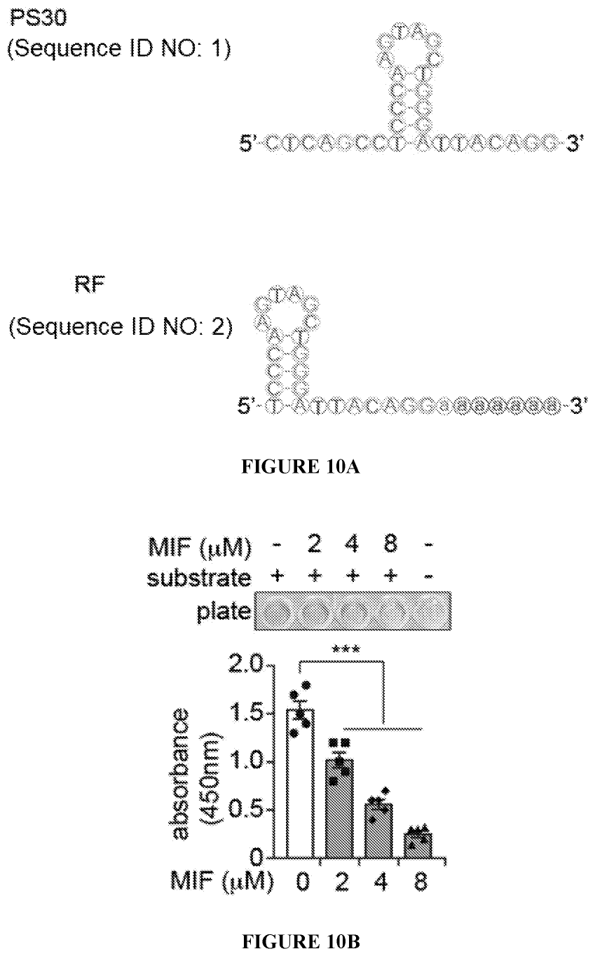

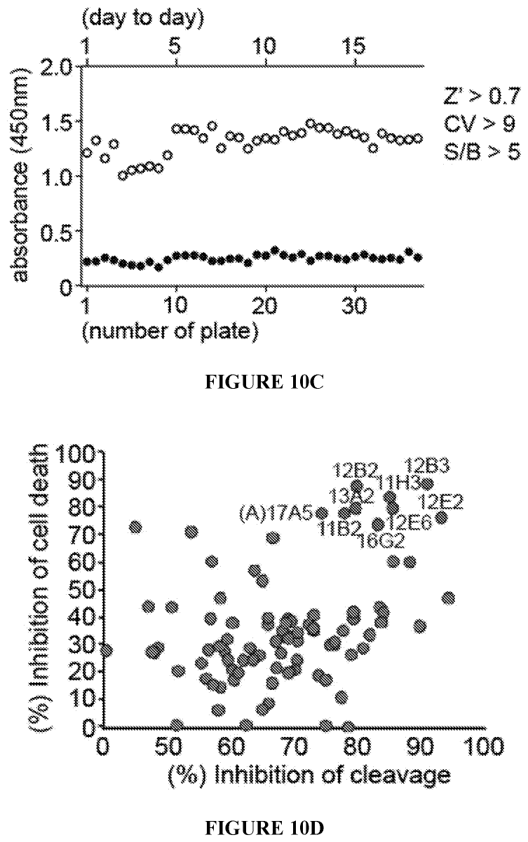

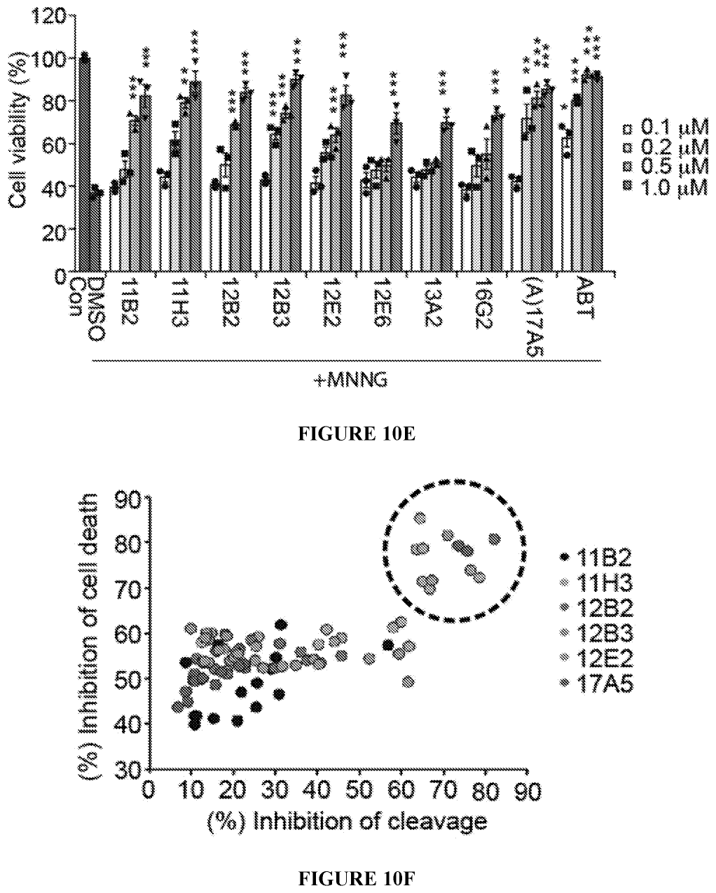

FIGS. 10A-10F illustrate high-throughput screening of MIF/PAAN inhibitors. FIG. 10A shows schematic diagram for MIF/PAAN substrate, PS30 and RF. FIG. 10B shows MIF/PAAN cleavage assay without or with MIF/PAAN protein in a concentration dependent manner. After MIF's cleavage, the remaining fragments were monitored by measuring absorbance at 450 nm. Representative colorimetric change is shown at the top of graph. The bars represent mean±s.e.m. ***P<0.0005, one-way ANOVA (n=5). FIG. 10C shows plate—to plate and day to day variability of the parameters (CV, S/B and Z′ factor) of MIF's cleavage screening assay. Thirty-eight replicates in 96-well plates of signal with MIF/PAAN protein (black circles) or without MIF/PAAN protein (white circles) were investigated. FIG. 10D shows the result of secondary screening from the primary screening pools that inhibited MIF/PAAN nuclease activity by at least 60%. Scatter plot of the percentage the inhibition of MIF/PAAN nuclease activity (X axis) and the inhibition of MNNG-induced cell death (Y axis). FIG. 10E shows Alamar blue cell viability assay from SH-SY5Y cells pre-incubated with 9 pools selected by secondary screening in a concentration-dependent manner (0.1 μM, 0.2 μM, 0.5 μM and 1 μM) in response to 50 μM MNNG for 15 min. A PARP inhibitor, ABT-888 is used for the positive control. Bars represent mean±s.e.m. *P<0.05, ***P<0.0005, two-way ANOVA followed by Tukey's post hoc test (n=3). FIG. 10F shows the result of the individual compounds screening (˜90) of 6 pools candidates from the secondary screening.

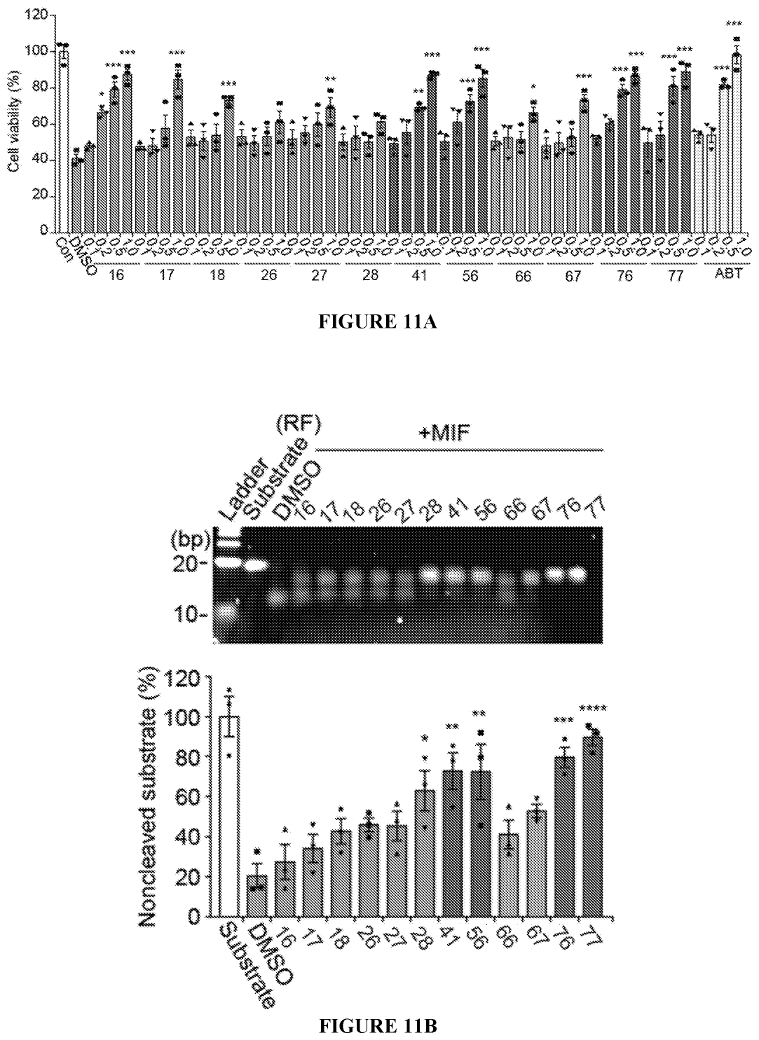

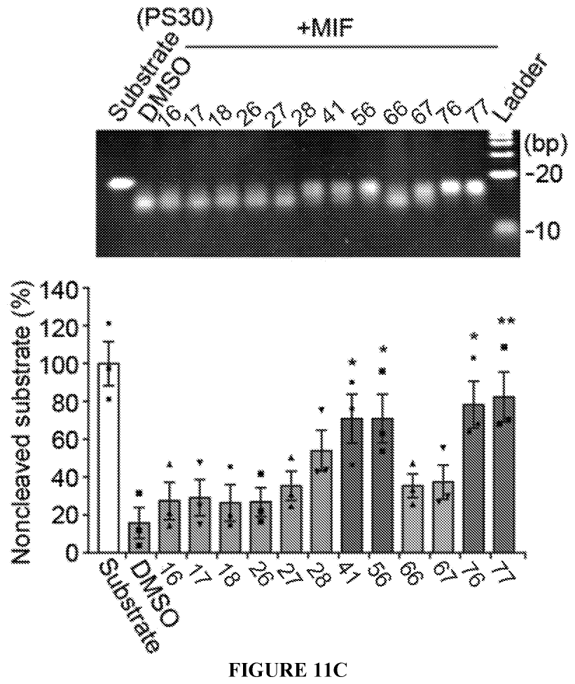

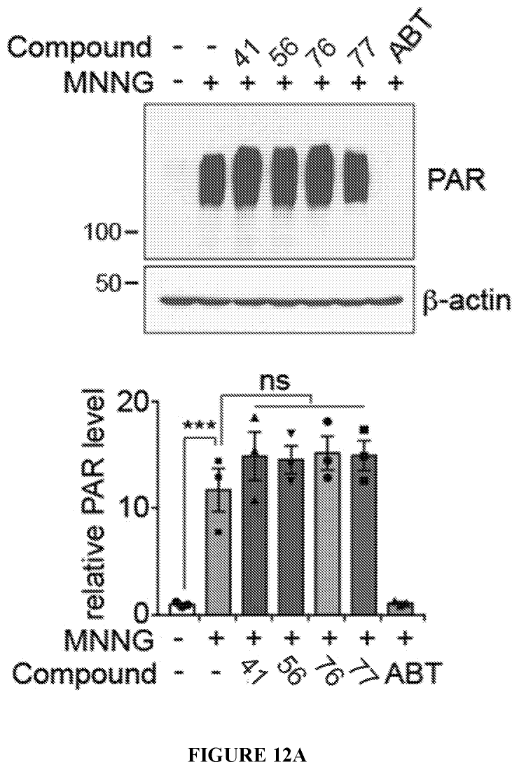

FIGS. 11A-11C demonstrate validation of 12 MIF/PAAN inhibitors from individual screening, including compounds 16-18, 26-28, 41, 56, 66, 67, 76, and 77. FIG. 11A shows Alamar blue cell viability assay from SH-SY5Y cells pre-incubated with 12 MIF/PAAN inhibitors selected by individual screening in a concentration-dependent manner (0.1 μM, 0.2 μM, 0.5 μM and 1 μM) in response to 50 μM MNNG for 15 min. A PARP inhibitor, ABT-888 is used for the positive control. Bars represent mean±s.e.m. **P<0.005, ***P<0.0005, two-way ANOVA followed by Tukey's post hoc test (n=3). FIGS. 11B and 11C show in vitro MIF's nuclease assay with 12 MIF/PAAN inhibitors using RF substrates or PS30 substrates. Quantification of noncleaved genomic DNA. Bars represent mean±s.e.m. *P<0.05, **P<0.005, ***P<0.0005, one-way ANOVA followed by Tukey's post hoc test (n=3).

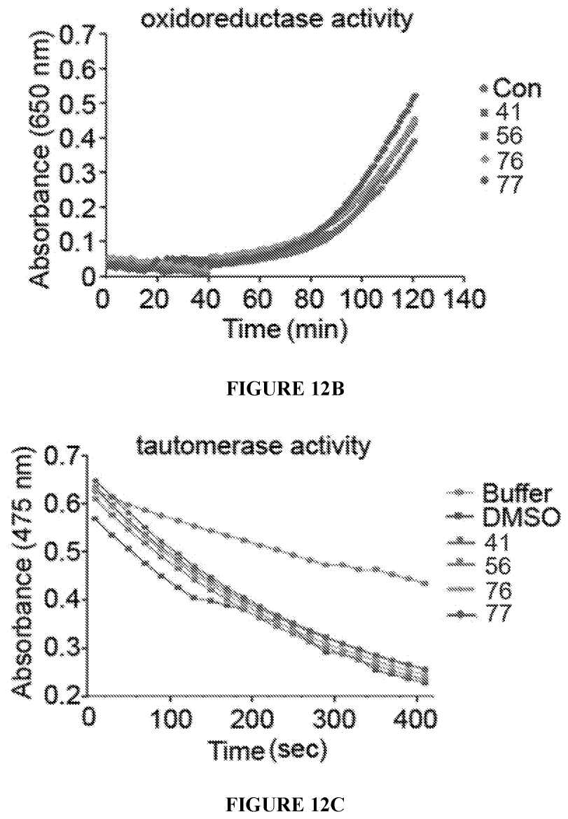

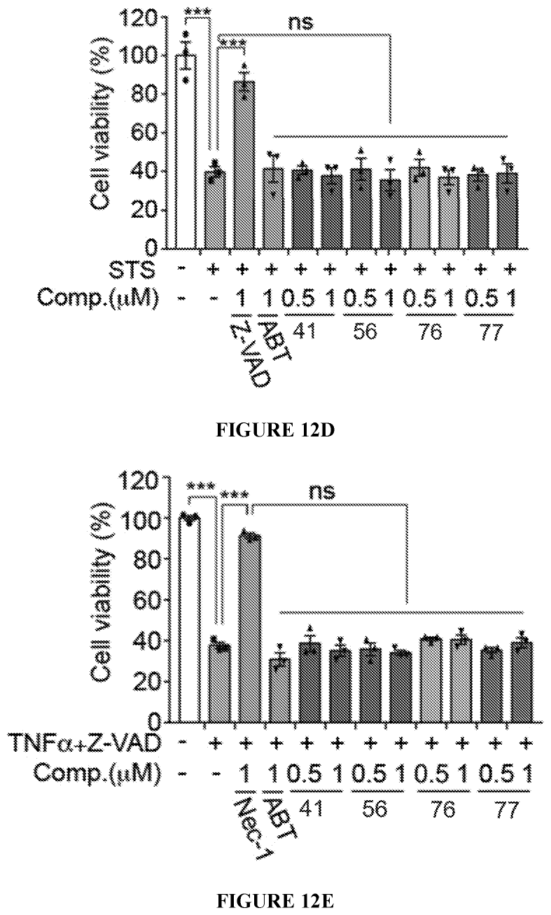

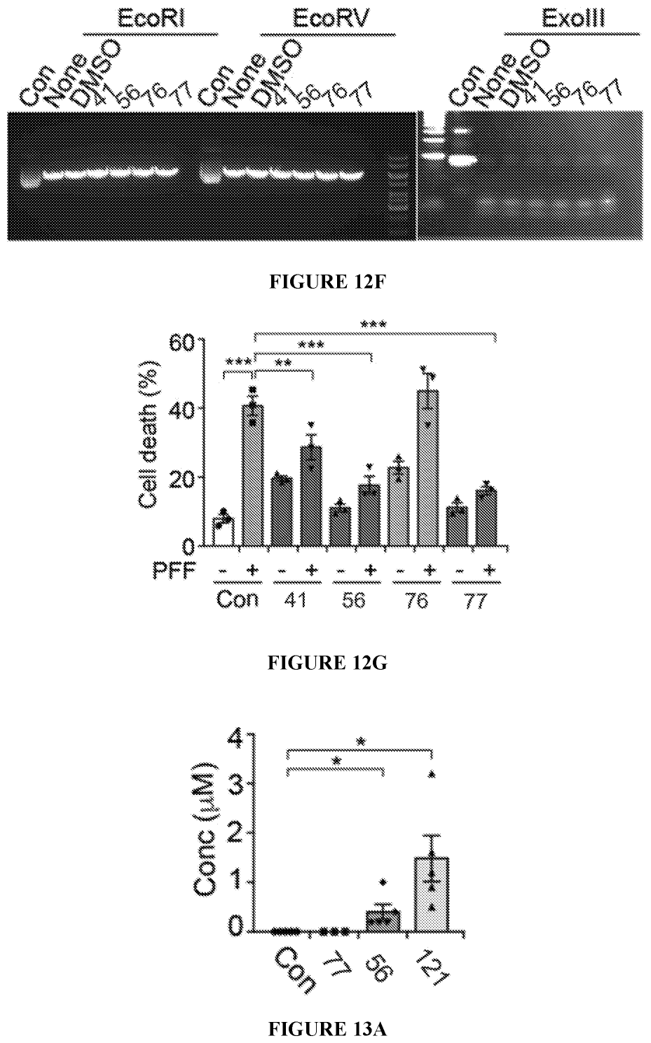

FIGS. 12A-12G demonstrate validation of compounds 41, 56, 76, and 77 as MIF/PAAN inhibitors. FIG. 12A shows representative western blot analysis and quantification of the levels of MNNG-induced PAR accumulation in the presence and absence of MIF/PAAN inhibitors (compounds 41, 56, 76, and 77). Graphs represent mean±s.e.m. ***P<0.0005, two-way ANOVA followed by Tukey's post hoc test (n=3). A PARP inhibitor, ABT-888 is used for the positive control. FIGS. 12B and 12C show in vitro oxidoreductase and tautomerase activity of MIF/PAAN with compound 41, 56, 76, or 77. FIGS. 12D and 12E show Alamar blue cell viability assay from SH-SY5Y cells pre-incubated with compound 41, 56, 76, or 77 in response to 0.5 μM staurosporine (STS) or from HT 22 cells incubated with 1 ng/ml of TNF-α with 50 μM z-VAD. A pan caspase inhibitor, z-VAD or necrostatin (Nec-1) is used for the positive control and PARP inhibitor, ABT-888 is used for negative control. Bars represent mean±s.e.m. ***P<0.0005, two-way ANOVA followed by Tukey's post hoc test (n=3). FIG. 12F shows in vitro nuclease assay of EcoRI and EcoRV with pcDNA3 as a substrate and ExoIII with 18 bp of double-strand DNA as a substrate in the absence or presence of MIF/PAAN inhibitors (compound 41, 56, 76, or 77). FIG. 12G shows quantification of cell death from Hoechst and propidium iodide (PI) staining from primary cortical neurons pre-treated with 1 μM of compound 41, 56, 76, or 77, followed by further incubation with α-syn PFF. Bars represent mean±s.e.m. **P<0.005, ***P<0.0005, two-way ANOVA followed by Tukey's post hoc test (n=3).

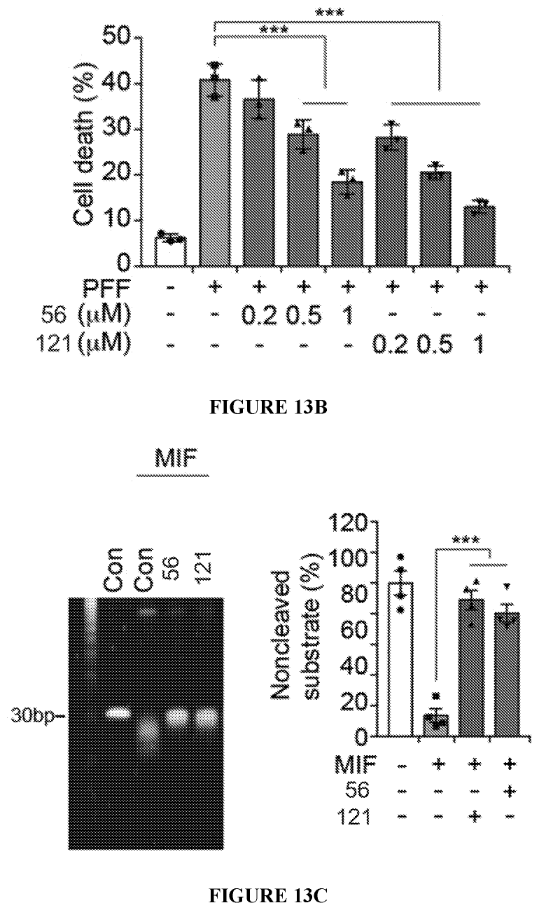

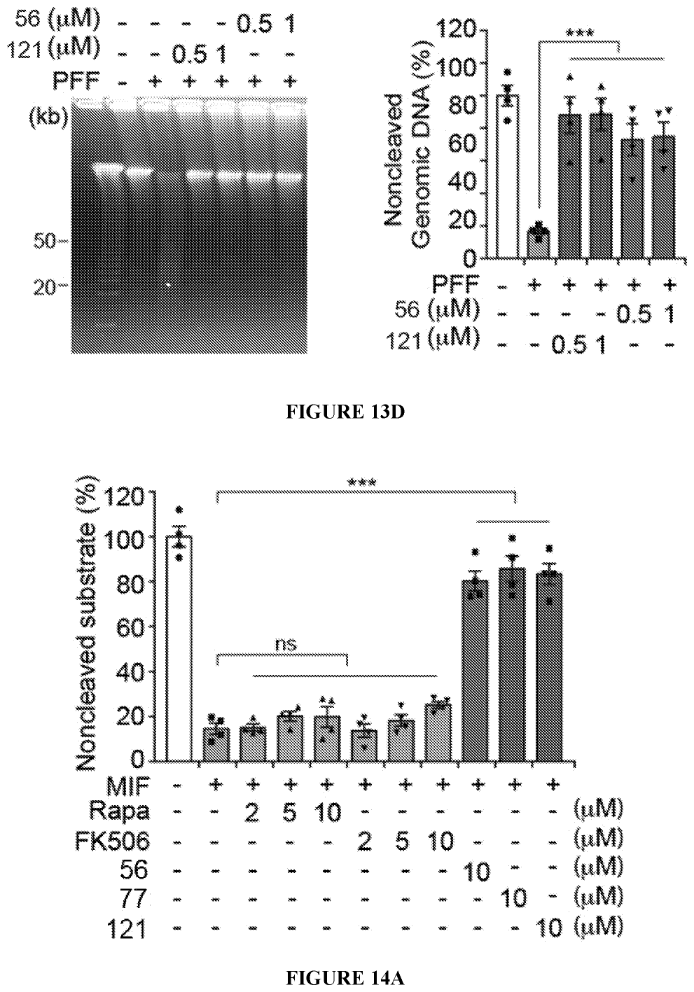

FIGS. 13A-D demonstrate the identification of compound 121. FIG. 13A shows C57BL/6 mice were orally administrated with 10 mg/kg of vehicle, compound 56, 77 or 121 for 2h. The concentrations of compounds in brain tissue were measured by LC/MS. FIG. 13B shows quantification of cell death. Bars represent means±s.e.m. ***P<0.0005, two-way ANOVA followed by Tukey's post hoc test (n=3). FIG. 13C shows in vitro MIF's nuclease assay with compound 56 or 121. Quantification of noncleaved substrate. Bars represent mean±s.e.m. ***P<0.0005, two-way ANOVA followed by Tukey's post hoc test (n=4). FIG. 13D shows Pulsed-field gel electrophoresis of α-syn PFF-induced DNA damage in mouse cortical neurons treated with compound 56 or 121. Intensity of noncleaved genomic DNA is shown in the graph. Bars represent mean±s.e.m. ***P<0.0005, two-way ANOVA followed by Tukey's post hoc test (n=4).

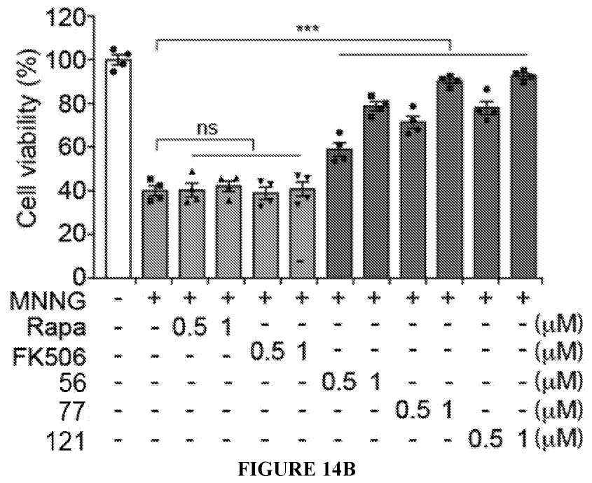

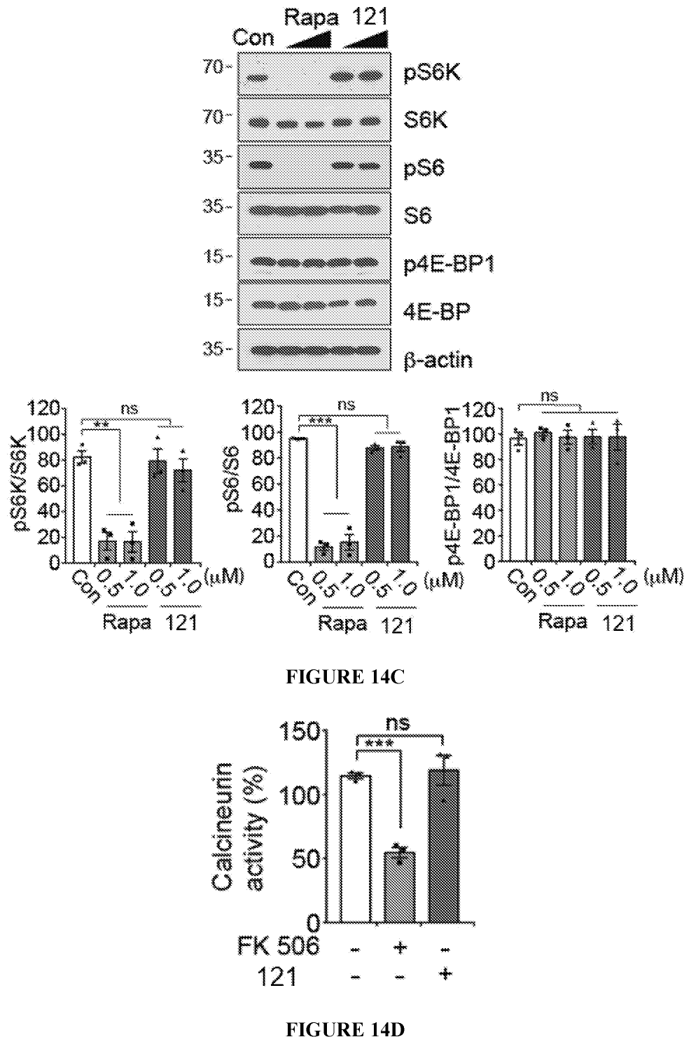

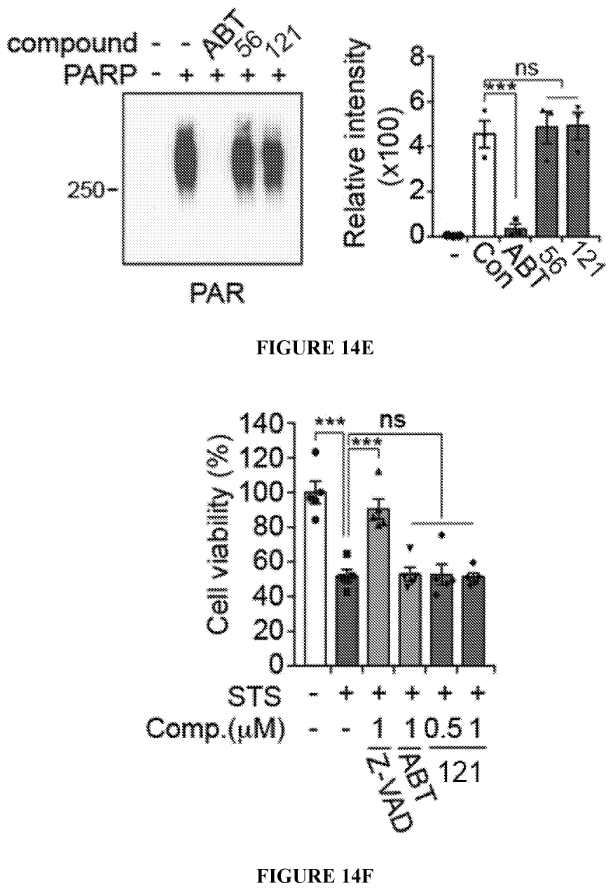

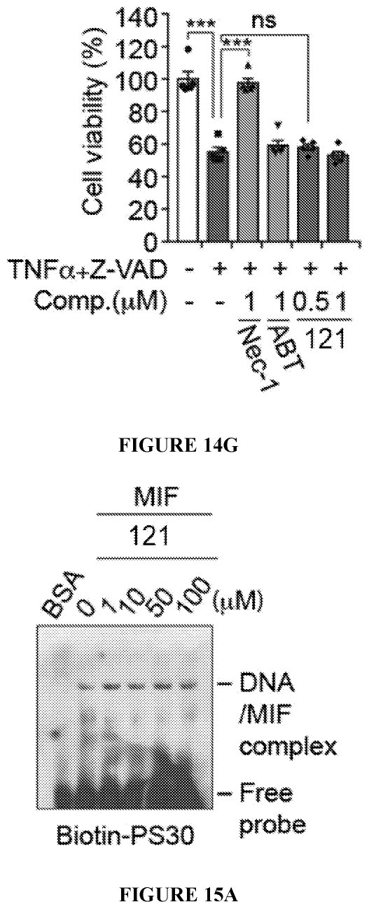

FIGS. 14A-14G show the characterization of compound 121. FIG. 14A shows quantification of noncleaved substrate DNA from in vitro MIF's nuclease assay with indicated dose of rapamycin, FK506 or MIF/PAAN inhibitors (compound 56, 77, or 121) using the PS 30 substrate. Bars represent means±s.e.m. ***P<0.0005, two-way ANOVA followed by Tukey's post hoc test (n=4). FIG. 14B shows Alamar blue cell viability assay from SH-SY5Y cells pre-incubated with 0.5 or 1 μM of rapamycin, FK506 or MIF/PAAN inhibitors (compound 56, 77, or 121) in response to 50 μM MNNG for 15 min. Bars represent mean±s.e.m. ***P<0.0005, two-way ANOVA followed by Tukey's post hoc test (n=4). FIG. 14C shows representative immunoblots (left) and quantification (right) of pS6K, pS6 and p4E-BP1 levels in SH-SY5Y cells incubated with 0.5 or 1 μM of Rapamycin or compound 121 for 3 h. Bars represent means±s.e.m. **P<0.005, ***P<0.0005, one-way ANOVA followed by Tukey's post hoc test (n=3). FIG. 14D shows calcineurin activity assay from SH-SY5Y cells treated with 1 μM of FK506 or compound 121 for 6 h. Bar represent mean±s.e.m. ***P<0.0005, one-way ANOVA followed by Tukey's post hoc test (n=3). FIG. 14E shows in vitro ribosylation assay (IVRA) of PARP-1 with compound 56, 121 or ABT-888. Quantification of inhibition of PARP-1 activity by compound 56, compound 121 and ABT-888 is shown. Graphs represent mean±s.e.m. ***P<0.0005, two-way ANOVA followed by Tukey's post hoc test (n=3). FIGS. 14F and 14G show Alamar blue cell viability assay from SH-SY5Y cells pre-incubated with either compound 56 or 121 in response to (14F) 0.5 μM staurosporine (STS) or (14G) from HT 22 cells incubated with 1 ng/ml of TNF-α with 50 μM z-VAD. Bars represent mean±s.e.m. ***P<0.0005, two-way ANOVA followed by Tukey's post hoc test (n=5).

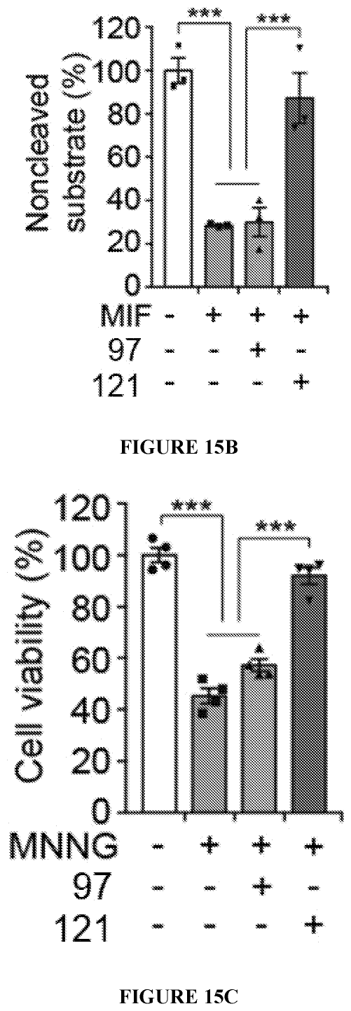

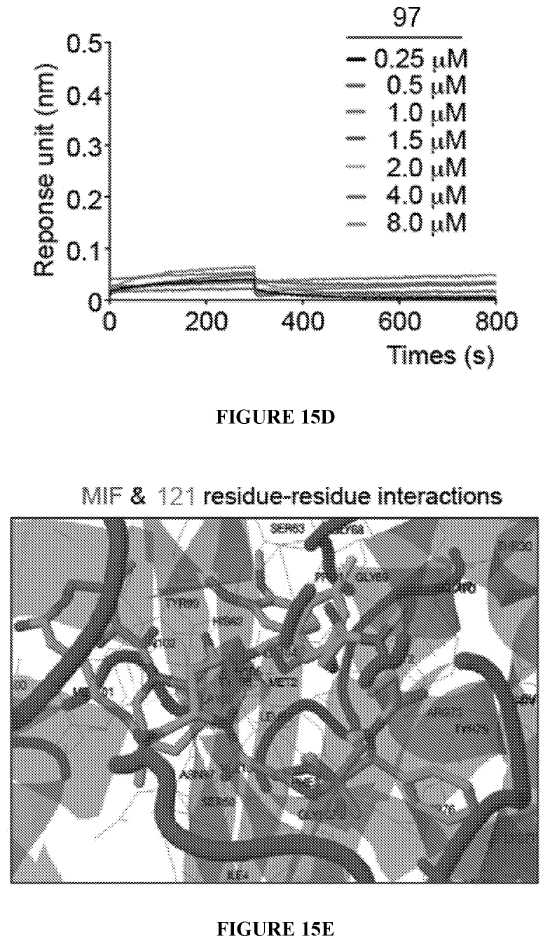

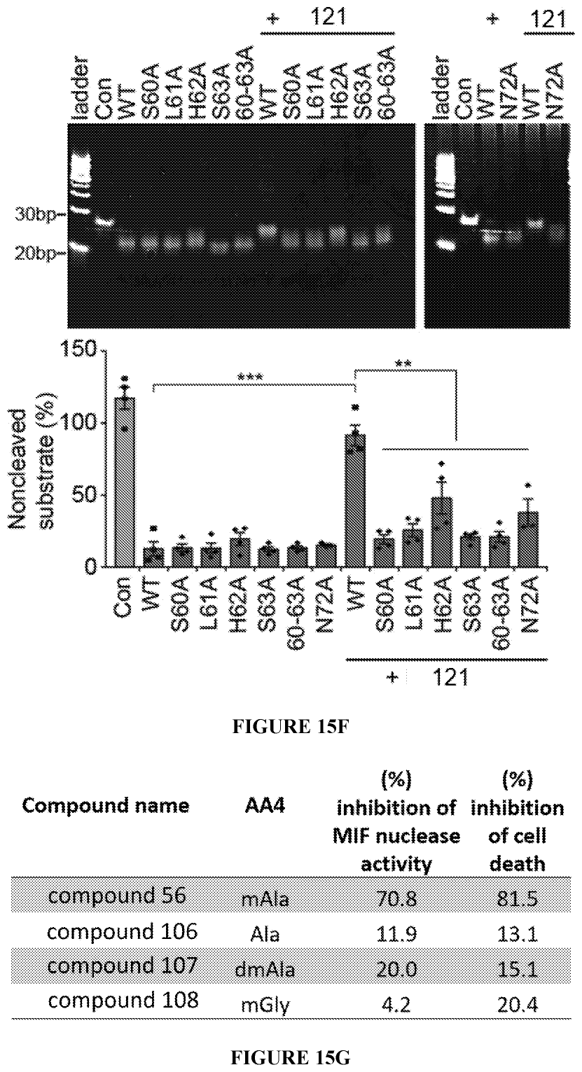

FIGS. 15A to 15G demonstrate interaction between compound 121 and MIF. FIG. 15A shows effect of compound 121 on binding of MIF/PAAN with biotin-labeled small DNA substrate (PS30) in an EMSA assay. Arrow indicates the DNA-MIF/PAAN complex. FIGS. 15B and 15C illustrate effect of compound 97 on MIF nuclease activity and MNNG-induced cell death. FIG. 15D shows binding affinities of MIF/PAAN WT to non-bound compound 97 as determined by biolayer interferometry (ForteBio Octet) assay. Data are representative of three independent experiments. FIG. 15E shows 3D model of MIF/PAAN with compound 121. Compound 121 does not bind to DNA nor the MIF/PAAN DNA binding pocket. FIG. 15F shows effect of compound 121 in MIF/PAAN nuclease assay with different MIF/PAAN mutants using PS30 substrate. FIG. 15G shows effect of modification of position of N-methylalanine in compound 56 on its activities. Quantification of noncleaved substrate DNA. Bars represent mean±s.e.m. **P<0.005, ***P<0.0005, two-way ANOVA followed by Tukey's post hoc test (n=4).

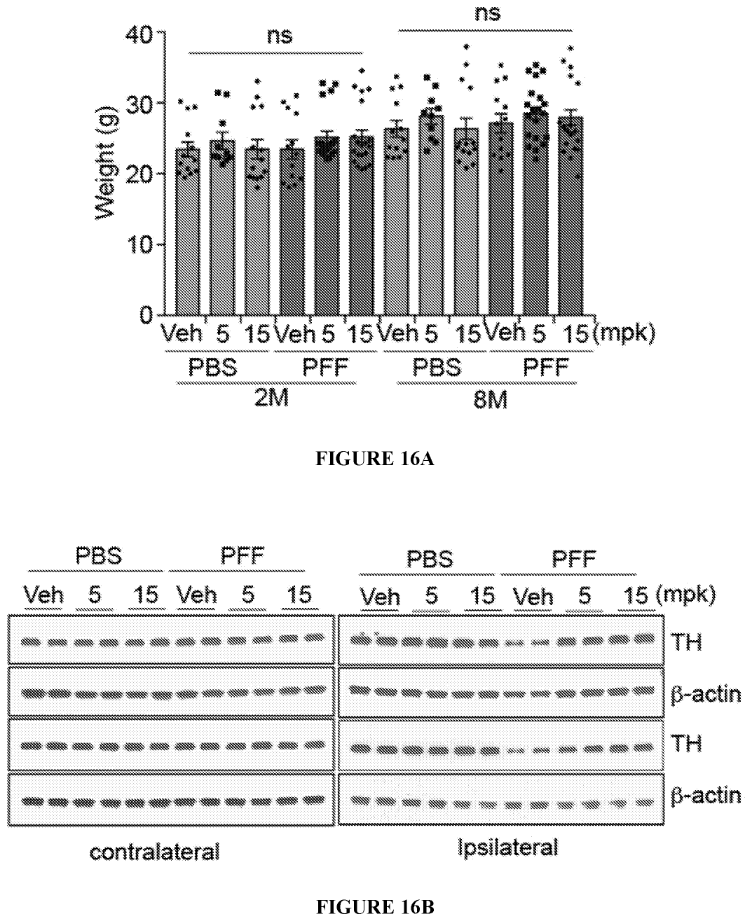

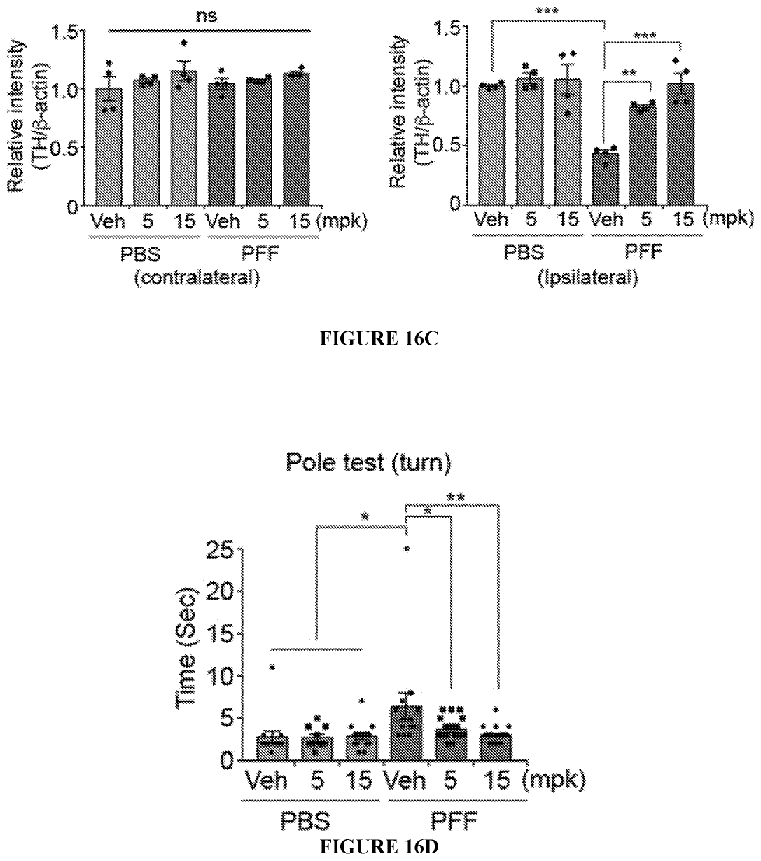

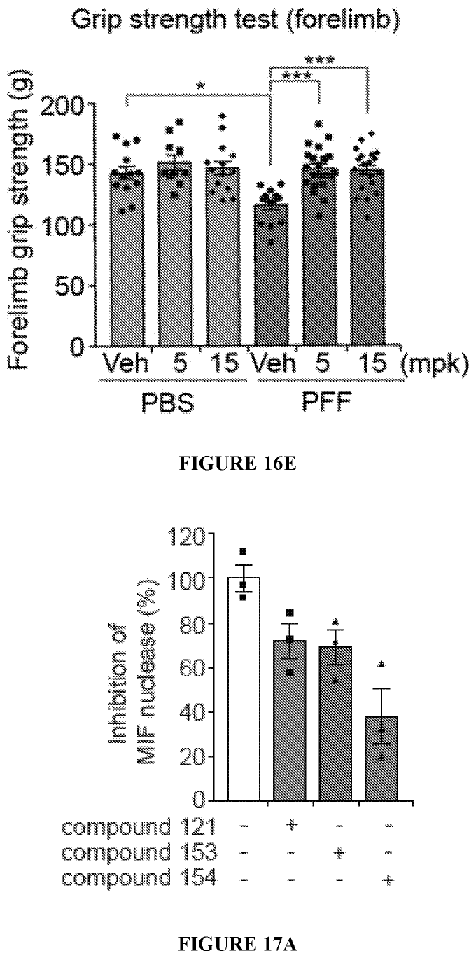

FIGS. 16A-16E demonstrate effect of compound 121 on α-syn PFF-induced pathology in vivo. FIG. 16A shows measurement of weight from PBS or α-syn PFF-injected WT mice before or after administration of vehicle and compound 121. FIG. 16B shows representative immunoblots of TH and f-actin in the contralateral and ipsilateral striatum of PBS or α-syn PFF-injected WT mice delivered vehicle or compound 121 for 5 months. FIG. 16C shows quantification of TH levels in the striatum normalized to β-actin. Bars represent the mean±s.e.m. **P<0.005, ***P<0.0005, two-way ANOVA followed by Tukey's post hoc test (n=4). FIGS. 16D and 16E show behavioral defects of PBS and α-syn PFF-injected mice administrated vehicle or compound 121 for 5 months measured by (16D) pole test and (16E) grip strength test. Bars are the means±s.e.m. *P<0.05, **P<0.005, ***P<0.0005, two-way ANOVA followed by Tukey's post hoc test.

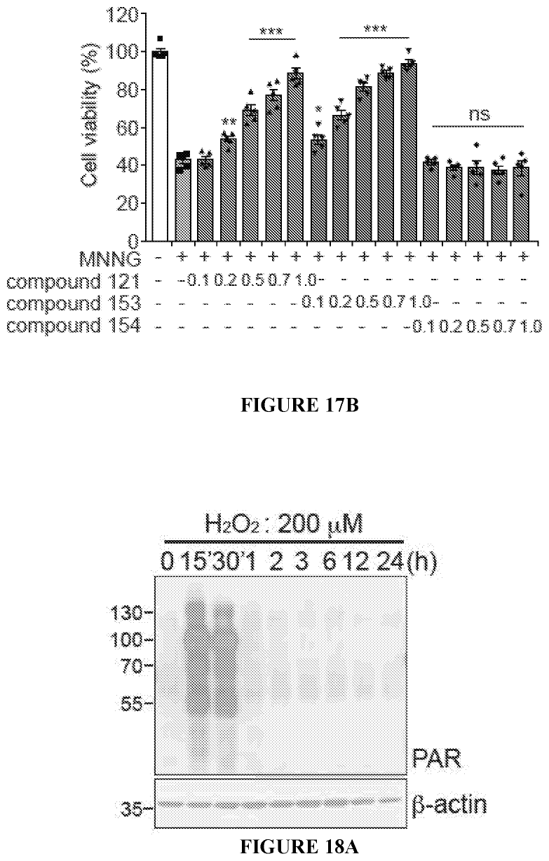

FIG. 17A shows colorimetric assay for MIF nuclease activity using 10 μM of compound 121, which is racemic mixture of compounds 153 and 154, compound 153, and compound 154. FIG. 17B shows MNNG-induced cell death assay for compound 121, compound 153, and 154. SH-SY5Y cells were pre-incubated compound 121, compound 153, 154 with concentrations as indicated for 1 h, followed by 50 μM MNNG for 15 min. After 24 h, cell viability was measured by Alamar blue. Bars represent mean±s.e.m. ***P<0.0005, two-way ANOVA followed by Tukey's post hoc test (n=5).

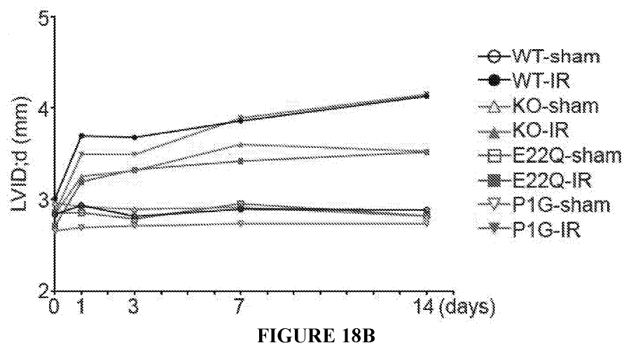

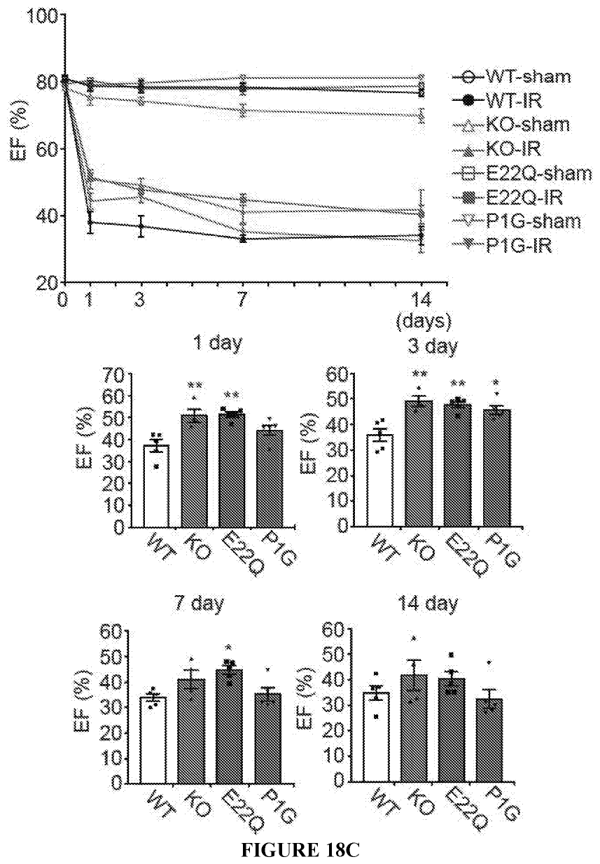

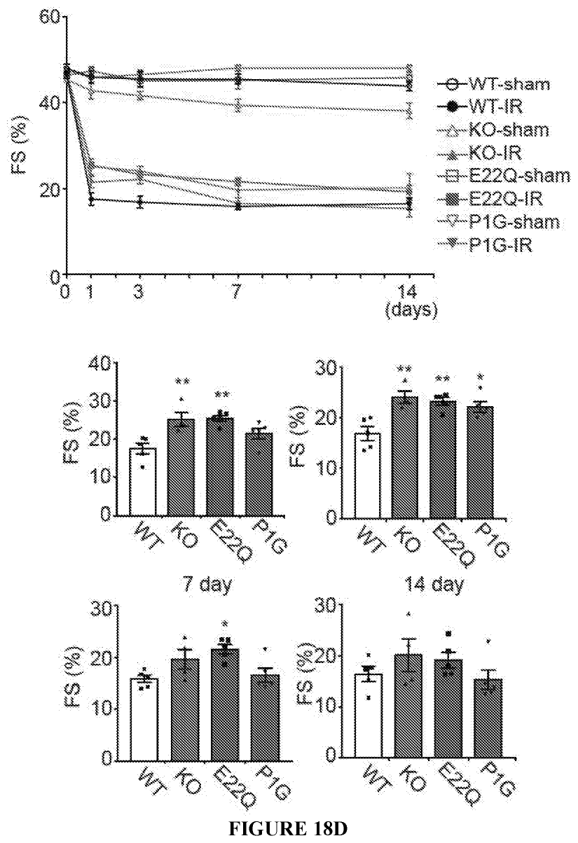

FIGS. 18A-D show MIF KO and MIF E22Q KI attenuate the/R-induced cardiac dysfunction. FIG. 18A shows activation of PARP-1 in H202-treated primary rat cardiac myocytes. FIGS. 18B-D show cardiac function examined using two-dimensional echocardiography at indicated time after surgery. I/R group undergoes the occlusion of the left coronary artery (LCA) for 45 minutes followed by reperfusion. LV systolic diameter (b, LVID), ejection fraction (c, EF), and shortening fraction (d, SF) were measured. Bars represent summarized measurement at each time point. Two-way ANOVA. *P<0.05, **P<0.005.

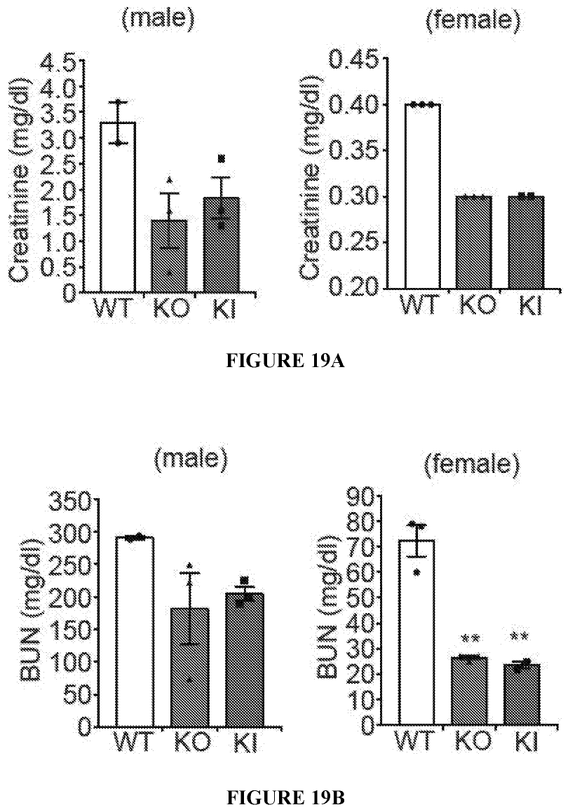

FIGS. 19A-B show renal ischemia model, which indicate protective phenotype against I/R in MIF KO and MIF E22Q KI mice. FIG. 19A shows serum creatinine levels in MIF WT, MIF KO and MIF KI male or female mice after kidney I/R (45 min of ischemia and 48 h of reperfusion). FIG. 19B shows Blood urea nitrogen (BUN) levels in MIF WT, MIF KO and MIF KI male or female mice after kidney I/R (45 min of ischemia and 48 h of reperfusion).

To determine whether PAAN is essential for pathologic α-syn induced degeneration, recombinant α-synuclein preformed fibrils (PFFs) were stereotaxically injected into the striatum of wild type (WT) and PAAN/MIF knockout (KO) mice. The absence of PAAN/MIF prevents the loss of dopamine (DA) neurons as assessed by stereologic counting of tyrosine hydroxylase (TH) immunoreactivity and Nissl staining (FIGS. 2A and 2B), immunoblot analysis of TH and DA transporter (DAT) levels (FIGS. 6A and 6B), high-performance liquid chromatography (HPLC) assessment of DA (FIG. 2C) and its metabolite, 3,4-dihydroxyphenylacetic acid (DOPAC) (FIG. 2D) and the accompanying behavioral deficits including hindlimb clasping test (FIG. 6C), pole test (FIGS. 2E and 6D) and grip strength test (FIGS. 2F and 6E).

PAAN/MIF was originally identified as an atypical secreted cytokine that exhibits tautomerase activity that accounts for some but not all of MIF's pleiotropic actions. When PAAN/MIF is recruited to the nucleus it acquires a gain of function activity as a nuclease. Its nuclease activity is distinct from it actions as a cytokine and tautomerase. Accordingly, the relative contribution of PAAN/MIF's nuclease activity versus MIF's tautomerase in pathologic α-syn induced degeneration was evaluated. Mice lacking PAAN/MIF nuclease activity (MIF E22Q) are resistant to α-syn PFF-induced neurodegeneration, while mice lacking MIF's tautomerase activity (MIF P1G) are as sensitive as WT mice in α-syn PFF-induced neurodegeneration (FIGS. 2G-2K and FIGS. 7A-7F). Pulse gel electrophoresis indicates that α-syn PFF induces large scale genomic DNA cleavage in WT and MIF P1G mice, while it is significantly attenuated in PAAN/MIF E22Q mice (FIGS. 7G and 7H). Viral transgenesis of Adeno associated viral (AAV) expression of PAAN/MIF WT, PAAN/MIF E22Q and MIF P1G in MIF KO mice confirmed that PAAN/MIF's nuclease activity, but not MIF's tautomerase activity is required for α-syn PFF-induced neurodegeneration in vivo (FIGS. 8A-8H).

In primary neuronal cultures α-syn PFF induces nuclear translocation of PAAN/MIF and AIF that is attenuated by the poly (ADP-ribose) polymerase (PARP) inhibitor, ABT-888 (FIG. 3A). See Donawho, et al. (2007) Clin. Cancer Res. 13:2728-37. Co-immunoprecipitation indicates that AIF and PAAN/MIF interact following α-syn PFF administration (FIG. 3B) and they co-localize in the nucleus (FIG. 3C). AAV expression of PAAN/MIF WT, PAAN/MIF E22Q and MIF P1G and the AIF binding deficient mutant PAAN/MIF E22A in MIF KO neuronal cultures indicates that PAAN/MIF's nuclease activity and its binding to AIF as well as its nuclear translocation is required for cleavage of genomic DNA induced by α-syn PFF (FIG. 3D and FIGS. 9A-9C). Neuronal cell death in response to α-syn PFF is also attenuated in PAAN/MIF KO neuronal cultures and KO neurons transduced with PAAN/MIF E22Q and E22A, while α-syn PFF kills MIF KO neurons transduced with AAV PAAN/MIF WT and MIF P1G (FIGS. 3E and 3F). Similar results were obtained in PAAN/MIF E22Q and MIF P1G knock in neuronal cultures (FIGS. 3G and 3H). Together, these results strongly suggest that the nuclease, but not the tautomerase, activity of PAAN/MIF is required for α-syn PFF-induced neurodegeneration in cultures and in vivo.

We developed an assay to screen for PAAN/MIF nuclease inhibitors. Single-stranded PAAN/MIF nuclease DNA substrate (FIG. 10A) was amine modified on the 5′ end and immobilized on a plate. Hybridization with a biotin-labeled complementary oligonucleotide followed by addition of streptavidin conjugated horseradish peroxidase enzyme (HRP) leads to retention of HRP on the surface within each well in the presence of intact PAAN/MIF nuclease DNA substrate, which can be quantitatively detected through colorimetric change at 450 nm using the HRP substrate 3,3′,5,5′-tetramethylbenzidine (TMB). In the presence of WT PAAN/MIF, there is reduced signal, indicative of cleavage of the PAAN/MIF nuclease substrate, while the signal is not attenuated without recombinant PAAN/MIF (FIGS. 1 and 10B). The rapamycin-inspired macrocycle library, known as the “rapafucins”, bearing a conserved FKBP-binding domain and a variable tetrapeptide effector domain was recently developed and shown to possess novel target specificity and enhanced pharmacological activity. Thus, the rapafucin library containing 45,000 macrocyclic compounds in 3,000 pools of 15 compounds was screened using this assay. The Z′ factors were within the 0.5 ˜ 1 range, indicating that minimal variations within days and between plates were observed (FIG. 10C). Several pools were identified that inhibit PAAN/MIF nuclease activity from the primary screen (FIG. 4A). Pools that inhibited PAAN/MIF nuclease by at least 60% were rescreened for both inhibition of PAAN/MIF nuclease activity and prevention of parthanatic cell death induced by 15 min of 50 μM N-Methyl-N-nitro-N-nitrosoguanidine (MNNG) in SH-SY5Y cells assessed 24 h later. Nine pools of compounds were identified that both inhibit PAAN/MIF cleavage of the substrate and protect against MNNG-induced parthanatic cell death by greater than 70% (FIG. 10D). The 9 pools were narrowed to 6 pools since only 6 of the pools exhibited a dose response for prevention of MNNG-induced parthanatic cell death (FIG. 10E). Next, the 90 individual compounds in the 6 pools were synthesized and further assessed for inhibition of PAAN/MIF nuclease activity and MNNG-induced parthanatic cell death. Among them 12 compounds (compounds 16-18, 26-28, 41, 56, 66, 67, 76, and 77) were identified that block MIF nuclease activity and MNNG-induced parthanatic cell death by greater than 60% (FIG. 10F). Dose dependence (0.1 to 1.0 μM) were assessed for the 12 compounds against MNNG-induced parthanatic cell death (FIG. 11A and Table 2) and inhibition of PAAN MIF nuclease activity against the RF (FIG. 111B) and PS30 substrates (FIG. 11C). Compounds 41, 56, 76, and 77 were selected for further study because they prominently blocked both MNNG-induced parthanatic cell death and PAAN/MIF nuclease activity.

Compounds 41, 56, 76, and 77 did not affect MMNG-induced PARP activation, indicating that they do not have cross-reactivity to PARP inhibition to prevent cell death (FIG. 12A). Neither did they inhibit MIF's oxidoreductase activity (FIG. 12B) nor its tautomerase activity (FIG. 12C). In addition, compounds 41, 56, 76, and 77 and the PARP inhibitor ABT-888 do not inhibit staurosporine (STS)-induced apoptotic cell death, while a pan-caspase inhibitor Z-VAD prevents STS-induced cell death (FIG. 12D). TNFα and Z-VAD-induced necroptosis is also not prevented by compounds 41, 56, 76, 77, and ABT-888 while Nec-1 prevents necroptosis (FIG. 12E). Compounds 41, 56, 76, and 77 do not inhibit the nuclease activity of the structurally related nucleases, EcoRI, EcoRV and ExoIII (FIG. 12F). Taken together, compounds 41, 56, 76, and 77 selectively prevent parthanatos via inhibiting PAAN/MIF nuclease activity. Compounds 41, 56, and 77 attenuated α-syn PFF-induced cell death in mouse cortical neurons, while C11 had no effect (FIG. 12G). Both compounds 41 and 76 exhibited some toxicity when applied to primary cortical neurons (FIG. 12G) and were not advanced for further study. Both compounds 56 and 77 protected against cell death similar to ABT-888 (FIG. 4B) and prevented genomic DNA cleavage (FIG. 4C) in human cortical neurons treated with human α-syn PFF. Compounds 56 and 77 were further evaluated with regards to their ability to cross the blood brain barrier (BBB) and only compound 56 crossed the BBB after oral gavage (FIG. 13A).

Although compound 56 is capable of crossing the BBB, the achievable concentration (<500 nM) is below that required for inhibition of α-syn PFF-induced cell death (FIG. 13A and 13B). To optimize the potency compound 56 and increase its CNS penetration, we designed and synthesized new derivatives of compound 56 by modifying the tetrapeptide effector domain and the FKBP-binding domain (FKBD), in tandem. The compound 56 analogs were evaluated for the ability to inhibit PAAN/MIF nuclease activity, cell death and the ability to cross the BBB. We identified one compound, designated compound 121 that inhibits PAAN/MIF nuclease activity and parthanatic cell death (Table 2). Compound 56 inhibits MNNG-induced parthanatic cell death with an IC₅₀ of 0.52 μM and compound 121 shows an IC₅₀ of 0.28 μM (FIG. 4D). Moreover, levels of compound 121 detected in the brain after oral gavage are almost 3-fold higher than compound 56 (FIG. 13A), underscoring beneficial pharmacokinetic properties endowed from fluorination of compound 56. Both compounds 121 and 56 protected against α-syn PFF-induced cell death in mouse cortical neurons with minor differences in the potency (FIG. 13B). Also, compounds 121 and 56 inhibited PAAN/MIF nuclease activity against its substrates and protected against genomic DNA cleavage in mouse cortical neurons treated with α-syn PFF (FIGS. 13C and 13D). Since the rapafucins contain the FK506 binding protein (FKBP) binding domain, rapamycin and FK506 that have the same structural feature were evaluated against PAAN/MIF nuclease activity and MNNG-induced parthanatic cell death. Both rapamycin and FK506 failed to inhibit PAAN/MIF nuclease activity and MNNG-induced parthanatic cell death, while compound 121 blocked PAAN/MIF nuclease activity (FIG. 14A) and MNNG-induced parthanatic cell death (FIG. 14B). In addition, compound 121 failed to inhibit rapamycin sensitive pS6 kinase activity (FIG. 14C) and FK506 sensitive calcineurin activity (FIG. 14D). Compound 121, like compound 56, failed to inhibit ABT-888 sensitive PARP activity, STS-induced apoptotic cell death and TNFα and Z-VAD-induced necroptosis (FIGS. 14E-14G). Taken together, compound 121 is a potent and selective inhibitor for PAAN/MIF nuclease that prevents α-syn PFF-induced cell death.

Next, we attempted to gain deeper insight how compound 121 inhibits PAAN/MIF nuclease. Compound 121 does not disrupt the interaction of PAAN/MIF with its substrate, thus it is likely acting as an allosteric inhibitor (FIG. 15A). The binding affinity of PAAN/MIF for compound 121 was determined using biolayer interferometry with a K_{d} value of 1.56 μM (FIG. 4E). Compound 97, an analogue of compound 56 has no effect on PAAN/MIF nuclease activity and MNNG-induced parthanatic cell death (FIGS. 15B and 15C), consistent with its lack of binding to PAAN/MIF (FIG. 15D). A 3-D representational model of the PAAN/MIF trimer, an active form of PAAN/MIF nuclease, bound to compound 121 was constructed (FIG. 15E). Based on the model, PAAN/MIF amino acids 60-63, 66-67, 72, 97-99 and 105 are potential compound 121 binding sites. Alanine substitutions were made for each residue surrounding the putative binding site. The nuclease activity of PAAN/MIF S60A, L61A, H62A and S63A (MIF 60-63A) and PAAN/MIF N72A became resistant to compound 121, while that of K66A and I67A (MIF 66-67A) or N97A, Y98A and Y99A (MIF 97-99A) remained sensitive to inhibition by compound 121 as the wild type PAAN/MIF. PAAN/MIF N105A is devoid of PAAN/MIF nuclease activity (FIGS. 4F and 15F). Individual PAAN/MIF mutants (S60A, L61A, H62A, S63A, K66A, I67A, N72A, N97A, Y98A, Y99A, N105A) were made and tested against MNNG-induced parthanatic cell death treated with compound 121 in PAAN/MIF KO SH-SY5Y cells in the absence and presence of the latter mutants in comparison to WT, PAAN/MIF 60-63A, PAAN/MIF 66-67A and PAAN/MIF 97-99A. Compound 121 protection is significantly reduced in the PAAN/MIF L61A, PAAN/MIF 60-63A and PAAN/MIF N72A mutants (FIG. 4G). The direct binding of the PAAN/MIF WT and alanine mutants was evaluated against compound 121. PAAN/MIF WT and PAAN/Y99A mutant exhibited binding to compound 121 while the binding was remarkably decreased for the mutants L61A, N72A and 60-63A. These results indicate that L61 and N72 residues are involved in the direct binding of PAAN/MIF with compound 121 (FIG. 4H).

Two doses (5 and 15 mg/kg/day) of compound 121 were evaluated in the intrastriatal α-syn PFF model (FIG. 5A). Mice tolerated the compound 121 with no overt toxicity or weight loss (FIG. 16A). Compound 121 at both doses prevents the loss of DA neurons as assessed by stereologic counting of TH immunoreactivity (FIGS. 5B and 5C) and Nissl staining (FIGS. 5B and 5D), immunoblot analysis of TH levels (FIGS. 11B and 11C), HPLC assessment of DA (FIG. 5E) and DOPAC (FIG. 5F) and the accompanying behavioral deficits including the pole test (FIGS. 5G and 16D) and grip strength test (FIGS. 5H and 16E). Pulse gel electrophoresis indicates that α-syn PFF induces large scale genomic DNA cleavage in WT, while it is significantly attenuated in mice treated with compound 121 (FIG. 5I). Taken together, PAAN/MIF nuclease inhibition by compound 121 protects against α-syn PFF-induced neurodegeneration in PD.

Prevention of tissue injury following renal or cardiac ischemia is a large unmet medical problem. PARP inhibitors or genetic deletion of PARP-1 were found to reduce tissue injury following renal or cardiac ischemia. The present disclosure explored whether the PARP-1 dependent AIF-associated nuclease (PAAN), Macrophage Migration Inhibitory Factor (MIF) plays a role in renal and cardiac ischemia. We find that MIF knockout or MIF mutant lacking nuclease activity (MIF E22Q) attenuates ischemia reperfusion induced cardiac dysfunction (FIGS. 18A-D). We also find that MIF knockout or MIF mutant lacking nuclease activity (MIF E22Q) attenuates ischemia reperfusion induced renal dysfunction (FIGS. 19A-B). In summary, MIF's nuclease activity is a potential critical therapeutic target for preventing the cell death and tissue injury that occurs in renal and cardiac ischemia.

The present disclosure discloses that pathologic α-syn cell death is mediated through PAAN/MIF nuclease activity. We have demonstrated that inhibition of PAAN/MIF nuclease activity both genetically and pharmacologically, while retaining MIF's tautomerase activity prevents pathologic α-syn toxicity both in vitro and in vivo, indicating that PAAN/MIF's nuclease activity is a novel and attractive therapeutic target for PD and other disorders where parthanatos plays a role. The first class of PAAN/MIF nuclease inhibitors is reported and is shown to prevent pathologic α-syn-induced loss of DA neurons and the accompanying behavioral deficits. This PAAN/MIF nuclease inhibitor, compound 121 appears to act as an allosteric inhibitor since it interacts with amino acids L61 and N72 that are way from the nuclease active site E22 in preventing PAAN/MIF nuclease activity. Compound 121 clearly shows a unique ability in inhibiting parthanatos since it has no effect on other types of cell death such as apoptosis and necroptosis. Furthermore, it does not affect the upstream activator of parthanatos, PARP-1, thus it is not likely to affect DNA damage repair, gene transcription and cell proliferation. PAAN/MIF does not share homology with other human DNases and its nuclease activity is separated from its tautomerase activity making it possible to develop a specific therapeutic target that should have a high safety profile. Since parthanatos play a prominent role in a wide range of neurologic diseases, including stroke, Parkinson's disease and Alzheimer's disease, the therapeutic utility of PAAN/MIF nuclease inhibition will likely extend to other forms of neurodegeneration and cell death where parthanatos plays a role.

The following example is provided to further illustrate the advantages and features of the present disclosure, but it is not intended to limit the scope of the disclosure. While this example is typical of those that might be used, other procedures, methodologies, or techniques known to those skilled in the art may alternatively be used.

### EXAMPLES

### Example 1

Compound Synthesis

Reactions were carried out in oven-dried glassware. All reagents were purchased from commercial sources and were used without further purification unless noted. Unless stated otherwise, all solution-phase reactions were carried out under a positive pressure of argon monitored by Merck precoated silica gel 60F-254 plates and visualized using 254 nm UV light or mass spectra. Solid-phase reactions were carried out on chlorotrityl polystyrene resin purchased from Rapp Polymere. Column chromatography was performed on silica gel (RediSep Rf Flash columns, ISCO). The ratio between silica gel and crude product ranged from 100 to 50:1 (w/w). Mass spectra and purity values were obtained with an Agilent 1260 HPLC-MS system with an Agilent 6120 single quadrupole mass detector, and an Agilent Pursuit XRs 100 Å diphenyl column. The eluant consisted of acetonitrile and water, both with 0.1% formic acid.

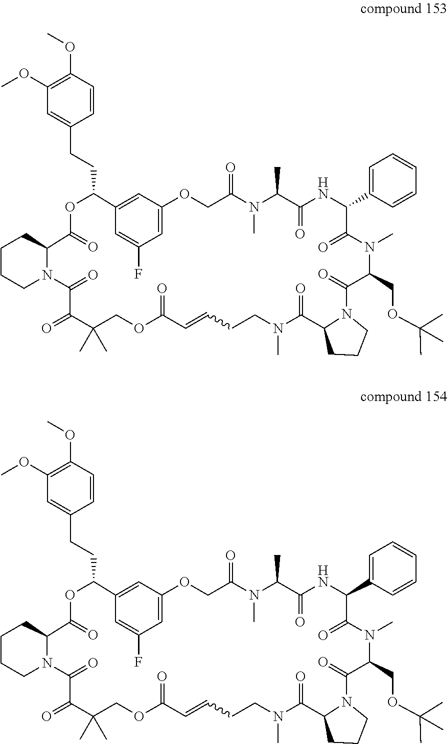 [SMILES: COc1ccc(CC[C@H]2OC(=O)[C@@H]3CCCCN3C(=O)C(=O)C(C)(C)COC(=O)C=CCCN(C)C(=O)[C@@H]3CCCN3C(=O)[C@H](COC(C)(C)C)N(C)C(=O)[C@@H](c3ccccc3)NC(=O)[C@H](C)N(C)C(=O)COc3cc(F)cc2c3)cc1OC ; COc1ccc(CC[C@H]2OC(=O)[C@@H]3CCCCN3C(=O)C(=O)C(C)(C)COC(=O)C=CCCN(C)C(=O)[C@@H]3CCCN3C(=O)[C@H](COC(C)(C)C)N(C)C(=O)[C@H](c3ccccc3)NC(=O)[C@H](C)N(C)C(=O)COc3cc(F)cc2c3)cc1OC]

Synthesis and spectroscopic data of compounds 153 and 154. [SMILES CODE: O═C([C@@H]1CCCCN1C(C(C(C)(C)COC(/C≡CCCN(C)C([C@H]2N(C([C@@H]3COC(C)(C)C)═O)CCC2)═O)═O)═O)═O)O[C@@H](C4═CC(F)═CC(OCC(N(C)[C@@H](C)C(N[C@@H](C5═CC═CC≡C5)C(N3C)═O)═O)═O)═C4)CCC6═CC(OC)═C(OC)C≡C6 and O═C(O[C@@H](C1═CC(OCC(N2CCC[C@H]2C(N(C)[C@H](C(N[C@H](C₃═CC═CC≡C₃)C4 ═O)═O)COC(C)(C)C)═O)═O)═CC(F)═C1)CCC5═CC(OC)═C(OC)C≡C5) [C@H]6N(C(C(C(C)(C )COC(C═CCCN(C([C@@H](N4C)C)═O)C)═O)═O)═O)CCCC6]

20 g of cis-C6 linker loaded resin (Loading Capacity=0.289 mmol/g) was taken in a 250 ml of SPPS vessel and swelled for 30 min with DCM (100 ml) on laboratory shaker (Kamush® LP360AMP, 360°, speed 6), then filtered and washed with DMF (200 ml×2) and dried for 5 min. For each amino acid, a solution of Fmoc-AA (3eq) and HATU (3eq) in 50 ml of DMF was added to the resin in 50 ml of DMF. Then DIEA (6eq) in 25 ml of DMF was added and shaken for 3 hrs. Solvent was filtered and washed with DMF (100 ml×5) and DCM (100 ml×5)* and dried, if necessary, stored at <4° C. 100 ml of 20% Piperidine in DMF was added and shaken for 20-30 min, filtered and again 100 ml of 20% Piperidine in DMF was added and shaken for 20-30 min. Solvent was filtered and washed carefully with DMF (100 ml×5) and dried, then immediately taken for next Fmoc-AA coupling. The first amino acid was double coupled. The Fmoc group from the Tetrapetide was deprotected (20% Piperidine in DMF) and peptide was removed from the resin using 3% TFA in DCM for 5 min (8 g of resin X 3). Obtained light yellow crude (3 individual batches) was subjected in to reversed phase column chromatography (130 g X 3 times) using 5% to 20% of ACN (20 to 30 CVs) in water to separate the diastereomers, R (compound 153) and S (compound 154).

A solution of 1.2eq FKBD and HATU in 10 ml of DMF/DCM (10 ml) was added to the solution of 711 mg of Tetrapeptide Amine in 10 ml of DCM. DIEA was added and stirred for 3 hrs at RT. After confirming reaction completion with LCMS, reaction mixture was diluted with 100 ml of EtOAc and washed with water (100 ml×2) and Brine (50 ml). The organic layer was dried over anhydrous sodium sulphate and concentrated to dryness and was subjected to column chromatography using hexane/EtOAc (1:1) mixture. An off-white foam was dissolved in degassed EtOAc (100 ml), Zhan 1B cat (10 mol %) was added and refluxed for 3 hrs. The catalyst was filtered and EtOAc layer was washed with water and brine (100 ml), then dried and concentrated to dryness. The residue was subjected to normal phase column chromatography (0 to 8% MeOH in DCM, 80 g column) and further purified using reverse phase column chromatography (10% to 90% ACN in Water, 130 g C18). Pure fractions were pooled and concentrated to get off-white powder. The powder was dissolved in 5-6 ml of Me-THF and carefully dripped into 50 ml of Heptane. The obtained precipitate was filtered and dried to get white powder of desired compound.

### Example 2

Therapeutic Potential of PAAN Inhibition for Parkinson's Disease

Animals. C57BL/6 WT, PAAN/MIF KO, MIF P1G KI mice were obtained from the Jackson Laboratories. For generation of PAAN/MIF E22Q KI mice, the linearized targeting vector containing the mutated site (E22Q) of PAAN/MIF in exon1 (pDTA-LC-069, AscI) were microinjected into the ES cells and transferred into pseudo-pregnant female mice as previously described. Using genomic DNA prepared from tail snip (Proteinase K, Roche Diagnotics; direct PCR lysis, Viagen), pups were genotyped by PCR (GoTag Green Master Mix, Promega) using primers (forward: GGGAGAAATTAATAGTGTGCTCCAG; reverse: CTCAGGGACCTGCTGTGATT G). Positive founders were further confirmed by Sanger sequencing. All housing, breeding, and procedures were performed according to the NIH Guide for the Care and Use of Experimental Animals and approved by Johns Hopkins University Animal Care and Use Committee.

Preparation of α-syn PFF. Recombinant mouse α-synuclein protein were prepared as previously described. See Kam et al. (2018) Science 362:eaat8407. After purification, bacterial endotoxins were eliminated by Toxineraser endotoxin removal kit (GeneScript). α-syn PFF were prepared by agitating (1,000 rpm at 37° C.) for 7 days and sonicated for 30 s (0.5 sec pulse on/off) at 10% amplitude (Branson Digital Sonifier).

Stereotaxic injection of α-syn PFF. Two to 3-month-old mice were anaesthetized with a mixture of ketamine (100 mg/kg) and xylazine (20 mg/kg) and fixed in a stereotaxic instrument. PBS or α-syn PFF (5 μg/2 μl) was unilaterally injected into striatum [anteroposterior (AP)=+0.2 mm, mediolateral (ML)=+2.0 mm, dorsoventral (DV)=+2.8 mm from bregma]. The infusion was performed at a rate of 0.4 μl/min and the needle was left in place for 5 min for a complete absorption of the solution. After surgery, recovery of animals were monitored the following day of injection. Behavioral tests were performed 6 months after injection and mice were euthanized for biochemical and histological analysis. For biochemical analysis, tissues were immediately dissected and frozen at −80° C. For histological analysis, animal were perfused with ice-cold PBS followed by 4% paraformaldehyde. Brains were post-fixed with 4% paraformaldehyde and cryoprotected in 30% sucrose.

Behavior tests. The behavioral deficits in α-syn PFF injected WT, PAAN/MIF KO, E22Q KI or P1G KI mice, in α-syn PFF injected WT or PAAN/MIF KO mice expressing AAV2-Flag-PAAN/MIF WT, E22Q or P1G and in α-syn PFF injected WT mice delivered with compound 121 were assessed by the pole test, grip strength test or clasping test 1 week prior to sacrifice. The experimenters were blinded to genotype or treatment condition.

Pole test. The 9 mm diameter pole is a 2.5 ft metal rod wrapped with bandage gauze. Before the actual test, the mice were trained for two consecutive days and each training session consisted of three test trials. On the day of the test, mice were placed 3 inch from the top of the pole facing head-up. The time to turn and total time to reach the base of the pole were recorded. The maximum cutoff of time to stop the test and recording was 60 s.

Grip strength test. Neuromuscular strength was measured by maximum holding force developed by the mice using a grip-strength meter (Bioseb). Mice were placed onto a metal grid to grasp with either fore or both limbs that are recorded as ‘fore limb’ and ‘fore and hindlimb’, respectively. The tail was gently pulled and the maximum holding force was recorded by the force transducer before the mice released their grasp on the grid. The peak holding strength was digitally recorded and displayed as force in grams (g).

Clasping test. The hindlimb clasping test is used to show decrease of motor function. The test was performed by grasping the mouse tail and hindlimb clasping was monitored for 10 s. See Taylor et al. (2010) Behav. Brain Res. 211:1-10. Hindlimb clasping was scored as follows: 0, normal (hindlimbs consistently splayed outward, away from the abdomen); 1, one or two hindlimbs partially retracted toward the abdomen for more than 50% of the time; 2, both hindlimbs fully retracted toward the abdomen for more than 50% of the time; 3, hindlimbs pulled into the body and clasped by the forelimbs.

Measurement of dopamine and derivatives. Biogenic amine concentrations were measured by high-performance liquid chromatography (HPLC) with electrochemical detection (HPLC-ECD). Briefly, mice were decapitated and the striatum was quickly removed from the brain. Striatal tissue was weighed and sonicated in ice-cold 10 μM perchloric acid containing 0.01% EDTA (wt/vol). The 60 ng of 3,4- dihydroxybenzylamine (DHBA) was used as an internal standard. After centrifugation (15,000×g, 30 min, 4° C.), the supernatant was cleaned through a 0.2 μm filter and 20 μl of the supernatant was injected into HPLC column (3 mm×150 mm C-18 reverse phase column, Acclaim™ Polar Advantage II, Thermo Scientific) and analyzed by a dual channel Coulchem III electrochemical detector (Model 5300, ESA, Inc. Chelmsford, MA). The protein concentrations of tissue homogenates were measured using the BCA protein assay kit (Pierce). Data were normalized to protein concentrations (ng/mg of neurotransmitters/tissue).

Immunohistochemistry (IHC) and immunofluorescence (IF) were performed on 50 μm thick serial brain sections including substantia nigra and striatum. Every 4th section was utilized for analysis. Primary antibodies and working dilutions are detailed in Table 3. For histological studies, free-floating sections were blocked with 10% goat serum in PBS with 0.2% Triton X-100 and incubated with a 1:1000 dilution of rabbit polyclonal anti-TH (Novus) and visualized with biotinylated goat anti-rabbit IgG, followed by streptavidin-conjugated horseradish peroxidase (HRP) (Vectastain ABC kit, Vector Laboratories). The sections were visualized with SigmaFast DAB peroxidase substrate (Sigma-Aldrich). Sections were counterstained with Nissl (0.09% thionin). Total numbers of TH- and Nissl-stained neurons in the substantia nigra pars compacta were counted by an investigator who was blind to genotypes or treatment condition with randomly allocated group using the optical fractionators, the unbiased method for cell counting by the computer-assisted image analysis system consisting of an Axiophot photomicroscope (Carl Zeiss) equipped with a computer controlled motorized stage (Ludl Electronics), a Hitachi HV C20 camera, and Stereo Investigator software (MicroBright-Field). For Nissl counting, a cell was defined as bright blue-stained neuronal perikarya with a nucleolus. See Karuppagounder et al. (2014) Sci. Rep. 4:4874. For immunofluorescent studies, sections with 1:1000 dilution of mouse monoclonal anti-Flag (Clone M1, Sigma) antibodies were incubated with a Alexa-fluor 488-conjugated secondary antibodies (Invitrogen). The fluorescent images were acquired by confocal scanning microscopy (LSM710, Carl Zeiss). All the images were processed by the Zen software (Carl Zeiss).

TABLE 3
The antibodies used in this disclosure.

| Antibody | Source | Identifier | Dilution |
|---|---|---|---|
| PAR | Dawson lab | N/A | 1:2,000 (WB) |
| PARP-1 | BD Bioscience | 611039 | 1:2,000 (WB) |
| PAAN/MIF | Abcam | 7207 | 1:3,000 (WB) |
| | | | 1:500 (IF) |
| AIF | Santa Cruz | 13116 | 1:3,000 (WB) |
| | | | 1:500 (IF) |
| HSP60 | Cell Signaling | D307 | 1:1,000 (WB) |
| Flag | Sigma | Clone M1 | 1:3,000 (WB) |
| | | | 1:1000 (IHC) |
| pS6K | Cell Signaling | 9205 | 1:3,000 (WB) |
| S6K | Cell Signaling | 2708 | 1:3,000 (WB) |
| pS6 | Cell Signaling | 2215 | 1:1,000 (WB) |
| S6 | Cell Signaling | 2217 | 1:3,000 (WB) |
| p4E-BP1 | Cell Signaling | 2855 | 1:1,000 (WB) |
| 4E-BP1 | Cell Signaling | 9644 | 1:3,000 (WB) |
| TH | Novus Biologicals | NB300-19 | 1:2,000 (WB) |
| | | | 1:1,000 (IHC, IF) |
| DAT | Sigma | D6944 | 1:1,000 (WB) |
| β-actin-HRP | Sigma | A3854 | 1:20,000 (WB) |

Below is a illustration of the abbreviations used in Table 3. PAR, Poly (ADP-ribose); PARP-1, Poly (ADP-ribose) polymerase-1; MIF, Macrophagy migration inhibitory factor; AIF, Apoptosis inducing factor; HSP60, Heat shock protein 60; pS6K, phospho-p70 S6 kinase; S6K, p70 S6 kinase; pS6, phopho-S6 ribosomal protein; S6, S6 ribosomal protein; p4E-BP1, phospho-4E-BP1; TH, Tyrosine hydroxylase; DAT, Dopamine transporter; HRP, Horseradish peroxidase; WB, western blot; IHC, immunohistochemistry; IF, immunofluorescence; N/A, not applicable.

Tissue lysate preparation and western blot analysis. Dissected brain regions of interest were homogenized and prepared in lysis buffer [50 mM Tris-HCl (pH 7.4), 150 mM NaCl, 1 mM EDTA, 1% Triton x-100, 0.5% SDS, 0.5% sodium-deoxycholate, phosphatase inhibitor mixture I and II (Sigma-Aldrich, St. Louis, Mo.), and complete protease inhibitor mixture (Roche, Indianapolis, Ind.)]. The samples were rotated at 4° C. for 30 min for the complete lysis and centrifugated at 15,000×g for 30 min. Protein concentrations were determined using the BCA assay (Pierce) and samples were separated using SDS polyacrylamide gels and transferred onto nitrocellulose membranes. The blots were blocked with 5% non-fat milk in TBS-T (Tris buffered saline with 0.1% Tween—20) for 1 h, probed using various primary antibodies. Target antigens were incubated with appropriate HRP-conjugated secondary antibodies (Cell signaling) and were visualized by ECL substrate.

Preparation and quantification of genomic DNA. The striatum was rapidly removed from the brain and lysed by repetitive pipetting with a micropipette in lysis buffer [100 mM NaCl, 10 mM Tris-HCl (pH 8.0), 25 mM EDTA (pH 8.0), 0.5% SDS] with 0.2 mg/ml proteinase K (Roche Diagnotics). Samples were incubated 55° C. for overnight and further incubated 85° C. for 45 m to inactivate proteinase K. The genomic DNA were immediately separated on a 1.2% pulse field certified agarose in 0.5×TBE buffer with initial switch time of 1.5 s and a final switch time of 3.5 s for 12 h at 6 V/cm. The gel was then stained with 0.5 mg/ml Ethidium Bromide (EtBr) and visualized on UV light. Noncleaved genomic DNA was quantified as percentage (%) of the total genomic DNA that included both noncleaved genomic DNA and cleaved genomic DNA in each individual group.

Cell culture, transfection, primary neuronal culture and treatment. SH-SY5Y cells (ATCC) were cultured in DMEM containing 10% fetal bovine serum and penicillin/streptomycin at 37° C. under 5% C02. The cells were transfected using PolyFect reagent (Qiagen). Parthanatos was induced by the treatment of 50 μM MNNG for 15 min in SH-SY5Y cells. ABT-888 or PAAN/MIF inhibitors were pretreated with indicated concentration 1 h before MNNG treatment and cell death were assessed after 24 h. Primary cortical neurons from WT, PAAN/MIF KO, E22Q KI or P1G KI embryos were prepared as previously described See Kam et al. (2018) Science 362:eaat8407. Briefly, the cortex was dissected and cultured at embryonic day 16 in neurobasal media supplemented with B-27, 0.5 mM L-glutamine, penicillin and streptomycin (Invitrogen). Primary neurons were infected with AAV2-control, AAV2-PAAN/MIF WT, AAV2-PAAN/MIF E22Q, AAV2-PAAN/MIF E22A or AAV2-MIF P1G (ViGene Biosciences) at days in vitro (DIV) 4-5. ABT-888 or PAAN/MIF inhibitors were applied to neurons 1 h before α-syn PFF treatment. The neuron growth medium was replaced with fresh medium alone or including inhibitors every 3-4 days. α-syn PFF was added at DIV 7 and further incubated for indicated times followed by the cell death assay or biochemical experiments.

Preparation of human cortical neurons. Human H1 embryonic stem cells (ESCs) were differentiated into cortical neurons as previously described. See Xu et al. (2016) Sci. Transl. Med. 8:333ra48. Briefly, ESC colonies detached from mouse embryonic fibroblasts (MEFs) were grown in suspension in human ESC medium without FGF2 for 6 days in low-attachment six-well plates (Corning). On day 7, free-floating embyroid bodies (EB)s were transferred to Matrigel coated plates to allow the complete attachment of EB and formation of rosette neuronal aggregates (RONAs). RONAs were manually microisolated and maintained as neurospheres for 1 day and then dissociated into single cells and plated on laminin/poly-D-lysine-coated plates for further experiments. For neuronal differentiation, retinoic acid (2 μM), SHH (50 ng/ml), purmorphamine (2 μM), or the combination of retinoic acid, SHH, and purmorphamine was supplemented in neural differentiation medium containing Neurobasal/B27 (NB/B27; Invitrogen), brain-derived neurotrophic factor (BDNF; 20 ng/ml; PeproTech), glial cell line-derived neurotrophic factor (GDNF; 20 ng/ml; PeproTech), ascorbic acid (0.2 mM; Sigma), dibutyryl adenosine 3′, 5′-monophosphate (cAMP; 0.5 mM; Sigma).

Cell death and viability assessment. Primary cultured cortical neurons were treated with 5 μg/ml of α-syn PFF for 14 days and cell death was determined by staining of all nuclei with 7 μM Hoechst 33342 and dead cell nuclei with 2 μM propidium iodide (PI) (Invitrogen). The numbers of total and dead cells were counted with the Axiovision 4.6 software (Carl Zeiss). Cell viability was determined by fluorescence at an excitation wavelength 570 nm and an emission wavelength 585 nm using Alamar Blue (Invitrogen).

Immunoprecipitation (IP). 1 mg of whole-cell lysates were incubated with AIF antibody (1 mg/ml, Santa Cruz Biotechnology) overnight, followed by incubation with PureProteone kappa Ig binder magnetic beads (Millipore) for 3 h, at 4° C. The IP complexes were washed 5 times and then denatured by boiling for 5 min after adding 2× Laemlli buffer plus β-mercaptoethanol. The samples were analyzed by western blot analysis with mouse anti-Flag antibody (Clone M1, Sigma) or rabbit anti-PAAN/MIF (Abcarn).

Subcellular fractionation. Subcellular fraction and quantification of relative levels of AIF and PAAN/MIF were performed as previously described. Se Wang et al. (2016) Science 354:6308. Briefly, primary cortical neurons treated with α-syn PFF or PAAN/MIF KO neurons transduced with AAV2-PAAN/MIF WT, PAAN/MIF E22Q or PAAN/MIF E22 Å were subjected to subcellular fractionation into nuclear extracts (N) and postnuclear cell extracts (PN), which is the fraction prepared from whole-cell lysates after removing nuclear proteins using hypotonic buffer. See Yu e al. (2002) Science 297:259-263. Each fractions were monitored by PARP-1 antibody (BD) for the nuclear fraction and HSP60 antibody (cell signaling) for the postnuclear fraction.

Screening strategy. Amine modified PAAN/MIF RF substrate (Sequence ID NO 2:5′—NH₂-TCCCAAGTAGCTGGGATTACAGGAAAA AAA-3′) was immobilized on 96-well DNA-BIND plates (Corning) at a concentration of 100 nM in 100 μl of binding buffer [50 mM Na₂PO₄ (pH 8.5), 1 mM EDTA] at 37° C. After 1 h, the plates were washed three times with PBS to remove the uncoupled substrate and the plates were blocked by adding 200 μl of 3% BSA in binding buffer for 30 min at 37° C. A mixture of 8 μM PAAN/MIF protein and each of 10 μM compound in enzyme reaction buffer [10 mM Tris-C₁ (pH 7.0) and 10 mM MgCl₂] was added. After 1 h incubation for PAAN/MIF's enzyme reaction, the plates were washed three times with PBS followed by a hybridization reaction adding the biotin labeled complementary DNA (Sequence ID NO 3:5′-biotin-TTTTTTTCCTGTAA-3′) at a 100 nM concentration in 100 μl of hybridization solution (5×SSC and 0.1% SDS) for 1 h at 55° C. The plates were washed harshly with preheated washing solution (2×SSC and 0.1% SDS) three times, soaked for 5 m and blocked with 3% BSA in binding buffer for 30 m. The biotin labeled complementary DNA were incubated with 1:1000 dilution of horseradish peroxidase (HRP)-conjugated Streptavidin (Thermo scientific) for 30 m at 37° C. and were monitored for colorimetric changes by adding 1-step Ultra TMB-ELISA substrate solution (Thermo scientific). Each plate contains three reactions with or without PAAN/MIF protein as controls. In order to assess high-throughput screening (HTS) readiness and robustness of our assay, coefficient of variation (CV), signal-to-background ratio (S/B) and Z′ factor were calculated by the value of the reaction without PAAN/MIF and the reaction with PAAN/MIF in each plate. For the secondary screening, SH-SY5Y cells were plated into 96 well plates with 10,000 cells/well. On the next day, each compound was added 1 h before MNNG treatment. Cells were treated with MNNG (50 μM) for 15 min and then further incubated in normal medium including compounds for 24 h. Cell viability was determined using Alamar Blue as described above.

Nuclease assay. Purified PAAN/MIF protein (4 μM) was incubated with compounds (10 μM) in the nuclease buffer [10 mM Tris-C₁ (pH 7.0) and 10 mM MgCl₂] for 20 m on ice. Then samples were added with 1 μM of PAAN/MIF substrate (PS30 or RF) and incubated for 1 h at 37° C. The reaction was terminated with loading buffer containing 10 mM EDTA and separated on 15% TBE-urea polyacrylamide (PAGE) gel or 20% TBE PAGE gel. For other nucleases (EcoRI and EcoRV) assays, pcDNA3 (500 ng/reaction) was used as a substrate and 0.01 unit of Exonulcase III were incubated with small dsDNA (Sequence ID NO 4:5′-GTCACCGTCATACGACTC-3′ and Sequence ID NO 5:5′-GAGTCGTATGACGGTGAC-3′).

Purification of PAAN/MIF recombinant proteins. Human PAAN/MIF (NM_002415) cDNA and its variants were subcloned into pGEX-6β-1 vector (GE Healthcare) and mutants were generated using a QuikChange site-directed mutagenesis kit (Stratagene). The sequences were confirmed by automated DNA sequencing. The protein was expressed and purified from Escherichia coli by glutathione sepharose (GE Healthcare). The GST tag was subsequently proteolytically removed. GST protein was used as a negative control in the nuclease assay and PAAN/MIF proteins purified by FPLC using Superdex 200 10/300GL column (GE Healthcare, Life Sciences) were also used in the nuclease assays.

Biolayer interferometry assay. The dose-dependent binding of compound 121 for PAAN/MIF WT or PAAN/MIF variants were determined by a biolayer interferometry assay using Octet RED96 (ForteBio). See Guo et al. (2018) Nat. Chem. 11:254-63. All the proteins used in this assay were tagged with GST. Anti-GST biosensor tips (ForteBio) were used to immobilize the GST proteins after prewetting with 1X kinetic buffer (ForteBio). The equilibrated GST biosensors were loaded with PAAN/MIF WT or variants (25 μg/ml). Background binding controls were measured by sensors that were incubated in buffer without proteins. All assays were performed by a standard protocol in 96-well black plates (Greiner Bio) with a total volume of 200 μl per well. All the data were analyzed by Octet data analysis software.

Electrophoretic mobility shift assay (EMSA). EMSA assays were performed using the Light-Shift Chemiluminescent EMSA kit (Thermo Scientific) following the manufactures instruction. Recombinant PAAN/MIF protein (2 μM) was incubated with 1-100 μM PAANIB-1 as indicated in the binding buffer containing 10 mM MgCl₂ for 10 min and further incubated with biotin-labeled DNA substrates (10 nM) for 30 min on ice. The mixture were separated on 6% retardation polyacrylamide and transferred to a Nylon membrane. Immunoblot analysis was performed to detect the biotin-labeled DNA using the Chemiluminescent Nucleic Acid Detection Module (Thermo Scientific).

In vitro ribosylation assay of PARP-1. 1 mg of recombinant PARP-1, activated DNA and NAD+(Trevigen) in the presence or absence of 1 μM of PARP-1 inhibitor, ABT-888 or PAAN/MIF inhibitors (compound 56 or 121) were incubated in the PARP assay buffer (Trevigen) for 30 m at room temperature and analyzed by immunoblot analysis using PAR antibody.

Calcineurin activity assay. Calcineurin activity was measured using a calcineurin cellular activity assay (Enzo Life Sciences) according to manufacturer's protocols. See Sangadala et al. (2019) Int. J. Mol. Sci. 20:1900. SH-SY5Y cells treated with FK506 or compound 121 were placed in a desalting column to remove excess phosphates and nucleotides. Total phosphate activity was determined in the samples by incubating with a calcineurin-specific substrate supplied in the manufacturer and measured by monitoring absorbance at 620 nm. Calcineurin activity was calculated as the difference between total phosphatase activity minus the phosphatase activity in the presence of 10 mM EGTA that blocks calcineurin activity.

Oxidoreductase activity assay. The thiol-protein oxidoreductase activity of PAAN/MIF was measured using insulin as the substrate as described previously. See Kudrin et al. (2006) J. Biol. Chem. 281:29641-51. Briefly, the insulin assay is based on the reduction of insulin and subsequent insolubilization of the insulin β-chain. The time-dependent increase in turbidity is then measured spectrophotometrically at 650 nm. The reaction was started by adding 5 μM PAAN/MIF WT in the presence or absence of 10 μM compounds (compounds 41, 56, 76, and 77) dissolved in 20 mM sodium phosphate buffer (pH 7.2), and 200 mM reduced glutathione (GSH) to ice-cold reaction mixture containing 1 mg/ml insulin, 100 mM sodium phosphate buffer (pH 7.2) and 2 mM EDTA. Insulin reduction was measured against the control solution (containing GSH) in the same experiment.

Tautomerase activity assay. The keto-enol tautomeric conversion of D-dopachrome methyl ester by PAAN/MIF was used as described previously. See Bendraft et al. (1997) Biochemistry 36:15356-62. Briefly, a fresh solution of D-dopachrome methyl ester was prepared by mixing 2 mM_{L}-3,4 dihydroxyphenylalanine methyl ester with 4 mM sodium peroxidate for 5 m at room temperature and then placed directly on ice before use. The enzymatic reaction was initiated at 25° C. by adding 20 μl of the dopachrome methyl ester substrate to 200 μl of PAAN/MIF WT (final concentration 5 μM) with or without 10 μM compounds (compounds 41, 56, 76, and 77) in tautomerase assay buffer (50 mM potassium phosphate, 1 mM EDTA, pH 6.0). To enhance substrate stability, excess sodium meta-periodate was removed using compound 56 disposable columns (Waters). The activity was determined by the decrease in absorbance at 475 nm using a spectrophotometer (Molecular Devices).

Preparation of the rapafucin compounds. Rapafucins, including compound 56 and compound 121 and other analogues were synthesized using solid-phase peptide synthesis and catalytic ring-closing metathesis and purified on a silica gel column. FKBD and cis-C6 linker conjugated resin was prepared as previously described. See Guo et al. (2018) Nat. Chem. 11:254-63. Modified FKBDs, including eFKBD-E1A24168 were prepared by BioDuro. Compound 121 used for in vivo studies was further purified using reverse-phase column chromatography to remove any metal catalyst residues. HPLC-MS was used to analyze purity and identification for all rapafucin compounds synthesized. HRMS was measured for key compounds for further verification. HRMS for compound 56 [M+H]+C62H83N6015, calculated: 1151.5916, observed: 1151.5900, HRMS for compound 121 [M+H]+C62H82FN6015, calculated: 1169.5822, observed: 1169.5802. Compound 121 or other PAAN/MIF inhibitors were dissolved in dimethyl sulfoxide (DMSO) (Sigma-Aldrich) for in vitro assays. Compound 121 was dissolved in a vehicle solution of 1% Tween 80 (Sigma-Aldrich), 10% polyethylene glycol (PEG 400, Fluka) and 1% hydroxy propyl methyl cellulose (HPMC, Sigma-Aldrich) for oral administration.

Measurement of compound 121 in brain. Compound 56, compound 77, and compound 121 were resuspended in a vehicle solution and were administered at 10 mg/kg by oral gavage. After 2 h, mice were perfused with PBS and whole brain was isolated, weighed and grounded with a pestle to a very fine powder with liquid nitrogen. Then, 2-folds of acetonitrile were added to brain tissue sample and the mixture was centrifuged at 14,000 rpm, 4° C. for 10 m. The supernatant was used for analysis. The concentration of compounds in brain tissue were measured using HPLC-MS on a C-18 reverse phase HPLC column. Separations were achieved using a linear gradient of buffer B from 40% to 95% in A (A=0.1% formic acid in H20; B=0.1% formic acid in CH₃CN) at a flow rate of 1 ml/min. Intracerebroventricular (ICV) injected brain samples at 0.5, 1, 2, 4 or 8 μM concentration were used for the standard curve.

Statistical analysis. All data are represented as mean s.e.m. with at least 3 independent experiments. Statistical analysis was performed using GraphPad Prism 7 and statistical significance were reported in the relevant Figures and Figure legends. Differences between 2 means and among multiple means were assessed by unpaired two-tailed student t test and ANOVA followed by Tukey's post hoc test, respectively.

### Example 3

Compound Screening

A rapafucin library was synthesized as described in WO2017/136708 and Guo et al. (2018) Nat. Chem. 11:254-63. The hybrid macrocyclic library consisted of 45,000 compounds in pools of 15 individual compounds. The library compounds were screened for MIF inhibitory activity using a nuclease assay as detailed in FIG. 1 and below.

Nuclease assay: Human genomic DNA (200 ng/reaction, Promega), pcDNA (200 ng/reaction) or PS³⁰ and its related and non-related substrates (1 μM) was incubated with wild type MIF or its variants at a final concentration of 0.25-8 μM as indicated in 10 mM Tris-HCl buffer (pH 7.0) containing 10 mM MgCl₂ and 1 mM DTT or specific buffer as indicated, for 1 h (with pcDNA and small DNA substrates) or 4 h (with human genomic DNA) at 37° C. The reaction was terminated with loading buffer containing 10 mM EDTA and incubation on ice. The human genomic DNA samples were immediately separated on a 1.2% pulse field certified agarose in 0.5×TBE buffer with initial switch time of 1.5 s and a final switch time of 3.5 s for 12 h at 6 V/cm. pcDNA samples were determined by 1% agarose gel. Small DNA substrates were separated on 15% or 25% TBE-urea polyacrylamide (PAGE) gel or 20% TBE PAGE gel. Then gel was stained with 0.5 μg/ml Ethidium Bromide (EtBr) followed by electrophoretic transfer to nylon membrane. Then, Biotin-labeled DNA is further detected by chemiluminescence using Chemiluminescent Nucleic Acid Detection Module (Thermo Scientific).

Structure-activity relationship (SAR) analysis was then conducted on the compounds identified as having MIF inhibitory activity. Compounds having the structure of Formula (I) were then synthesized as shown in Example 1 and tested for MIF inhibitory activity using the nuclease assay described herein and neuronal protection via a cell death assay as detailed below.

Cell death assay: 200 μl of cells (HeLa cells or neuronal cells) culture (1×10⁴ cells/well) were plated into the wells of a sterile 96-well cell culture plate. The cells were incubated for 18 hours at 37° C. The medium was removed and changed with 100 μl/well of 1 μM test compound. Incubate for 1 hours at 37° C. Ten μl of 500 μM MNNG (N-Methyl-N′-nitro-N-nitrosoguanidine) was added to each well. The medium was aspirated well and and incubated for 15 min at 37° C. The cells were washed with the fresh medium. The medium was changed with 1 μM of test compound and incubated for 18 hours at 37° C. 10 μl/well of alamarBlue reagent was added directly to the well and incubated for 1 to 4 hours at 37° C. Fluorescence was measured using a fluorescence excitation wavelength of 560 nm and emission at 590 nm. The percentage of cell death was determined as the ratio of live to dead cells compared with the percentage of cell death in control wells to account for cell death attributed to mechanical stimulation of the cultures.

The results of both assays are set forth in Table 4 for compounds of the disclosure.

TABLE 4
The pharmacological data of the chemical
compounds disclosed herein.

| | (%) inhibition | |
|---|---|---|
| Compound | of MIF nuclease | (%) inhibition |
| No. | activity | of cell death |
| 1 | 25.3 | 43.6 |
| 2 | 15.3 | 41.1 |
| 3 | 21.0 | 40.6 |
| 4 | 10.8 | 39.7 |
| 5 | 10.7 | 41.7 |
| 6 | 30.8 | 46.5 |
| 7 | 30.1 | 54.5 |
| 8 | 31.3 | 61.6 |
| 9 | 16.5 | 57.4 |
| 10 | 56.8 | 57.2 |
| 11 | 25.6 | 48.9 |
| 12 | 28.9 | 52.0 |
| 13 | 21.9 | 46.7 |
| 14 | 11.2 | 41.6 |
| 15 | 8.7 | 53.4 |
| 16 | 64.3 | 85.2 |
| 17 | 63.7 | 78.3 |
| 18 | 65.0 | 78.4 |
| 19 | 59.9 | 62.4 |
| 20 | 42.2 | 60.6 |
| 21 | 57.9 | 61.0 |
| 22 | 61.8 | 56.9 |
| 23 | 45.7 | 58.7 |
| 24 | 40.3 | 57.4 |
| 25 | 25.8 | 59.1 |
| 26 | 64.9 | 71.2 |
| 27 | 78.4 | 72.1 |
| 28 | 66.6 | 69.6 |
| 29 | 12.4 | 57.8 |
| 30 | 18.6 | 51.5 |
| 31 | 22.9 | 52.3 |
| 32 | 11.2 | 54.3 |
| 33 | 12.6 | 53.4 |
| 34 | 24.2 | 58.4 |
| 35 | 21.5 | 56.3 |
| 36 | 18.5 | 51.0 |
| 37 | 18.0 | 59.6 |
| 38 | 35.9 | 55.7 |
| 39 | 31.1 | 57.5 |
| 40 | 37.4 | 53.8 |
| 41 | 82.0 | 80.6 |
| 42 | 40.8 | 53.2 |
| 43 | 45.6 | 54.8 |
| 44 | 30.0 | 52.2 |
| 45 | 15.4 | 51.9 |
| 46 | 21.7 | 52.5 |
| 47 | 52.3 | 54.2 |
| 48 | 61.5 | 49.2 |
| 49 | 14.7 | 60.0 |
| 50 | 18.0 | 56.4 |
| 51 | 14.2 | 53.3 |
| 52 | 9.9 | 61.0 |
| 53 | 14.1 | 58.3 |
| 54 | 15.9 | 56.0 |
| 55 | 13.6 | 59.9 |
| 56 | 70.8 | 81.5 |
| 57 | 18.4 | 59.3 |
| 58 | 16.5 | 56.1 |
| 59 | 13.3 | 58.9 |
| 60 | 59.4 | 55.4 |
| 61 | 19.2 | 53.8 |
| 62 | 13.1 | 53.7 |
| 63 | 39.0 | 54.1 |
| 64 | 27.2 | 52.3 |
| 65 | 20.1 | 54.4 |
| 66 | 67.0 | 71.4 |
| 67 | 76.3 | 73.7 |
| 68 | 44.0 | 58.0 |
| 69 | 20.7 | 55.7 |
| 70 | 25.2 | 53.8 |
| 71 | 25.3 | 57.0 |
| 72 | 21.2 | 55.3 |
| 73 | 40.2 | 53.0 |
| 74 | 31.5 | 52.6 |
| 75 | 34.9 | 52.8 |
| 76 | 73.5 | 79.2 |
| 77 | 75.5 | 77.9 |
| 78 | 9.0 | 44.7 |
| 79 | 6.7 | 43.4 |
| 80 | 10.3 | 49.5 |
| 81 | 12.3 | 49.8 |
| 82 | 8.5 | 47.0 |
| 83 | 12.0 | 49.8 |
| 84 | 10.8 | 51.0 |
| 85 | 10.7 | 49.4 |
| 86 | 22.7 | 53.0 |
| 87 | 17.1 | 51.3 |
| 88 | 21.3 | 52.7 |
| 89 | 12.6 | 49.9 |
| 90 | 15.8 | 48.5 |
| 91 | 25.8 | 62.7 |
| 92 | 9.0 | 72.1 |
| 93 | 14.6 | 66.8 |
| 94 | 12.0 | 12.1 |
| 95 | 17.5 | 23.9 |
| 96 | 32.7 | 69.1 |
| 97 | −5.3 | 41.4 |
| 98 | 5.1 | 29.6 |
| 99 | 14.0 | 32.3 |
| 100 | 13.4 | 26.1 |
| 101 | 9.1 | 31.5 |
| 102 | 59.5 | 77.7 |
| 103 | 16.8 | 22.5 |
| 104 | 13.2 | 20.4 |
| 105 | 57.2 | 66.3 |
| 106 | 11.9 | 13.1 |
| 107 | 20.0 | 15.1 |
| 108 | 4.2 | 20.4 |
| 109 | 9.3 | 52.9 |
| 110 | 20.9 | 59.3 |
| 111 | 6.8 | 48.9 |
| 112 | 19.2 | 48.3 |
| 113 | 26.8 | 66.1 |
| 114 | −2.4 | 44.9 |
| 115 | 8.7 | 47.9 |
| 116 | −4.7 | 50.9 |
| 117 | −5.1 | 51.8 |
| 118 | 9.0 | 50.8 |
| 119 | 19.7 | 60.7 |
| 120 | 7.1 | 45.8 |
| 121 | 71.2 | 90.1 |
| 122 | 10.7 | 64.3 |
| 123 | −5.1 | 82.4 |
| 124 | −2.7 | 55.9 |
| 125 | 2.4 | 63.4 |
| 126 | 6.7 | 46.8 |
| 127 | 5.0 | 46.4 |
| 128 | 11.0 | 47.4 |
| 129 | 29.5 | 43.0 |
| 130 | — | 48.2 |
| 131 | 26.9 | 43.0 |
| 132 | 20.8 | 43.0 |
| 133 | 16.2 | 42.1 |
| 134 | 11.9 | 41.7 |
| 135 | −5.9 | 44.8 |
| 136 | 21.3 | 41.9 |
| 137 | 9.4 | 44.5 |
| 138 | −4.8 | 47.0 |
| 139 | 7.1 | 48.6 |
| 140 | 0.0 | 51.0 |
| 141 | 9.2 | 46.8 |
| 142 | −4.7 | 42.5 |
| 143 | −1.1 | 46.7 |
| 144 | 10.7 | 51.3 |
| 145 | 6.7 | 46.6 |
| 146 | −1.1 | 43.9 |
| 147 | −4.7 | 46.2 |
| 148 | 12.0 | 50.7 |
| 149 | −11.8 | 51.0 |
| 150 | 10.7 | 50.8 |
| 151 | 6.8 | 45.7 |
| 152 | −2.4 | 43.0 |
| 153 | 71.7 | 93.2 |
| 154 | 38.2 | 46.2 |

A protection against parthanatos (cell death) greater than 2000 is considered as a meaningful protection against parthanatic cell death, since this would have meaningful clinical outcome in acute neurologic injury or in a chronic neurodegenerative disease.

All the rapapaanins that inhibit MIF nuclease activity greater than 2000 protect against parthanatos (cell death). There are three compounds, including compounds 92, 123, and 125, that have clear dissociation between protection against parthanatos (cell death) and MIF nuclease activity (i.e. they protect against parthanatos, but do not inhibit MIF nuclease activity).

### PROPHETIC EXAMPLES

DNA-Encoding Library

### Prophetic Example 1—Preparation of a Rapafucin DNA-Encoding Library Via Split-and-Pool Cycles

A rapafucin DNA-encoding library is synthesized by a sequence of split-and-pool cycles wherein the oligonucleotide is attached to the FKBD. First, an initial oligonucleotide of Formula (C) is synthesized and HPLC purified. A first building block comprising an FKBD building block is then covalently bound to the oligonucleotide of Formula (C) via click chemistry. Subsequently, a second oligonucleotide, encoding the first building block, is appended to the oligonucleotide of Formula (C). The resulting product is pooled and split into a second set of separate reaction vessels and a second building block comprising an effector domain building block is coupled to the first building block using a ring-closing reaction. The reaction is then encoded by the attachment of a unique oligonucleotide sequence to the unique oligonucleotide attached to the first building block. The encoded two-building-block molecules yields the final library.

### Prophetic Example 2—Preparation of a Rapafucin DNA-Encoding Library Via Split-and-Pool Cycles

A rapafucin DNA-encoding library is synthesized by a sequence of split-and-pool cycles wherein the oligonucleotide is attached to a linking region. First, an initial oligonucleotide of Formula (C) is synthesized and HPLC purified. Then, the oligonucleotide of Formula (C) is covalently bound to a first linking region via click chemistry. A first building block comprising an FKBD building block is encoded by a second oligonucleotide which is appended to the initial oligonucleotide of Formula (C). The resulting product is pooled and split into a second set of separate reaction vessels and a second building block comprising an effector domain building block is coupled to the first building block using a ring-closing reaction. The reaction is then encoded by the attachment of a unique oligonucleotide sequence to the unique oligonucleotide attached to the first building block. The encoded two-building-block molecules yields the final library.

### Prophetic Example 3—Preparation of a Rapafucin DNA-Encoding Library Via DNA-Recorded Synthesis and Ligation

A rapafucin DNA-encoding library is synthesized by DNA-recorded synthesis wherein the oligonucleotide is attached to the FKBD. First, an initial oligonucleotide of Formula (C) is synthesized and HPLC purified. A first building block comprising an FKBD building block is then covalently bound to the oligonucleotide of Formula (C) via click chemistry. Then, a second building block comprising an effector domain building block is coupled to the first building block via the first and second linking region through a ring-closing reaction. The reaction is encoded by DNA-recorded synthesis by ligation of a unique oligonucleotide to the initial oligonucleotide of formula (C).

### Prophetic Example 4—Preparation of a Rapafucin DNA-Encoding Library Via DNA-Recorded Synthesis and Enzymatic Reactions

A rapafucin DNA-encoding library is synthesized by DNA-recorded synthesis wherein the oligonucleotide is attached to the FKBD. First, an initial oligonucleotide of Formula (C) is synthesized and HPLC purified. A first building block comprising an FKBD building block is then covalently bound to the oligonucleotide of Formula (C) via click chemistry. Then, a second building block comprising an effector domain building block is coupled to the first building block via the first and second linking region through a ring-closing reaction. The reaction is then encoded by DNA-recorded synthesis by polymerase-catalyzed fill-in reactions.

### Prophetic Example 5—Preparation of a Rapafucin DNA-Encoding Library Via DNA-Templated Synthesis

A rapafucin DNA-encoding library is synthesized by DNA-templated synthesis. First, a second building block comprising an effector domain building block is coupled to the first building block comprising the FKBD via the first and second linking regions. Then, the reaction is encoded by DNA-templated synthesis, wherein a plurality of conjugate molecules of oligonucleotide-tagged building blocks are prepared and the spatial proximity of the two distinct oligonucleotides of Formula (C) facilitates the bimolecular chemical reactions between the two building blocks.

Those skilled in the art will recognize, or be able to ascertain, using no more than routine experimentation, numerous equivalents to the specific composition and procedures described herein. Such equivalents are considered to be within the scope of this disclosure, and are covered by the following claims.

1. A compound according to Formula (VII): 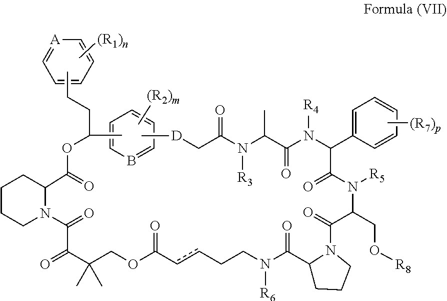
or an optically pure stereoisomer, pharmaceutically acceptable salt, or solvate thereof,
wherein
each A and B is independently CH or N;
D is selected from the group consisting of —O(CH₂)_{q}—, —S(CH₂)_{q}—, —COO(CH₂)_{q}—, —
CONR₃(CH₂)_{q}—, and —NR₃(CH₂)_{q}—;
q is an integer selected from 0 to 4;
each n, m, and p is independently an integer selected from 0 to 4;
“ [symbol] ” is a single bond or double bond;
each R₁, R₂, and R₇ is independently selected from the group consisting of H, F, Br, C₁, CF₃, CN, N₃, NH₂, NO₂, OH, OCH₃, methyl, ethyl, and propyl;
each R₃, R₄, R₅, and R₆ is independently selected from the group consisting of H, methyl, ethyl, propyl, and isopropyl; and
R₈ is selected from the group consisting of H, methyl, ethyl, n-propyl, isopropyl, n-butyl, isobutyl, sec-butyl, and tert-butyl.

2. The compound of claim 1, wherein the carbon connected to 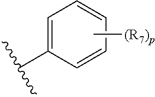
features R or S stereochemistry.

3. The compound of claim 1, wherein the compound is compound 121 with the following structure:

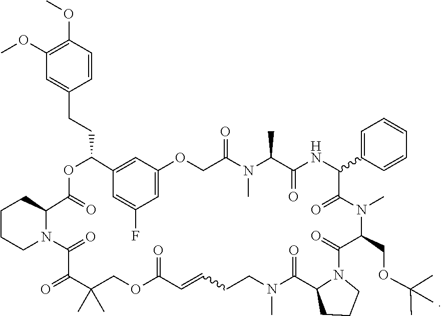 [SMILES: COc1ccc(CC[C@H]2OC(=O)[C@@H]3CCCCN3C(=O)C(=O)C(C)(C)COC(=O)C=CCCN(C)C(=O)[C@@H]3CCCN3C(=O)[C@H](COC(C)(C)C)N(C)C(=O)C(c3ccccc3)NC(=O)[C@H](C)N(C)C(=O)COc3cc(F)cc2c3)cc1OC]

4. The compound of claim 1, wherein the compound is compound 153 with the following structure:

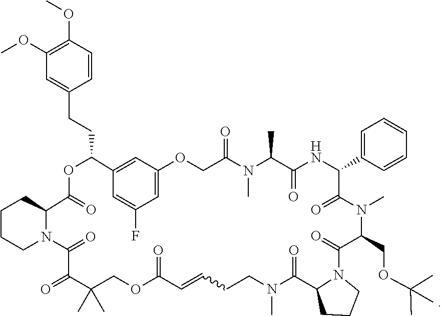 [SMILES: COc1ccc(CC[C@H]2OC(=O)[C@@H]3CCCCN3C(=O)C(=O)C(C)(C)COC(=O)C=CCCN(C)C(=O)[C@@H]3CCCN3C(=O)[C@H](COC(C)(C)C)N(C)C(=O)[C@@H](c3ccccc3)NC(=O)[C@H](C)N(C)C(=O)COc3cc(F)cc2c3)cc1OC]

5. The compound of claim 1, wherein the compound is compound 154 with the following structure:

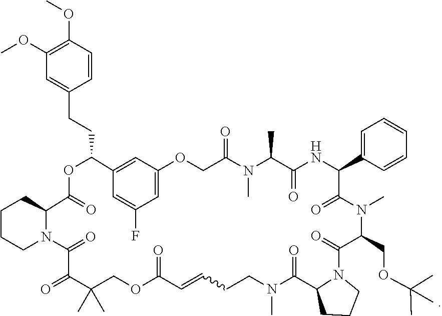 [SMILES: COc1ccc(CC[C@H]2OC(=O)[C@@H]3CCCCN3C(=O)C(=O)C(C)(C)COC(=O)C=CCCN(C)C(=O)[C@@H]3CCCN3C(=O)[C@H](COC(C)(C)C)N(C)C(=O)[C@H](c3ccccc3)NC(=O)[C@H](C)N(C)C(=O)COc3cc(F)cc2c3)cc1OC]

6. The compound of claim 1, wherein the compound is an inhibitor of macrophage migration inhibitory factor (MIF) nuclease activity and an inhibitor of parthanatos mediated cell death.

7. The compound of claim 1, wherein the compound is an inhibitor of parthanatos mediated cell death.

8. A pharmaceutical formulation, comprising an effective amount of the compound according to claim 1 and a pharmaceutically acceptable carrier.

9. A method of inducing a neuroprotective response in a cell, the method comprising contacting the cell with a therapeutically effective amount of the compound according to claim 1.

10. The method of claim 9, wherein the cell is a neuronal cell.

11. The method of claim 9, wherein the cell is a dopaminergic cell.

12. (canceled)

13. The method of claim 9, wherein the cell is human.

14. (canceled)

15. (canceled)

16. The method of claim 9, wherein the compound is an inhibitor of macrophage migration inhibitory factor (MIF) nuclease activity and/or parthanatos mediated cell death.

17. The method of claim 9, wherein the compound is compound 121, compound 153, or compound 154.

18-33. (canceled)

34. A method of inhibiting or treating neurodegeneration in a subject, the method comprising administering to the subject a therapeutically effective amount of the compound according to claim 1.

35. The method of claim 34, wherein the compound is an inhibitor of macrophage migration inhibitory factor (MIF) nuclease activity and/or parthanatos mediated cell death.

36. The method of claim 34, wherein the subject is human.

37. The method of claim 34, wherein the neurodegenerative disease is selected from the group consisting of Alzheimer's disease, Parkinson's disease, Lewy Body Dementia, Multi-Systems Atrophy, Gehrig's disease (Amyotrophic Lateral Sclerosis), Huntington's disease, Multiple Sclerosis, senile dementia, subcortical dementia, arteriosclerotic dementia, AIDS-associated dementia, other dementias, cerebral vasculitis, epilepsy, Tourette's syndrome, Guillain Bane Syndrome, Wilson's disease, Pick's disease, encephalitis, encephalomyelitis, meningitis, prion diseases, cerebellar ataxias, cerebellar degeneration, spinocerebellar degeneration syndromes, Friedrich's ataxia, ataxia telangiectasia, spinal dysmyotrophy, progressive supranuclear palsy, dystonia, muscle spasticity, tremor, retinitis pigmentosa, striatonigral degeneration, mitochondrial encephalomyopathies and neuronal ceroid lipofuscinosis.

38. The method of claim 34, wherein the neurodegeneration results from a myocardial infarction.

39. A compound according to Formula (I): 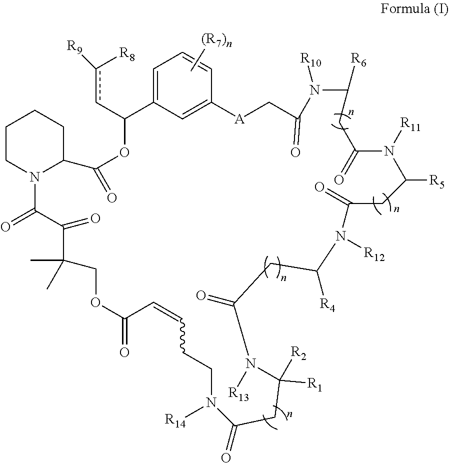
or an optically pure stereoisomer, pharmaceutically acceptable salt, or solvate thereof, wherein
each R₁ and R₂ is independently selected from the group consisting of H, halogen, hydroxyl, C₁₋₂₀ alkyl, substituted C₁₋₂₀ alkyl, OC₁₋₂₀alkyl, substituted OC₁₋₂₀ alkyl, N₃, NH₂, NO₂, CF₃, OCF₃, OCHF₂, COC₁₋₂₀alkyl, CO₂C₁₋₂₀alkyl, C₃₋₈cycloalkyl, substituted C₃₋₈cycloalkyl, C₆₋₁₅aryl, substituted C₆₋₁₅aryl, and a cyclic substituent formed with the adjacent nitrogen, the cyclic substituent is selected from the group consisting of 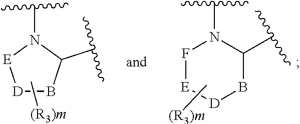
R₃ is selected from H, halogen, hydroxyl, N₃, NH₂, NO₂, CF₃, C₁₋₁₀alkyl, substituted C₁₋₁₀alkyl, C₁₋₁₀alkoxy, substituted C₁₋₁₀alkoxy, acyl, acylamino, acyloxy, acyl C₁₋₁₀alkyloxy, amino, substituted amino, aminoacyl, aminocarbonyl C₁₋₁₀alkyl, aminocarbonylamino, aminodicarbonylamino, aminocarbonyloxy, aminosulfonyl, C₆₋₁₅aryl, substituted C₆₋₁₅aryl, C₆₋₁₅aryloxy, substituted C₆₋₁₅aryloxy, C₆₋₁₅arylthio, substituted C₆₋₁₅arylthio, carboxyl, carboxyester, (carboxyester)amino, (carboxyester)oxy, cyano, C₃₋₈cycloalkyl, substituted C₃₋₈cycloalkyl, (C₃₋₈cycloalkyl)oxy, substituted (C₃₋₈cycloalkyl)oxy, (C₃₋₈cycloalkyl)thio, substituted (C₃₋₈cycloalkyl)thio, C₁₋₁₀heteroaryl, substituted C₁₋₁₀heteroaryl, C₁₋₁₀heteroaryloxy, substituted C₁₋₁₀heteroaryloxy, C₁₋₁₀heteroarylthio, substituted C₁₋₁₀heteroarylthio, C₂₋₁₀heterocyclyl, C₂₋₁₀substituted heterocyclyl, C₂₋₁₀heterocyclyloxy, substituted C₂₋₁₀heterocyclyloxy, C₂₋₁₀heterocyclylthio, substituted C₂₋₁₀heterocyclylthio, imino, oxo, sulfonyl, sulfonylamino, thiol, C₁₋₁₀alkylthio, substituted C₁₋₁₀alkylthio, and thiocarbonyl, or two R₃ substituents, together with the atom to which each is bound, may form ring selected from a C₆₋₁₅aryl, substituted C₆₋₁₅aryl, C₃₋₈cycloalkyl, substituted C₃₋₈cycloalkyl, C₁₋₁₀heteroaryl, substituted C₁₋₁₀heteroaryl, C₂₋₁₀substituted heterocyclyl, C₂₋₁₀heterocyclyloxy, and substituted C₂₋₁₀heterocyclyloxy;
each n and m is independently an integer selected from 0 to 5;
A is oxygen or —NH—CO—CH₂—;
each of B, D, E, and F independently is selected from the group consisting of carbon, nitrogen, oxygen, and sulfur;
R₄ is selected from the group consisting of H, halogen, hydroxyl, C₁₋₂₀alkyl, N₃, NH₂, NO₂, CF₃, OCF₃, OCHF₂, COC₁₋₂₀alkyl, CO₂C₁₋₂₀alkyl, and a cyclic substituent formed with the adjacent nitrogen, the cyclic substituent is selected from the group consisting of 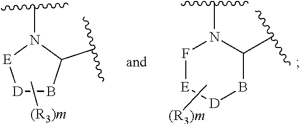
R₅ is selected from the group consisting of C₆₋₁₅aryl and C₁₋₁₀heteroaryl optionally substituted with H, halogen, hydroxyl, N₃, NH₂, NO₂, CF₃, C₁₋₁₀alkyl, substituted C₁₋₁₀alkyl, C₁₋₁₀alkoxy, substituted C₁₋₁₀alkoxy, acyl, acylamino, acyloxy, acyl C₁₋₁₀alkyloxy, amino, substituted amino, aminoacyl, aminocarbonyl C₁₋₁₀alkyl, aminocarbonylamino, aminodicarbonylamino, aminocarbonyloxy, aminosulfonyl, C₆₋₁₅aryl, substituted C₆₋₁₅aryl, C₆₋₁₅aryloxy, substituted C₆₋₁₅aryloxy, C₆₋₁₅arylthio, substituted C₆₋₁₅arylthio, carboxyl, carboxyester, (carboxyester)amino, (carboxyester)oxy, cyano, C₃₋₈cycloalkyl, substituted C₃₋₈cycloalkyl, (C₃₋₈cycloalkyl)oxy, substituted (C₃₋₈cycloalkyl)oxy, (C₃₋₈cycloalkyl)thio, substituted (C₃₋₈cycloalkyl)thio, halo, hydroxyl, C₁₋₁₀heteroaryl, substituted C₁₋₁₀heteroaryl, C₁₋₁₀heteroaryloxy, substituted C₁₋₁₀heteroaryloxy, C₁₋₁₀heteroarylthio, substituted C₁₋₁₀heteroarylthio, C₂₋₁₀heterocyclyl, C₂₋₁₀substituted heterocyclyl, C₂₋₁₀heterocyclyloxy, substituted C₂₋₁₀heterocyclyloxy, C₂₋₁₀heterocyclylthio, substituted C₂₋₁₀heterocyclylthio, imino, oxo, sulfonyl, sulfonylamino, thiol, C₁₋₁₀alkylthio, substituted C₁₋₁₀alkylthio, and thiocarbonyl,
R₆ is selected from the group consisting of H, halogen, hydroxyl, C₁₋₂₀ alkyl, C₁₋₁₀alkyloxy, substituted C₁₋₁₀alkyloxy, N₃, NH₂, NO₂, CF₃, OCF₃, OCHF₂, COC₁₋₂₀alkyl, CO₂C₁₋₂₀alkyl, C₃₋₈cycloalkyl, substituted C₃₋₈cycloalkyl, amino, substituted amino, aminoacyl, aminocarbonyl C₁₋₁₀alkyl, aminocarbonylamino, aminodicarbonylamino, aminocarbonyloxy, aminosulfonyl, thiol, C₁₋₁₀alkylthio, substituted C₁₋₁₀alkylthio, and a cyclic substituent formed with the adjacent nitrogen, the cyclic substituent is selected from the group consisting of 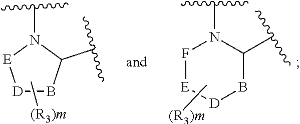
each R₇ and R₈ is independently selected from the group consisting of H, halogen, hydroxyl, C₁₋₂₀ alkyl, N₃, NH₂, NO₂, CF₃, OCF₃, OCHF₂, COC₁₋₂₀alkyl, and CO₂C₁₋₂₀alkyl; [symbol]
represents a single or a double bond;
R₉ is selected from the group consisting of C₆₋₁₅aryl and C₁₋₁₀heteroaryl optionally substituted with H, halogen, hydroxyl, N₃, NH₂, NO₂, CF₃, C₁₋₁₀alkyl, substituted C₁₋₁₀alkyl, C₁₋₁₀alkoxy, substituted C₁₋₁₀alkoxy, acyl, acylamino, acyloxy, acyl C₁₋₁₀alkyloxy, amino, substituted amino, aminoacyl, aminocarbonyl C₁₋₁₀alkyl, aminocarbonylamino, aminodicarbonylamino, aminocarbonyloxy, aminosulfonyl, C₆₋₁₅aryl, substituted C₆₋₁₅aryl, C₆₋₁₅ aryloxy, substituted C₆₋₁₅aryloxy, C₆₋₁₅arylthio, substituted C₆₋₁₅arylthio, carboxyl, carboxyester, (carboxyester)amino, (carboxyester)oxy, cyano, C₃₋₈cycloalkyl, substituted C₃₋₈cycloalkyl, (C₃₋₈cycloalkyl)oxy, substituted (C₃₋₈cycloalkyl)oxy, (C₃₋₈cycloalkyl)thio, substituted (C₃₋₈cycloalkyl)thio, C₁₋₁₀heteroaryl, substituted C₁₋₁₀heteroaryl, C₁₋₁₀heteroaryloxy, substituted C₁₋₁₀heteroaryloxy, C₁₋₁₀heteroarylthio, substituted C₁₋₁₀heteroarylthio, C₂₋₁₀heterocyclyl, C₂₋₁₀ substituted heterocyclyl, C₂₋₁₀heterocyclyloxy, substituted C₂₋₁₀heterocyclyloxy, C₂₋₁₀heterocyclylthio, substituted C₂₋₁₀heterocyclylthio, imino, oxo, sulfonyl, sulfonylamino, thiol, C₁₋₁₀alkylthio, substituted C₁₋₁₀alkylthio, and thiocarbonyl; and
each R₁₀, R₁₁, R₁₂, R₁₃, and R₁₄ is independently selected from the group consisting of H, methyl, ethyl, propyl, and isopropyl.

40. The compound of claim 39, wherein each R₁ and R₂ is independently selected from the group consisting of

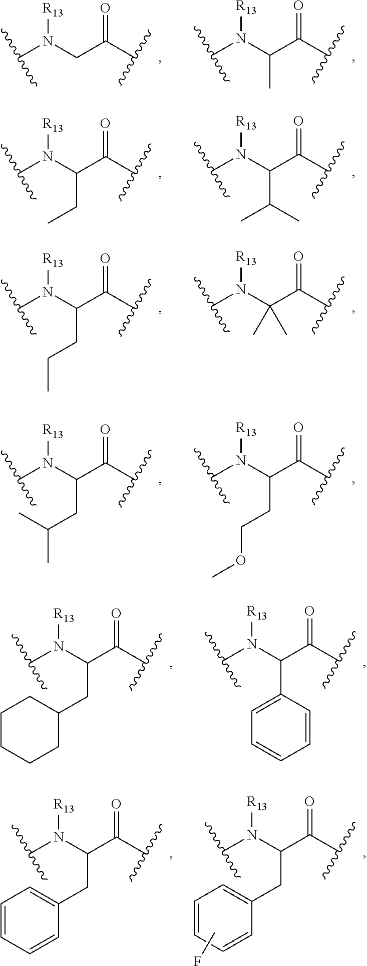

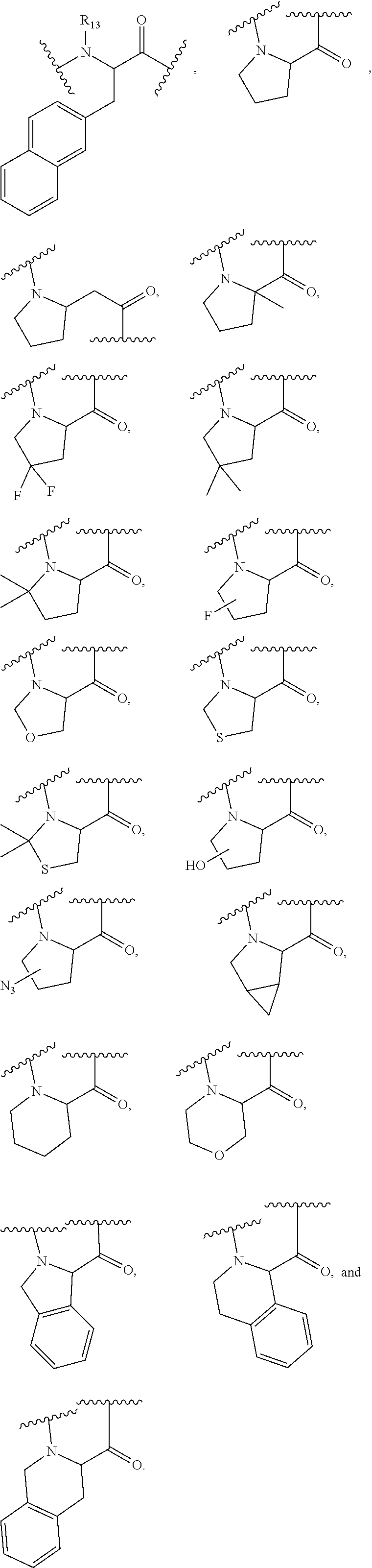

41. The compound of claim 39, wherein R₄ is selected from the group consisting of

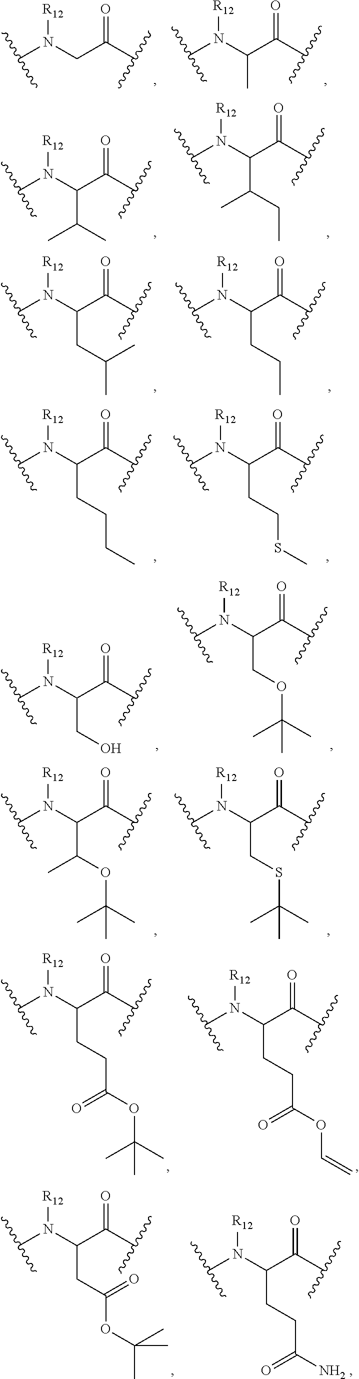

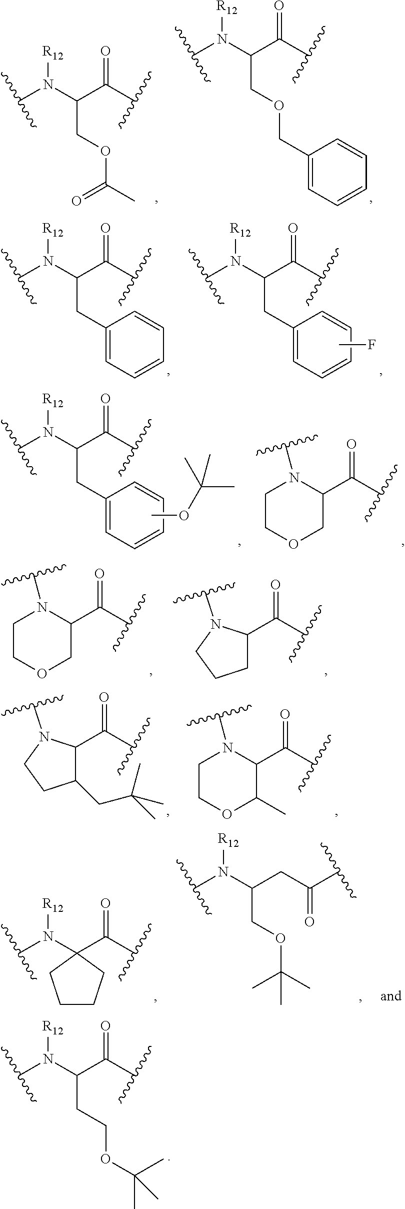

42. The compound of claim 39, wherein R₅ is selected from the group consisting of

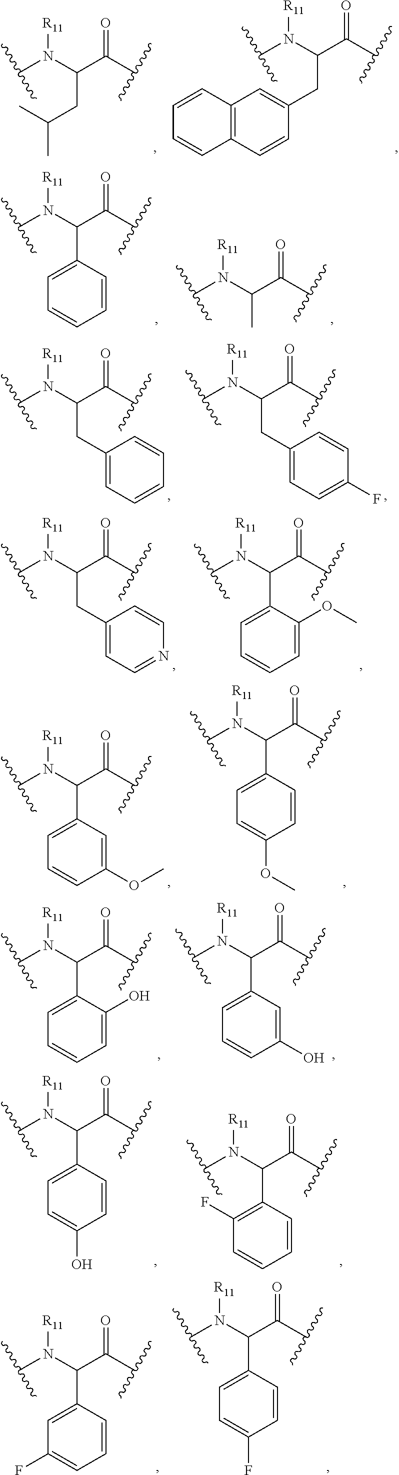

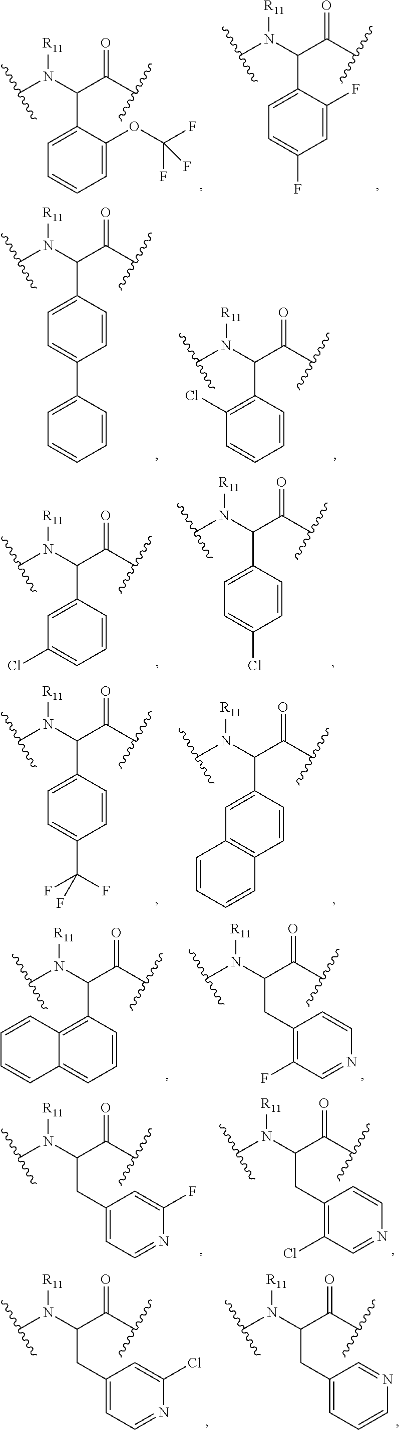

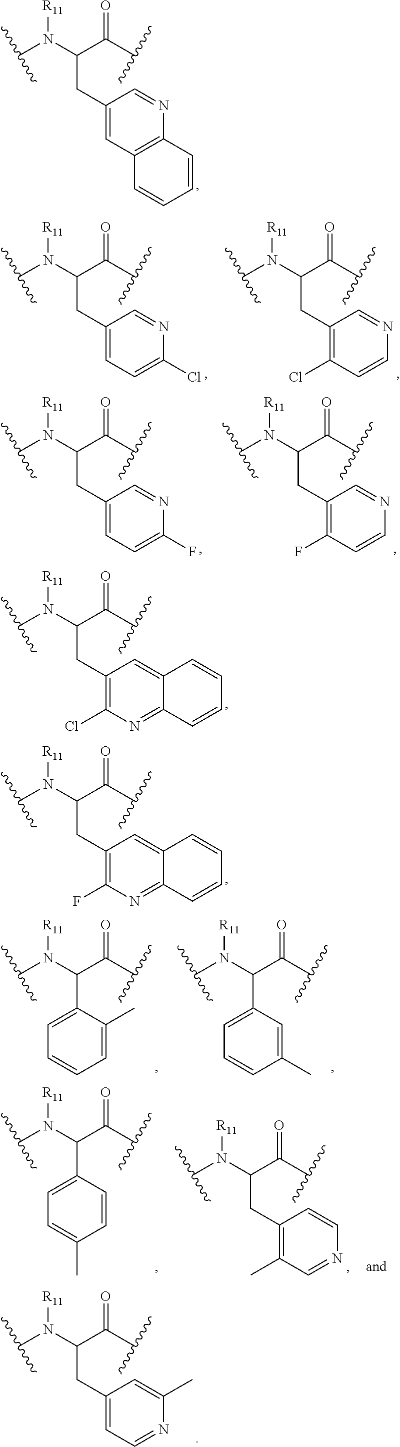

43. The compound of claim 39, wherein R₆ is selected from the group consisting of

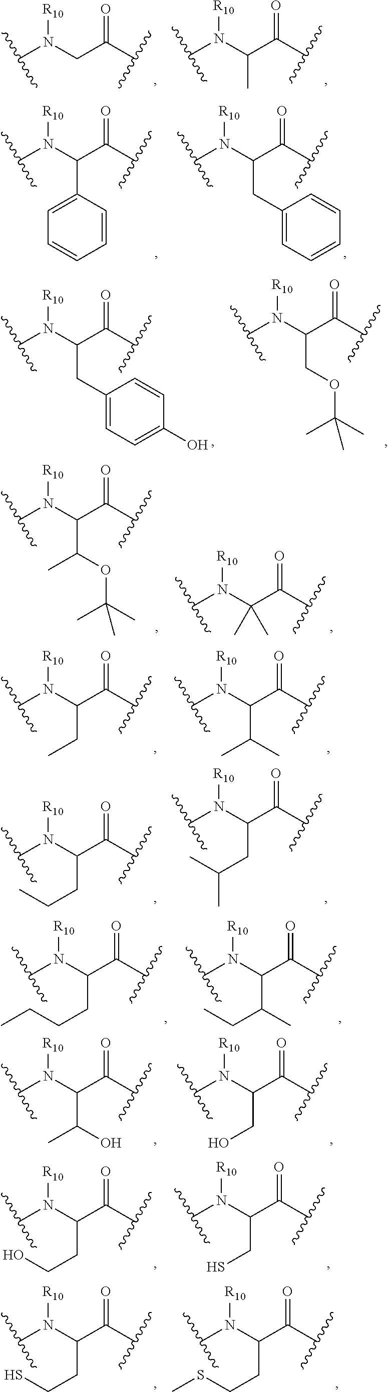

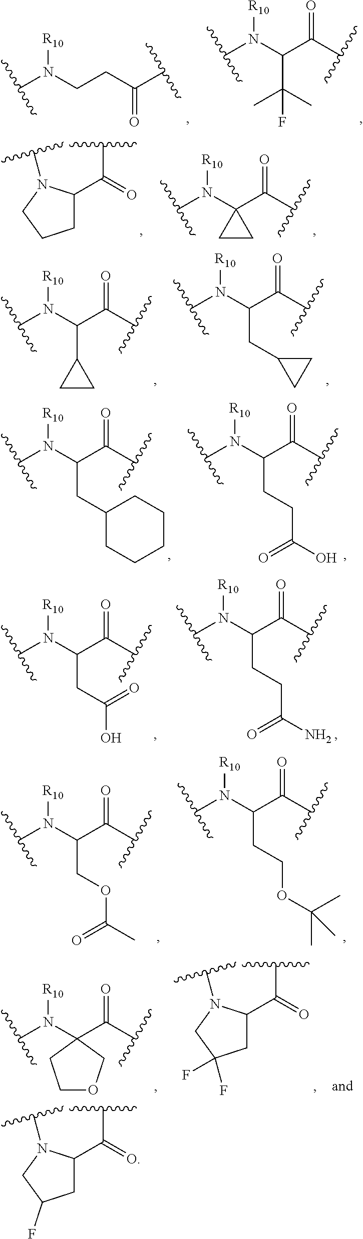

44. The compound of claim 39, wherein R₉ is selected from the group consisting of 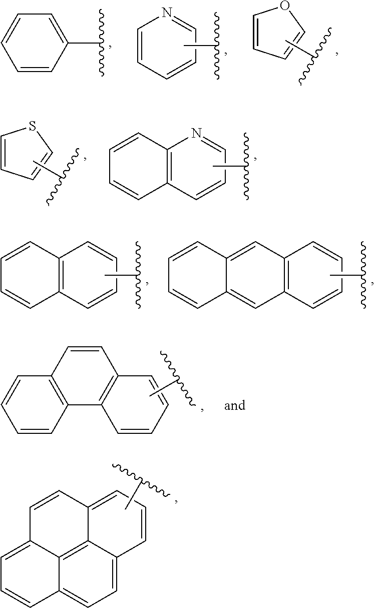
optionally substituted with halogen, hydroxyl, or alkoxyl.


SEQUENCE LISTING (5 sequences)

SEQ ID NO: 1 - DNA, 30 bp, Artificial Sequence
  Synthetic
ctcagcctcccaagtagctgggattacagg

SEQ ID NO: 2 - DNA, 29 bp, Artificial Sequence
  Synthetic
tcccaagtagctgggattacaggaaaaaa

SEQ ID NO: 3 - DNA, 14 bp, Artificial Sequence
  Synthetic
tttttttcctgtaa

SEQ ID NO: 4 - DNA, 18 bp, Artificial Sequence
  Synthetic
gtcaccgtcatacgactc

SEQ ID NO: 5 - DNA, 18 bp, Artificial Sequence
  Synthetic
gagtcgtatgacggtgac